(19) United States
(12) Patent Application Publication
(10) Pub. No.: US 2025/0120989 A1
(43) Pub. Date: Apr. 17, 2025

(54) BRIDGED TRICYCLIC CARBAMOYLPYRIDONE COMPOUNDS AND USES THEREOF

(71) Applicant: Gilead Sciences, Inc., Foster City, CA (US)

(72) Inventors: Megan K. Armstrong, San Francisco, CA (US); Chienhung Chou, Dublin, CA (US); Ana Z. Gonzalez Buenrostro, San Mateo, CA (US); Xiaochun Han, San Jose, CA (US); Lan Jiang, Foster City, CA (US); Jiayao Li, Foster City, CA (US); Michael L. Mitchell, Castro Valley, CA (US); Gregg M. Schwarzwalder, Redwood City, CA (US); Aaliyah B. Shodeinde, San Leandro, CA (US); Murali Subramanian, Foster City, CA (US); Qiaoyin Wu, Foster City, CA (US); Hai Yang, San Mateo, CA (US)

(21) Appl. No.: 18/911,340

(22) Filed: Oct. 10, 2024

Related U.S. Application Data
(60) Provisional application No. 63/702,723, filed on Oct. 3, 2024.
(60) Provisional application No. 63/659,857, filed on Jun. 14, 2024.
(60) Provisional application No. 63/543,700, filed on Oct. 11, 2023.

(51) Int. Cl. A61K 31/675; A61K 31/55; A61K 45/06; A61P 31/18; C07D 471/18; C07F 9/6561

(52) U.S. Cl. CPC A61K 31/675; A61K 31/55; A61K 45/06; A61P 31/18; C07D 471/18; C07F 9/6561

(57) ABSTRACT

The present disclosure relates generally to compounds, of Formula I:

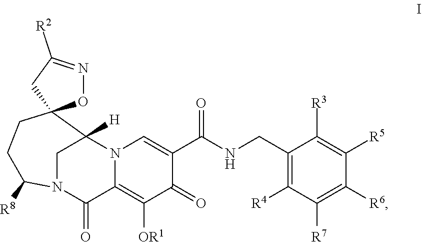

Also disclosed are pharmaceutical compositions comprising said compounds and methods of making said compounds. The compounds of the disclosure are useful in treating or preventing human immunodeficiency virus (HIV) infection.

### CROSS REFERENCE TO RELATED APPLICATIONS

This application claims priority to U.S. Provisional Application No. 63/543,700, filed Oct. 11, 2023, U.S. Provisional Application No. 63/659,857, filed Jun. 14, 2024; and U.S. Provisional Application No. 63/702,723, filed Oct. 3, 2024. The entire contents of these applications are incorporated herein by references in their entirety for all purposes.

### FIELD

Compounds, compositions, and methods that may be used for treating or preventing human immunodeficiency virus (HIV) infection are disclosed. In particular, novel spirocyclic substituted bridged tricyclic carbamoylpyridone compounds and methods for their preparation and use as therapeutic or prophylactic agents are disclosed.

### BACKGROUND

Human immunodeficiency virus infection and related diseases are a major public health problem worldwide. Human immunodeficiency virus encodes three enzymes which are required for viral replication: reverse transcriptase, protease, and integrase. Although drugs targeting reverse transcriptase and protease are in wide use and have shown effectiveness, particularly when employed in combination, toxicity and development of resistant strains may limit their usefulness (Palella, et al. N. Engl. J Med. (1998) 338:853-860, Richman, D. D. Nature (2001) 410:995-1001). Accordingly, there is a need for new agents that inhibit the replication of HIV.

A goal of antiretroviral therapy is to achieve viral suppression in the HIV infected patient. Current treatment guidelines published by the United States Department of Health and Human Services provide that achievement of viral suppression requires the use of combination therapies, i.e., several drugs from at least two or more drug classes (Panel on Antiretroviral Guidelines for Adults and Adolescents. Guidelines for the Use of Antiretroviral Agents in Adults and Adolescents Living with HIV. Department of Health and Human Services. Available at http://www.aidsinfo.nih.gov/ContentFiles/AdultandAdolescentGL.pdf. Accessed Feb. 12, 2019). In addition, decisions regarding the treatment of HIV infected patients are complicated when the patient requires treatment for other medical conditions (Id. at F-8). Because the standard of care requires the use of multiple different drugs to suppress HIV, as well as to treat other conditions the patient may be experiencing, the potential for drug interaction is a criterion for selection of a drug regimen. As such, there is a need for antiretroviral therapies having a decreased potential for drug interactions.

In addition, the HIV virus is known to mutate in infected subjects (Tang, et al. Drugs (2012) 72 (9) e1-e25). Because of the proclivity of the HIV virus to mutate, there is a need for anti-HIV drugs to be effective against a range of known HIV variants (Hurt, et al. HIV AIDS CID (2014) 58, 423-431).

For certain patients, for example, those with difficult or limited access to health care, adherence to daily oral treatment or prophylactic regimens can be challenging. Drugs that offer favorable pharmaceutical properties (for example, improved potency, long-acting pharmacokinetics, low clearance, and/or other properties) are amenable to less frequent administration and provide for better patient compliance. For example, these favorable pharmaceutical properties may facilitate a reduced dose, a reduced pill size and/or a reduced pill count for better patient adherence. Such improvements can, in turn, optimize drug exposure and limit the emergence of drug resistance.

In some cases, compatible combination agents may be needed to modulate PK properties of a drug (such as dose, AUC, Cmax, and oral bioavailability) to achieve the target long acting dosing regimens. There is thus a need for improved combination therapies.

### SUMMARY

The present disclosure is directed to novel compounds having antiviral activity and pharmaceutically acceptable salts thereof. In some embodiments, the compounds may be used to treat HIV infections, to inhibit the activity of HIV integrase and/or to reduce HIV replication. In some embodiments, compounds disclosed herein may be effective against a range of known drug-resistant HIV mutants. In some embodiments, compounds disclosed herein may have a decreased propensity to cause drug-drug interactions when co-administered with other drugs. In some embodiments, compounds disclosed herein may be administered with less than daily frequency, for example, at weekly, monthly, once every three months, once every six months, or longer intervals.

In one embodiment, the disclosure provides a compound of Formula I:

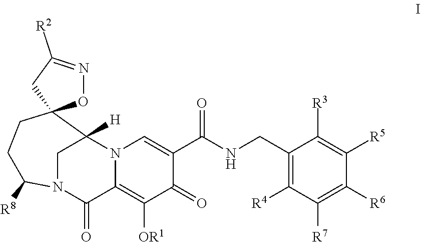

or a pharmaceutically acceptable salt thereof, wherein:
R¹ is —(CR^{1A}R^{1B}O)ₐ(Y)_{b}(CR^{1C}R^{1D})_{d}X;
   wherein a is 0 or 1;
   b is 0 or 1
   d is 0, 1, 2, 3, 4 or 5;
   R^{1A} is H or C₁₋₃alkyl;
   R^{1B} is H or C₁₋₃alkyl;
   each R^{1C} is independently H or C₁₋₃alkyl;
   each R^{1D} is independently H or C₁₋₃alkyl; or
   optionally R^{1C} and R^{1D} on the same carbon atom are joined to form a spiro cyclopropyl group;
   Y is —C(O)—, —C(O)O—, —C(O)NH— or —C(O)NR^{1H}—;
   R^{1H} is C₁₋₄alkyl optionally substituted with one or two substituents independently selected from the group consisting of —COOH, —OH, —NH₂, —CONH₂, —P(O)(OH)₂, and —S(O)₂(OH);
   X is selected from the group consisting of:
   (a) —O—P(O)(OR^{1E})₂,
      wherein each R^{1E} is independently H or phenyl;
   (b) —N(R^{1F})₂,
      wherein each R^{1F} is independently H, —COO(CR^{1I}R^{1J})ₑOPO(OH)₂, or C₁₋₄alkyl; wherein the C₁₋₄ alkyl is optionally substituted with one or two substituents independently selected from the group consisting of —COOH, —S(O)₂OH, —P(O)(OH)₂, —OH, —NH₂, and —CONH₂;
      e is 1, 2, or 3:
      each R^{1I} is independently H or C₁₋₃alkyl;
      each R^{1J} is independently H or C₁₋₃alkyl; or
      optionally R^{1I} and R^{1J} on the same carbon atom are joined to form a spiro cyclopropyl group; and
   (c) —N⁺(R^{1G})₃Z⁻,
      wherein each R^{1G} is independently H or C₁₋₄alkyl; wherein the C₁₋₄ alkyl is optionally substituted with one or two substituents independently selected from the group consisting of —COOH, —OH, —NH₂, and —CONH₂; Z⁻ is a counterion;
R² is C₁₋₃ alkyl or C₁₋₃ alkoxy;
each R³, R⁴, R⁵, R⁶ and R⁷ is independently H or halo; and
R⁸ is H or C₁₋₃alkyl.

In one embodiment, a pharmaceutical composition is provided comprising a therapeutically effective amount of a compound of Formula I or a pharmaceutically acceptable salt thereof, and a pharmaceutically acceptable excipient.

In another embodiment, a kit or an article of manufacture is provided comprising a compound of Formula I, or a pharmaceutically acceptable salt thereof, and instructions for use.

In another embodiment, a method of treating or preventing an HIV infection in a human having or at risk of having the infection, by administering to the human a therapeutically effective amount of a compound of Formula I or a pharmaceutically acceptable salt thereof, or a pharmaceutical composition of a compound of Formula I, or a pharmaceutically acceptable salt thereof, is provided.

In another embodiment, use of a compound of Formula I, or a pharmaceutically acceptable salt thereof, or a pharmaceutical composition of a compound of Formula I or a pharmaceutically acceptable salt thereof, for treating an HIV infection in a human having or at risk of having the infection is provided.

In another embodiment, use of a compound of Formula I, or a pharmaceutically acceptable salt thereof, or a pharmaceutical composition of a compound of Formula I or a pharmaceutically acceptable salt thereof, in the manufacture of a medicament for treating an HIV infection in a human having or at risk of having the infection is provided.

In another embodiment, a compound of Formula I or a pharmaceutically acceptable salt thereof, or a pharmaceutical composition of a compound of formula I or a pharmaceutically acceptable salt thereof, for use in medical therapy is provided.

In another embodiment, a compound of Formula I or a pharmaceutically acceptable salt thereof, or a pharmaceutical composition of a compound of formula I or a pharmaceutically acceptable salt thereof, for use in treating an HIV infection is provided.

Other embodiments, objects, features, and advantages may be set forth in the detailed description of the embodiments that follows, and in part may be apparent from the description, or may be learned by practice, of the claimed embodiments. These objects and advantages may be realized and attained by the processes and compositions particularly pointed out in the description and claims thereof. The foregoing Summary has been made with the understanding that it is to be considered as a brief and general synopsis of some of the embodiments disclosed herein, is provided for the benefit and convenience of the reader, and is not intended to limit in any manner the scope, or range of equivalents, to which the appended claims are lawfully entitled.

In another embodiment, combination therapies including compounds having antiviral activity and pharmaceutically acceptable salts thereof, and PGP inhibitors are provided. Administration of combinations of the invention has been shown in the example section to increase the exposure and bioavailability of the compound having antiviral activity. In some embodiments, the present disclosure provides a method of treating or preventing HIV infection in a patient comprising, administering to the patient a combination as disclosed herein comprising a compound of Formula I, or a pharmaceutically acceptable salt thereof, wherein exposure of a metabolite of the compound of Formula I, or a pharmaceutically acceptable salt thereof, is increased when the compound of Formula I, or a pharmaceutically acceptable salt thereof, is co-administered with a P-glycoprotein inhibitor, relative to the exposure of the metabolite of the compound of Formula I, or a pharmaceutically acceptable salt thereof, when the compound of Formula I, or a pharmaceutically acceptable salt therefore, is dosed the same but in the absence of the P-glycoprotein inhibitor.

### BRIEF DESCRIPTION OF THE DRAWINGS

FIG. 1 shows the plasma concentrations in beagle dogs of Intermediate B following oral administration of Intermediate B with and without encequidar mesylate pretreatment.

FIG. 2 shows the plasma concentrations in beagle dogs of Intermediate C following oral administration of Intermediate C with and without encequidar mesylate pretreatment.

FIG. 3 shows the plasma concentrations in beagle dogs of Intermediate C following oral administration of Compound 3 with and without encequidar mesylate pretreatment.

### DETAILED DESCRIPTION

In the following description, certain specific details are set forth in order to provide a thorough understanding of various embodiments disclosed herein. However, one skilled in the art will understand that the embodiments disclosed herein may be practiced without these details. The description below of several embodiments is made with the understanding that the present disclosure is to be considered as an exemplification of the claimed subject matter, and is not intended to limit the appended claims to the specific embodiments illustrated. The headings used throughout this disclosure are provided for convenience only and are not to be construed to limit the claims in any way. Embodiments illustrated under any heading may be combined with embodiments illustrated under any other heading.

### I. Definitions

Unless the context requires otherwise, throughout the present disclosure and claims, the word “comprise” and variations thereof, such as, “comprises” and “comprising” are to be construed in an open, inclusive sense, that is as “including, but not limited to”.

Reference throughout this specification to “one embodiment” or “an embodiment” means that a particular feature, structure or characteristic described in connection with the embodiment is included in at least one embodiment disclosed herein. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

“Amino” refers to the —NH₂ radical.

“Hydroxy” or “hydroxyl” refers to the —OH radical.

The term “C₁₋ₙ alkyl” as used herein, wherein n is an integer, either alone or in combination with another radical, is intended to mean acyclic, straight or branched chain alkyl radicals containing from 1 to n carbon atoms. “C₁₋₆ alkyl” includes, but is not limited to, methyl, ethyl, propyl (n-propyl), butyl (n-butyl), 1-methylethyl (isopropyl), 1-methylpropyl (sec-butyl), 2-methylpropyl (iso-butyl), 1,1-dimethylethyl (tertbutyl), pentyl and hexyl. The abbreviation Me denotes a methyl group; Et denotes an ethyl group, Pr denotes a propyl group, iPr denotes a 1-methylethyl group, Bu denotes a butyl group and tBu denotes a 1,1-dimethylethyl group.

“Alkyl” is hydrocarbon containing normal, secondary or tertiary atoms. For example, an alkyl group can have 1 to 20 carbon atoms (i.e., C₁₋₂₀ alkyl), 1 to 10 carbon atoms (i.e., C₁₋₁₀ alkyl), 1 to 8 carbon atoms (i.e., C₁₋₈ alkyl) or 1 to 6 carbon atoms (i.e., C₁₋₆ alkyl). Examples of suitable alkyl groups include, but are not limited to, methyl (Me, —CH₃), ethyl (Et, CH₂CH₃), 1-propyl (n-Pr, n-propyl, —CH₂CH₂CH₃), 2-propyl (i-Pr, ipropyl, CH(CH₃)₂), 1-butyl (n-Bu, n-butyl, —CH₂CH₂CH₂CH₃), 2-methyl1-propyl (i-Bu, ibutyl, —CH₂CH(CH₃)₂), 2-butyl (g-Bu, 5-butyl, CH(CH₃)CH₂CH₃), 2-methyl-2-propyl (t-Bu, tbutyl, —C(CH₃)₃), 1-pentyl (n-pentyl, CH₂CH₂CH₂CH₂CH₃), 2-pentyl (CH(CH₃)CH₂CH₂CH₃), 3-pentyl (CH(CH₂CH₃)₂), 2-methyl-2-butyl (C(CH₃)₂CH₂CH₃), 3-methyl-2-butyl (CH(CH₃)CH(CH₃)₂), 3-methyl1butyl (CH₂CH₂CH(CH₃)₂), 2-methyl-1-butyl (CH₂CH(CH₃)CH₂CH₃), 1-hexyl (CH₂CH₂CH₂CH₂CH₂CH₃), 2-hexyl (CH(CH₃)CH₂CH₂CH₂CH₃), 3-hexyl (CH(CH₂CH;)(CH₂CH₂CH₃)), 2-methyl-2-pentyl (C(CH₃)₂CH₂CH₂CH₃), 3-methyl-2-pentyl (CH(CH₃)CH(CH₃)CH₂CH₃), 4-methyl-2-pentyl (CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (C(CH₃)CH(CH₃)₂), 3,3-dimethyl-2-butyl (—CH(CH₃)C(CH₃)₃, and octyl ((CH₂)₇CH₃). “Alkyl” also refers to a saturated, branched or straight chain hydrocarbon radical having two monovalent radical centers derived by the removal of two hydrogen atoms from the same or two different carbon atoms of a parent alkane. For example, an alkyl group can have 1 to 10 carbon atoms (i.e., C₁₋₁₀ alkyl), or 1 to 6 carbon atoms (i.e., C₁₋₆ alkyl) or 1-3 carbon atoms (i.e., C₁₋₃ alkyl). Typical alkyl radicals include, but are not limited to, methylene (CH₂), 1,1-ethyl (CH(CH₃)), 1,2-ethyl (CH₂CH₂), 1,1-propyl (CH(CH₂CH₃)), 1,2-propyl (CH₂CH(CH₃)), 1,3-propyl (CH₂CH₂CH₂), 1,4-butyl (CH₂CH₂CH₂CH₂), and the like.

The term “alkenyl” as used herein refers to a straight or branched hydrocarbon containing normal, secondary or tertiary carbon atoms with at least one site of unsaturation, i.e., a carbon-carbon, sp² double bond. For example, an alkenyl group can have 2 to 20 carbon atoms (i.e., C₂-C₂₀ alkenyl, or C₂₋₂₀ alkenyl), 2 to 8 carbon atoms (i.e., C₂-C₈ alkenyl or C₂-s), or 2 to 6 carbon atoms (i.e., C₂-C₆ alkenyl, or C₂₋₆ alkenyl). Examples of suitable alkenyl groups include, but are not limited to, ethylene or vinyl (CH═CH₂), allyl (CH₂CH═CH₂), cyclopentenyl (C₅H₇), and 5-hexenyl (CH₂CH₂CH₂CH₂CH═CH₂).

The term “C₂₋ₙalkenyl”, as used herein, wherein n is an integer, either alone or in combination with another radical, is intended to mean an unsaturated, acyclic straight or branched chain radical containing two to n carbon atoms, at least two of which are bonded to each other by a double bond. Examples of such radicals include, but are not limited to, ethenyl (vinyl), 1propenyl, 2propenyl, and 1butenyl. Unless specified otherwise, the term “C₂₋ₙalkenyl” is understood to encompass individual stereoisomers where possible, including but not limited to (E) and (Z) isomers, and mixtures thereof. When a C₂₋ₙalkenyl group is substituted, it is understood to be substituted on any carbon atom thereof which would otherwise bear a hydrogen atom, unless specified otherwise, such that the substitution would give rise to a chemically stable compound, such as are recognized by those skilled in the art.

The term “halo” or “halogen” as used herein refers to fluoro, chloro, bromo and iodo.

The term “C₃₋ₘ cycloalkyl” as used herein, wherein m is an integer, either alone or in combination with another radical, is intended to mean a cycloalkyl substituent containing from 3 to m carbon atoms and includes, but is not limited to, cyclopropyl, cyclobutyl, cyclopentyl, cyclohexyl and cycloheptyl. The term includes fully saturated as well as partially unsaturated rings.

It is to be understood that when a variable is substituted, for example, as described by the phrase “C₁₋₆ alkyl, either alone or as part of a group, is optionally substituted”, the phrase means that the variable C₁₋₆ alkyl can be substituted when it is alone and that it can also be substituted when the variable “C₁₋₆ alkyl” is part of a larger group. Similarly, when stated, other variables can also be substituted “either alone or as part of a group.”

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers or axes of chirality and whose molecules are not mirror images of one another. Diastereomers typically have different physical properties, e.g., melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

The term “treatment” or “treating.” to the extent it relates to a disease or condition includes preventing the disease or condition from occurring, inhibiting or ameliorating the disease or condition (e.g., arresting or slowing its development), eliminating the disease or condition (e.g., causing regression or cure of the disease or condition), and/or relieving one or more symptoms of the disease or condition. In the case of HIV, treatment includes reducing the level of HIV viral load in a patient.

In some embodiments, the term “treatment” refers to the administration of a compound or composition according to the present invention to alleviate or eliminate symptoms of HIV infection and/or to reduce viral load in a patient. The term “treatment” also encompasses the administration of a compound or composition according to the present invention before the exposure of the individual to the virus, postexposure of the individual to the virus but before the appearance of symptoms of the disease, and/or prior to the detection of the virus in the blood, to prevent the appearance of symptoms of the disease and/or to prevent the virus from reaching detectible levels in the blood, and the administration of a compound or composition according to the present invention to prevent perinatal transmission of HIV from mother to baby, by administration to the mother before giving birth and to the child within the first days of life.

The term “combination therapy” as used herein refers to the use of two or more treatments, such as pharmaceutically active agents (i.e., a “combination”), to treat or prevent a single disease or condition. The pharmaceutically active agents can be administered together or separately, as well as simultaneously or sequentially, usually so that their functionalities coincide and have overlapping effects on a patient, resulting in a desired therapeutic or prophylactic effect on the patient. In some embodiments, the pharmaceutically active agents are from at least two or more drug classes.

“Protecting group” refers to a moiety of a compound that masks or alters the properties of a functional group or the properties of the compound as a whole. Chemical protecting groups and strategies for protection/deprotection are well known in the art. See e.g., Protective Groups in Organic Chemistry, Theodora W. Greene, John Wiley & Sons, Inc., New York, 1991. Protecting groups are often utilized to mask the reactivity of certain functional groups, to assist in the efficiency of desired chemical reactions. e.g., making and breaking chemical bonds in an ordered and planned fashion. Protection of functional groups of a compound alters other physical properties besides the reactivity of the protected functional group, such as the polarity, lipophilicity (hydrophobicity), and other properties which can be measured by common analytical tools. Chemically protected intermediates may themselves be biologically active or inactive.

Protected compounds may also exhibit altered, and in some cases, optimized properties in vitro and in vivo, such as passage through cellular membranes and resistance to enzymatic degradation or sequestration. In this role, protected compounds with intended therapeutic effects may be referred to as prodrugs. Another function of a protecting group is to convert the parental drug into a prodrug, whereby the parental drug is released upon conversion of the prodrug in vivo. Because active prodrugs may be absorbed more effectively than the parental drug, prodrugs may possess greater potency in vivo than the parental drug. Protecting groups are removed either in vitro, in the instance of chemical intermediates, or in vivo, in the case of prodrugs. With chemical intermediates, it is not particularly important that the resulting products after deprotection, e.g., alcohols, be physiologically acceptable, although in general it is more desirable if the products are pharmacologically innocuous.

Protecting groups are available, commonly known and used, and are optionally used to prevent side reactions with the protected group during synthetic procedures, i.e., routes or methods to prepare the compounds of the invention. For the most part the decision as to which groups to protect, when to do so, and the nature of the chemical protecting group “PG” will be dependent upon the chemistry of the reaction to be protected against (e.g., acidic, basic, oxidative, reductive or other conditions) and the intended direction of the synthesis. PGs do not need to be, and generally are not, the same if the compound is substituted with multiple PG. In general, PG will be used to protect functional groups such as carboxyl, hydroxyl, thio, or amino groups and to thus prevent side reactions or to otherwise facilitate the synthetic efficiency. The order of deprotection to yield free deprotected groups is dependent upon the intended direction of the synthesis and the reaction conditions to be encountered, and may occur in any order as determined by the artisan.

Various functional groups of the compounds of the invention may be protected. For example, protecting groups for —OH groups (whether hydroxyl, carboxylic acid, phosphonic acid, or other functions) include “ether- or ester-forming groups”. Ether- or ester-forming groups are capable of functioning as chemical protecting groups in the synthetic schemes set forth herein. However, some hydroxyl and thio protecting groups are neither ether- nor ester-forming groups, as will be understood by those skilled in the art, and are included with amides, discussed below.

A very large number of hydroxyl protecting groups and amide-forming groups and corresponding chemical cleavage reactions are described in Protective Groups in Organic Synthesis, Theodora W. Greene (John Wiley & Sons, Inc., New York, 1991, ISBN 0-471-62301-6) (“Greene”). See also Kocienski, Philip J.; Protecting Groups (Georg Thieme Verlag Stuttgart, New York, 1994), which is incorporated by reference in its entirety herein. In particular Chapter 1, Protecting Groups. An Overview, pages 1-20, Chapter 2, Hydroxyl Protecting Groups, pages 21-94, Chapter 3, Diol Protecting Groups, pages 95-117, Chapter 4, Carboxyl Protecting Groups, pages 118-154, Chapter 5, Carbonyl Protecting Groups, pages 155-184. For protecting groups for carboxylic acid, phosphonic acid, phosphonate, sulfonic acid and other protecting groups for acids see Greene as set forth below.

The term “solvate” refers to a crystalline solid containing amounts of a solvent incorporated within the crystal structure. As used herein, the term “solvate” includes hydrates.

The term “non-solvate” refers to a crystalline solid in which no solvent molecules occupy a specific crystallographic site.

The term “pharmaceutically acceptable” with respect to a substance as used herein means that substance which is, within the scope of sound medical judgment, suitable for use in contact with the tissues of humans and lower animals without undue toxicity, irritation, allergic response, and the like, commensurate with a reasonable benefit/risk ratio, and effective for the intended use when the substance is used in a pharmaceutical composition.

The term “pharmaceutically acceptable salt” as used herein is intended to mean a salt of a compound according to the invention which is, within the scope of sound medical judgment, suitable for use in contact with the tissues of humans and lower animals without undue toxicity, irritation, allergic response, and the like, commensurate with a reasonable benefit/risk ratio, generally water or oil-soluble or dispersible, and effective for their intended use. The term includes pharmaceutically-acceptable acid addition salts and pharmaceutically-acceptable base addition salts. Lists of suitable salts are found in, for example, S. M. Birge et al., J. Pharm. Sci., 1977, 66, pp. 1-19.

The term “pharmaceutically-acceptable acid addition salt” as used herein is intended to mean those salts which retain the biological effectiveness and properties of the free bases and which are not biologically or otherwise undesirable, formed with inorganic acids including but not limited to hydrochloric acid, hydrobromic acid, sulfuric acid, sulfamic acid, nitric acid, phosphoric acid and the like, and organic acids including but not limited to acetic acid, trifluoroacetic acid, adipic acid, ascorbic acid, aspartic acid, benzenesulfonic acid, benzoic acid, butyric acid, camphoric acid, camphorsulfonic acid, cinnamic acid, citric acid, digluconic acid, ethanesulfonic acid, glutamic acid, glycolic acid, glycerophosphoric acid, hemisulfic acid, hexanoic acid, formic acid, fumaric acid, 2-hydroxyethanesulfonic acid (isethionic acid), lactic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, pivalic acid, propionic acid, pyruvic acid, salicylic acid, stearic acid, succinic acid, sulfanilic acid, tartaric acid, p-toluenesulfonic acid, undecanoic acid and the like.

The term “pharmaceutically-acceptable base addition salt” as used herein is intended to mean those salts which retain the biological effectiveness and properties of the free acids and which are not biologically or otherwise undesirable, formed with inorganic bases including but not limited to ammonia or the hydroxide, carbonate, or bicarbonate of ammonium or a metal cation such as sodium, potassium, lithium, calcium, magnesium, iron, zinc, copper, manganese, aluminum and the like. Particularly preferred are the ammonium, potassium, sodium, calcium, and magnesium salts. Salts derived from pharmaceutically-acceptable organic nontoxic bases include but are not limited to salts of primary, secondary, and tertiary amines, quaternary amine compounds, substituted amines including naturally occurring substituted amines, cyclic amines and basic ion-exchange resins, such as methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, methylglucamine, theobromine, purines, piperazine, piperidine, N-ethylpiperidine, tetramethylammonium compounds, tetraethylammonium compounds, pyridine, N,N-dimethylaniline, N-methylpiperidine, N-methylmorpholine, dicyclohexylamine, dibenzylamine. N,N-dibenzylphenethylamine, 1-ephenamine, N,N′-dibenzylethylenediamine, polyamine resins and the like. Particularly preferred organic nontoxic bases are isopropylamine, diethylamine, ethanolamine, trimethylamine, dicyclohexylamine, choline, and caffeine.

The term “antiviral agent” as used herein is intended to mean an agent that is effective to inhibit the formation and/or replication of a virus in a mammal, including but not limited to agents that interfere with either host or viral mechanisms necessary for the formation and/or replication of a virus in a mammal. The term “antiviral agent” includes, for example, an HIV integrase catalytic site inhibitor selected from the group consisting: raltegravir (ISENTRESSV; Merck); elvitegravir (Gilead), soltegravir (GSK; ViiV); GSK 1265744 (GSK: ViiV) and dolutegravir; an HIV nucleoside reverse transcriptase inhibitor selected from the group consisting of: abacavir (ZIAGEN®; GSK); didanosine (VIDEX®; BMS); tenofovir (VIREAD®; Gilead); emtricitabine (EMTRIVA®; Gilead); lamivudine (EPIVIR®; GSK/Shire); stavudine (ZERIT®; BMS); zidovudine (RETROVIR®; GSK); elvucitabine (Achillion); and festinavir (Oncolys); an HIV non-nucleoside reverse transcriptase inhibitor selected from the group consisting of: nevirapine (VIRAMUNE®; BI); efavirenz (SUSTIVA®; BMS); etravirine (INTELENCE®; J&J); rilpivirine (TMC278, R278474; J&J); fosdevirine (GSK/ViiV); and lersivirine (Pfizer/ViiV); an HIV protease inhibitor selected from the group consisting of: atazanavir (REYATAZ*; BMS); darunavir (PREZISTA®; J&J); indinavir (CRIXIVAN®; Merck); lopinavir (KELETRA®; Abbott); nelfinavir (VIRACEPT®; Pfizer); saquinavir (INVIRASEt®; Hoffmann-LaRoche); tipranavir (APTIVUS®; BI); ritonavir (NORVIR®; Abbott); and fosamprenavir (LEXIVA®; GSK/Vertex); an HIV entry inhibitor selected from: maraviroc (SELZENTRY®; Pfizer); enfuvirtide (FUZEON®; Trimeris); and BMS-663068 (BMS); and an HIV maturation inhibitor selected from: bevirimat (Myriad Genetics).

The term “inhibitor of HIV replication” as used herein is intended to mean an agent capable of reducing or eliminating the ability of HIV to replicate in a host cell, whether in vitro, ex vivo or in vivo.

The term “substituent”, as used herein and unless specified otherwise, is intended to mean an atom, radical or group which may be bonded to a carbon atom, a heteroatom or any other atom which may form part of a molecule or fragment thereof, which would otherwise be bonded to at least one hydrogen atom. Substituents contemplated in the context of a specific molecule or fragment thereof are those which give rise to chemically stable compounds, such as are recognized by those skilled in the art.

The term “heteroatom” as used herein is intended to mean O, S or N.

The terms “O—C₁₋ₙ alkyl” or “C₁₋ₙ alkoxy” as used herein interchangeably, wherein n is an integer, either alone or in combination with another radical, is intended to mean an oxygen atom further bonded to an alkyl radical having 1 to n carbon atoms as defined above. Examples of C₁₋ₙ alkoxy include but are not limited to methoxy (CH₃O—), ethoxy (CH₃CH₂O—), propoxy (CH₃CH₂CH₂O—), 1-methylethoxy (iso-propoxy; (CH₃)₂CHO) and 1,1-dimethylethoxy (tert-butoxy; (CH₃)₃CO). When an C₁₋ₙ alkoxy is substituted, it is understood to be substituted on the alkyl portion thereof, such that the substitution would give rise to a chemically stable compound, such as are recognized by those skilled in the art.

The term “mammal” as used herein is intended to encompass humans, as well as non-human mammals which are susceptible to infection by HIV. Non-human mammals include but are not limited to domestic animals, such as cows, pigs, horses, dogs, cats, rabbits, rats and mice, and non-domestic animals.

The embodiments disclosed herein are also meant to encompass all pharmaceutically acceptable compounds of Formula I or Formula II being isotopically-labeled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. In certain embodiments, these radiolabeled compounds are useful to help determine or measure the effectiveness of the compounds, by characterizing, for example, the site or mode of action, or binding affinity to pharmacologically important site of action. Certain isotopically-labeled compounds of Formula I or Formula II, for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection.

In certain embodiments, substitution with heavier isotopes such as deuterium, i.e., 2H, may afford certain therapeutic advantages resulting from greater metabolic stability. For example, in vivo half-life may increase or dosage requirements may be reduced. Thus, heavier isotopes may be preferred in some circumstances.

Substitution with positron emitting isotopes, such as ¹¹C, ¹⁸F, ¹⁵O, and ¹³N, can be useful in Positron Emission Topography (PET) studies for examining substrate receptor occupancy. Isotopically-labeled compounds of the compounds disclosed herein can be prepared by techniques known to those skilled in the art or by processes analogous to those described in the Examples as set out below using an appropriate isotopically-labeled reagent in place of the non-labeled reagent previously employed.

The methods, compositions, kits and articles of manufacture provided herein use or include compounds of Formula I or Formula II or pharmaceutically acceptable salts thereof, in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such compounds increase resistance to metabolism, and thus are useful for increasing the half-life of compounds or pharmaceutically acceptable salts thereof, when administered to a mammal. See, e.g., Foster, “Deuterium Isotope Effects in Studies of Drug Metabolism”, Trends Pharmacol. Sci., 5(12):524-527 (1984). Such compounds can be synthesized by means known in the art, for example by employing starting materials in which one or more hydrogen atoms have been replaced by deuterium.

The embodiments disclosed herein are also meant to encompass the in vivo metabolic products of the disclosed compounds of Formula I or Formula II. Such products may result from, for example, the oxidation, reduction, hydrolysis, amidation, esterification, and the like of the administered compound, primarily due to enzymatic processes. Accordingly, the embodiments disclosed herein include compounds of Formula I or Formula II produced by a process comprising administering a compound according to the embodiments disclosed herein to a mammal for a period of time sufficient to yield a metabolic product thereof. Such products are typically identified by administering a radiolabeled compound according to the embodiments disclosed herein in a detectable dose to an animal, such as rat, mouse, guinea pig, monkey, or to human, allowing sufficient time for metabolism to occur, and isolating its conversion products from the urine, blood or other biological samples.

The compounds of Formula I or Formula II disclosed herein, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic, scalemic, and optically pure forms. Optically active (+) and (−), (R)- and (S)-, or (D)- and (L)-isomers may be prepared using chiral synthons or chiral reagents, or resolved using methods such as chromatography and fractional crystallization. Techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

“Optional” or “optionally” means that the subsequently described event or circumstances may or may not occur, and that the description includes instances where said event or circumstance occurs and instances in which it does not. For example, “optionally substituted heterocyclyl” means that the heterocyclyl radical may or may not be substituted and that the description includes both substituted heterocyclyl radicals and heterocyclyl radicals having no substitution.

### II. Compounds of Formula I

In some embodiments, the disclosure provides a compound of Formula I:

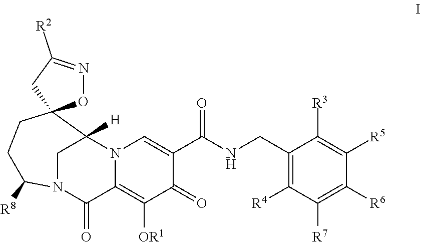

or a pharmaceutically acceptable salt thereof, wherein:
R¹ is —(CR^{1A}R^{1B}O)ₐ(Y)_{b}(CR^{1C}R^{1D})_{d}X;
   wherein a is 0 or 1;
   b is 0 or 1;
   d is 0, 1, 2, 3, 4 or 5;
   R^{1A} is H or C₁₋₃alkyl;
   R^{1B} is H or C₁₋₃alkyl;
   each R^{1C} is independently H or C₁₋₃alkyl;
   each R^{1D} is independently H or C₁₋₃alkyl; or
   optionally R^{1C} and R^{1D} on the same carbon atom are joined to form a spiro cyclopropyl group;
   Y is —C(O)—, —C(O)O—, —C(O)NH— or —C(O)NR^{1H}—;
   R^{1H} C₁₋₄alkyl optionally substituted with one or two substituents independently selected from the group consisting of —COOH, —OH, —NH₂, —CONH₂, —P(O)(OH)₂, and —S(O)₂(OH);
   X is selected from the group consisting of:
   (a) —O—P(O)(OR^{1E})₂,
      wherein each R^{1E} is independently H or phenyl;
   (b) —N(R^{1F})₂,
      wherein each R^{1F} is independently H, —COO(CR^{1I}R^{1J})ₑOPO(OH)₂, or C₁₋₄alkyl; wherein the C₁₋₄ alkyl is optionally substituted with one or two substituents independently selected from the group consisting of —COOH, —S(O)₂(OH), —P(O)(OH)₂, —OH, —NH₂, and —CONH₂;
      e is 1, 2, or 3;
      each R^{1I} is independently H or C₁₋₃alkyl;
      each R^{1J} is independently H or C₁₋₃alkyl; or
      optionally R^{1I} and R^{1J} on the same carbon atom are joined to form a spiro cyclopropyl group; and
   (c) —N⁺(R^{1G})₃Z⁻,
      wherein each R^{1G} is independently H or C₁₋₄alkyl; wherein the C₁₋₄ alkyl is optionally substituted with one or two substituents independently selected from the group consisting of —COOH, —OH, —NH₂, and —CONH₂;
      Z⁻ is a counterion;
R² is C₁₋₃ alkyl or C₁₋₃ alkoxy;
each R³, R⁴, R⁵, R⁶ and R⁷ is independently H or halo; and
R⁸ is H or C₁₋₃alkyl.

In some embodiments, a is 0. In some embodiments, a is 1.

In some embodiments, b is 0. In some embodiments, b is 1.

In some embodiments, d is 1, 2, 3, or 4. In some embodiments, d is 1, 2, or 3. In some embodiments, d is 1. In some embodiments, d is 2. In some embodiments, d is 3. In some embodiments, d is 4.

In some embodiments, each R^{1A} is H. In some embodiments, each R^{1B} is H. In some embodiments, each R^{1A} and R^{1B} is H.

In some embodiments, each RIA is C₁₋₃alkyl and each R^{1B} is H. In some embodiments, each R^{1A} is methyl. In some embodiments, each R^{1A} is methyl and each R^{1B} is H.

In some embodiments, each R^{1C} is H. In some embodiments, each R^{1D} is H. In some embodiments, each R^{1C} and R^{1D} is H. In some embodiments, each R^{1C} is independently H or methyl. In some embodiments, each R^{1D} is independently H or methyl. In some embodiments, each R^{1C} is independently H or methyl and each R^{1D} is H.

In some embodiments, Y is —C(O)—, —C(O)O—, —C(O)NH— or —C(O)NCH₃—. In some embodiments, Y is —C(O)—, —C(O)O— or —C(O)NCH₃—. In some embodiments, Y is —C(O)—. In some embodiments, Y is —C(O)O—. In some embodiments, Y is —C(O)NCH₃—.

In some embodiments, Y is —C(O)NR^{1H}—. In some embodiments, R^{1H} is C₁₋₂alkyl optionally substituted with —COOH, —P(O)(OH)₂, or —S(O)₂(OH).

In some embodiments, X is —O—P(O)(OR^{1E})₂. In some embodiments, each R^{1E} is phenyl In some embodiments, each R^{1E} is H.

In some embodiments, X is —N(R^{1F})₂. In some embodiments, each R^{1F} is independently —COO(CR^{1I}R^{1J})ₑOPO(OH)₂ or C₁₋₄alkyl; wherein the C₁₋₄ alkyl is optionally substituted with one or two —COOH. In some embodiments, each R^{1F} is independently —COO(CR^{1I}R^{1J})ₑOPO(OH)₂ or C₁₋₄alkyl; wherein the C₁₋₄ alkyl is optionally substituted with one —COOH.

In some embodiments, e is 1 or 2. In some embodiments, e is 1. In some embodiments, e is 2.

In some embodiments, each R^{1I} is H. In some embodiments, each R^{1J} is H. In some embodiments, each R^{1I} and each R^{1J} is H. In some embodiments, each R^{1J} is C₁₋₃alkyl and each R^{1I} is H.

In some embodiments, each R^{1F} is independently —COOCH₂OPO(OH)₂ or C₁₋₄alkyl; wherein the C₁₋₄ alkyl is optionally substituted with one or two —COOH. In some embodiments, each R^{1F} is independently —COOCH₂OPO(OH)₂ or C₁₋₄alkyl; wherein the C₁₋₄ alkyl is optionally substituted with one —COOH.

In some embodiments, each R^{1F} is independently —COO(CH₂)₂OPO(OH)₂, —COOCH₂OPO(OH)₂, —CH₃, or —CH₂COOH. In some embodiments, each R^{1F} is independently —COOCH₂OPO(OH)₂, —CH₃, or —CH₂COOH.

In some embodiments, X is —N⁺(R^{1G})₃Z⁻.

In some embodiments, Z⁻ is selected from the group consisting of acetate, ascorbate, aspartate, besylate, benzoate, bromide, bicarbonate, carbonate, cinnamate, citrate, chloride, formate, fumarate, gluconate, glutamate, glycolate, lactate, malate, maleate, malonate, mandelate, mesylate, nicotinate, nitrate, oxalate, dihydrogen phosphate, hydrogen phosphate, phosphate, propionate, tosylate, pyroglutamate, salicylate, succinate, bisulfate, sulfate, tartrate, thiocyanate, triflate, and trifluoroacetate.

In some embodiments, each R^{1G} is independently C₁₋₄alkyl. In some embodiments, each R^{1G} is —CH₄.

In some embodiments, R¹ is selected from the group consisting of:

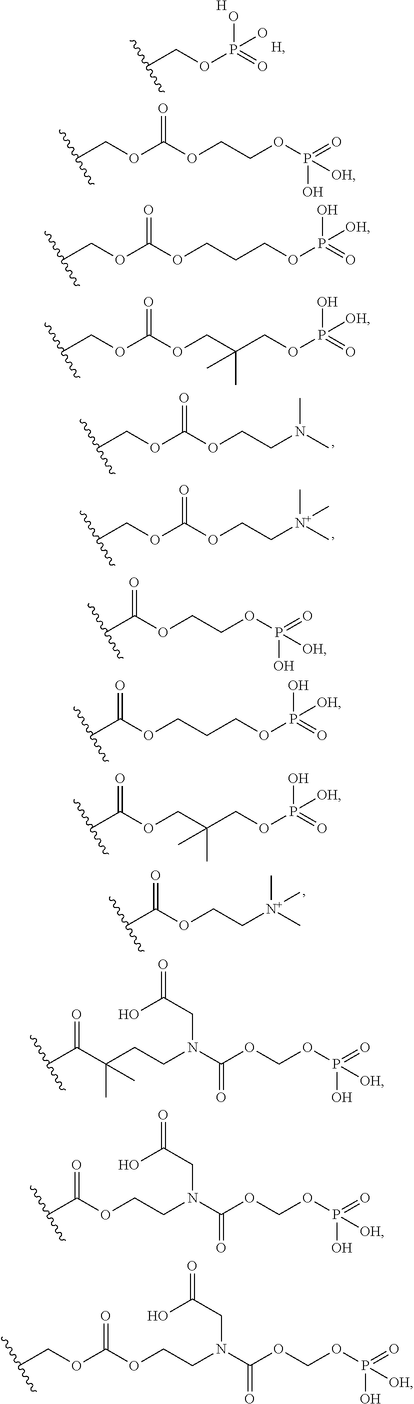

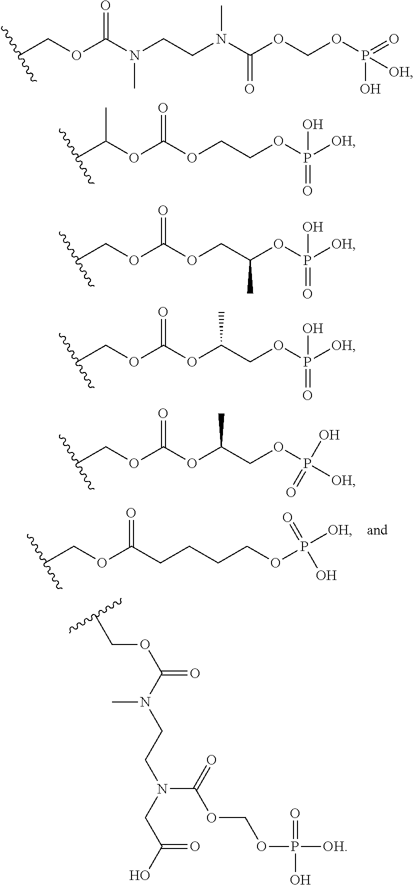

In some embodiments, R¹ is selected from the group consisting of:

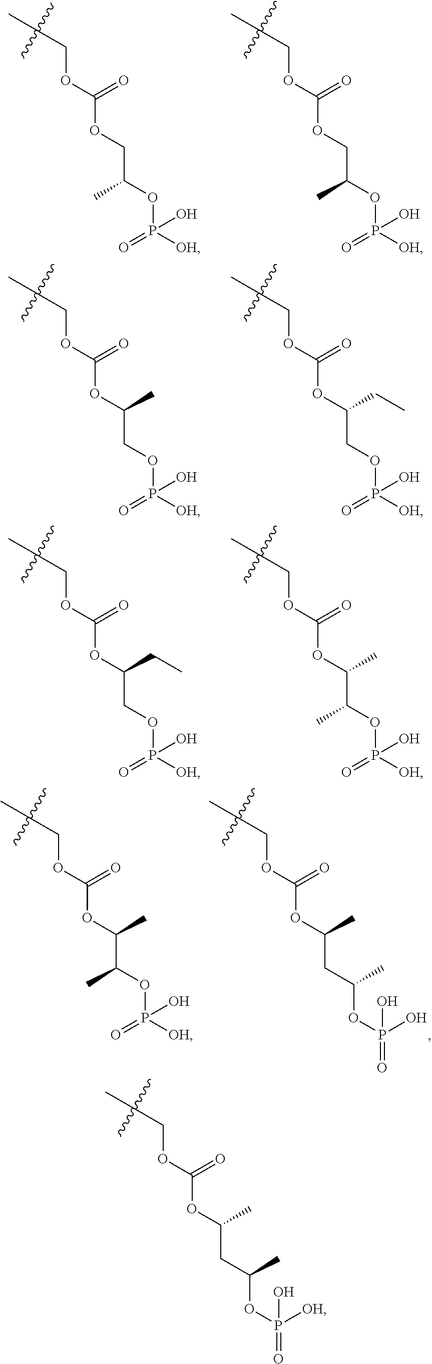

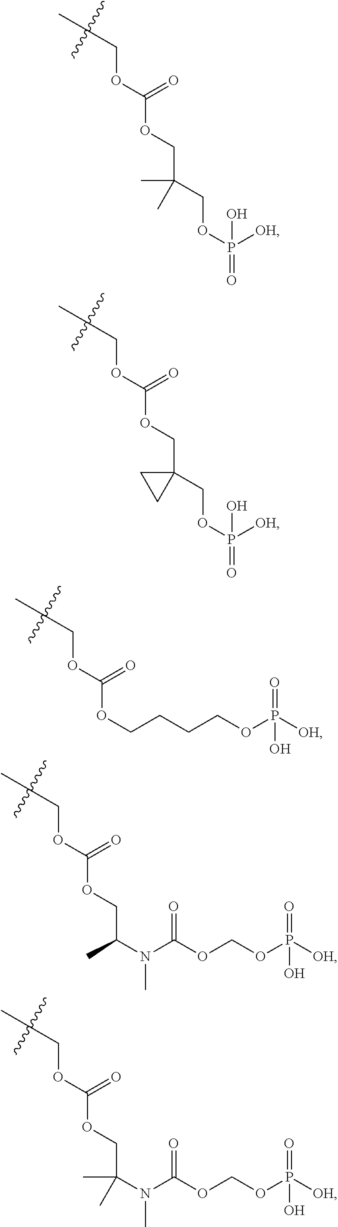 [SMILES: *COC(=O)OCC(C)(C)COP(=O)(O)O ; *COC(=O)OCC1(COP(=O)(O)O)CC1 ; *COC(=O)OCCCCOP(=O)(O)O ; *COC(=O)OC[C@H](C)N(C)C(=O)OCOP(=O)(O)O ; *COC(=O)OCC(C)(C)N(C)C(=O)OCOP(=O)(O)O]

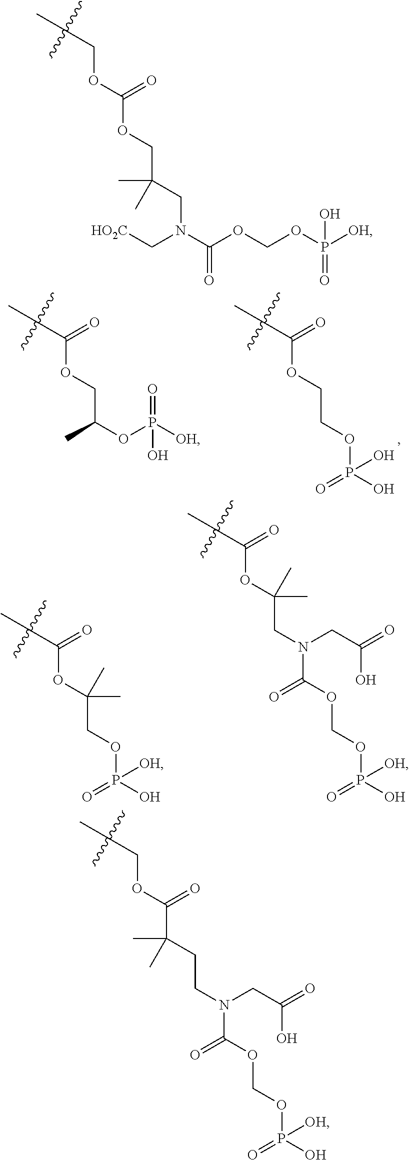

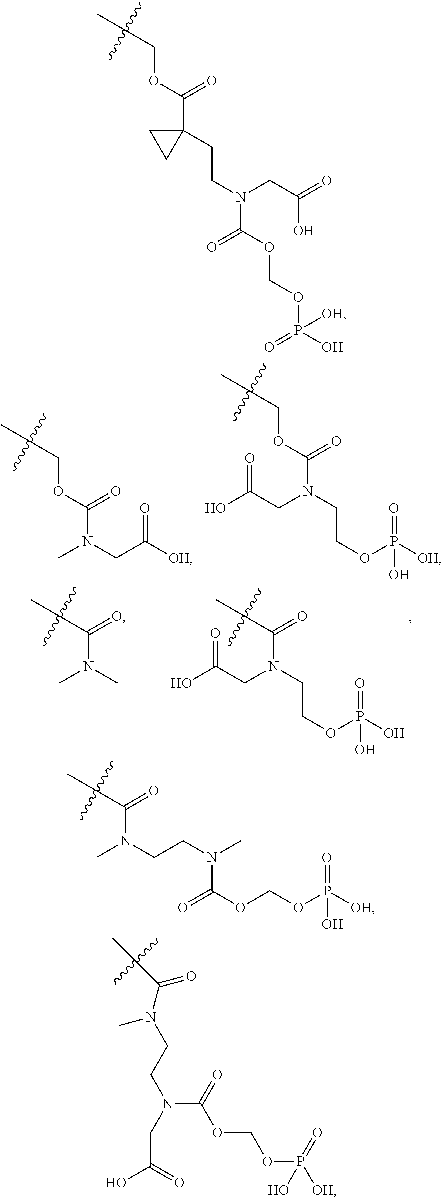

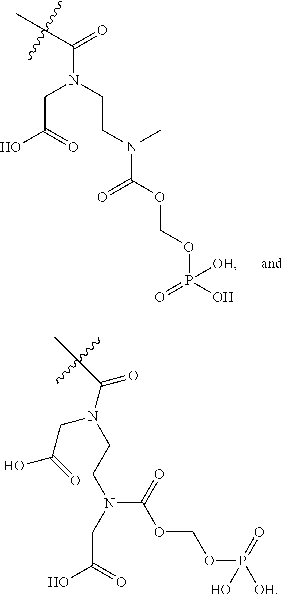 [SMILES: *C(=O)N(CCN(C)C(=O)OCOP(=O)(O)O)CC(=O)O ; *C(=O)N(CCN(CC(=O)O)C(=O)OCOP(=O)(O)O)CC(=O)O]

In some embodiments, R¹ is selected from the group consisting of

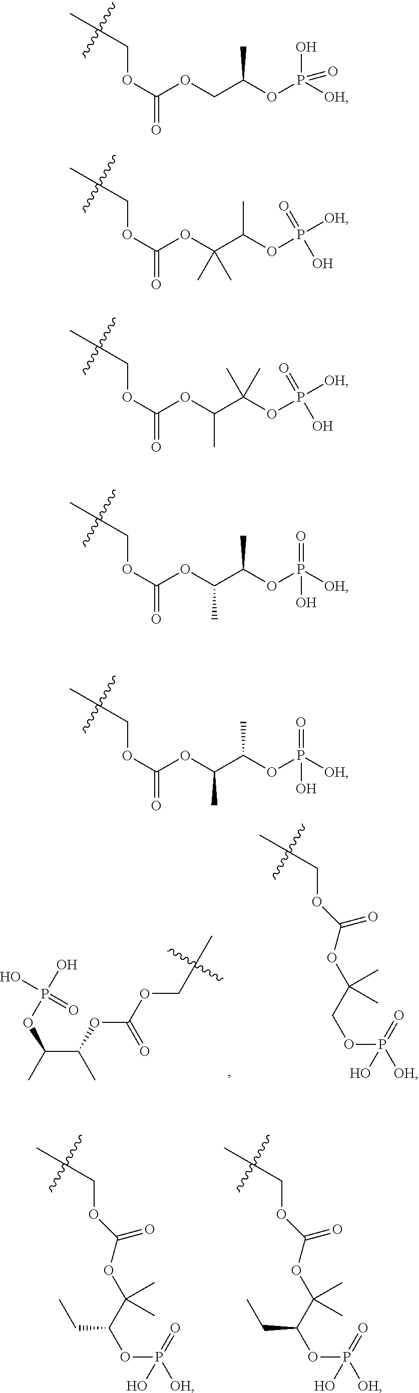

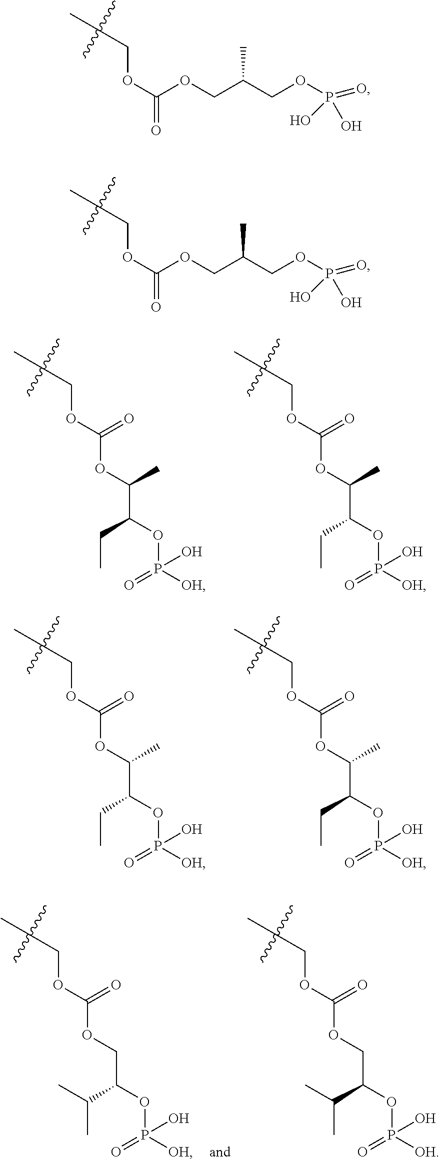

In some embodiments, R² is methyl or methoxy. In some embodiments. R² is methyl. In some embodiments, R² is methoxy.

In some embodiments, R³ and R⁶ are each independently a halo. In some embodiments, R³ and R⁶ are each F.

In some embodiments, R⁴, R⁵ and R⁷ are each H.

In some embodiments, R⁸ is C₁₋₃alkyl. In some embodiments, R⁸ is methyl.

In some embodiments, the compound of Formula I is selected from the group consisting of:

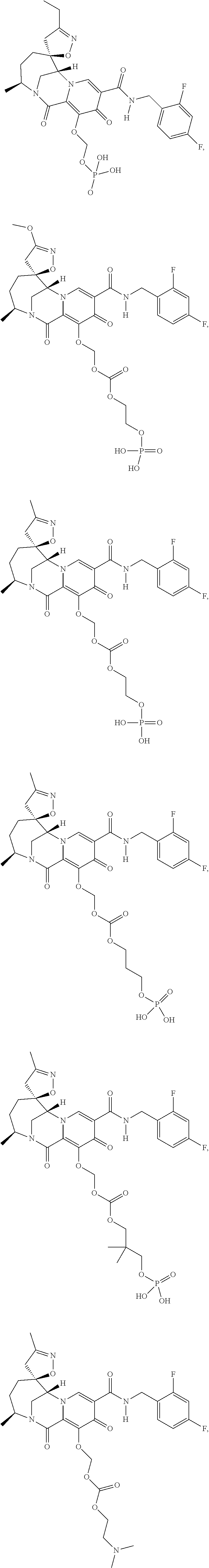

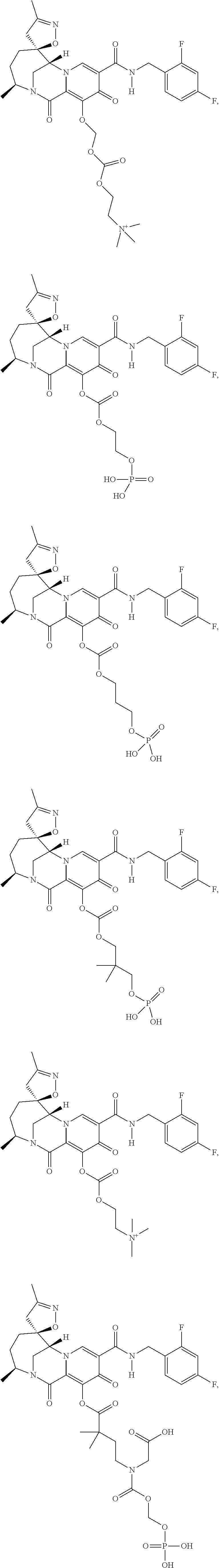

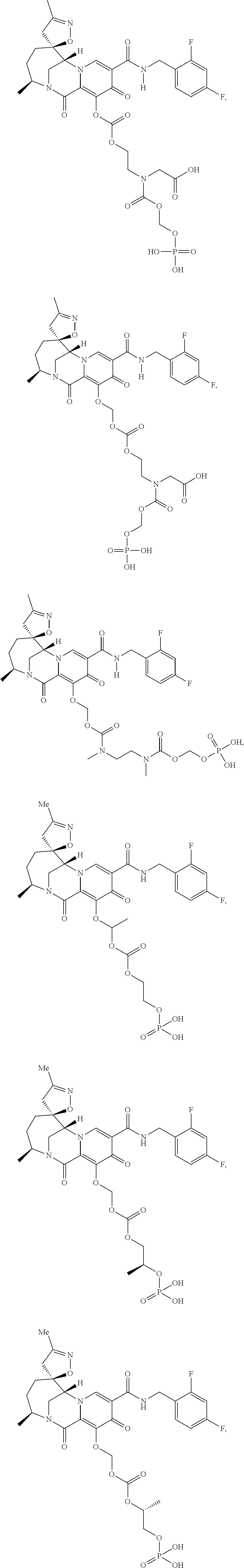

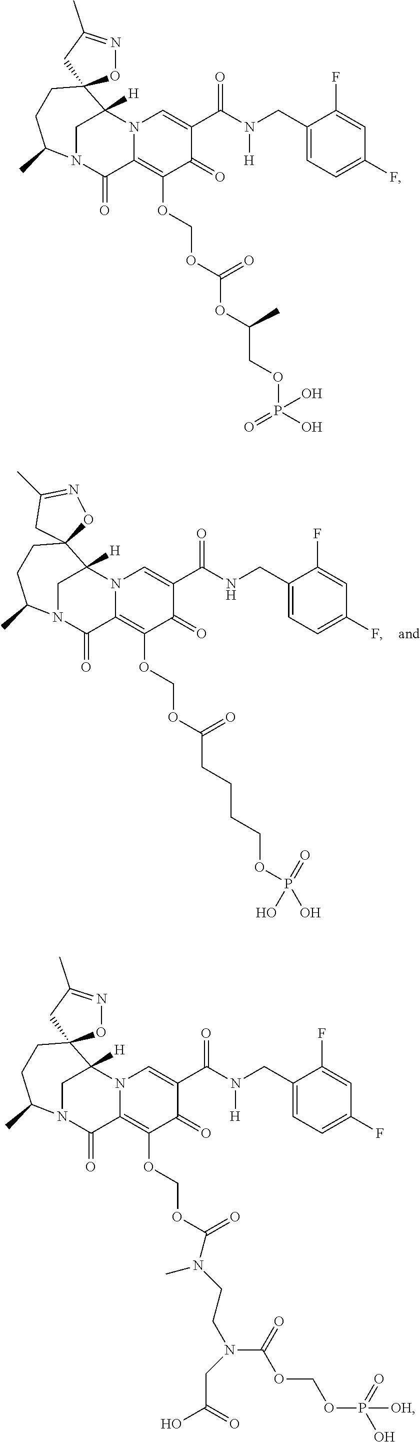 [SMILES: CC1=NO[C@@]2(CC[C@H](C)N3C[C@H]2n2cc(C(=O)NCc4ccc(F)cc4F)c(=O)c(OCOC(=O)O[C@@H](C)COP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)CCCCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)N(C)CCN(CC(=O)O)C(=O)OCOP(=O)(O)O)c2C3=O)C1]

or a pharmaceutically acceptable salt thereof.

In some embodiments, the compound of Formula I is selected from the group consisting of:

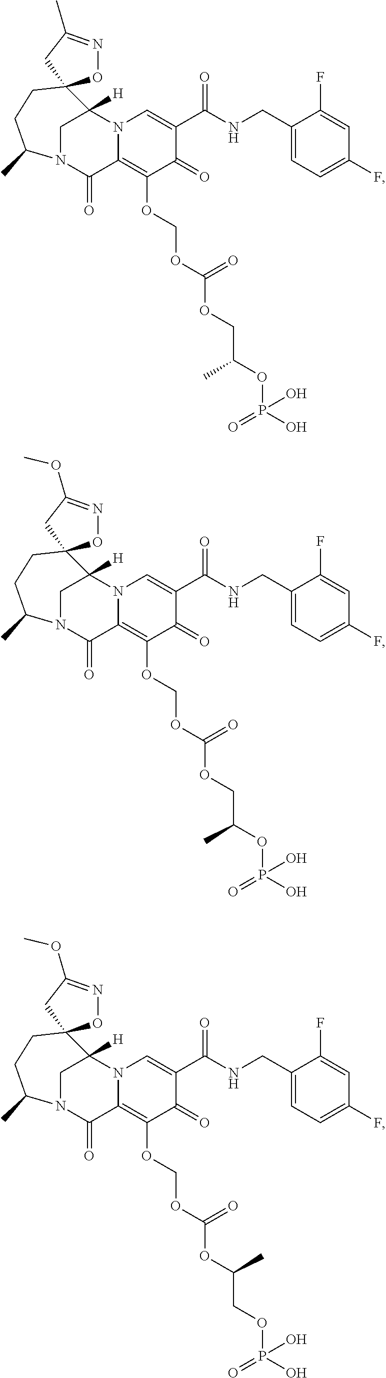 [SMILES: CC1=NO[C@@]2(CC[C@H](C)N3C[C@H]2n2cc(C(=O)NCc4ccc(F)cc4F)c(=O)c(OCOC(=O)OC[C@@H](C)OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)OC[C@H](C)OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)O[C@@H](C)COP(=O)(O)O)c2C3=O)C1]

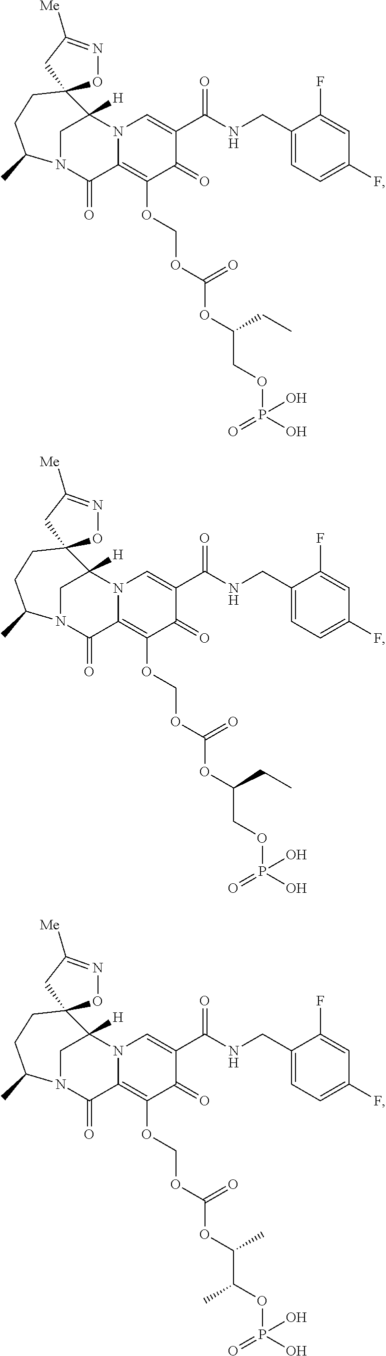 [SMILES: CC[C@H](COP(=O)(O)O)OC(=O)OCOc1c2n(cc(C(=O)NCc3ccc(F)cc3F)c1=O)[C@@H]1CN(C2=O)[C@@H](C)CC[C@]12CC(C)=NO2 ; CC[C@@H](COP(=O)(O)O)OC(=O)OCOc1c2n(cc(C(=O)NCc3ccc(F)cc3F)c1=O)[C@@H]1CN(C2=O)[C@@H](C)CC[C@]12CC(C)=NO2 ; CC1=NO[C@@]2(CC[C@H](C)N3C[C@H]2n2cc(C(=O)NCc4ccc(F)cc4F)c(=O)c(OCOC(=O)O[C@H](C)[C@@H](C)OP(=O)(O)O)c2C3=O)C1]

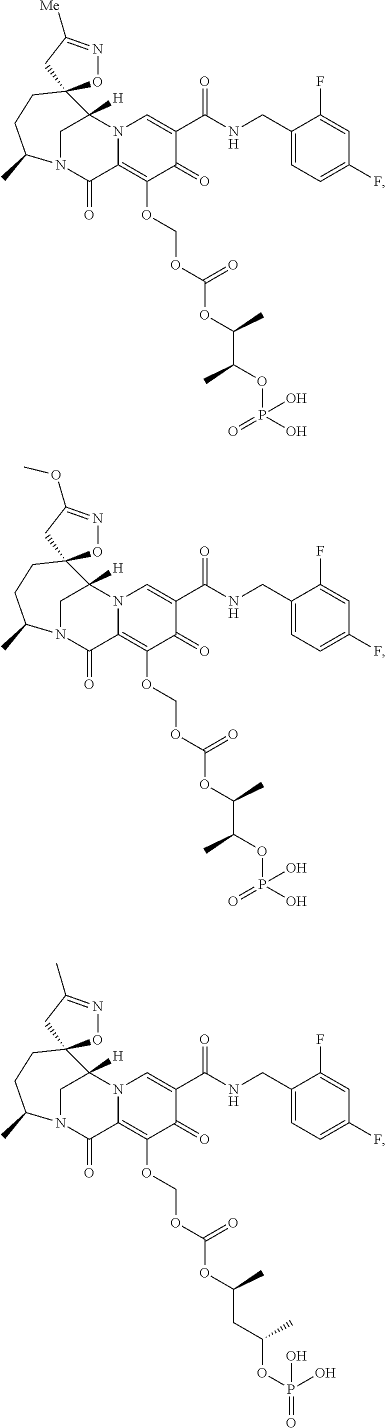 [SMILES: CC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)O[C@@H](C)C[C@H](C)OP(=O)(O)O)c2C3=O)C1]

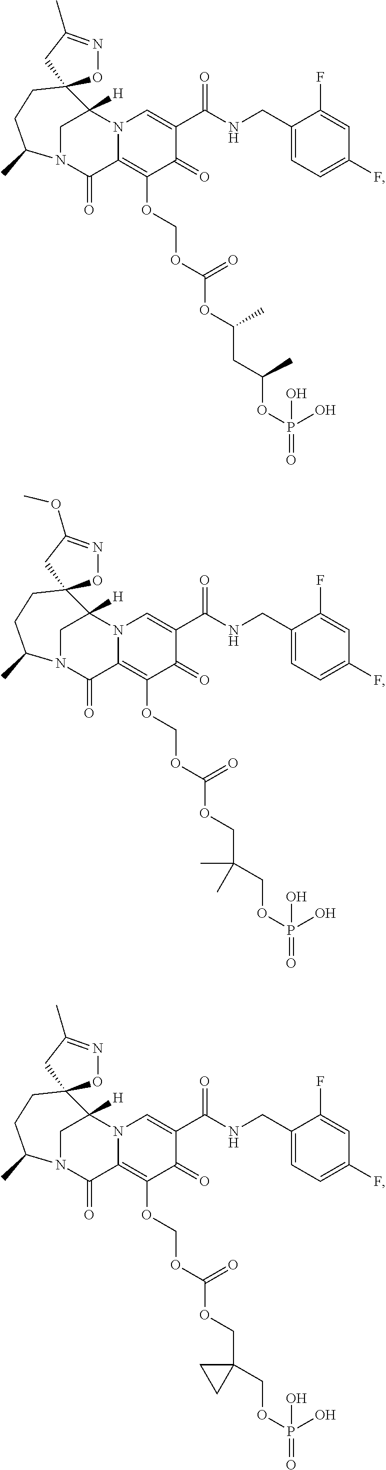 [SMILES: CC1=NO[C@@]2(CC[C@H](C)N3C[C@H]2n2cc(C(=O)NCc4ccc(F)cc4F)c(=O)c(OCOC(=O)O[C@H](C)C[C@@H](C)OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)OCC(C)(C)COP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OCC4(COP(=O)(O)O)CC4)c2C3=O)C1]

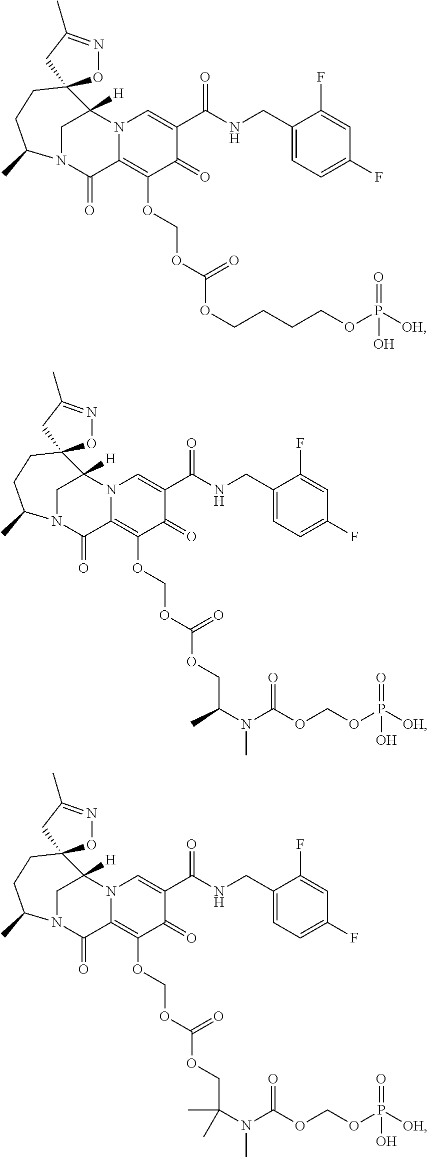 [SMILES: CC1=NO[C@@]2(CC[C@H](C)N3C[C@H]2n2cc(C(=O)NCc4ccc(F)cc4F)c(=O)c(OCOC(=O)OCCCCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OC[C@H](C)N(C)C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OCC(C)(C)N(C)C(=O)OCOP(=O)(O)O)c2C3=O)C1]

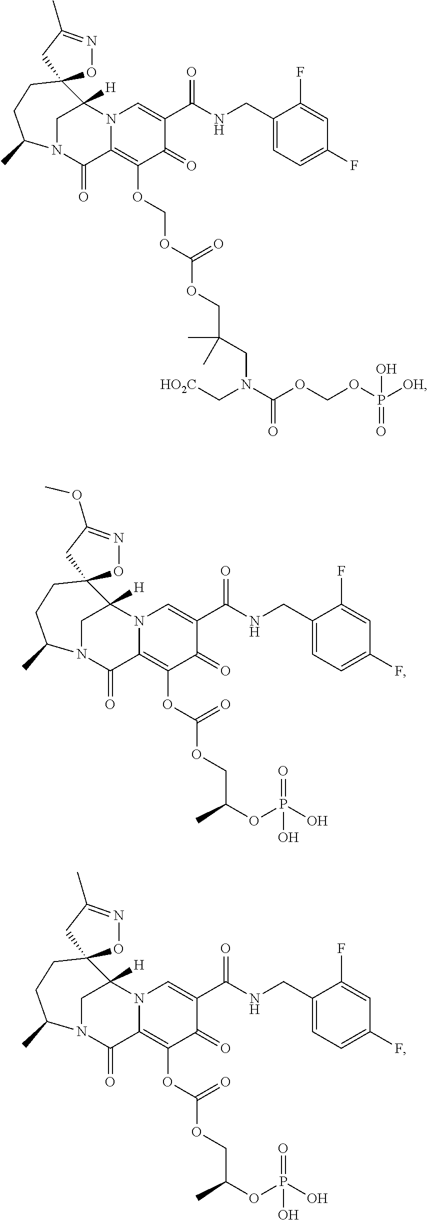 [SMILES: CC1=NO[C@@]2(CC[C@H](C)N3C[C@H]2n2cc(C(=O)NCc4ccc(F)cc4F)c(=O)c(OCOC(=O)OCC(C)(C)CN(CC(=O)O)C(=O)OCOP(=O)(O)O)c2C3=O)C1 ; COC1=NO[C@@]2(CC[C@H](C)N3C[C@H]2n2cc(C(=O)NCc4ccc(F)cc4F)c(=O)c(OC(=O)OC[C@H](C)OP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)OC[C@H](C)OP(=O)(O)O)c2C3=O)C1]

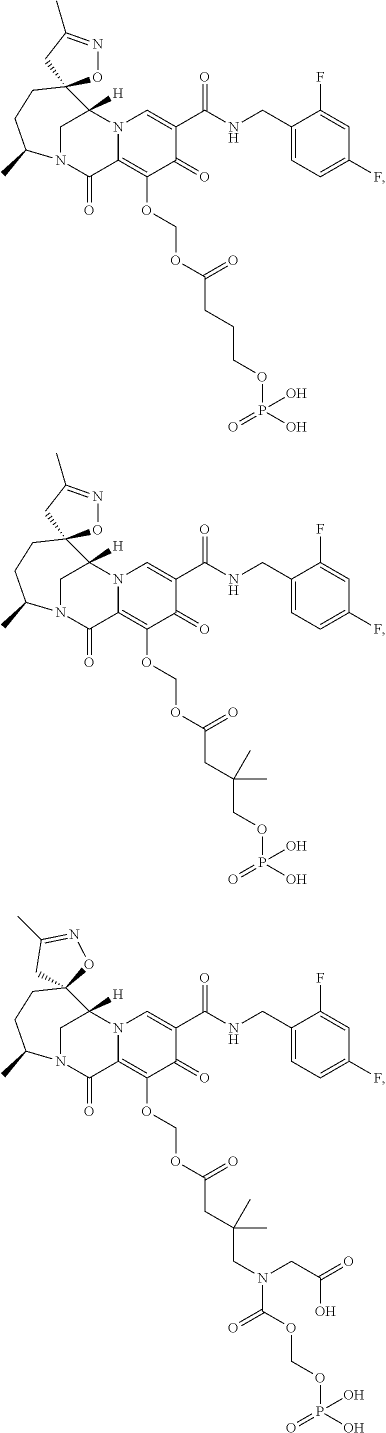 [SMILES: CC1=NO[C@@]2(CC[C@H](C)N3C[C@H]2n2cc(C(=O)NCc4ccc(F)cc4F)c(=O)c(OCOC(=O)CCCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)CC(C)(C)COP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)CC(C)(C)CN(CC(=O)O)C(=O)OCOP(=O)(O)O)c2C3=O)C1]

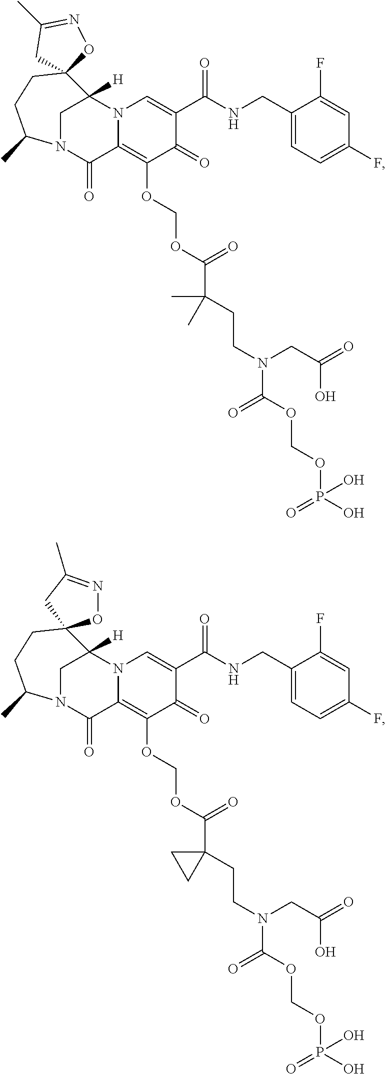 [SMILES: CC1=NO[C@@]2(CC[C@H](C)N3C[C@H]2n2cc(C(=O)NCc4ccc(F)cc4F)c(=O)c(OCOC(=O)C(C)(C)CCN(CC(=O)O)C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)C4(CCN(CC(=O)O)C(=O)OCOP(=O)(O)O)CC4)c2C3=O)C1]

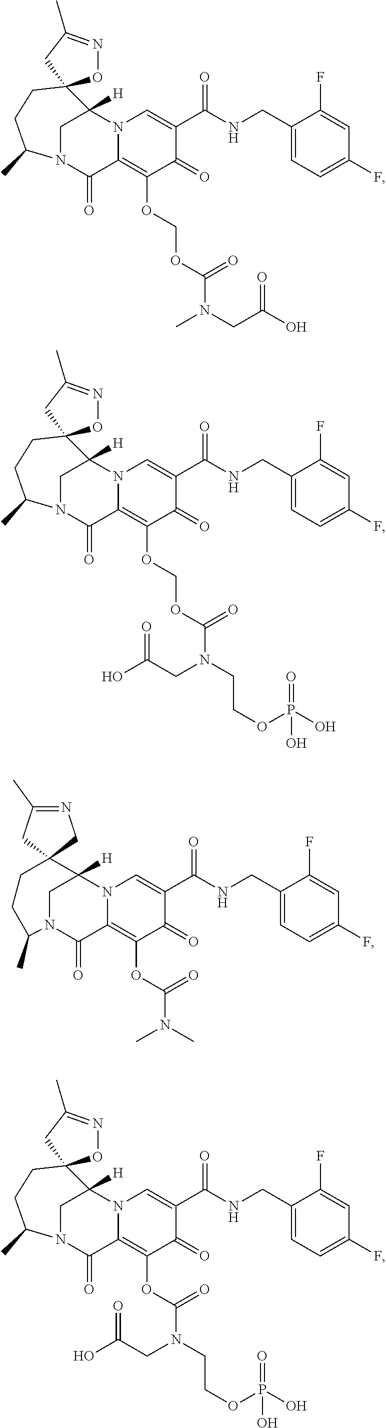 [SMILES: CC1=NO[C@@]2(CC[C@H](C)N3C[C@H]2n2cc(C(=O)NCc4ccc(F)cc4F)c(=O)c(OCOC(=O)N(C)CC(=O)O)c2C3=O)C1 ; CC1=NO[C@@]2(CC[C@H](C)N3C[C@H]2n2cc(C(=O)NCc4ccc(F)cc4F)c(=O)c(OCOC(=O)N(CCOP(=O)(O)O)CC(=O)O)c2C3=O)C1 ; CC1=NC[C@@]2(CC[C@H](C)N3C[C@H]2n2cc(C(=O)NCc4ccc(F)cc4F)c(=O)c(OC(=O)N(C)C)c2C3=O)C1 ; CC1=NO[C@@]2(CC[C@H](C)N3C[C@H]2n2cc(C(=O)NCc4ccc(F)cc4F)c(=O)c(OC(=O)N(CCOP(=O)(O)O)CC(=O)O)c2C3=O)C1]

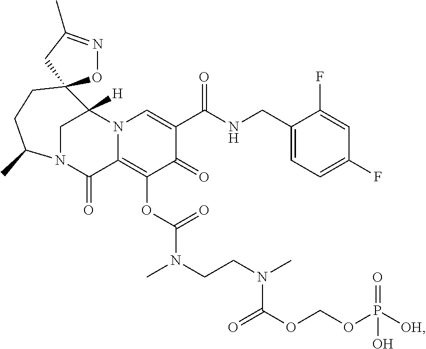 [SMILES: CC1=NO[C@@]2(CC[C@H](C)N3C[C@H]2n2cc(C(=O)NCc4ccc(F)cc4F)c(=O)c(OC(=O)N(C)CCN(C)C(=O)OCOP(=O)(O)O)c2C3=O)C1]

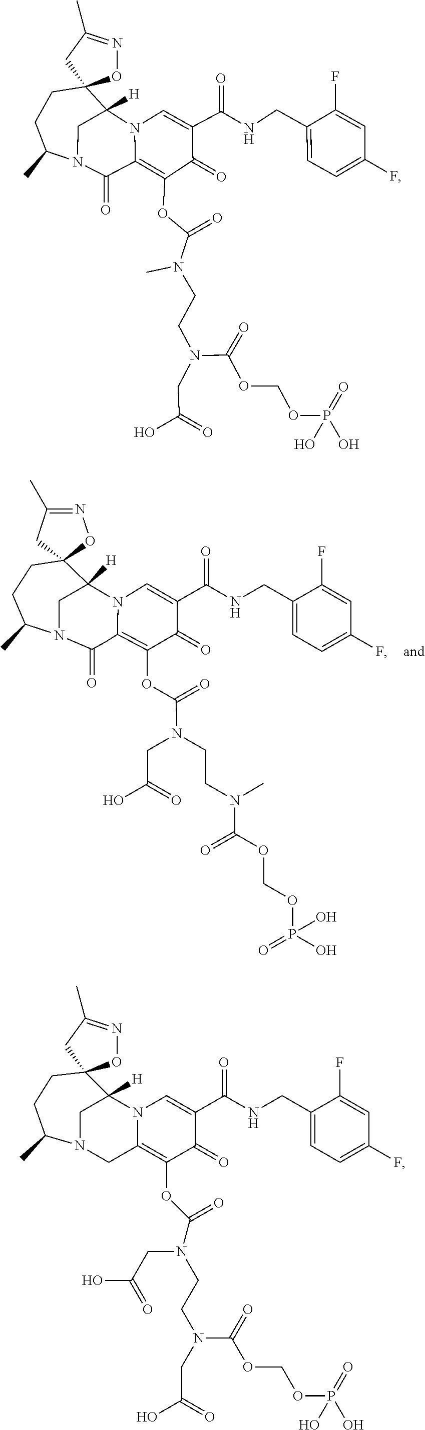 [SMILES: CC1=NO[C@@]2(CC[C@H](C)N3C[C@H]2n2cc(C(=O)NCc4ccc(F)cc4F)c(=O)c(OC(=O)N(C)CCN(CC(=O)O)C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)N(CCN(C)C(=O)OCOP(=O)(O)O)CC(=O)O)c2CC3=O)C1 ; CC1=NO[C@@]2(CC[C@H](C)N3Cc4c(OC(=O)N(CCN(CC(=O)O)C(=O)OCOP(=O)(O)O)CC(=O)O)c(=O)c(C(=O)NCc5ccc(F)cc5F)cn4[C@@H]2C3)C1]

or a pharmaceutically acceptable salt thereof.

In some embodiments, the compound of Formula I is selected from the group consisting of:

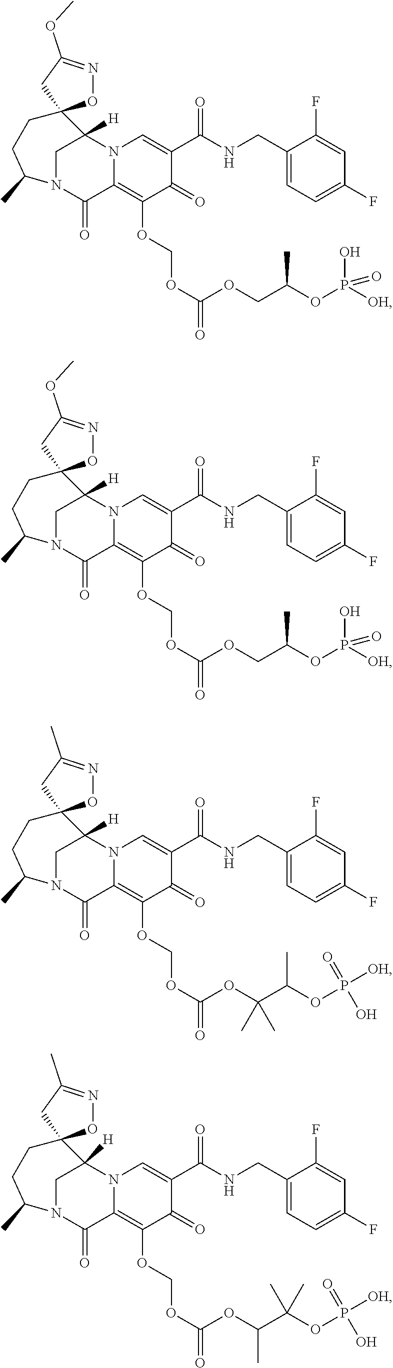 [SMILES: COC1=NO[C@@]2(CC[C@H](C)N3C[C@H]2n2cc(C(=O)NCc4ccc(F)cc4F)c(=O)c(OCOC(=O)OC[C@@H](C)OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)OC[C@@H](C)OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OC(C)(C)C(C)OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OC(C)C(C)(C)OP(=O)(O)O)c2C3=O)C1]

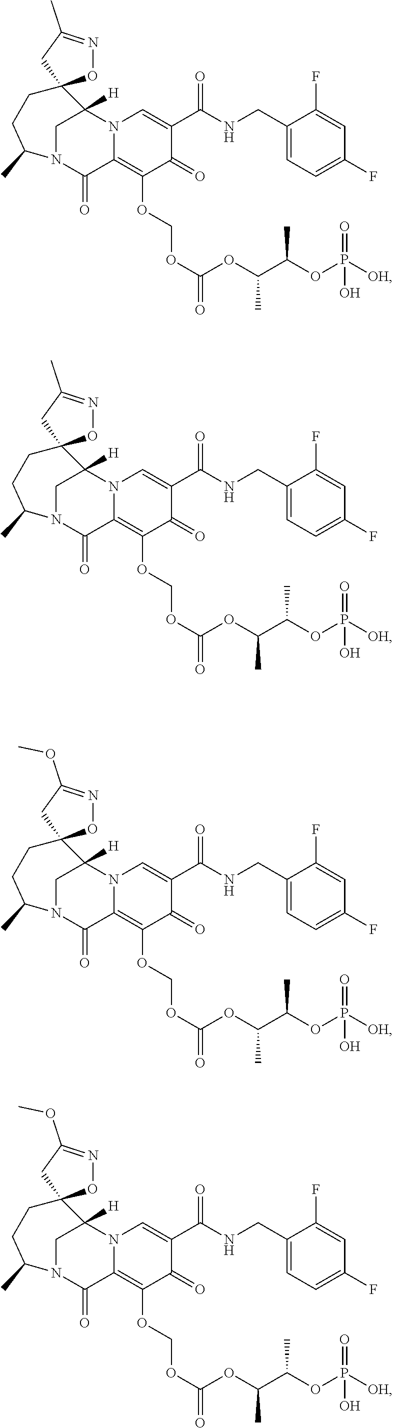 [SMILES: CC1=NO[C@@]2(CC[C@H](C)N3C[C@H]2n2cc(C(=O)NCc4ccc(F)cc4F)c(=O)c(OCOC(=O)O[C@@H](C)[C@@H](C)OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)O[C@H](C)[C@H](C)OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)O[C@@H](C)[C@@H](C)OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)O[C@H](C)[C@H](C)OP(=O)(O)O)c2C3=O)C1]

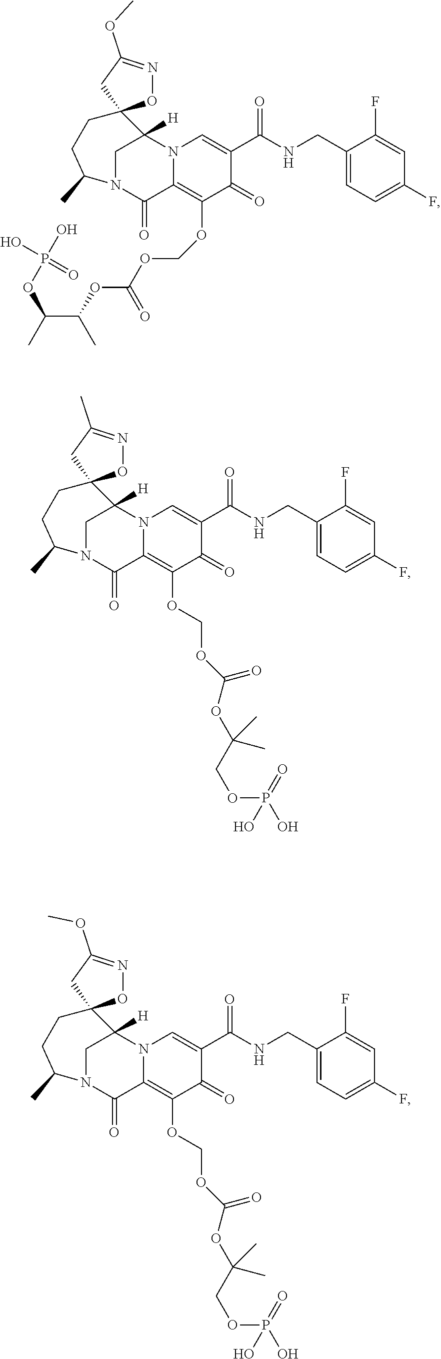 [SMILES: COC1=NO[C@@]2(CC[C@H](C)N3C[C@H]2n2cc(C(=O)NCc4ccc(F)cc4F)c(=O)c(OCOC(=O)O[C@H](C)[C@@H](C)OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OC(C)(C)COP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)OC(C)(C)COP(=O)(O)O)c2C3=O)C1]

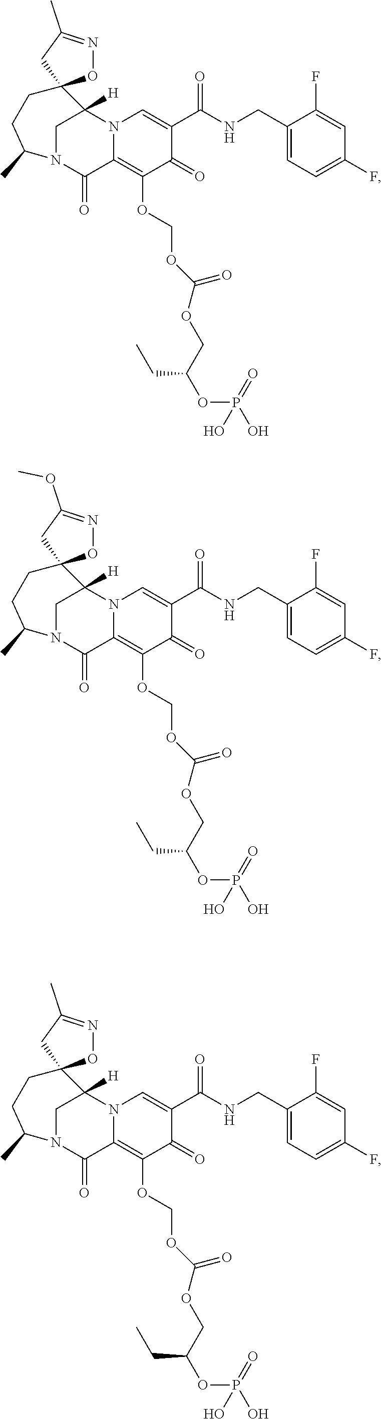 [SMILES: CC[C@H](COC(=O)OCOc1c2n(cc(C(=O)NCc3ccc(F)cc3F)c1=O)[C@@H]1CN(C2=O)[C@@H](C)CC[C@]12CC(C)=NO2)OP(=O)(O)O ; CC[C@H](COC(=O)OCOc1c2n(cc(C(=O)NCc3ccc(F)cc3F)c1=O)[C@@H]1CN(C2=O)[C@@H](C)CC[C@]12CC(OC)=NO2)OP(=O)(O)O ; CC[C@@H](COC(=O)OCOc1c2n(cc(C(=O)NCc3ccc(F)cc3F)c1=O)[C@@H]1CN(C2=O)[C@@H](C)CC[C@]12CC(C)=NO2)OP(=O)(O)O]

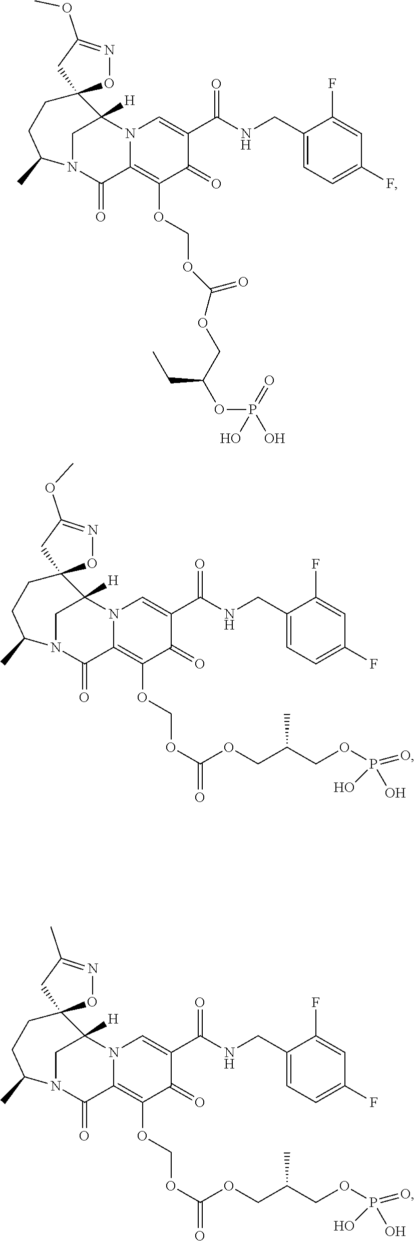 [SMILES: CC[C@@H](COC(=O)OCOc1c2n(cc(C(=O)NCc3ccc(F)cc3F)c1=O)[C@@H]1CN(C2=O)[C@@H](C)CC[C@]12CC(OC)=NO2)OP(=O)(O)O ; COC1=NO[C@@]2(CC[C@H](C)N3C[C@H]2n2cc(C(=O)NCc4ccc(F)cc4F)c(=O)c(OCOC(=O)OC[C@H](C)COP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OC[C@H](C)COP(=O)(O)O)c2C3=O)C1]

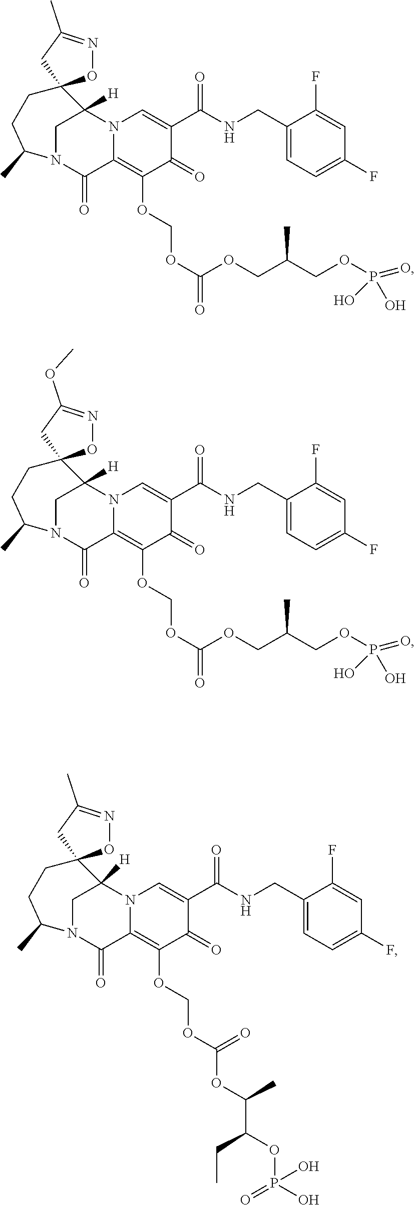 [SMILES: CC1=NO[C@@]2(CC[C@H](C)N3C[C@H]2n2cc(C(=O)NCc4ccc(F)cc4F)c(=O)c(OCOC(=O)OC[C@@H](C)COP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)OC[C@@H](C)COP(=O)(O)O)c2C3=O)C1 ; CC[C@H](OP(=O)(O)O)[C@H](C)OC(=O)OCOc1c2n(cc(C(=O)NCc3ccc(F)cc3F)c1=O)[C@@H]1CN(C2=O)[C@@H](C)CC[C@]12CC(C)=NO2]

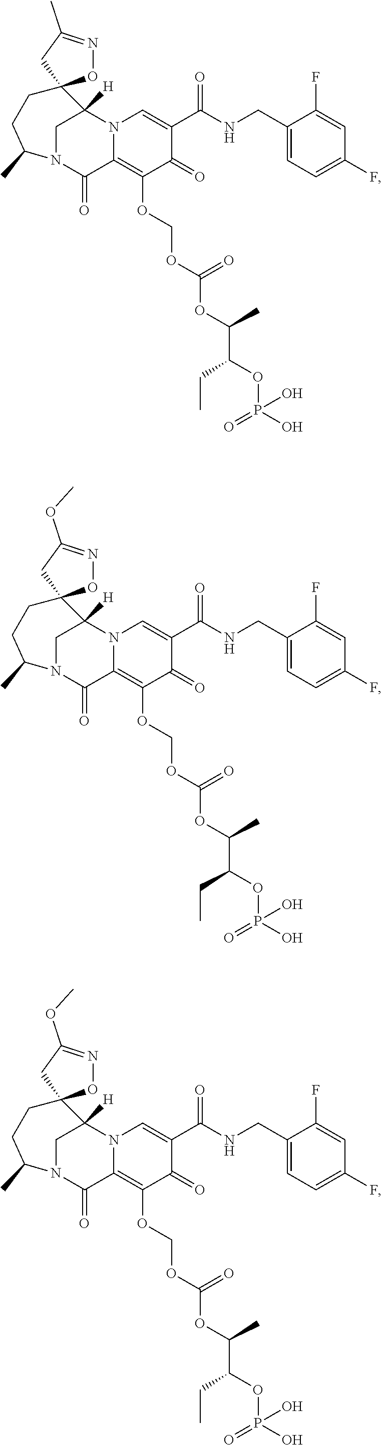 [SMILES: CC[C@@H](OP(=O)(O)O)[C@H](C)OC(=O)OCOc1c2n(cc(C(=O)NCc3ccc(F)cc3F)c1=O)[C@@H]1CN(C2=O)[C@@H](C)CC[C@]12CC(C)=NO2 ; CC[C@H](OP(=O)(O)O)[C@H](C)OC(=O)OCOc1c2n(cc(C(=O)NCc3ccc(F)cc3F)c1=O)[C@@H]1CN(C2=O)[C@@H](C)CC[C@]12CC(OC)=NO2 ; CC[C@@H](OP(=O)(O)O)[C@H](C)OC(=O)OCOc1c2n(cc(C(=O)NCc3ccc(F)cc3F)c1=O)[C@@H]1CN(C2=O)[C@@H](C)CC[C@]12CC(OC)=NO2]

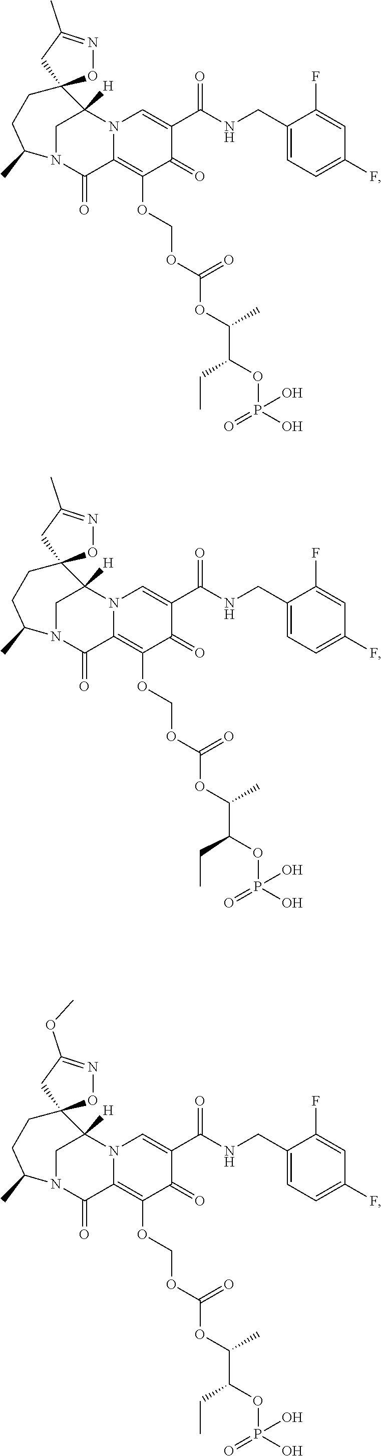 [SMILES: CC[C@@H](OP(=O)(O)O)[C@@H](C)OC(=O)OCOc1c2n(cc(C(=O)NCc3ccc(F)cc3F)c1=O)[C@@H]1CN(C2=O)[C@@H](C)CC[C@]12CC(C)=NO2 ; CC[C@H](OP(=O)(O)O)[C@@H](C)OC(=O)OCOc1c2n(cc(C(=O)NCc3ccc(F)cc3F)c1=O)[C@@H]1CN(C2=O)[C@@H](C)CC[C@]12CC(C)=NO2 ; CC[C@@H](OP(=O)(O)O)[C@@H](C)OC(=O)OCOc1c2n(cc(C(=O)NCc3ccc(F)cc3F)c1=O)[C@@H]1CN(C2=O)[C@@H](C)CC[C@]12CC(OC)=NO2]

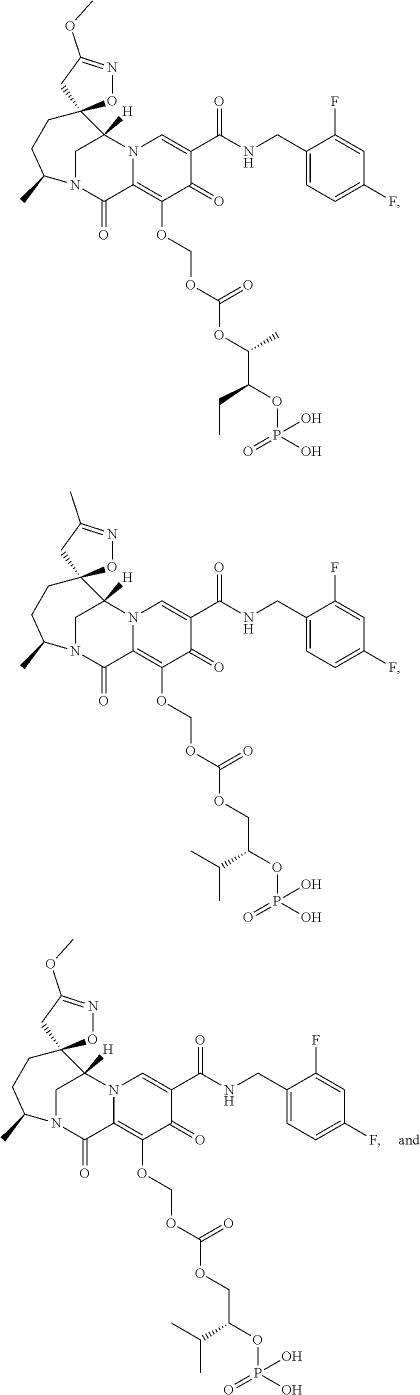 [SMILES: CC[C@H](OP(=O)(O)O)[C@@H](C)OC(=O)OCOc1c2n(cc(C(=O)NCc3ccc(F)cc3F)c1=O)[C@@H]1CN(C2=O)[C@@H](C)CC[C@]12CC(OC)=NO2 ; CC1=NO[C@@]2(CC[C@H](C)N3C[C@H]2n2cc(C(=O)NCc4ccc(F)cc4F)c(=O)c(OCOC(=O)OC[C@H](OP(=O)(O)O)C(C)C)c2C3=O)C1 ; COC1=NO[C@@]2(CC[C@H](C)N3C[C@H]2n2cc(C(=O)NCc4ccc(F)cc4F)c(=O)c(OCOC(=O)OC[C@H](OP(=O)(O)O)C(C)C)c2C3=O)C1 ; "and"]

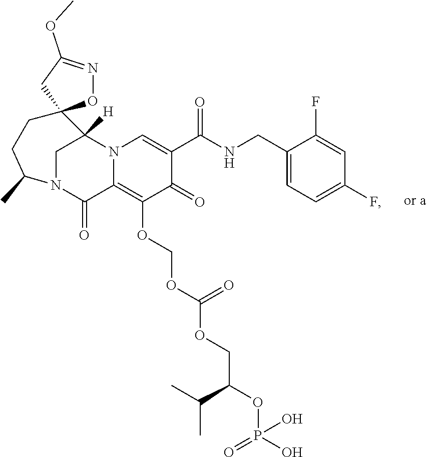

pharmaceutically acceptable salt thereof.

In some embodiments, the compound of Formula I is selected from:

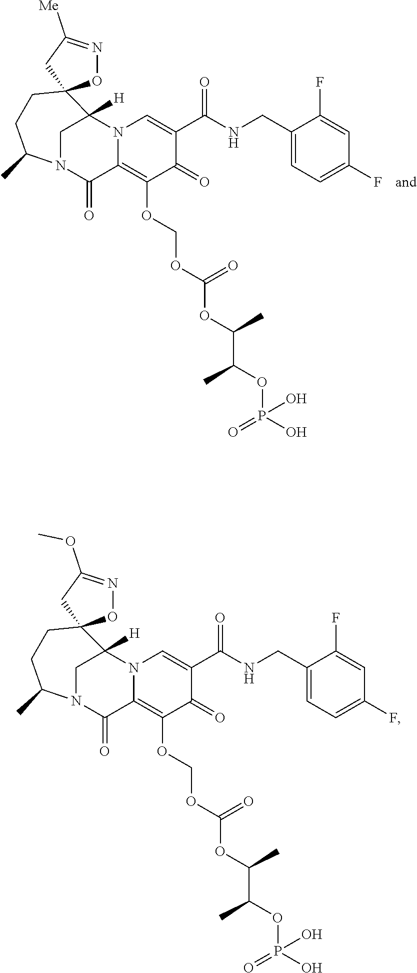 [SMILES: CC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1]

or a pharmaceutically acceptable salt thereof.

In some embodiments, the compound of Formula I is:

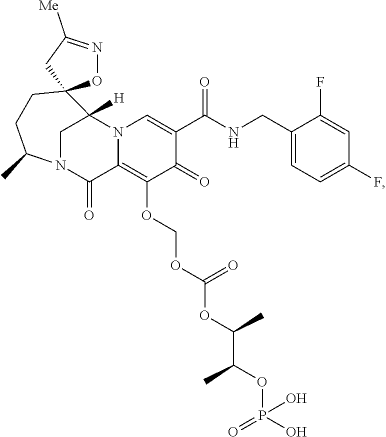 [SMILES: CC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1]

or a pharmaceutically acceptable salt thereof.

In some embodiments, the compound of Formula I is:

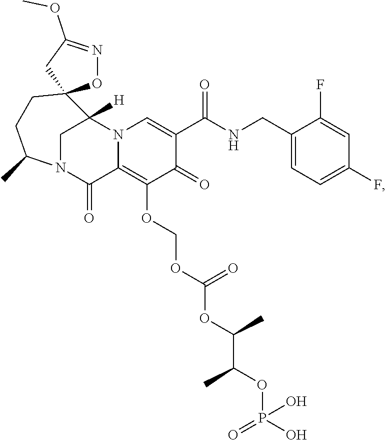 [SMILES: COC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1]

or a pharmaceutically acceptable salt thereof.

In some embodiments, the compounds disclosed herein have therapeutic activity. In some embodiments, the compounds disclosed herein are prodrugs, which upon administration to the human body can be converted to compounds having therapeutic activity. In some embodiments, the compounds disclosed herein are prodrugs of certain compounds disclosed in U.S. application Ser. Nos. 18/296,285, 18/334,588, and 18/334,611, and PCT Application No. PCT/US2023/065401, the disclosures of which are incorporated herein by reference in their entireties. For example, the compounds of Formula I disclosed herein can be converted to compounds of Formula II:

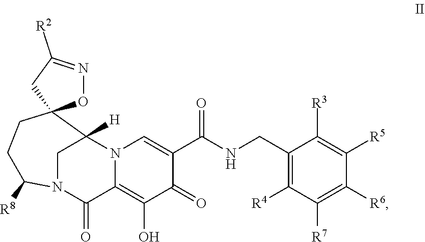

or pharmaceutically acceptable salts thereof, wherein R², R³, R⁴, R⁵, R⁶, R⁷, and R⁸ are as defined according to any other embodiment described herein.

In some embodiments, the compound of Formula II is

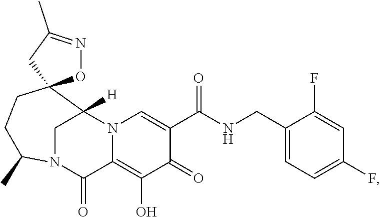 [SMILES: CC1=NO[C@@]2(CC[C@H](C)N3C[C@H]2n2cc(C(=O)NCc4ccc(F)cc4F)c(=O)c(O)c2C3=O)C1]

or a pharmaceutically acceptable salt thereof.

In some embodiments, the compound of Formula II is

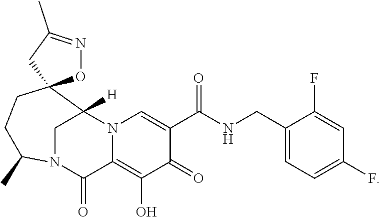 [SMILES: CC1=NO[C@@]2(CC[C@H](C)N3C[C@H]2n2cc(C(=O)NCc4ccc(F)cc4F)c(=O)c(O)c2C3=O)C1]

In some embodiments, the compound of Formula II is

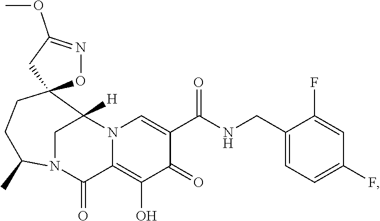 [SMILES: COC1=NO[C@@]2(CC[C@H](C)N3C[C@H]2n2cc(C(=O)NCc4ccc(F)cc4F)c(=O)c(O)c2C3=O)C1]

or a pharmaceutically acceptable salt thereof.

In some embodiments, the compound of Formula II is

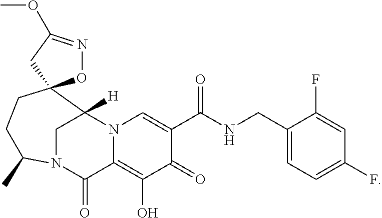 [SMILES: COC1=NO[C@@]2(CC[C@H](C)N3C[C@H]2n2cc(C(=O)NCc4ccc(F)cc4F)c(=O)c(O)c2C3=O)C1]

The present invention includes a method of converting a compound of Formula I, or a pharmaceutically acceptable salt thereof, to Formula II, or a pharmaceutically acceptable salt thereof. A compound of Formula I, or a pharmaceutically acceptable salt thereof, may be converted to a compound of Formula II, or a pharmaceutically acceptable salt thereof, by (1) contacting the compound of Formula I, or a pharmaceutically acceptable salt thereof, with cell-containing aqueous media capable of converting —OR¹ to —OH (e.g., either enzymatically or chemically through acid or base hydrolysis); or (2) administering the compound to a patient whereby the compound of Formula I, or pharmaceutically acceptable salt thereof, is converted to a compound of Formula II, or a pharmaceutically acceptable salt thereof, through biologic pathways (e.g., enzymes) or through contact with biologic fluids and/or tissues conducive to converting —OR¹ to —OH (e.g., through acid or base hydrolysis).

The present invention further provides the treatment or prophylaxis of HIV infection in a patient in need thereof, by contacting the patient with a compound of Formula II, or a pharmaceutically acceptable salt thereof, whereby the compound of Formula II, or a pharmaceutically acceptable salt thereof, is generated within the patient upon administration to the patient of a compound of Formula I, or a pharmaceutically acceptable salt thereof. In some embodiments, the compound of Formula II that is generated within the patient is Intermediate B or Intermediate C, as described below in the Example section. In some embodiments, the treatment or prophylaxis of HIV infection in a patient in need thereof uses a combination disclosed herein comprising a compound of Formula II, or a pharmaceutically acceptable salt thereof.

### III. Pharmaceutical Compositions

Compounds provided herein are usually administered in the form of pharmaceutical compositions. Thus, provided herein are also pharmaceutical compositions that comprise one or more of the compounds provided herein or pharmaceutically acceptable salts, isomer, or a mixture thereof and one or more pharmaceutically acceptable vehicles selected from carriers, adjuvants and excipients. The compounds provided herein may be the sole active ingredient or one of the active ingredients of the pharmaceutical compositions. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes. Eds.).

In one aspect, provided herein are pharmaceutical compositions comprising a compound provided herein (e.g., a compound of Formula I or Formula II), or a pharmaceutically acceptable salt thereof, and a pharmaceutically acceptable excipient or carrier. In some embodiments, the pharmaceutical compositions comprise a therapeutically effective amount of a compound provided herein, or a pharmaceutically acceptable salt thereof, and a pharmaceutically acceptable excipient or carrier.

In some embodiments, the pharmaceutical compositions provided herein further comprise one or more (e.g., one, two, three, four, one or two, one to three, or one to four) additional therapeutic agents, or a pharmaceutically acceptable salt thereof. In some embodiments, the pharmaceutical compositions further comprise a therapeutically effective amount of the one or more (e.g., one, two, three, four, one or two, one to three, or one to four) additional therapeutic agents, or a pharmaceutically acceptable salt thereof. In some embodiments, the pharmaceutical compositions further comprise one, two, three, or four additional therapeutic agents.

The pharmaceutical compositions may be administered in either single or multiple doses. The pharmaceutical compositions may be administered by various methods including, for example, rectal, buccal, intranasal and transdermal routes. In some embodiments, the pharmaceutical compositions may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds provided herein. Administration may be via, for example, capsule or enteric coated tablets. In making the pharmaceutical compositions that include at least one compound provided herein or pharmaceutically acceptable salts, isomer, or a mixture thereof, the active ingredient (such as a compound provided herein) is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the pharmaceutical compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose, Vitamin E-Tocopherol polyethylene glycol succinate (Vitamin E-TPGS), and polyethoxylated castor oil (also known as Cremophor EL or Kolliphor EL), or any combinations thereof. The pharmaceutical compositions can additionally include lubricating agents such as talc, magnesium stearate, and mineral oil; wetting agents; emulsifying and suspending agents; preserving agents such as methyl and propylhydroxy-benzoates; sweetening agents; and flavoring agents; or any combinations thereof.

The pharmaceutical compositions that include at least one compound described herein or pharmaceutically acceptable salts, isomer, or a mixture thereof can be formulated so as to provide quick, sustained or delayed release of the active ingredient (such as a compound provided herein) after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present disclosure employs transdermal delivery devices (“patches”). Such transdermal patches may be used to provide continuous or discontinuous infusion of the compounds provided herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or pharmaceutically acceptable salts, isomer, or a mixture thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with materials such as shellac, cetyl alcohol, and cellulose acetate.

Pharmaceutical compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

In some embodiments, the compounds of Formula I or Formula II, or pharmaceutically acceptable salts thereof, and PGP inhibitors described herein are provided in the form of pharmaceutical compositions.

A compound of Formula I or Formula II, or a pharmaceutically acceptable salt thereof, described herein may be present in the same pharmaceutical composition as the PGP inhibitor or may be present in a different pharmaceutical composition to the PGP inhibitor. In one embodiment, the compound of Formula I or Formula II, or a pharmaceutically acceptable salt thereof, are formulated in the same pharmaceutical composition as the PGP inhibitor e.g. as a fixed combination.

In one embodiment, the compound of Formula I or Formula II, or a pharmaceutically acceptable salt thereof, and the PGP inhibitor are formulated in different pharmaceutical compositions e.g. for separate, simultaneous, or sequential administration. When formulated separately, the compound of Formula I or Formula II, or a pharmaceutically acceptable salt thereof, and the PGP inhibitors provided herein may be the sole active ingredient of the pharmaceutical compositions.

Each pharmaceutical composition may comprise one or more pharmaceutically acceptable vehicles selected from carriers, adjuvants and excipients.

### V. Methods of Treatment

In one embodiment, methods of treating an HIV (e.g., HIV-1 and/or HIV-2) infection in a human having or at risk of having the infection comprising administering to the human a therapeutically effective amount of a compound of Formula I, or a pharmaceutically acceptable salt thereof, or a pharmaceutical composition of a compound of Formula I, or a pharmaceutically acceptable salt thereof, are provided.

In some embodiments, the methods further comprise administering to the human a therapeutically effective amount of one, two, three, or four additional therapeutic agents. In certain embodiments, the additional therapeutic agent or agents are anti-HIV agents. In particular embodiments, the additional therapeutic agent or agents are HIV protease inhibitors, HIV non-nucleoside or non-nucleotide inhibitors of reverse transcriptase, HIV nucleoside or nucleotide inhibitors of reverse transcriptase, HIV capsid inhibitors, gp41 inhibitors, CXCR4 inhibitors, gp120 inhibitors, CCR5 inhibitors, latency reversing agents, capsid polymerization inhibitors, HIV bNAbs (broadly neutralizing HIV antibodies), TLR7 agonists, pharmacokinetic enhancers, other drugs for treating HIV, or combinations thereof.

In some embodiments, the additional therapeutic agent or agents are HIV protease inhibitors. HIV non-nucleoside or non-nucleotide inhibitors of reverse transcriptase, HIV nucleoside or nucleotide inhibitors of reverse transcriptase and/or translocation, HIV capsid inhibitors, gp41 inhibitors, CXCR4 inhibitors, gp120 inhibitors, CCR5 inhibitors, latency reversing agents, capsid polymerization inhibitors, HIV bNAbs (broadly neutralizing HIV antibodies). TLR7 agonists, pharmacokinetic enhancers, other drugs for treating HIV, or combinations thereof.

In some embodiments, the additional therapeutic agent or agents are abacavir, tenofovir alafenamide, tenofovir disoproxil, N—((S)-1-(3-(4-chloro-3-(methylsulfonamido)-1-(2,2,2-trifluoroethyl)-1H-indazol-7-yl)-6-(3-methyl-3-(methylsulfonyl)but-1-yn-1-yl)-pyridin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide, or a pharmaceutically acceptable salt thereof. In one embodiment, the additional therapeutic agent or agents are abacavir, tenofovir alafenamide, tenofovir disoproxil, lenacapavir, or a pharmaceutically acceptable salt thereof. In one embodiment, the additional therapeutic agent or agents are abacavir, tenofovir alafenamide, tenofovir disoproxil, lenacapavir, GS-5894, islatravir, or a pharmaceutically acceptable salt thereof. In some embodiments, the additional therapeutic agent or agents are lenacapavir, islatravir. In some embodiments, the additional therapeutic agent is lenacapavir. In some embodiments, the additional therapeutic agent is islatravir.

In another embodiment, a use of a compound of Formula I, or a pharmaceutically acceptable salt thereof, or a pharmaceutical composition of a compound of Formula I, or a pharmaceutically acceptable salt thereof, for treating an HIV (e.g., HIV-1 and/or HIV-2) infection in a human having or at risk of having the infection is provided.

In another embodiment, a compound of Formula I, or a pharmaceutically acceptable salt thereof, or a pharmaceutical composition of a compound of Formula I, or a pharmaceutically acceptable salt thereof, for use in medical therapy is provided.

In another embodiment, a compound of Formula I, or a pharmaceutically acceptable salt thereof, or a pharmaceutical composition of Formula I, or pharmaceutically acceptable salt thereof, for use in treating an HIV infection is provided.

In another embodiment, a compound of Formula I, or a pharmaceutically acceptable salt thereof, or a pharmaceutical composition of a compound of Formula I, or a pharmaceutically acceptable salt thereof for use in a method of treating an HIV infection in a human having or at risk of having the infection, is provided.

In another embodiment, a compound of Formula I, or a pharmaceutically acceptable salt thereof, or a pharmaceutical composition of a compound of Formula I, or a pharmaceutically acceptable salt thereof for use in a method of treating an HIV infection in a human having or at risk of having the infection, is provided wherein said method further comprises administering to the human one, two, three, or four additional therapeutic agents.

In another embodiment, a compound of I, or a pharmaceutically acceptable salt thereof, or a pharmaceutical composition of a compound of Formula I, or a pharmaceutically acceptable salt thereof for use in a method of treating an HIV infection in a human having or at risk of having the infection, is provided wherein said method further comprises administering to the human one, two, three, or four additional therapeutic agents selected from the group consisting of HIV protease inhibitors, HIV non-nucleoside or non-nucleotide inhibitors of reverse transcriptase, HIV nucleoside or nucleotide inhibitors of reverse transcriptase, HIV capsid inhibitors, gp41 inhibitors, CXCR4 inhibitors, gp120 inhibitors, CCR5 inhibitors, latency reversing agents, capsid polymerization inhibitors, HIV bNAbs, TLR7 agonists, pharmacokinetic enhancers, other drugs for treating HIV, or combinations thereof. In one embodiment, the one, two, three, or four additional therapeutic agents are selected from HIV protease inhibitors, HIV non-nucleoside inhibitors of reverse transcriptase, HIV nucleoside or nucleotide inhibitors of reverse transcriptase, latency reversing agents. HIV capsid inhibitors. HIV bNAbs, TLR7 agonists, and combinations thereof.

In another embodiment, a compound of Formula I, or a pharmaceutically acceptable salt thereof, or a pharmaceutical composition of a compound of Formula I, or a pharmaceutically acceptable salt thereof for use in a method of treating an HIV infection in a human having or at risk of having the infection, is provided wherein said method further comprises administering to the human a therapeutically effective amount of tenofovir disoproxil and emtricitabine.

In another embodiment, a compound of Formula I, or a pharmaceutically acceptable salt thereof, or a pharmaceutical composition of a compound of Formula I, or a pharmaceutically acceptable salt thereof for use in a method of treating an HIV infection in a human having or at risk of having the infection, is provided wherein said method further comprises administering to the human a therapeutically effective amount of tenofovir alafenamide and emtricitabine.

In another embodiment, a compound of Formula I, or a pharmaceutically acceptable salt thereof, or a pharmaceutical composition of a compound of I, or a pharmaceutically acceptable salt thereof for use in a method of treating an HIV infection in a human having or at risk of having the infection, is provided wherein said method further comprises administering to the human a therapeutically effective amount of tenofovir disoproxil.

In another embodiment, a compound of Formula I, or a pharmaceutically acceptable salt thereof, or a pharmaceutical composition of a compound of Formula I, or a pharmaceutically acceptable salt thereof for use in a method of treating an HIV infection in a human having or at risk of having the infection, is provided wherein said method further comprises administering to the human a therapeutically effective amount of tenofovir alafenamide.

In another embodiment, a method of using a compound of Formula I, in therapy is provided. In particular, a method of treating the proliferation of the HIV virus, treating AIDS, or delaying the onset of AIDS or ARC symptoms in a mammal (e.g. a human) is provided, comprising administering to the mammal a compound of Formula I, or a pharmaceutically acceptable salt thereof, and a pharmaceutically acceptable excipient.

In another embodiment, a composition comprising a compound of Formula I. or pharmaceutically acceptable salt thereof, and a pharmaceutically acceptable excipient, for use in a method of treating the proliferation of the HIV virus, treating AIDS, or delaying the onset of AIDS or ARC symptoms in a mammal (e.g., a human) is provided.

In one embodiment, a compound of Formula I, or a pharmaceutically acceptable salt thereof, or a pharmaceutical composition of a compound of Formula I, or a pharmaceutically acceptable salt thereof, is provided for use in preventing HIV infection.

In some embodiments, a compound of Formula I, or a pharmaceutically acceptable salt thereof, or a pharmaceutical composition of a compound of Formula I, or a pharmaceutically acceptable salt thereof, is provided for use in preventing an HIV infection prior to and/or after an event that would expose an individual to HIV or that would otherwise increase the individual's risk of acquiring HIV, e.g., as pre-exposure prophylaxis (PrEP) and/or as post-exposure prophylaxis (PEP).

For example, in one embodiment, a compound of Formula I, or a pharmaceutically acceptable salt thereof, or a pharmaceutical composition of a compound of Formula I, or a pharmaceutically acceptable salt thereof, is provided for use in pre-exposure prophylaxis (PrEP), i.e., before the exposure of the individual to the HIV virus to prevent HIV infection from taking hold if the individual is exposed to the virus and/or to keep the virus from establishing a permanent infection and/or to prevent the appearance of symptoms of the disease and/or to prevent the virus from reaching detectable levels in the blood.

In another embodiment, a compound of Formula I, or a pharmaceutically acceptable salt thereof, or a composition comprising a compound of Formula I, or a pharmaceutically acceptable salt thereof, is provided for use in post-exposure prophylaxis (PEP), i.e., after the exposure of the individual to the HIV virus to prevent HIV infection from taking hold and/or to keep the virus from establishing a permanent infection and/or to prevent the appearance of symptoms of the disease and/or to prevent the virus from reaching detectable levels in the blood.

In another embodiment, the use of a compound of Formula I, or a pharmaceutically acceptable salt thereof, for the manufacture of a medicament for treating an HIV infection in a human being having or at risk of having the infection is disclosed.

In another embodiment, the use of a compound of Formula I, or a pharmaceutically acceptable salt thereof, as a research tool is disclosed.

In another embodiment, an article of manufacture comprising a composition effective to treat an HIV infection; and packaging material comprising a label which indicates that the composition can be used to treat infection by HIV is disclosed. Exemplary compositions comprise a compound of Formula I, or a pharmaceutically acceptable salt thereof.

In still another embodiment, a method of inhibiting the replication of HIV is disclosed. The method comprises exposing the virus to an effective amount of the compound of Formula I, or a pharmaceutically acceptable salt thereof, under conditions where replication of HIV is inhibited.

In another embodiment, the use of a compound of Formula I, or a pharmaceutically acceptable salt thereof, to inhibit the activity of the HIV integrase enzyme is disclosed.

In another embodiment, the use of a compound of Formula I, or a pharmaceutically acceptable salt thereof, to inhibit the replication of HIV is disclosed.

### VI. Administration

The compounds disclosed herein can be administered by any route appropriate to the condition to be treated. Suitable routes include oral, rectal, nasal, topical (including buccal and sublingual), transdermal, vaginal and parenteral (including subcutaneous, intramuscular, intravenous, intradermal, intrathecal and epidural), and the like. In some embodiments, the administration is oral, intravenous, subcutaneous, or intramuscular. It will be appreciated that the preferred route may vary with, for example, the condition of the recipient. An advantage of certain compounds disclosed herein is that they are orally bioavailable and can be dosed orally.

A compound of the present disclosure may be administered to an individual in accordance with an effective dosing regimen for a desired period of time or duration, such as at least about one month, at least about 2 months, at least about 3 months, at least about 6 months, or at least about 12 months or longer. In some embodiments, the compound is administered on a daily or intermittent schedule for the duration of the individual's life.

The specific dose level of a compound of the present disclosure for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The dosage may also be described as a total amount of a compound described herein administered per dose. Dosage of a compound of Formula I, or a pharmaceutically acceptable salt or pharmaceutically acceptable tautomer thereof, may be between about 1 mg and 4,000 mg, between about 2,000 to 4,000 mg, between about 1 to 2,000 mg, between about 1 to 1,000 mg, between about 10 to 500 mg, between about 20 to 500 mg, between about 50 to 300 mg, between about 75 to 200 mg, or between about 15 to 150 mg.

Dosage of a compound of Formula II, or a pharmaceutically acceptable salt or pharmaceutically acceptable tautomer thereof, may be between about 1 mg and 4,000 mg, between about 2,000 to 4,000 mg, between about 1 to 2,000 mg, between about 1 to 1,000 mg, between about 10 to 500 mg, between about 20 to 500 mg, between about 50 to 300 mg, between about 75 to 200 mg, or between about 15 to 150 mg.

Dosage of a PGP inhibitor may be between about 1 mg and 4,000 mg, between about 2,000 to 4,000 mg, between about 1 to 2,000 mg, between about 1 to 1,000 mg, between about 1 to 500 mg, between about 10 to 500 mg, between about 20 to 500 mg, between about 1 to 300 mg, between about 10 to 300 mg, between about 20 to 300 mg, between about 1 to 200 mg, between about 10 to 200 mg, between about 20 to 200 mg, between about 1 to 100 mg, between about 10 to 100 mg, between about 20 to 100 mg, between about 50 to 300 mg, between about 75 to 200 mg, or between about 15 to 150 mg. For example, dosage of a PGP inhibitor may be about 15 mg.

The dosage or dosing frequency of a compound of the present disclosure may be adjusted over the course of the treatment, based on the judgment of the administering physician.

The compounds of the present disclosure may be administered to an individual (e.g., a human) in a therapeutically effective amount. In some embodiments, the compound is administered once daily. In some embodiments, the compound is administered once every week. In some embodiments, the compound is administered once every month. In some embodiments, the compound is administered every two months. In some embodiments, the compound is administered every three months. In some embodiments, the compound is administered every four months. In some embodiments, the compound is administered every five months. In some embodiments, the compound is administered every six months. In some embodiments, the compound is administered every seven months. In some embodiments, the compound is administered every eight months. In some embodiments, the compound is administered every nine months. In some embodiments, the compound is administered every ten months. In some embodiments, the compound is administered every eleven months. In some embodiments, the compound is administered every year.

The compounds provided herein can be administered by any useful route and means, such as by oral or parenteral (e.g., intravenous) administration. Therapeutically effective amounts of the compound may include from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day. In some embodiments, a therapeutically effective amount of the compounds provided herein include from about 0.3 mg to about 30 mg per dose, or from about 30 mg to about 300 mg per dose, or from about 0.3 μg to about 30 mg per dose, or from about 30 μg to about 300 μg per dose.

A compound of the present disclosure may be combined with one or more additional therapeutic agents in any dosage amount of the compound of the present disclosure (e.g., from 1 mg to 1000 mg of compound). Therapeutically effective amounts may include from about 0.1 mg per dose to about 1000 mg per dose, such as from about 50 mg per dose to about 500 mg per dose, or such as from about 100 mg per dose to about 400 mg per dose, or such as from about 150 mg per dose to about 350 mg per dose, or such as from about 200 mg per dose to about 300 mg per dose, or such as from about 0.01 mg per dose to about 1000 mg per dose, or such as from about 0.01 mg per dose to about 100 mg per dose, or such as from about 0.1 mg per dose to about 100 mg per dose, or such as from about 1 mg per dose to about 100 mg per dose, or such as from about 1 mg per dose to about 10 mg per dose, or such as from about 1 mg per dose to about 1000 mg per dose. Other therapeutically effective amounts of the compound of Formula I are about 1 mg per dose, or about 2, 3, 4, 5, 6, 7, 8, 9, 10, 15, 20, 25, 30, 35, 40, 45, 50, 55, 60, 65, 70, 75, 80, 85, 90, 95, or about 100 mg per dose. Other therapeutically effective amounts of the compound of the present disclosure are about 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or about 1000 mg per dose.

In some embodiments, the methods described herein comprise administering to the subject an initial daily dose of about 1 to 500 mg of a compound disclosed herein and increasing the dose by increments until clinical efficacy is achieved. Increments of about 5, 10, 25, 50, or 100 mg can be used to increase the dose. The dosage can be increased daily, every other day, twice per week, once per week, once every two weeks, once every three weeks, or once a month.

When administered orally, the total daily dosage for a human subject may be between about 1 mg and 1,000 mg, between about 10-500 mg/day, between about 50-300 mg/day, between about 75-200 mg/day, or between about 100-150 mg/day. In some embodiments, the total daily dosage for a human subject may be about 100, 200, 300, 400, 500, 600, 700, 800, 900, or 1000 mg/day administered in a single dose. In some embodiments, the total daily dosage for a human subject may be about 200, 300, 400, 500, 600, 700, or 800 mg/day administered in a single dose. In some embodiments, the total daily dosage for a human subject may be about 300, 400, 500, or 600 mg/day administered in a single dose.

In some embodiments, the total daily dosage for a human subject may be about 100 mg/day administered in a single dose. In some embodiments, the total daily dosage for a human subject may be about 150 mg/day administered in a single dose. In some embodiments, the total daily dosage for a human subject may be about 200 mg/day administered in a single dose. In some embodiments, the total daily dosage for a human subject may be about 250 mg/day administered in a single dose. In some embodiments, the total daily dosage for a human subject may be about 300 mg/day administered in a single dose. In some embodiments, the total daily dosage for a human subject may be about 350 mg/day administered in a single dose. In some embodiments, the total daily dosage for a human subject may be about 400 mg/day administered in a single dose. In some embodiments, the total daily dosage for a human subject may be about 450 mg/day administered in a single dose. In some embodiments, the total daily dosage for a human subject may be about 500 mg/day administered in a single dose. In some embodiments, the total daily dosage for a human subject may be about 550 mg/day administered in a single dose. In some embodiments, the total daily dosage for a human subject may be about 600 mg/day administered in a single dose. In some embodiments, the total daily dosage for a human subject may be about 650 mg/day administered in a single dose. In some embodiments, the total daily dosage for a human subject may be about 700 mg/day administered in a single dose. In some embodiments, the total daily dosage for a human subject may be about 750 mg/day administered in a single dose. In some embodiments, the total daily dosage for a human subject may be about 800 mg/day administered in a single dose. In some embodiments, the total daily dosage for a human subject may be about 850 mg/day administered in a single dose. In some embodiments, the total daily dosage for a human subject may be about 900 mg/day administered in a single dose. In some embodiments, the total daily dosage for a human subject may be about 950 mg/day administered in a single dose. In some embodiments, the total daily dosage for a human subject may be about 1000 mg/day administered in a single dose.

A single dose can be administered hourly, daily, weekly, or monthly. For example, a single dose can be administered once every 1 hour, 2, 3, 4, 6, 8, 12, 16 or once every 24 hours. A single dose can also be administered once every 1 day, 2, 3, 4, 5, 6, or once every 7 days. A single dose can also be administered once every 1 week, 2, 3, or once every 4 weeks. In certain embodiments, a single dose can be administered once every week. A single dose can also be administered once every month. In some embodiments, a compound disclosed herein is administered once daily in a method disclosed herein. In some embodiments, a compound disclosed herein is administered twice daily in a method disclosed herein.

In some embodiments, a compound disclosed herein is administered once every 10 days. In some embodiments, a compound disclosed herein is administered once every 15 days. In some embodiments, a compound disclosed herein is administered once every 20 days. In some embodiments, a compound disclosed herein is administered once every 10-15 days. In some embodiments, a compound disclosed herein is administered once every 15-20 days. In some embodiments, a compound disclosed herein is administered once every 10-20 days. In some embodiments, a compound disclosed herein is administered once every month. In some embodiments, a compound disclosed herein is administered once every 2 months. In some embodiments, a compound disclosed herein is administered once every 3 months. In some embodiments, a compound disclosed herein is administered once every 4 months. In some embodiments, a compound disclosed herein is administered once every 5 months. In some embodiments, a compound disclosed herein is administered once every 6 months. In some embodiments, a compound disclosed herein is administered once every 8 months. In some embodiments, a compound disclosed herein is administered once every 10 months. In some embodiments, a compound disclosed herein is administered once every year.

The frequency of dosage of the compound of the present disclosure will be determined by the needs of the individual patient and can be, for example, once per day, once per week, once per two weeks, once per month, once per two months, once per three months, once per four months, once per six months, or less. Administration of the compound continues for as long as necessary to treat the HIV infection.

### VII. Kits and Articles of Manufacture

In one aspect, provided herein are kits that comprise a compound provided herein, (e.g., a compound of Formula I), or a pharmaceutically acceptable salt, stereoisomer, prodrug, or solvate thereof, and suitable packaging. In some embodiments, the kit further comprises instructions for use. In some embodiments, the kit comprises a compound provided herein (e.g., a compound of Formula I), or a pharmaceutically acceptable salt, stereoisomer, prodrug, or solvate thereof, and a label and/or instructions for use of the compounds in the treatment of the indications, including the diseases or conditions, described herein.

In some embodiments, the kits further comprise one or more (e.g., one, two, three, four, one or two, one to three, or one to four) additional therapeutic agents, or a pharmaceutically acceptable salt thereof.

In one aspect, provided herein are articles of manufacture that comprise a compound described herein or pharmaceutically acceptable salts, isomer, or a mixture thereof in a suitable container. In some embodiments, the container may be a vial, jar, ampoule, preloaded syringe, or intravenous bag.

In one aspect, provided herein are kits that comprise (i) a compound of Formula I or Formula II provided herein, (e.g., a compound of Formula I), or a pharmaceutically acceptable salt, stereoisomer, prodrug, or solvate thereof, (ii) a P-glycoprotein inhibitor and suitable packaging. In some embodiments, the kit further comprises instructions for use. In some embodiments, the kit comprises (i) a compound of Formula I or Formula II provided herein (e.g., a compound of Formula I), or a pharmaceutically acceptable salt, stereoisomer, prodrug, or solvate thereof, (ii) a P-glycoprotein inhibitor, and a label and/or instructions for use of the compounds in the treatment of the indications, including the diseases or conditions, described herein.

In some embodiments, the kit comprises (i) a pharmaceutical composition comprising the compound of Formula I, or a pharmaceutically acceptable salt thereof, or the compound of Formula II, or a pharmaceutically acceptable salt thereof, (ii) a pharmaceutical composition comprising the P-glycoprotein inhibitor, and (iii) instructions for use.

In some embodiments, the kit comprises (i) a pharmaceutical composition comprising the compound of Formula I, or a pharmaceutically acceptable salt thereof, or the compound of Formula II, or a pharmaceutically acceptable salt thereof, and the P-glycoprotein inhibitor, and (ii) instructions for use.

In some embodiments, the kits further comprise one or more (e.g., one, two, three, four, one or two, one to three, or one to four) additional therapeutic agents, or a pharmaceutically acceptable salt thereof.

In one aspect, provided herein are articles of manufacture that comprise (i) a compound of Formula I or Formula II described herein or pharmaceutically acceptable salts, isomer, or a mixture thereof, and (ii) a P-glycoprotein inhibitor in a suitable container. In some embodiments, the container may be a vial, jar, ampoule, preloaded syringe, or intravenous bag.

### VII. Combination Therapy

In certain embodiments, a method for treating an HIV infection is provided, comprising administering to the human a therapeutically effective amount of a compound disclosed herein, or a pharmaceutically acceptable salt thereof, in combination with a therapeutically effective amount of one, two, three, or four additional therapeutic agents. In one embodiment, a method for treating an HIV infection is provided, comprising administering to the human a therapeutically effective amount of a compound disclosed herein, or a pharmaceutically acceptable salt thereof, in combination with a therapeutically effective amount of one, two, three, or four additional therapeutic agents.

In one embodiment, pharmaceutical compositions comprising a compound disclosed herein, or a pharmaceutically acceptable salt thereof, in combination with one, two, three, or four additional therapeutic agents, and a pharmaceutically acceptable carrier, diluent, or excipient are provided.

In certain embodiments, the present disclosure provides a method for treating an HIV infection, comprising administering to a subject in need thereof a therapeutically effective amount of a compound disclosed herein, or a pharmaceutically acceptable salt thereof, in combination with a therapeutically effective amount of one, two, three, or four additional therapeutic agents which are suitable for treating an HIV infection.

In certain embodiments, a compound disclosed herein, or a pharmaceutically acceptable salt thereof, is combined with one, two, three, four, or more additional therapeutic agents. In certain embodiments, a compound disclosed herein, or a pharmaceutically acceptable salt thereof, is combined with one, two, three, or four additional therapeutic agents. In certain embodiments, a compound disclosed herein, or a pharmaceutically acceptable salt thereof, is combined with two additional therapeutic agents. In other embodiments, a compound disclosed herein, or a pharmaceutically acceptable salt thereof, is combined with three additional therapeutic agents. In further embodiments, a compound disclosed herein, or a pharmaceutically acceptable salt thereof, is combined with four additional therapeutic agents. The one, two, three, four, or more additional therapeutic agents can be different therapeutic agents selected from the same class of therapeutic agents, and/or they can be selected from different classes of therapeutic agents.

### Administration of HIV Combination Therapy

In certain embodiments, a compound disclosed herein is administered with one, two, three, or four additional therapeutic agents. Co-administration of a compound disclosed herein with one, two, three, or four additional therapeutic agents generally refers to simultaneous or sequential administration of a compound disclosed herein and one, two, three, or four additional therapeutic agents, such that therapeutically effective amounts of the compound disclosed herein and the one, two, three, or four additional therapeutic agents are both present in the body of the patient. When administered sequentially, the combination may be administered in two or more administrations.

Co-administration includes administration of unit dosages of the compounds disclosed herein before or after administration of unit dosages of one, two, three, or four additional therapeutic agents. For example, the compound disclosed herein may be administered within seconds, minutes, or hours of the administration of the one, two, three, or four additional therapeutic agents. In some embodiments, a unit dose of a compound disclosed herein is administered first, followed within seconds or minutes by administration of a unit dose of one, two, three, or four additional therapeutic agents. Alternatively, a unit dose of one, two, three, or four additional therapeutic agents is administered first, followed by administration of a unit dose of a compound disclosed herein within seconds or minutes. In other embodiments, a unit dose of a compound disclosed herein is administered first, followed, after a period of hours (e.g., 1-12 hours), by administration of a unit dose of one, two, three, or four additional therapeutic agents. In yet other embodiments, a unit dose of one, two, three, or four additional therapeutic agents is administered first, followed, after a period of hours (e.g., 1-12 hours), by administration of a unit dose of a compound disclosed herein.

In certain embodiments, a kit comprising a compound disclosed herein (e.g., a compound of Formula I, or a pharmaceutically acceptable salt thereof, and one or more (e.g., one, two, three, or four) additional therapeutic agents is provided.

In a specific embodiment, the kit includes a compound disclosed herein, or a pharmaceutically acceptable salt thereof, an HIV nucleoside or nucleotide inhibitor of reverse transcriptase and an HIV capsid inhibitor or an HIV capsid polymerization inhibitor.

### HIV Combination Therapy

In the above embodiments, the additional therapeutic agent or agents may be an anti-HIV agent. In some instances, the additional therapeutic agent can be HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors. HIV entry inhibitors. HIV maturation inhibitors, HIV capsid inhibitors, nucleocapsid protein 7 (NCp7) inhibitors. HIV Tat or Rev inhibitors, inhibitors of Tat-TAR-P-TEFb, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell. CAR-T, and engineered T-cell receptors, TCR-T, autologous T-cell therapies, engineered B cells, NK cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists. HIV-1 viral infectivity factor inhibitors, HIV-1 Nef modulators, TNF alpha ligand inhibitors, HIV Nef inhibitors, Hck tyrosine kinase modulators, mixed lineage kinase-3 (MLK-3) inhibitors, HIV-1 splicing inhibitors, integrin antagonists, nucleoprotein inhibitors, splicing factor modulators. COMM domain containing protein 1 modulators, HIV ribonuclease H inhibitors, IFN antagonists, retrocyclin modulators, CD3 antagonists, CDK-4 inhibitors, CDK-6 inhibitors, CDK-9 inhibitors, Cytochrome P450 3 inhibitors, CXCR4 modulators, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, HPK1 (MAP4K1) inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, RNA polymerase modulators, TAT protein inhibitors. Prolyl endopeptidase inhibitors, Phospholipase A2 inhibitors, pharmacokinetic enhancers, HIV gene therapy, HIV vaccines, anti-HIV peptides, and combinations thereof.

In some embodiments, the additional therapeutic agent or agents are selected from combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies. PI3K inhibitors, HIV antibodies, and bispecific antibodies, and “antibody-like” therapeutic proteins, and combinations thereof.

In some embodiments, the additional therapeutic agent is selected from the group consisting of combination drugs for HIV, other drugs for treating HIV. HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent or agents are chosen from HIV protease inhibitors, HIV non-nucleoside or non-nucleotide inhibitors of reverse transcriptase, HIV nucleoside or nucleotide inhibitors of reverse transcriptase. HIV integrase inhibitors. HIV capsid inhibitors, gp41 inhibitors, CXCR4 inhibitors, gp120 inhibitors, CCR5 inhibitors, Nef inhibitors, latency reversing agents, HIV bNAbs, agonists of TLR7. TLR8, and TLR9, HIV vaccines, cytokines, immune checkpoint inhibitors, FLT3 ligands, T cell and NK cell recruiting bispecific antibodies, chimeric T cell receptors targeting HIV antigens, pharmacokinetic enhancers, and other drugs for treating HIV, and combinations thereof.

In some embodiments, the additional therapeutic agent or agents are chosen from dolutegravir, cabotegravir, islatravir, darunavir, bictegravir, elsulfavirine, rilpivirine, and lenacapavir, and combinations thereof.

### HIV Combination Drugs

Examples of combination drugs include, but are not limited to, ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); darunavir, tenofovir alafenamide hemifumarate, emtricitabine, and cobicistat; efavirenz, lamivudine, and tenofovir disoproxil fumarate; lamivudine and tenofovir disoproxil fumarate; tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; tenofovir analog; COMBIVIR® (zidovudine and lamivudine; AZT+3TC); EPZICOM® (LIVEXA®; abacavir sulfate and lamivudine; ABC+3TC); KALETRA® (ALUVIA®; lopinavir and ritonavir); TRIUMEQ® (dolutegravir, abacavir, and lamivudine); BIKTARVY® (bictegravir+emtricitabine+tenofovir alafenamide), DOVATO® (dolutegravir+lamivudine), TRIZIVIR® (abacavir sulfate, zidovudine, and lanivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dolutegravir+lamivudine, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, and tenofovir disoproxil fumarate+emtricitabine+rilpivirine hydrochloride, lopinavir, ritonavir, zidovudine, lopinavir+ritonavir+abacavir+lamivudine, lamivudine, cabotegravir+rilpivirine, 3-BNC117+albuvirtide, elpida (elsulfavirine, VM-1500), and VM-1500A, lenacapavir+islatravir (oral, injectable), and dual-target HIV-1 reverse transcriptase/nucleocapsid protein 7 inhibitors.

### Other HIV Drugs

Examples of other drugs for treating HIV include, but are not limited to, aspernigrin C, acemannan, alisporivir, BanLec, deferiprone, Gamimune, metenkefalin, naltrexone, Prolastin, REP 9, RPI-MN, VSSP, H1viral, SB-728-T, 1,5-dicaffeoylquinic acid, rHIV7-shl-TAR-CCR5RZ. AAV-eCD4-Ig gene therapy, MazF gene therapy, BlockAide, bevirimat derivatives, ABBV-382, ABX-464, AG-1105, APH-0812, APH0202, bryostatin-1, bryostatin analogs, BIT-225, BRII-732, BRII-778, CYT-107, CS-TATI-1, fluoro-beta-D-arabinose nucleic acid (FANA)-modified antisense oligonucleotides, FX-101, griffithsin, GSK-3739937, GSK-3739937 (long-acting), HGTV-43, HPH-116, HS-10234, hydroxychloroquine, IMB-10035, IMO-3100, IND-02, JL-18008, LADAVRU, MK-1376, MK-2048, MK-4250, MK-8507, MK-8558, MK-8591 (islatravir), NOV-205, OB-002H, ODE-Bn-TFV, PA-1050040 (PA-040), PC-707, PGN-007, QF-036, 5-648414, SCY-635, SB-9200, SCB-719, TR-452, TEV-90110, TEV-90112, TEV-90111, TEV-90113, RN-18, DIACC-1010, Fasnall, Immuglo, 2-CLIPS peptide, HRF-4467, thrombospondin analogs, TBL-1004HI, VG-1177, xl-081, AVI-CO-004, rfhSP-D, [18F]-MC-225, URMC-099-C, RES-529, Verdinexor, IMC-M113V, IML-106, antiviral fc conjugate (AVC), WP-1096, WP-1097, Gammora, ISR-CO48, ISR-48, ISR-49, MK-8527, cannabinoids, ENOB-HV-32, HiviCide-I, T-1144, VIR-576, nipamovir, Covimro, and ABBV-1882.

### HIV Protease Inhibitors

Examples of HIV protease inhibitors include, but are not limited to, amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, ASC-09+ritonavir, AEBL-2, DG-17, GS-1156, TMB-657 (PPL-100), T-169, BL-008, MK-8122, TMB-607, GRL-02031, and TMC-310911.

Additional examples of HIV protease inhibitors are described, e.g., in U.S. Pat. No. 10,294,234, and U.S. Patent Application Publication Nos. US2020030327 and US2019210978.

### HIV Gag Protein Inhibitors

Examples of HIV Gag protein inhibitors include, but are not limited to, HRF-10071.

### HIV Ribonuclease H Inhibitors

Examples of HIV ribonuclease H inhibitors include, but are not limited to, NSC-727447.

### HIV Nef Inhibitors

Examples of HIV Nef inhibitors include, but are not limited to, FP-1.

### HIV Reverse Transcriptase Inhibitors

Examples of HIV non-nucleoside or non-nucleotide inhibitors of reverse transcriptase include, but are not limited to, dapivirine, delavirdine, delavirdine mesylate, doravirine, efavirenz, etravirine, lentinan, nevirapine, rilpivirine, ACC-007, ACC-008, AIC-292, F-18, KM-023, PC-1005, M1-TFV, M2-TFV, VM-1500A-LAI, PF-3450074, elsulfavirine (sustained release oral. HIV infection), doravirine+islatravir (fixed dose combination/oral tablet formulation, HIV-1 infection), elsulfavirine (long acting injectable nanosuspension, HIV infection), and elsulfavirine (VM-1500).

Examples of HIV nucleoside or nucleotide inhibitors of reverse transcriptase include, but are not limited to, adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir octadecyloxyethyl ester (AGX-1009), tenofovir disoproxil hemifumarate, VIDEX® and VIDEX EC9® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, lamivudine, phosphazid, stavudine, zalcitabine, zidovudine, rovafovir etalafenamide (GS-9131), GS-9148, MK-8504, islatravir, MK-8583, VM-2500, and KP-1461.

Additional examples of HIV nucleoside or nucleotide inhibitors of reverse transcriptase include, but are not limited to, those described in patent publications US2007049754, US2016250215, US2016237062, US2016251347, US2002119443, US2013065856, US2013090473, US2014221356, and WO04096286.

### HIV Integrase Inhibitors

Examples of HIV integrase inhibitors include, but are not limited to, elvitegravir, elvitegravir (extended-release microcapsules), curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, PEGylated raltegravir, dolutegravir, JTK-351, bictegravir, AVX-15567, cabotegravir (long acting injectable), diketo quinolin-4-1 derivatives, integrase-LEDGF inhibitor, ledgins, M-522, M-532, MK-0536, NSC-310217, NSC-371056, NSC-48240, NSC-642710, NSC-699171, NSC-699172, NSC-699173, NSC-699174, stilbenedisulfonic acid, T169, STP-0404, VM-3500, XVIR-110, and ACC-017.

Examples of HIV non-catalytic site, or allosteric, integrase inhibitors (NCINI) include, but are not limited to, CX-05045, CX-05168, and CX-14442.

Additional examples of HIV capsid inhibitors include, but are not limited to, those described in U.S. Patent Application Publication Nos. US20200317689, US20210284642, US2014221356 and US2016016973.

### HIV Viral Infectivity Factor Inhibitors

Examples of HIV viral infectivity factor inhibitors include, but are not limited to, 2-amino-N-(2-methoxyphenyl)-6-((4-nitrophenyl)thio)benzamide derivatives, and Irino-L.

### HIV Entry Inhibitors

Examples of HIV entry (fusion) inhibitors include, but are not limited to, AAR-501, LBT-5001, cenicriviroc, CCR5 inhibitors, gp41 inhibitors, CD4 attachment inhibitors, gp120 inhibitors, gp160 inhibitors, and CXCR4 inhibitors.

Examples of CCR5 inhibitors include, but are not limited to, aplaviroc, vicriviroc, maraviroc, maraviroc (long acting injectable nanoemulsion), cenicriviroc, leronlimab (PRO-140), adaptavir (RAP-10l), nifeviroc (TD-0232), anti-GP120/CD4 or CCR5 bispecific antibodies, B-07, MB-66, polypeptide C25P. TD-0680, thioraviroc and vMIP (Haimipu).

Examples of gp41 inhibitors include, but are not limited to, albuvirtide, enfuvirtide, griffithsin (gp41/gp120/gp160 inhibitor), BMS-986197, enfuvirtide biobetter, enfuvirtide biosimilar, HIV-1 fusion inhibitors (P26-Bapc), ITV-1, ITV-2, ITV-3, ITV-4, CPT-31, C13hmAb, lipuvirtide, PIE-12 trimer and sifuvirtide.

Examples of CD4 attachment inhibitors include, but are not limited to, ibalizumab and CADA analogs.

Examples of gp120 inhibitors include, but are not limited to, anti-HIV microbicide, Radha-108 (receptol) 3B3-PE38, BMS818251, BanLec, bentonite-based nanomedicine, fostemsavir tromethamine, IQP-0831, VVX-004, and BMS-663068.

Examples of gp160 inhibitors include, but are not limited to, fangchinoline.

Examples of CXCR4 inhibitors include, but are not limited to, plerixafor, ALT-1188, N15 peptide, and vMIP (Haimipu).

### HIV Maturation Inhibitors

Examples of HIV maturation inhibitors include, but are not limited to, BMS-955176, GSK-3640254 and GSK-2838232.

### Latency Reversing Agents

Examples of latency reversing agents include, but are not limited to, toll-like receptor (TLR) agonists (including TLR7 agonists, e.g., GS-9620, TLR8 agonists, and TLR9 agonists), histone deacetylase (HDAC) inhibitors, proteasome inhibitors such as velcade, protein kinase C (PKC) activators. Smyd2 inhibitors, BET-bromodomain 4 (BRD4) inhibitors (such as ZL-0580, apabetalone), ionomycin, IAP antagonists (inhibitor of apoptosis proteins, such as APG-1387, LBW-242), SMAC mimetics (including TL32711, LCL 161, GDC-0917, HGS1029, AT-406, Debio-1143), PMA, SAHA (suberanilohydroxamic acid, or suberoyl, anilide, and hydroxamic acid), NIZ-985, IL-15 modulating antibodies (including IL-15, IL-15 fusion proteins, and IL-15 receptor agonists), JQ1, disulfiram, amphotericin B, and ubiquitin inhibitors such as largazole analogs, APH-0812, and GSK-343. Examples of PKC activators include, but are not limited to, indolactam, prostratin, ingenol B. and DAG-lactones.

Additional examples of TLR7 agonists include, but are not limited to, those described in U.S. Patent Application Publication No. US2010143301.

Additional examples of TLR8 agonists include, but are not limited to, those described in U.S. Patent Application Publication No. US2017071944.

### Histone Deacetylase (HDAC) Inhibitors

In some embodiments, the agents as described herein are combined with an inhibitor of a histone deacetylase, e.g., histone deacetylase 1, histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include without limitation, abexinostat, ACY-241, AR-42, BEBT-908, belinostat, CKD-581, CS-055 (HBI-8000), CT-101, CUDC-907 (fimepinostat), entinostat, givinostat, mocetinostat, panobinostat, pracinostat, quisinostat (JNJ-26481585), resminostat, ricolinostat, romidepsin, SHP-141, TMB-ADC, valproic acid (VAL-001), vorinostat, tinostamustine, remetinostat, and entinostat.

### Capsid Inhibitors

Examples of capsid inhibitors include, but are not limited to, capsid polymerization inhibitors or capsid disrupting compounds, HIV nucleocapsid p7 (NCp7) inhibitors such as azodicarbonamide, HIV p24 capsid protein inhibitors, lenacapavir (GS-6207) and prodrugs thereof, GS-CA1, AVI-621, AVI-101, AVI-201, AVI-301, and AVI-CAN1-15 series, PF-3450074, HIV-1 capsid inhibitors (HIV-1 infection, Shandong University), and compounds described in (GSK WO2019/087016).

Additional examples of capsid inhibitors include, but not limited to, those described in U.S. Patent Application Publication Nos. US2018051005 and US2016108030.

### Cytochrome P450 3 Inhibitors

Examples of Cytochrome P450 3 inhibitors include, but are not limited to, those described in U.S. Pat. No. 7,939,553.

### RNA Polymerase Modulators

Examples of RNA polymerase modulators include, but are not limited to, those described in U.S. Pat. Nos. 10,065,958 and 8,008,264.

### Immune Checkpoint Modulators

In various embodiments, the agents as described herein, are combined with one or more blockers or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of infected cells. Activation or stimulation of stimulatory immune check points can augment the effect of immune checkpoint inhibitors in infective therapeutics. In various embodiments, the immune checkpoint proteins or receptors regulate T cell responses (e.g., reviewed in Xu et al., J Exp Clin Cancer Res. (2018) 37:110). In various embodiments, the immune checkpoint proteins or receptors regulate NK cell responses (e.g., reviewed in Davis et al., Semin Immunol. (2017) 31:64-75 and Chiossone et al., Nat Rev Immunol. (2018) 18(11):671-688).

Examples of immune checkpoint proteins or receptors include without limitation CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)), TNFRSF17 (BCMA, CD269), TNFSF13B (BAFF); TNFRSF18 (GITR), TNFSF18 (GITRL); MI-IC class I poly peptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CD112R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL 16 binding protein 1 (ULBP1); UL16 binding protein 2 (ULBP2); UL16 binding protein 3 (ULBP3); retinoic acid early transcript 1E (RAET1E; ULBP4); retinoic acid early transcript IG (RAET1G; ULBP5); retinoic acid early transcript IL (RAET1L; ULBP6); lymphocyte activating 3 (CD223); killer cell immunoglobulin like receptor, three Ig domains and long cytoplasmic tail 1 (KIR, CD158E1); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DLI); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1); SLAM family member 7 (SLAMF7); and Hematopoietic Progenitor Kinase 1 (HPK1, MAP4K1).

In various embodiments, the agents described herein are combined with one or more blockers or inhibitors of one or more T-cell inhibitory immune checkpoint proteins or receptors. Illustrative T-cell inhibitory immune checkpoint proteins or receptors include without limitation CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CDI 12R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DLI); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In various embodiments, the agents, as described herein, are combined with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR). TNFSF18 (GITRL); CD80 (B7-1), CD28; nectin cell adhesion molecule 2 (NECTIN2, CD 112); CD226 (DNAM-1); CD244 (2B4, SLAMF4), Poliovirus receptor (PVR) cell adhesion molecule (PVR, CD155). Se, e.g., Xu et al., J Exp Clin Cancer Res. (2018) 37:110.

In various embodiments, the agents as described herein, are combined with one or more blockers or inhibitors of one or more NK-cell inhibitory immune checkpoint proteins or receptors. Illustrative NK-cell inhibitory immune checkpoint proteins or receptors include without limitation killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1), killer cell lectin like receptor C1 (KLRC1. NKG2A, CD159A); and killer cell lectin like receptor D1 (KLRD1, CD94). In various embodiments, the agents as described herein, are combined with one or more agonist or activators of one or more NK-cell stimulatory immune checkpoint proteins or receptors. Illustrative NK-cell stimulatory immune checkpoint proteins or receptors include without limitation CD16, CD226 (DNAM-1); CD244 (2B4, SLAMF4); killer cell lectin like receptor K1 (KLRK1, NKG2D, CD314); SLAM family member 7 (SLAMF7). See, e.g., Davis et al., Semin Immunol. (2017) 31:64-75; Fang et al., Semin Immunol. (2017) 31:37-54; and Chiossone et al., Nat Rev Immunol. (2018) 18(11):671-688.

In some embodiments, the one or more immune checkpoint inhibitors comprises a proteinaceous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4. In some embodiments, the small molecule inhibitor of CD274 or PDCD1 is selected from the group consisting of GS-4224, GS-4416. INCB086550 and MAX10181. In some embodiments, the small molecule inhibitor of CTLA4 comprises BPI-002.

Examples of inhibitors of CTLA4 that can be co-administered include without limitation ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BPI-002, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors of PD-L1 (CD274) or PD-1 (PDCD1) that can be co-administered include without limitation pembrolizumab, nivolumab, cemiplimab, pidilizumab, AMP-224. MED10680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034, JS-001 (toripalimab), JNJ-63723283, genolimzumab (CBT-501), LZM-009, BCD-100, LY-3300054, SHR-1201, SHR-1210 (camrelizumab), Sym-021, ABBV-181 (budigalimab), PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, GS-4224, GS-4416, INCB086550, MAX10181, as well as multi-specific inhibitors FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM3/PDL1), and INBRX-105 (4-1BB/PDL1).

In various embodiments, the agents as described herein are combined with anti-TIGIT antibodies, such as BMS-986207, RG-6058, and AGEN-1307.

### TNF Receptor Superfamily (TNFRSF) Member Agonists or Activators

In various embodiments, the agents as described herein are combined with an agonist of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40, NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF1IA (CD265, RANK, NCBI Gene ID: 8792), TNFRSF11B (NCBI Gene ID: 4982), TNFRSF12A (CD266. NCBI Gene ID: 51330), TNFRSF13B (CD267, NCBI Gene ID: 23495). TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504). TNFRSF21 (CD358, DR6, NCBI Gene ID: 27242), and TNFRSF25 (DR3, NCBI Gene ID: 8718).

Examples of anti-TNFRSF4 (OX40) antibodies that can be co-administered include without limitation, MED16469, MED16383, MEDI0562 (tavolixizumab), MOXR0916, PF-04518600, RG-7888, GSK-3174998, INCAGN1949. BMS-986178, GBR-8383, ABBV-368, and those described in WO2016179517, WO20170%179, WO20170%182, WO20170%281, and WO2018089628.

Examples of anti-TNFRSF5 (CD40) antibodies that can be co-administered include without limitation RG7876, SEA-CD40. APX-005M and ABBV-428.

In some embodiments, the anti-TNFRSF7 (CD27) antibody varlilumab (CDX-1127) is co-administered.

Examples of anti-TNFRSF9 (4-1BB, CD137) antibodies that can be co-administered include without limitation urelumab, utomilumab (PF-05082566), AGEN2373 and ADG-106.

Examples of anti-TNFRSF18 (GITR) antibodies that can be co-administered include without limitation, MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089628.

### Bi- and Tri-Specific Natural Killer (NK)-Cell Engagers

In various embodiments, the agents as described herein, are combined with a bi-specific NK-cell engager (BiKE) or a tri-specific NK-cell engager (TriKE) (e.g., not having an Fc) or bi-specific antibody (e.g., having an Fc) against an NK cell activating receptor, e.g., CD16A. C-type lectin receptors (CD94/NKG2C, NKG2D, NKG2E/H and NKG2F), natural cytotoxicity receptors (NKp30, NKp44 and NKp46), killer cell C-type lectin-like receptor (NKp65, NKp80), Fc receptor FcγR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (41BB). As appropriate, the anti-CD16 binding bi-specific molecules may or may not have an Fc. Illustrative bi-specific NK-cell engagers that can be co-administered target CD16 and one or more HIV-associated antigens as described herein. BiKEs and TriKEs are described, e.g., in Felices et al., Methods Mol Biol. (2016) 1441:333-346; Fang et al., Semin Immunol. (2017) 31:37-54. Examples of trispecific NK cell engagers (TRiKE) include, but are not limited to, OXS-3550, HIV-TriKE, and CD16-IL-15-B7H3 TriKe.

Indoleamine-pyrrole-2,3-dioxygenase (IDO1) Inhibitors

In various embodiments, the agents as described herein are combined with an inhibitor of indoleamine 2,3-dioxygenase 1 (IDO1; NCBI Gene ID: 3620). Examples of IDO1 inhibitors include without limitation, BLV-0801, epacadostat, F-001287, GBV-1012, GBV-1028, GDC-0919, indoximod, NKTR-218, NLG-919-based vaccine, PF-06840003, pyranonaphthoquinone derivatives (SN-35837), resminostat, SBLK-200802, BMS-986205, shIDO-ST, EOS-200271, KHK-2455, and LY-3381916.

### Toll-Like Receptor (TLR) Agonists

In various embodiments, the agents as described herein are combined with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333). TLR7 (NCBI Gene ID: 51284). TLR8 (NCBI Gene ID: 51311), TLR9 (NCBI Gene ID: 54106), and/or TLR 10 (NCBI Gene ID: 81793). Example TLR7 agonists that can be co-administered include without limitation AL-034, DSP-0509, GS-9620 (vesatolimod), vesatolimod analog, LHC-165, TMX-101 (imiquimod), GSK-2245035, resiquimod, DSR-6434, DSP-3025, IMO-4200, MCT-465, MEDI-9197, 3M-051. SB-9922, 3M-052, Limtop, TMX-30X, TMX-202. RG-7863, RG-7854, RG-7795, and the compounds disclosed in US20100143301 (Gilead Sciences), US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen). US20140350031 (Janssen). WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). TLR7/TLR8 agonists include without limitation NKTR-262, telratolimod and BDB-001. TLR8 agonists include without limitation E-6887, IMO-4200, IMO-8400, IMO-9200. MCT-465, MEDI-9197, motolimod, resiquimod, GS-9688, VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma). US20140088085 (Ventirx Pharma), US20140275167 (Novira Therapeutics), and US20130251673 (Novira Therapeutics). TLR9 agonists include without limitation AST-008, cobitolimod, CMP-001, IMO-2055, IMO-2125, S-540956, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. Examples of TLR3 agonist include rintatolimod, poly-ICLC, RIBOXXON®, Apoxxim, RIBOXXIM®, IPH-33, MCT-465, MCT-475, and ND-1.1. TLR4 agonists include, but are not limited to, G-100 and GSK-1795091.

### CDK Inhibitors or Antagonists

In some embodiments, the agents described herein are combined with an inhibitor or antagonist of CDK. In some embodiments, the CDK inhibitor or antagonist is selected from the group consisting of VS2-370.

### STING Agonists, RIG-I and NOD2 Modulators

In some embodiments, the agents described herein are combined with a stimulator of interferon genes (STING). In some embodiments, the STING receptor agonist or activator is selected from the group consisting of ADU-S100 (MIW-815), SB-11285, MK-1454, SR-8291, AdVCA0848, GSK-532, SYN-STING, MSA-1, SR-8291, STING agonist (latent HIV), 5,6-dimethylxanthenone-4-acetic acid (DMXAA), cyclic-GAMP (cGAMP) and cyclic-di-AMP. In some embodiments, the agents described herein are combined with a RIG-I modulator such as RGT-100, or NOD2 modulator, such as SB-9200, and IR-103.

### LAG-3 and TIM-3 Inhibitors

In certain embodiments, the agents as described herein are combined with an anti-TIM-3 antibody, such as TSR-022, LY-3321367, MBG-453, INCAGN-2390.

In certain embodiments, the antibodies or antigen-binding fragments described herein are combined with an anti LAG-3 (Lymphocyte-activation) antibody, such as relatlimab (ONO-4482), LAG-525, MK-4280, REGN-3767, INCAGN2385.

### Interleukin Agonists

In certain embodiments, the agents described herein are combined with an interleukin agonist, such as IL-2, IL-7, IL-15, IL-10, IL-12 agonists; examples of IL-2 agonists such as proleukin (aldesleukin, IL-2); BC-IL (Cel-Sci), pegylated IL-2 (e.g., NKTR-214); modified variants of IL-2 (e.g., THOR-707), bempegaldesleukin, AIC-284, ALKS-4230, CUI-101, Neo-2/15; examples of IL-15 agonists, such as ALT-803, NKTR-255, and hetIL-15, interleukin-15/Fc fusion protein, AM-0015, NIZ-985, SO—C101, IL-15 Synthorin (pegylated 11-15), P-22339, and a IL-15-PD-1 fusion protein N-809; examples of IL-7 include without limitation CYT-107.

Examples of additional immune-based therapies that can be combined with an agent of this disclosure include, but are not limited to, interferon alfa, interferon alfa-2b, interferon alfa-n3, pegylated interferon alfa, interferon gamma, FLT3 agonists such as CDX-301, GS-3583, gepon, normferon, peginterferon alfa-2a, peginterferon alfa-2b, and RPI-MN.

### Phosphatidylinositol 3-Kinase (PI3K) Inhibitors

Examples of PI3K inhibitors include, but are not limited to, idelalisib, alpelisib, buparlisib. CAT orotate, copanlisib, duvelisib, gedatolisib, neratinib, panulisib, perifosine, pictilisib, pilaralisib, puquitinib mesylate, rigosertib, rigosertib sodium, sonolisib, taselisib, AMG-319, AZD-8186, BAY-1082439, CLR-1401, CLR-457, CUDC-907, DS-7423, EN-3342, GSK-2126458, GSK-2269577, GSK-2636771, INCB-040093, LY-3023414, MLN-1117, PQR-309, RG-7666, RP-6530, RV-1729, SAR-245409, SAR-260301, SF-1126, TGR-1202, UCB-5857, VS-5584, XL-765, and ZSTK-474.

### Alpha-4/Beta-7 Antagonists

Examples of Integrin alpha-4/beta-7 antagonists include, but are not limited to, PTG-100, TRK-170, abrilumab, etrolizumab, carotegrast methyl, and vedolizumab.

### HPK1 Inhibitors

Examples of HPK1 inhibitors include, but are not limited to, ZYF-0272, and ZYF-0057.

### HIV Targeting Antibodies

Examples of HIV antibodies, bispecific antibodies, and “antibody-like” therapeutic proteins include, but are not limited to, DARTs®, DUOBODIES®, BITES®, XmAbs®, TandAbs®, Fab derivatives, bNAbs (broadly neutralizing HIV-1 antibodies), TMB-360, TMB-370, and those targeting HIV gp120 or gp41, antibody-Recruiting Molecules targeting HIV, anti-CD63 monoclonal antibodies, anti-GB virus C antibodies, anti-GP120/CD4, gp120 bispecific monoclonal antibody, CCR5 bispecific antibodies, anti-Nef single domain antibodies, anti-Rev antibody, camelid derived anti-CD18 antibodies, camelid-derived anti-ICAM-1 antibodies, DCVax-001, gp140 targeted antibodies, gp41-based HIV therapeutic antibodies, human recombinant mAbs (PGT-121), PGT121.414.LS, ibalizumab, ibalizumab (second generation), Immuglo. MB-66, clone 3 human monoclonal antibody targeting KLIC (HIV infection), GS-9721, BG-HIV, VRC-HIVMAB091-00-AB.

Various bNAbs may be used. Examples include, but are not limited to, those described in U.S. Pat. Nos. 8,673,307, 9,493,549, 9,783,594, 10,239,935, US2018371086, US2020223907, WO2014/063059, WO2012/158948, WO2015/117008, and PCT/US2015/41272, and WO2017/096221, including antibodies 12A12, 12A21, NIH45-46, bANC131, 8ANC134, 1B2530, INC9, 8ANC195, 8ANC 196, 10-259, 10-303, 10-410, 10-847, 10-996, 10-1074, 10-1121, 10-1130, 10-1146, 10-1341, 10-1369, and 10-1074GM. Additional examples include those described in Klein et al., Nature, 492(7427): 118-22 (2012), Horwitz et al., Proc Natl Acad Sci USA, 110(41): 16538-43 (2013), Scheid et al., Science, 333: 1633-1637 (2011), Scheid et al., Nature, 458:636-640 (2009), Eroshkin et al, Nucleic Acids Res., 42 (Database issue):D1 133-9 (2014). Mascola et al., Immunol Rev., 254(1):225-44 (2013), such as 2F5, 4E10, M66.6, CAP206-CH12, 10E81 (all of which bind the MPER of gp41); PG9, PG16, CH01-04 (all of which bind V1V2-glycan), 2G12 (which binds to outer domain glycan); b12, HJ16, CH₁₀₃-106, VRC01-03, VRC-PG04, 04b, VRC-CH₃O-34, 3BNC62, 3BNC89, 3BNC91, 3BNC95, 3BNC104, 3BNC176, and 8ANCl 31 (all of which bind to the CD4 binding site).

Additional broadly neutralizing antibodies that can be used as a second therapeutic agent in a combination therapy are described, e.g., in U.S. Pat. Nos. 8,673,307; 9,493,549; 9,783,594; and WO 2012/154312; WO2012/158948; WO 2013/086533; WO 2013/142324; WO2014/063059; WO 2014/089152, WO 2015/048462: WO 2015/103549; WO 2015/117008; WO2016/014484; WO 2016/154003: WO 2016/196975; WO 2016/149710; WO2017/096221; WO 2017/133639; WO 2017/133640, which are hereby incorporated herein by reference in their entireties for all purposes. Additional examples include, but are not limited to, those described in Sajadi et al., Cell. (2018) 173(7):1783-1795; Sajadi et al., J Infect Dis. (2016) 213(1):156-64; Klein et al., Nature, 492(7427): 118-22 (2012), Horwitz et al., Proc Natl Acad Sci USA, 110(41): 16538-43 (2013), Scheid et al., Science, 333: 1633-1637 (2011), Scheid et al., Nature, 458:636-640 (2009), Eroshkin et al., Nucleic Acids Res., 42 (Database issue):D1 133-9 (2014), Mascola et al., Immunol Rev., 254(1):225-44 (2013), such as 2F5, 4E10, M66.6, CAP206-CH12, 10E8, 10E8v4, 10E8-5R-100cF, DH511.11P, 7b2, 10-1074, and LN01 (all of which bind the MPER of gp41).

Examples of additional antibodies include, but are not limited to, bavituximab, UB-421, BF520.1. BiIA-SG, CH₀₁, CH₅₉, C2F5, C4E10, C2F5+C2G12+C4E10, CAP256V2LS, 3BNC117, 3BNC117-LS, 3BNC60, DH270.1, DH270.6, DID2, 10-1074-LS, C13hmAb, GS-9722 (elipovimab), DH411-2, BG18, GS-9721, GS-9723, PGT145, PGT121, PGT-121.60, PGT-121.66, PGT122, PGT-123, PGT-124, PGT-125, PGT-126, PGT-151, PGT-130, PGT-133, PGT-134, PGT-135, PGT-128, PGT-136, PGT-137, PGT-138, PGT-139, MDX010 (ipilimumab), DH511, DH511-2, N6, N6LS, N49P6, N49P7, N49P7.1, N49P9, N49P11, N60P1.1, N60P25.1, N60P2.1, N60P31.1. N60P22, NIH 45-46, PGC14, PGG14, PGT-142, PGT-143, PGT-144, PGDM1400, PGDM12, PGDM21, PCDN-33A, 2Dm2m, 4Dm2m, 6Dm2m, PGDM1400, MDX010 (ipilimumab), VRC01, VRC-01-LS, A32, 7B2, 10E8, VRC-07-523, VRC07-523LS, VRC24, VRC41.01, 10E8VLS, 3810109, 10E8v4, IMC-HIV, iMabm36, eCD4-Ig, IOMA, CAP256-VRC26.25, DRVIA7, VRC-HIVMAB080-00-AB, VRC-HIVMAB060-00-AB, P2G12, VRC07, 354BG8, 354BG18, 354BG42, 354BG33, 354BG129, 354BG188, 354BG411, 354BG426, VRC29.03, CAP256, CAP256-VRC26.08, CAP256-VRC26.09, CAP256-VRC26.25, PCT64-24E and VRC38.01, PGT-151, CAP248-2B, 35022, ACS202, VRC34 and VRC34.01, 10E8, 10E8v4, 10E8-5R-100cF, 4E10, DH511.1IP, 2F5, 7b2, and LNO1.

Examples of HIV bispecific and trispecific antibodies include without limitation MGD014, B12BiTe, BiIA-SG. TMB-bispecific, SAR-441236, VRC-01/PGDM-1400/10E8v4, 10E8.4/iMab, 10E8v4/PGT121-VRC01.

Examples of in vivo delivered bNAbs include without limitation AAV8-VRC07; mRNA encoding anti-HIV antibody VRC01; and engineered B-cells encoding 3BNC117 (Hartweger et al., J. Exp. Med. 2019, 1301).

### Pharmacokinetic Enhancers

Examples of pharmacokinetic enhancers include, but are not limited to, cobicistat and ritonavir.

### Additional Therapeutic Agents

Examples of additional therapeutic agents include, but are not limited to, the compounds disclosed in WO 2004/096286 (Gilead Sciences), WO 2006/015261 (Gilead Sciences), WO 2006/110157 (Gilead Sciences), WO 2012/003497 (Gilead Sciences), WO 2012/003498 (Gilead Sciences), WO 2012/145728 (Gilead Sciences), WO 2013/006738 (Gilead Sciences), WO 2013/159064 (Gilead Sciences), WO 2014/100323 (Gilead Sciences), US 2013/0165489 (University of Pennsylvania), US 2014/0221378 (Japan Tobacco). US 2014/0221380 (Japan Tobacco), WO 2009/062285 (Boehringer Ingelheim), WO 2010/130034 (Boehringer Ingelheim), WO 2013/006792 (Pharma Resources), US 20140221356 (Gilead Sciences), US 20100143301 (Gilead Sciences) and WO 2013/091096 (Boehringer Ingelheim).

### HIV Vaccines

Examples of HIV vaccines include, but are not limited to, peptide vaccines, recombinant subunit protein vaccines, live vector vaccines, DNA vaccines, HIV MAG DNA vaccine, CD4-derived peptide vaccines, vaccine combinations, adenoviral vector vaccines (an adenoviral vector such as Ad5, Ad26 or Ad35), simian adenovirus (chimpanzee, gorilla, rhesus i.e., rhAd), adeno-associated virus vector vaccines, Chimpanzee adenoviral vaccines (e.g., ChAdOX1, ChAd68, ChAd3, ChAd63, ChAd83, ChAd155, ChAd157, Pan5, Pan6, Pan7, Pan9), Coxsackieviruses based vaccines, enteric virus based vaccines, Gorilla adenovirus vaccines, lentiviral vector based vaccine, arenavirus vaccines (such as LCMV, Pichinde), bi-segmented or tri-segmented arenavirus based vaccine, trimer-based HIV-1 vaccine, measles virus based vaccine, flavivirus vector based vaccines, tobacco mosaic virus vector based vaccine, Varicella-zoster virus based vaccine, Human parainfluenza virus 3 (PIV3) based vaccines, poxvirus based vaccine (modified vaccinia virus Ankara (MVA), orthopoxvirus-derived NYVAC, and avipoxvirus-derived ALVAC (canarypox virus) strains); fowlpox virus based vaccine, rhabdovirus-based vaccines, such as VSV and marabavirus; recombinant human CMV (rhCMV) based vaccine, alphavirus-based vaccines, such as semliki forest virus, venezuelan equine encephalitis virus and sindbis virus; (see Lauer, Clinical and Vaccine Immunology, 2017, DOI: 10.1128/CVI.00298-16); LNP formulated mRNA based therapeutic vaccines; LNP-formulated self-replicating RNA/self-amplifying RNA vaccines.

Examples of vaccines include: AAVLP-HIV vaccine, AE-298p, anti-CD40.Env-gp140 vaccine, Ad4-EnvC150, BG505 SOSIP.664 gp140 adjuvanted vaccine, BG505 SOSIP.GT1.1 gp140 adjuvanted vaccine, ChAdOx1.tHIVconsv1 vaccine, CMV-MVA triplex vaccine, ChAdOx1.HTI, Chimigen HIV vaccine, ConM SOSIP.v7 gp140, ALVAC HIV (vCP1521), AIDSVAX B/E (gp120), monomeric gp120 HIV-1 subtype C vaccine, MPER-656 liposome subunit vaccine, Remune, ITV-1, Contre Vir, Ad5-ENVA-48, DCVax-001 (CDX-2401), Vacc-4x, Vacc-C5, VAC-3S, multiclade DNA recombinant adenovirus-5 (rAdS), rAd5 gag-pol env A/B/C vaccine, Pennvax-G, Pennvax-GP, Pennvax-G/MVA-CMDR, HIV-TriMix-mRNA vaccine, HIV-LAMP-vax, Ad35, Ad35-GRIN, NAcGM3/VSSP ISA-51, poly-ICLC adjuvanted vaccines, TatImmune, GTU-multiHIV (FIT-06), ChAdV63.HIVconsv, gp140[delta]V2.TV1+MF-59, rVSVIN HIV-1 gag vaccine, SeV-EnvF, SeV-Gag vaccine, AT-20, DNK-4, ad35-Grin/ENV, TBC-M4, HIVAX, HIVAX-2, N123-VRC-34.01 inducing epitope-based HIV vaccine, NYVAC-HIV-PTI, NYVAC-HIV-PT4, DNA-HIV-PT123, rAAV1-PG9DP, GOVX-B11, GOVX-B21, GOVX-C55, TVI-HIV-1, Ad-4 (Ad4-env Clade C+Ad4-mGag), Paxvax, EN41-UGR7C, EN41-FPA2, ENOB-HV-11, ENOB-HV-12, PreVaxTat, AE-H, MYM-V101, CombiHIVvac, ADVAX, MYM-V201, MVA-CMDR, MagaVax, DNA-Ad5 gag/pol/nef/nev (HVTN505), MVATG-17401, ETV-01, CDX-1401, DNA and Sev vectors vaccine expressing SCaVII, rcAD26.MOS1.HIV-Env, Ad26.Mod.HIV vaccine, Ad26.Mod.HIV+MVA mosaic vaccine+gp140, AGS-004, AVX-101, AVX-201, PEP-6409, SAV-001, ThV-01, TL-01, TUTI-16, VGX-3300, VIR-1111, IHV-001, and virus-like particle vaccines such as pseudovirion vaccine, CombiVICHvac, LFn-p24 B/C fusion vaccine, GTU-based DNA vaccine, HIV gag/pol/nef/env DNA vaccine, anti-TAT HIV vaccine, conjugate polypeptides vaccine, dendritic-cell vaccines (such as DermaVir), gag-based DNA vaccine, GI-2010, gp4l HIV-1 vaccine, HIV vaccine (PIKA adjuvant), i-key/MHC class II epitope hybrid peptide vaccines. ITV-2, ITV-3, ITV-4, LIPO-5, multiclade Env vaccine, MVA vaccine, Pennvax-GP, pp7I-deficient HCMV vector HIV gag vaccine, rgp160 HIV vaccine, RNActive HIV vaccine, SCB-703, Tat Oyi vaccine, TBC-M4, UBI HIV gp120, Vacc-4x+romidepsin, variant gp120 polypeptide vaccine, rAd5 gag-pol env A/B/C vaccine, DNA.HTI and MVA.HTI, VRC-HIVDNA016-00-VP+VRC-HIVADV014-00-VP, INO-6145, JNJ-9220, gp145 C.6980, eOD-GT8 60mer based vaccine, PD-201401, env (A, B, C, A/E)/gag (C) DNA Vaccine, gp120 (A,B,C,A/E) protein vaccine, PDPHV-201401, Ad4-EnvCN54, EnvSeq-1 Envs HIV-1 vaccine (GLA-SE adjuvanted), HIV p24gag prime-boost plasmid DNA vaccine, HIV-1 igIb12 neutralizing VRC-01 antibody-stimulating anti-CD4 vaccine, arenavirus vector-based vaccines (Vaxwave, TheraT), MVA-BN HIV-1 vaccine regimen, mRNA based prophylactic vaccines, VPI-211, multimeric HIV gp120 vaccine (Fred Hutchinson cancer center), TBL-1203HI, CH505 TF chTrimer, CD40.HIVRI.Env vaccine, Drep-HIV-PT-1, mRNA-1644, and mRNA-1574.

### Birth Control (Contraceptive) Combination Therapy

In certain embodiments, the agents described herein are combined with a birth control or contraceptive regimen. Therapeutic agents used for birth control (contraceptive) that can be combined with an agent of this disclosure include without limitation cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

In a particular embodiment, a compound disclosed herein, or a pharmaceutically acceptable salt thereof, is combined with one, two, three, or four additional therapeutic agents selected from ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA®, (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); BIKTARVY® (bictegravir+emtricitabine+tenofovir alafenamide), adefovir; adefovir dipivoxil, cobicistat; emtricitabine; tenofovir; tenofovir alafenamide and elvitegravir; tenofovir alafenamide+elvitegravir (rectal formulation, HIV infection); tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; PEGylated raltegravir; raltegravir and lamivudine; lamivudine+lopinavir+ritonavir+abacavir; maraviroc; tenofovir+emtricitabine+maraviroc, enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC); TRIZIVIR® (abacavir sulfate, zidovudine, and lanivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

In some embodiments, an agent disclosed herein, or a pharmaceutical composition thereof, is combined with an HIV nucleoside or nucleotide inhibitor of reverse transcriptase and an HIV non-nucleoside inhibitor of reverse transcriptase. In another specific embodiment, an agent disclosed herein, or a pharmaceutical composition thereof, is combined with an HIV nucleoside or nucleotide inhibitor of reverse transcriptase, and an HIV protease inhibiting compound. In an additional embodiment, an agent disclosed herein, or a pharmaceutical composition thereof, is combined with an HIV nucleoside or nucleotide inhibitor of reverse transcriptase, an HIV non-nucleoside inhibitor of reverse transcriptase, and a pharmacokinetic enhancer. In certain embodiments, an agent disclosed herein, or a pharmaceutical composition thereof, is combined with at least one HIV nucleoside inhibitor of reverse transcriptase, an integrase inhibitor, and a pharmacokinetic enhancer. In another embodiment, an agent disclosed herein, or a pharmaceutical composition thereof, is combined with two HIV nucleoside or nucleotide inhibitors of reverse transcriptase.

In another embodiment, an agent disclosed herein, or a pharmaceutical composition thereof, is combined with a first additional therapeutic agent chosen from dolutegravir, cabotegravir, islatravir, darunavir, bictegravir, elsulfavirine, rilpivirine, and lenacapavir and a second additional therapeutic agent chosen from emtricitabine and lamivudine.

In some embodiments, an agent disclosed herein, or a pharmaceutical composition thereof, is combined with a first additional therapeutic agent (a contraceptive) selected from the group consisting of cyproterone acetate, desogestrel, dienogest, drospirenone, estradiol valerate, ethinyl Estradiol, ethynodiol, etonogestrel, levomefolate, levonorgestrel, lynestreno, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof

### Gene Therapy and Cell Therapy

In certain embodiments, the agents described herein are combined with a gene or cell therapy regimen. Gene therapy and cell therapy include without limitation the genetic modification to silence a gene; genetic approaches to directly kill the infected cells; the infusion of immune cells designed to replace most of the patient's own immune system to enhance the immune response to infected cells, or activate the patient's own immune system to kill infected cells, or find and kill the infected cells; genetic approaches to modify cellular activity to further alter endogenous immune responsiveness against the infection. Examples of cell therapy include without limitation LB-1903, ENOB-HV-01, ENOB-HV-21, ENOB-HV-31, GOVX-B01, HSPCs overexpressing ALDH1 (LV-800, HIV infection), AGT103-T, and SupT1 cell based therapy. Examples of dendritic cell therapy include without limitation AGS-004. CCR5 gene editing agents include without limitation SB-728T, SB-728-HSPC. CCR5 gene inhibitors include without limitation Cal-1, and lentivirus vector CCR5 shRNA/TRIM5alpha/TAR decoy-transduced autologous CD34-positive hematopoietic progenitor cells (HIV infection/HIV-related lymphoma). In some embodiments, C34-CCR5/C34-CXCR4 expressing CD4-positive T-cells are co-administered with one or more multi-specific antigen binding molecules. In some embodiments, the agents described herein are co-administered with AGT-103-transduced autologous T-cell therapy or AAV-eCD4-Ig gene therapy.

### Gene Editors

In certain embodiments, the agents described herein are combined with a gene editor, e.g., an HIV targeted gene editor. In various embodiments, the genome editing system can be selected from the group consisting of: a CRISPR/Cas9 complex, a zinc finger nuclease complex, a TALEN complex, a homing endonucleases complex, and a meganuclease complex. An illustrative HIV targeting CRISPR/Cas9 system includes without limitation EBT-101.

### CAR-T Cell Therapy

In some embodiments, the agents described herein can be co-administered with a population of immune effector cells engineered to express a chimeric antigen receptor (CAR), wherein the CAR comprises an HIV antigen binding domain. The HIV antigen include an HIV envelope protein or a portion thereof, gp120 or a portion thereof, a CD4 binding site on gp120, the CD4-induced binding site on gp120, N glycan on gp120, the V2 of gp120, the membrane proximal region on gp41. The immune effector cell is a T-cell or an NK cell. In some embodiments, the T-cell is a CD4+ T-cell, a CD8+ T-cell, or a combination thereof. Cells can be autologous or allogeneic. Examples of HIV CAR-T include A-1801, A-1902, convertible CAR-T, VC-CAR-T, CMV-N6-CART, anti-HIV duoCAR-T, anti-CD4 CART-cell therapy, CD4 CAR+C34-CXCR4+CCR5 ZFN T-cells, dual anti-CD4 CART-T cell therapy (CD4 CAR+C34-CXCR4 T-cells), anti-CD4 MicAbody antibody+anti-MicAbody CAR T-cell therapy (iNKG2D CAR, HIV infection), GP-120 CAR-T therapy, autologous hematopoietic stem cells genetically engineered to express a CD4 CAR and the C46 peptide.

### TCR T-Cell Therapy

In certain embodiments, the agents described herein are combined with a population of TCR-T-cells. TCR-T-cells are engineered to target HIV derived peptides present on the surface of virus-infected cells, for example, ImmTAV.

### B-Cell Therapy

In certain embodiments, the antibodies or antigen-binding fragments described herein are combined with a population of B cells genetically modified to express broadly neutralizing antibodies, such as 3BNC 117 (Hartweger et al., J. Exp. Med. 2019, 1301, Moffett et al., Sci. Immunol. 4, eaax0644 (2019) 17 May 2019.

A compound as disclosed herein (e.g., any compound of Formula I) may be combined with one, two, three, or four additional therapeutic agents in any dosage amount of the compound of Formula I (e.g., from 1 mg to 500 mg of compound).

In one embodiment, kits comprising a compound disclosed herein, or a pharmaceutically acceptable salt thereof, in combination with one or more (e.g., one, two, three, one or two, or one to three) additional therapeutic agents are provided.

In one embodiment, the additional therapeutic agent or agents of the kit is an anti-HIV agent, selected from HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, immunomodulators, immunotherapeutic agents, antibody-drug conjugates, gene modifiers, gene editors (such as CRISPR/Cas9, zinc finger nucleases, homing nucleases, synthetic nucleases. TALENs), cell therapies (such as chimeric antigen receptor T-cell. CAR-T, and engineered T cell receptors, TCR-T, autologous T cell therapies), compounds that target the HIV capsid, latency reversing agents, HIV bNAbs, immune-based therapies, phosphatidylinositol 3-kinase (PI3K) inhibitors, HIV antibodies, broadly neutralizing HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV viral infectivity factor inhibitors, TAT protein inhibitors, HIV Nef modulators, Hck tyrosine kinase modulators, mixed lineage kinase-3 (MLK-3) inhibitors, HIV splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, HIV vaccines, and combinations thereof.

In some embodiments, the additional therapeutic agent or agents of the kit are selected from combination drugs for HIV, other drugs for treating HIV, HIV protease inhibitors. HIV reverse transcriptase inhibitors, HIV integrase inhibitors. HIV non-catalytic site (or allosteric) integrase inhibitors, HIV entry (fusion) inhibitors, HIV maturation inhibitors, latency reversing agents, capsid inhibitors, immune-based therapies. PI3K inhibitors, HIV antibodies, and bispecific antibodies, and “antibody-like” therapeutic proteins, and combinations thereof.

In a specific embodiment, the kit includes a compound disclosed herein, or a pharmaceutically acceptable salt thereof, and an HIV nucleoside or nucleotide inhibitor of reverse transcriptase. In a specific embodiment, the kit includes a compound disclosed herein, or a pharmaceutically acceptable salt thereof, and an HIV nucleoside or nucleotide inhibitor of reverse transcriptase and an HIV non-nucleoside inhibitor of reverse transcriptase. In another specific embodiment, the kit includes a compound disclosed herein, or a pharmaceutically acceptable salt thereof, and an HIV nucleoside or nucleotide inhibitor of reverse transcriptase, and an HIV protease inhibiting compound. In an additional embodiment, the kit includes a compound disclosed herein, or a pharmaceutically acceptable salt thereof, an HIV nucleoside or nucleotide inhibitor of reverse transcriptase, an HIV non-nucleoside inhibitor of reverse transcriptase, and a pharmacokinetic enhancer. In certain embodiments, the kit includes a compound disclosed herein, or a pharmaceutically acceptable salt thereof, at least one HIV nucleoside inhibitor of reverse transcriptase, an integrase inhibitor, and a pharmacokinetic enhancer. In another embodiment, the kit includes a compound disclosed herein, or a pharmaceutically acceptable salt thereof, and two HIV nucleoside or nucleotide inhibitors of reverse transcriptase. In a specific embodiment, the kit includes a compound disclosed herein, or a pharmaceutically acceptable salt thereof, an HIV nucleoside or nucleotide inhibitor of reverse transcriptase and an HIV capsid inhibitor. In a specific embodiment, the kit includes a compound disclosed herein, or a pharmaceutically acceptable salt thereof, an HIV nucleoside inhibitor of reverse transcriptase and an HIV capsid inhibitor. In a specific embodiment, the kit includes a compound disclosed herein, or a pharmaceutically acceptable salt thereof, and an HIV capsid inhibitor. In a specific embodiment, the kit includes a compound disclosed herein, or a pharmaceutically acceptable salt thereof, and one, two, three or four HIV bNAbs. In a specific embodiment, the kit includes a compound disclosed herein, or a pharmaceutically acceptable salt thereof, one, two, three or four HIV bNAbs and an HIV capsid inhibitor. In a specific embodiment, the kit includes a compound disclosed herein, or a pharmaceutically acceptable salt thereof, one, two, three or four HIV bNAbs, an HIV capsid inhibitor, and an HIV nucleoside inhibitor of reverse transcriptase.

HIV Long acting Therapy

Examples of drugs that are being developed as long acting regimens include, but are not limited to, cabotegravir, rilpivirine, any integrase LA, VM-1500 LAI, maraviroc (LAI), tenofovir implant, islatravir implant, doravirine, raltegravir, and long acting dolutegravir.

Combinations with PGP Inhibitors

In some embodiments, the compounds described herein can be used in combination with a P-glycoprotein (PGP) inhibitor. Examples of PGP inhibitors include, but are not limited to, verapamil, dexverapamil, cyclosporine, zosuquidar, laniquidar, elacridar, tariquidar, and encequidar. In some embodiments, the compounds described herein can be used in combination with encequidar. In some embodiments, the compounds described herein can be used in combination with a pharmaceutically acceptable salt of encequidar. In some embodiments, the compounds described herein can be used in combination with a mesylate salt of encequidar.

In some embodiments, the compounds of Formula I or Formula II described herein can be co-administered with a P-glycoprotein (PGP) inhibitor. Examples of PGP inhibitors include, but are not limited to, verapamil, dexverapamil, cyclosporine, zosuquidar, laniquidar, elacridar, tariquidar, and encequidar. In some embodiments, the compounds described herein can be co-administered with encequidar. In some embodiments, the compounds described herein can be co-administered with a pharmaceutically acceptable salt of encequidar. In some embodiments, the compounds described herein can be co-administered with a mesylate salt of encequidar.

Increased intestinal expression of P-glycoprotein can reduce the absorption of drugs that are substrates for P-glycoprotein. PGP inhibitors may therefore be useful to modulate the PK properties of compounds disclosed herein.

In one embodiment, the PGP inhibitor is selected from the group consisting of verapamil, dexverapamil, cyclosporine, zosuquidar, laniquidar, elacridar, tariquidar, encequidar, and pharmaceutically acceptable salts thereof. In one embodiment, the PGP inhibitor is encequidar, or a pharmaceutically acceptable salt thereof. In one embodiment, the PGP inhibitor is a salt of encequidar. In one embodiment, the PGP inhibitor is a mesylate salt of encequidar.

In some embodiments, the present disclosure provides a combination comprising (i) a therapeutically effective amount of a compound of Formula I, or a pharmaceutically acceptable salt thereof, or a compound of Formula II, or a pharmaceutically acceptable salt thereof, and (ii) a P-glycoprotein inhibitor. In some embodiments, the combination comprises the compound of Formula I, or a pharmaceutically acceptable salt thereof.

In some embodiments of the combination, the compound of Formula I is selected from the group consisting of:

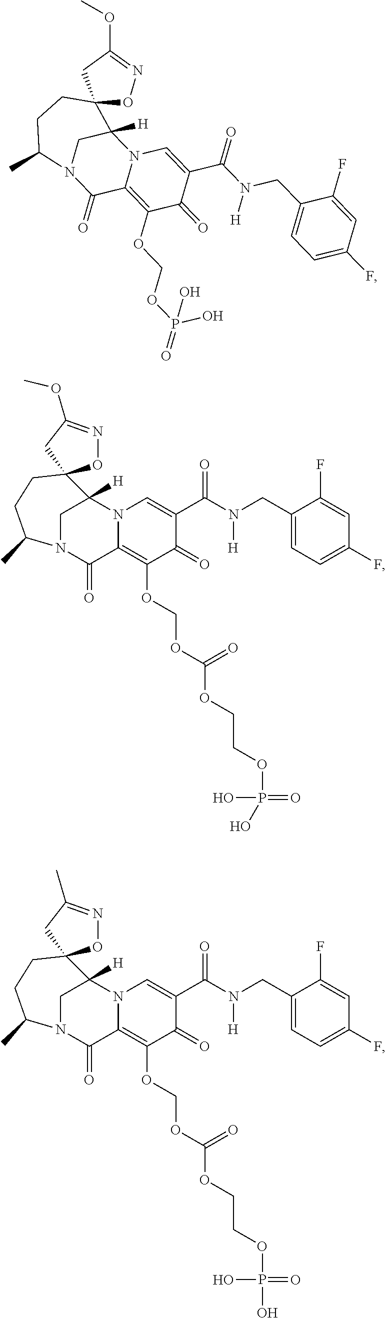 [SMILES: COC1=NO[C@@]2(CC[C@H](C)N3C[C@H]2n2cc(C(=O)NCc4ccc(F)cc4F)c(=O)c(OCOP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)OCCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OCCOP(=O)(O)O)c2C3=O)C1]

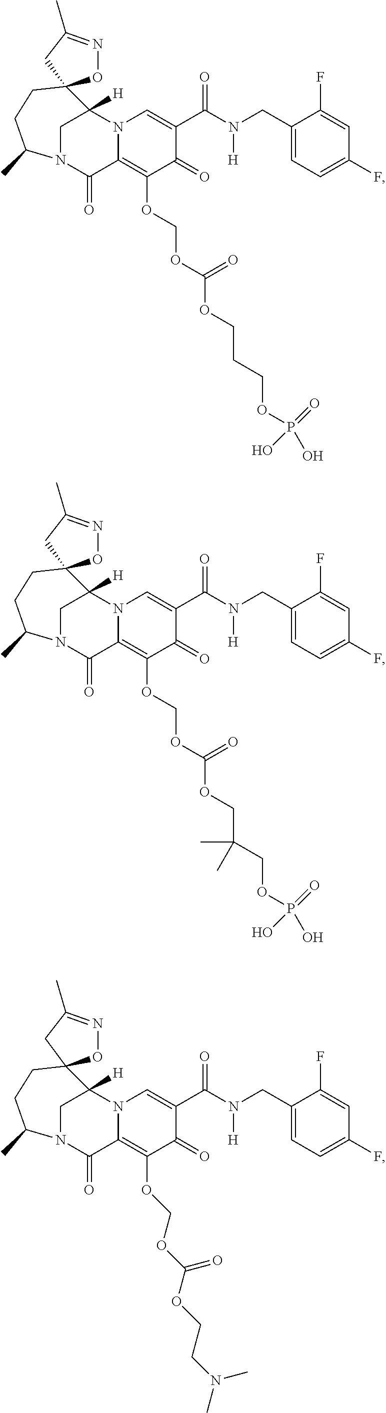 [SMILES: CC1=NO[C@@]2(CC[C@H](C)N3C[C@H]2n2cc(C(=O)NCc4ccc(F)cc4F)c(=O)c(OCOC(=O)OCCCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OCC(C)(C)COP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OCCN(C)C)c2C3=O)C1]

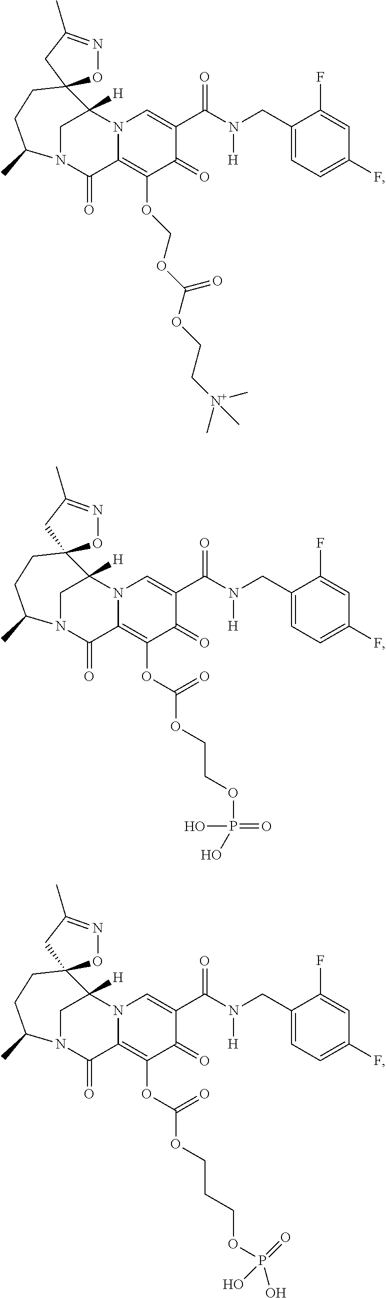 [SMILES: CC1=NO[C@@]2(CC[C@H](C)N3C[C@H]2n2cc(C(=O)NCc4ccc(F)cc4F)c(=O)c(OCOC(=O)OCC[N+](C)(C)C)c2C3=O)C1 ; CC1=NO[C@@]2(CC[C@H](C)N3C[C@H]2n2cc(C(=O)NCc4ccc(F)cc4F)c(=O)c(OC(=O)OCCOP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)OCCCOP(=O)(O)O)c2C3=O)C1]

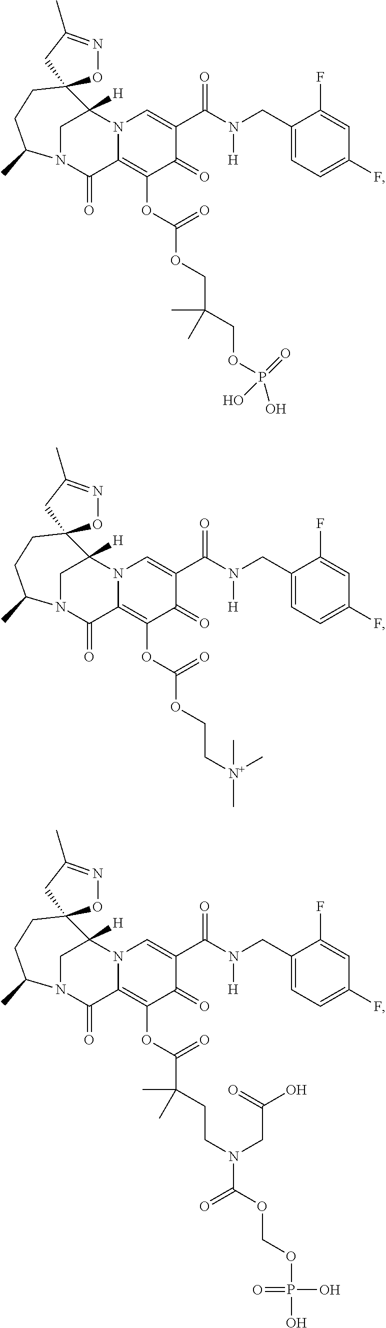 [SMILES: CC1=NO[C@@]2(CC[C@H](C)N3C[C@H]2n2cc(C(=O)NCc4ccc(F)cc4F)c(=O)c(OC(=O)OCC(C)(C)COP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)OCC[N+](C)(C)C)c2C3=O)C1 ; CC1=NO[C@@]2(CC[C@H](C)N3C[C@H]2n2cc(C(=O)NCc4ccc(F)cc4F)c(=O)c(OC(=O)C(C)(C)CCN(CC(=O)O)C(=O)OCOP(=O)(O)O)c2C3=O)C1]

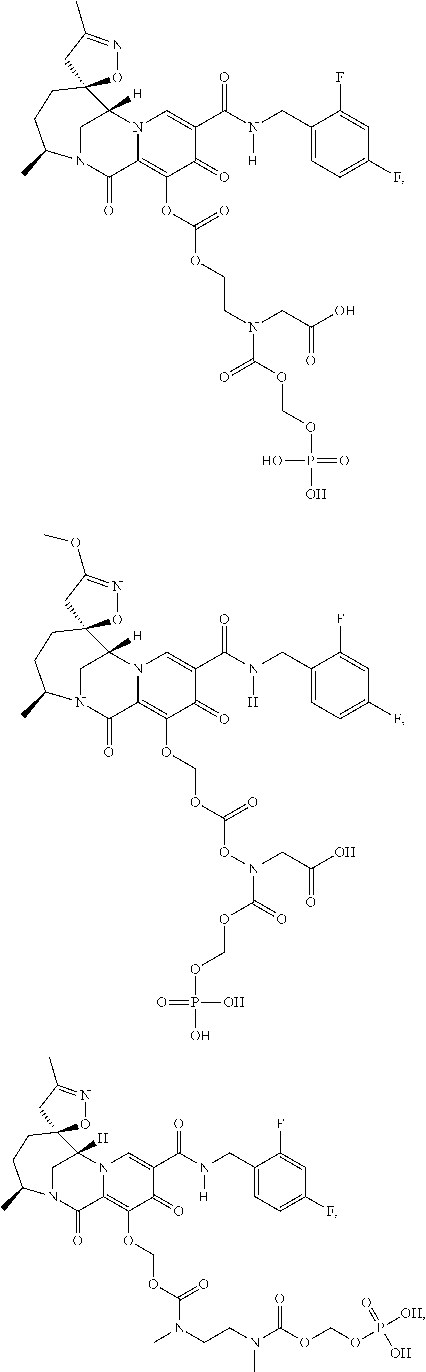 [SMILES: CC1=NO[C@@]2(CC[C@H](C)N3C[C@H]2n2cc(C(=O)NCc4ccc(F)cc4F)c(=O)c(OC(=O)OCCN(CC(=O)O)C(=O)OCOP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)ON(CC(=O)O)C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)N(C)CCN(C)C(=O)OCOP(=O)(O)O)c2C3=O)C1]

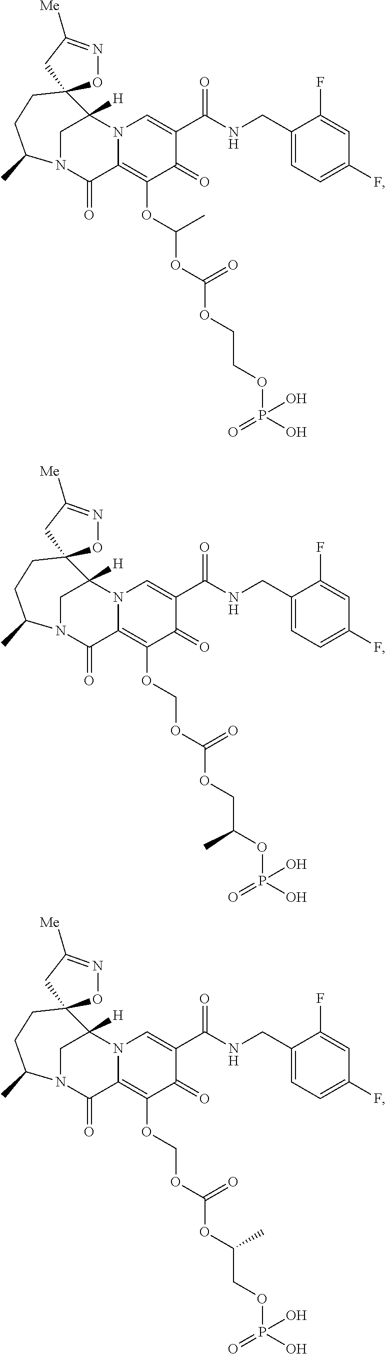 [SMILES: CC1=NO[C@@]2(CC[C@H](C)N3C[C@H]2n2cc(C(=O)NCc4ccc(F)cc4F)c(=O)c(OC(C)OC(=O)OCCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OC[C@H](C)OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)O[C@H](C)COP(=O)(O)O)c2C3=O)C1]

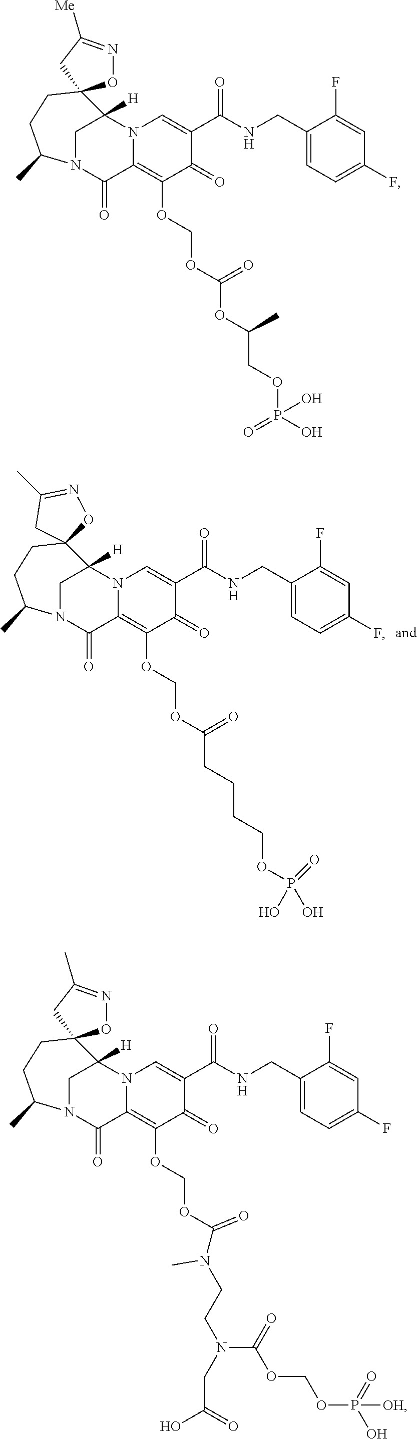 [SMILES: CC1=NO[C@@]2(CC[C@H](C)N3C[C@H]2n2cc(C(=O)NCc4ccc(F)cc4F)c(=O)c(OCOC(=O)O[C@@H](C)COP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)CCCCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)N(C)CCN(CC(=O)O)C(=O)OCOP(=O)(O)O)c2C3=O)C1]

or a pharmaceutically acceptable salt thereof.

In some embodiments, of the combination, the compound of Formula I is selected from the group consisting of:

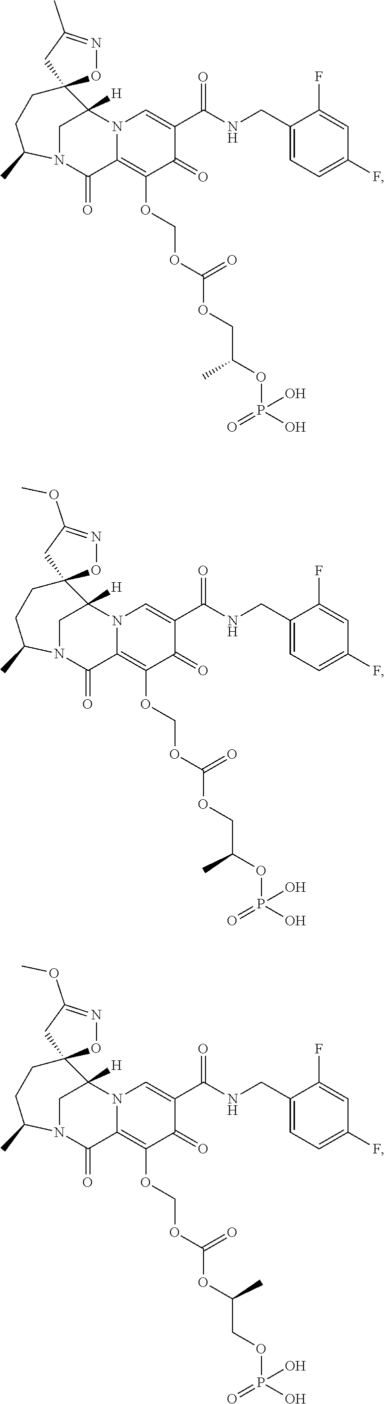 [SMILES: CC1=NO[C@@]2(CC[C@H](C)N3C[C@H]2n2cc(C(=O)NCc4ccc(F)cc4F)c(=O)c(OCOC(=O)OC[C@@H](C)OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)OC[C@H](C)OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)O[C@@H](C)COP(=O)(O)O)c2C3=O)C1]

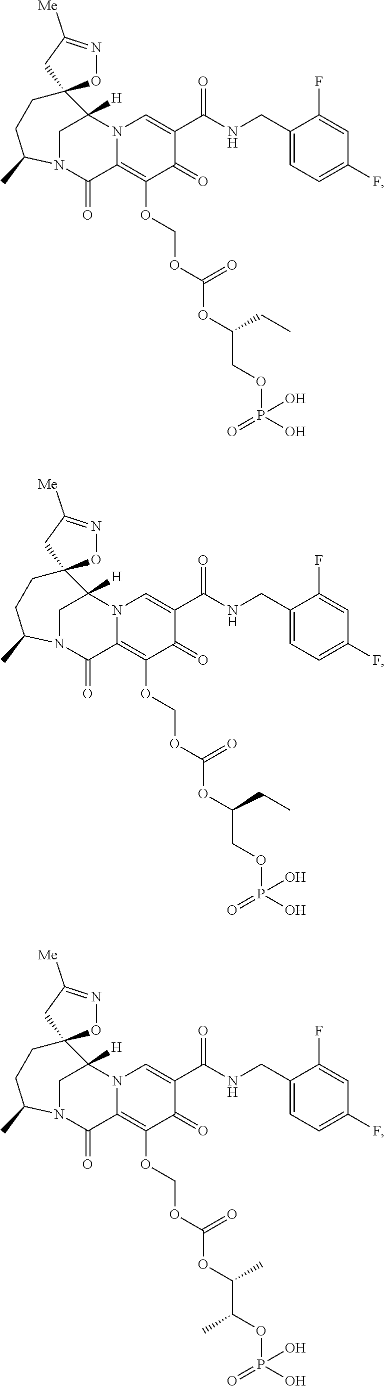 [SMILES: CC[C@H](COP(=O)(O)O)OC(=O)OCOc1c2n(cc(C(=O)NCc3ccc(F)cc3F)c1=O)[C@@H]1CN(C2=O)[C@@H](C)CC[C@]12CC(C)=NO2 ; CC[C@@H](COP(=O)(O)O)OC(=O)OCOc1c2n(cc(C(=O)NCc3ccc(F)cc3F)c1=O)[C@@H]1CN(C2=O)[C@@H](C)CC[C@]12CC(C)=NO2 ; CC1=NO[C@@]2(CC[C@H](C)N3C[C@H]2n2cc(C(=O)NCc4ccc(F)cc4F)c(=O)c(OCOC(=O)O[C@H](C)[C@@H](C)OP(=O)(O)O)c2C3=O)C1]

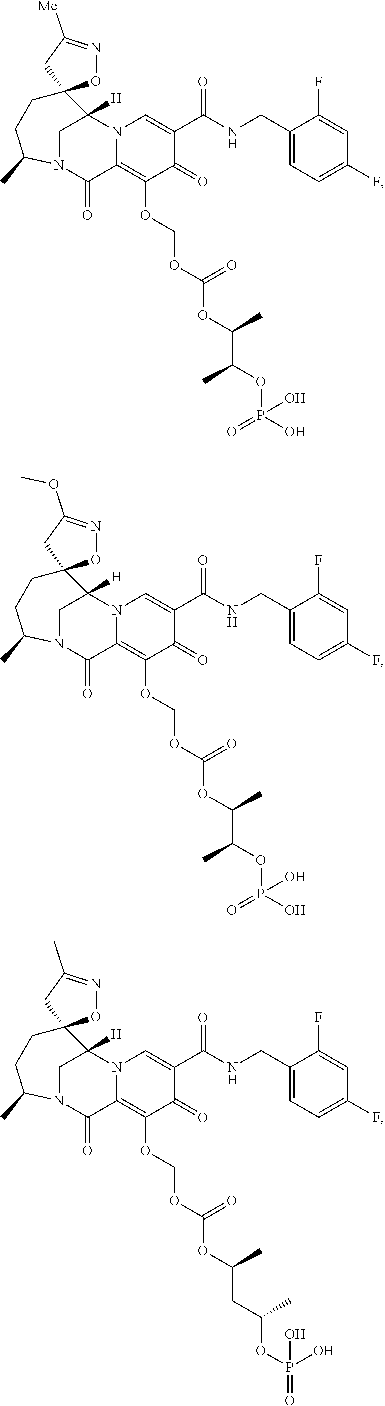 [SMILES: CC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)O[C@@H](C)C[C@H](C)OP(=O)(O)O)c2C3=O)C1]

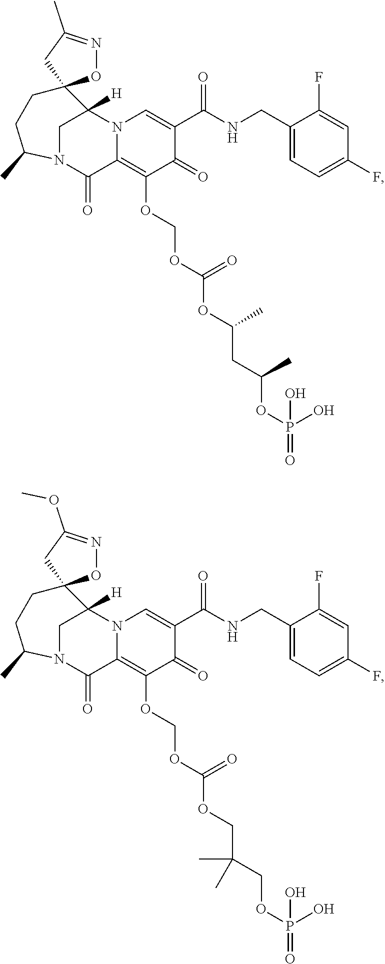 [SMILES: CC1=NO[C@@]2(CC[C@H](C)N3C[C@H]2n2cc(C(=O)NCc4ccc(F)cc4F)c(=O)c(OCOC(=O)O[C@H](C)C[C@@H](C)OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)OCC(C)(C)COP(=O)(O)O)c2C3=O)C1]

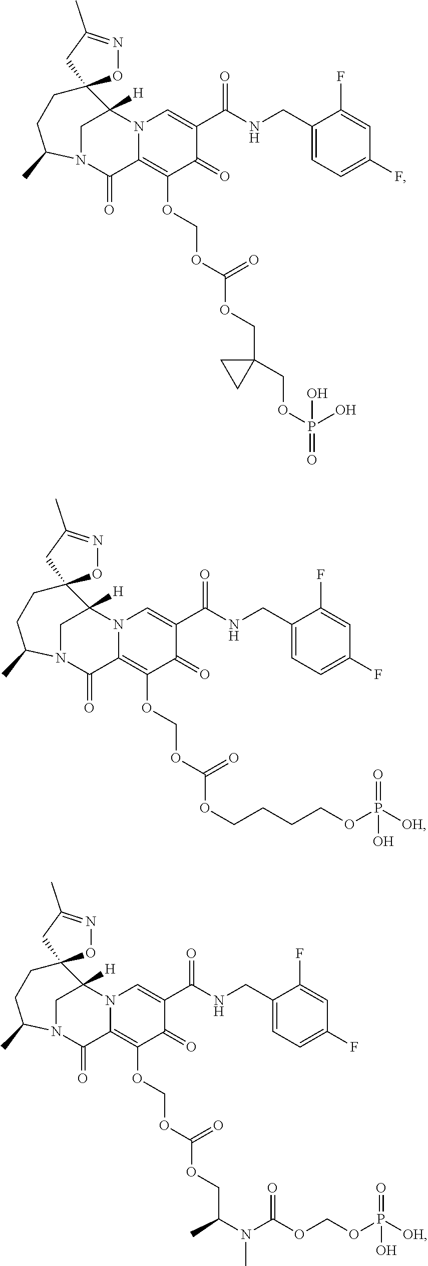 [SMILES: CC1=NO[C@@]2(CC[C@H](C)N3C[C@H]2n2cc(C(=O)NCc4ccc(F)cc4F)c(=O)c(OCOC(=O)OCC4(COP(=O)(O)O)CC4)c2C3=O)C1 ; CC1=NO[C@@]2(CC[C@H](C)N3C[C@H]2n2cc(C(=O)NCc4ccc(F)cc4F)c(=O)c(OCOC(=O)OCCCCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OC[C@H](C)N(C)C(=O)OCOP(=O)(O)O)c2C3=O)C1]

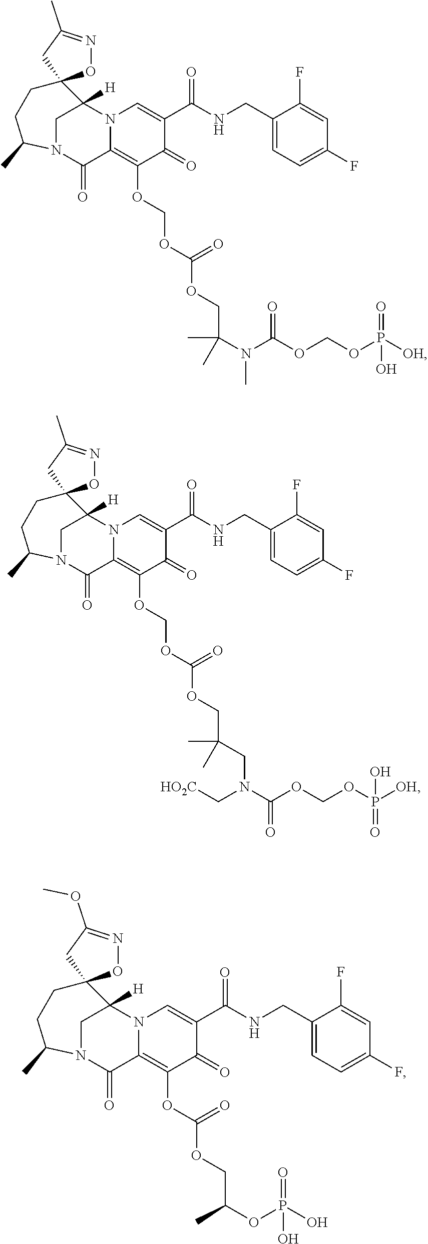 [SMILES: CC1=NO[C@@]2(CC[C@H](C)N3C[C@H]2n2cc(C(=O)NCc4ccc(F)cc4F)c(=O)c(OCOC(=O)OCC(C)(C)N(C)C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)OCC(C)(C)CN(CC(=O)O)C(=O)OCOP(=O)(O)O)c2C3=O)C1 ; COC1=NO[C@@]2(CC[C@H](C)N3C[C@H]2n2cc(C(=O)NCc4ccc(F)cc4F)c(=O)c(OC(=O)OC[C@H](C)OP(=O)(O)O)c2C3=O)C1]

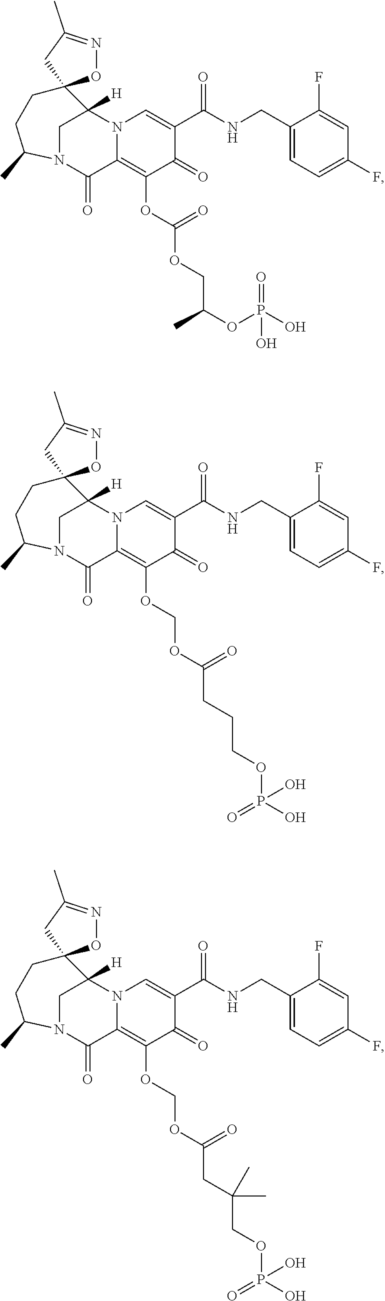 [SMILES: CC1=NO[C@@]2(CC[C@H](C)N3C[C@H]2n2cc(C(=O)NCc4ccc(F)cc4F)c(=O)c(OC(=O)OC[C@H](C)OP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)CCCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)CC(C)(C)COP(=O)(O)O)c2C3=O)C1]

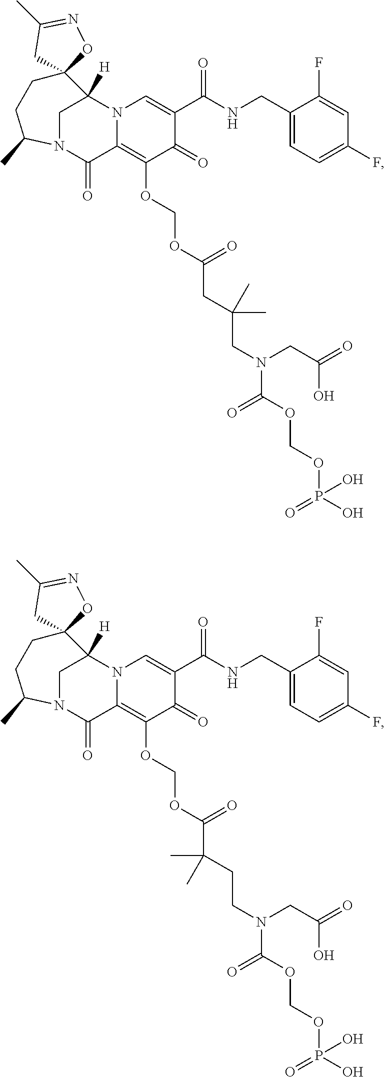 [SMILES: CC1=NO[C@@]2(CC[C@H](C)N3C[C@H]2n2cc(C(=O)NCc4ccc(F)cc4F)c(=O)c(OCOC(=O)CC(C)(C)CN(CC(=O)O)C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OCOC(=O)C(C)(C)CCN(CC(=O)O)C(=O)OCOP(=O)(O)O)c2C3=O)C1]

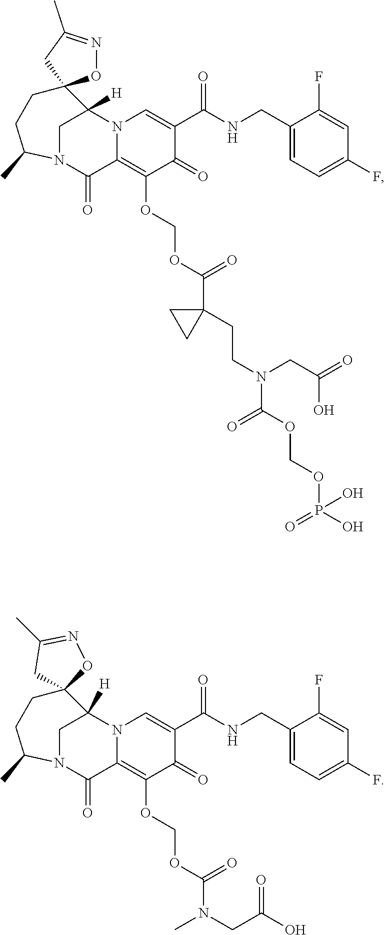 [SMILES: CC1=NO[C@@]2(CC[C@H](C)N3C[C@H]2n2cc(C(=O)NCc4ccc(F)cc4F)c(=O)c(OCOC(=O)C4(CCN(CC(=O)O)C(=O)OCOP(=O)(O)O)CC4)c2C3=O)C1 ; CC1=NO[C@@]2(CC[C@H](C)N3C[C@H]2n2cc(C(=O)NCc4ccc(F)cc4F)c(=O)c(OCOC(=O)N(C)CC(=O)O)c2C3=O)C1]

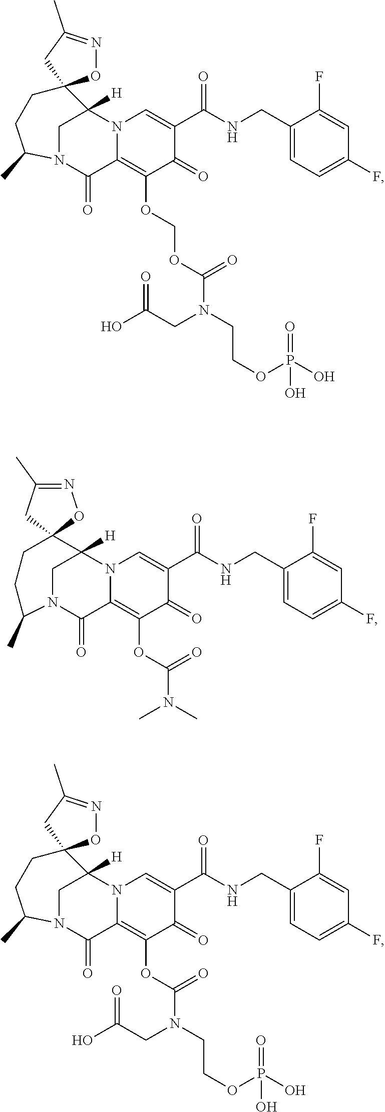 [SMILES: CC1=NO[C@@]2(CC[C@H](C)N3C[C@H]2n2cc(C(=O)NCc4ccc(F)cc4F)c(=O)c(OCOC(=O)N(CCOP(=O)(O)O)CC(=O)O)c2C3=O)C1 ; CC1=NO[C@@]2(CC[C@H](C)N3C[C@H]2n2cc(C(=O)NCc4ccc(F)cc4F)c(=O)c(OC(=O)N(C)C)c2C3=O)C1 ; CC1=NO[C@@]2(CC[C@H](C)N3C[C@H]2n2cc(C(=O)NCc4ccc(F)cc4F)c(=O)c(OC(=O)N(CCOP(=O)(O)O)CC(=O)O)c2C3=O)C1]

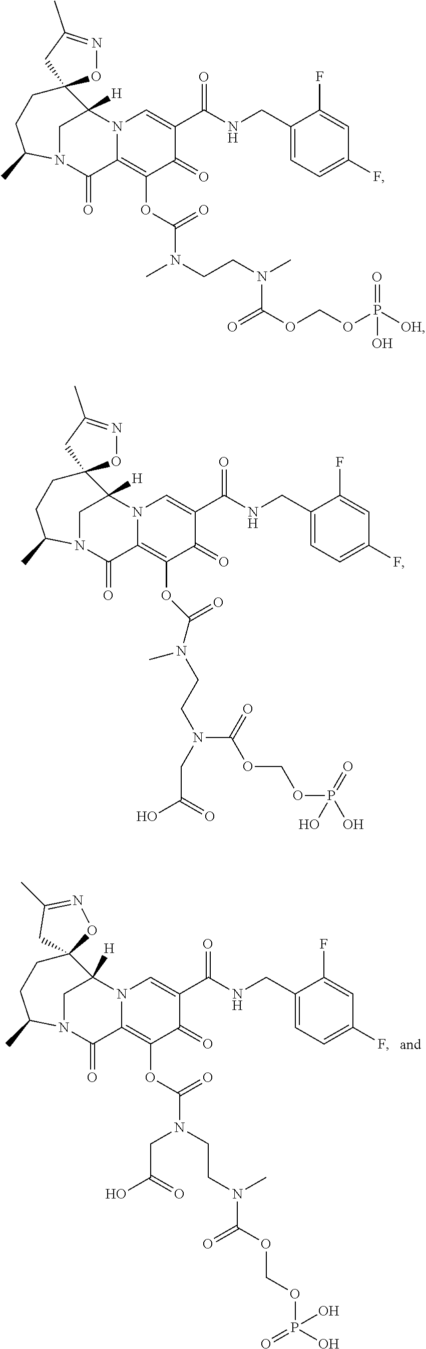 [SMILES: CC1=NO[C@@]2(CC[C@H](C)N3C[C@H]2n2cc(C(=O)NCc4ccc(F)cc4F)c(=O)c(OC(=O)N(C)CCN(C)C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)N(C)CCN(CC(=O)O)C(=O)OCOP(=O)(O)O)c2C3=O)C1 ; CC1=NO[C@@]2(CC[C@H](C)N3C[C@H]2n2cc(C(=O)NCc4ccc(F)cc4F)c(=O)c(OC(=O)N(CCN(C)C(=O)OCOP(=O)(O)O)CC(=O)O)c2C3=O)C1 ; "and"]

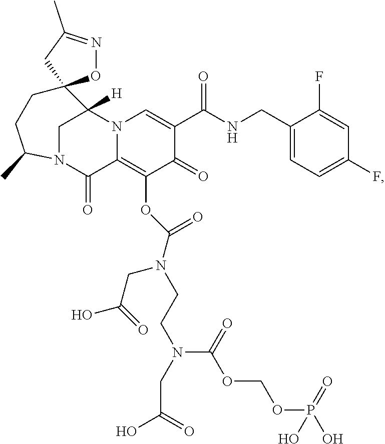 [SMILES: CC1=NO[C@@]2(CC[C@H](C)N3C[C@H]2n2cc(C(=O)NCc4ccc(F)cc4F)c(=O)c(OC(=O)N(CCN(CC(=O)O)C(=O)OCOP(=O)(O)O)CC(=O)O)c2C3=O)C1]

or a pharmaceutically acceptable salt thereof.

In some embodiments of the combination, the compound of Formula I is selected from the group consisting of:

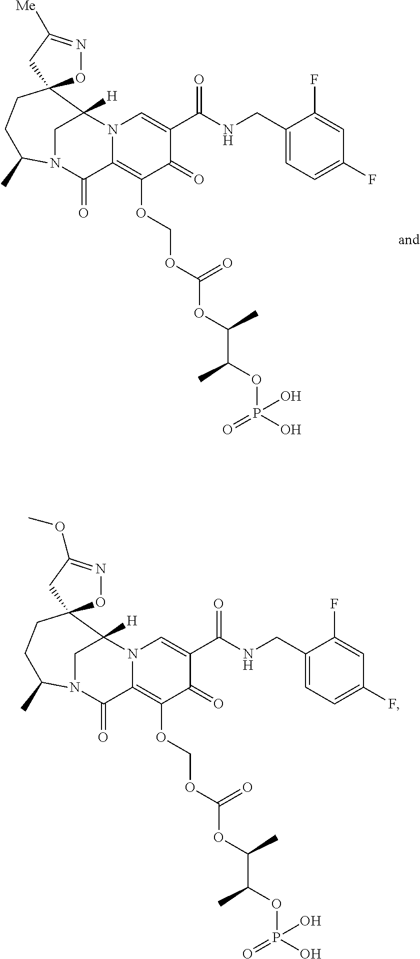 [SMILES: CC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1 ; COC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1]

or a pharmaceutically acceptable salt thereof.

In some embodiments of the combination, the compound of Formula I is:

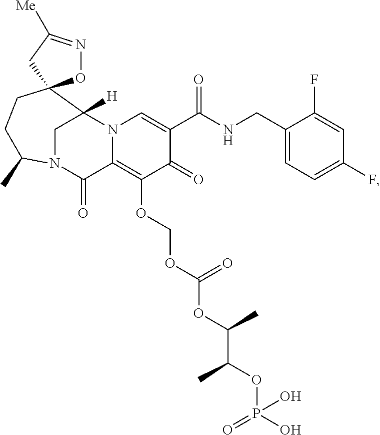 [SMILES: CC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1]

or a pharmaceutically acceptable salt thereof.

In some embodiments of the combination, the compound of Formula I is:

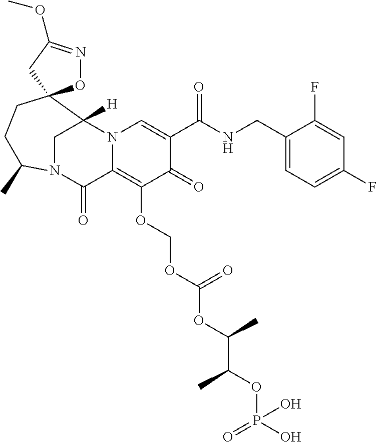 [SMILES: COC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1]

or a pharmaceutically acceptable salt thereof.

In some embodiments, the combination comprises the compound of Formula I.

In some embodiments of the combination, the compound of Formula I is:

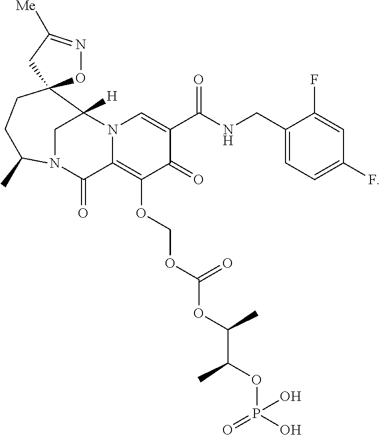 [SMILES: CC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1]

In some embodiments of the combination, the compound of Formula I is:

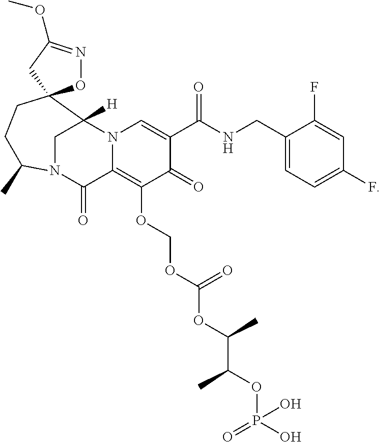 [SMILES: COC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1]

In some embodiments, the combination comprises the compound of Formula II, or a pharmaceutically acceptable salt thereof. In some embodiments, the compound of Formula II is:

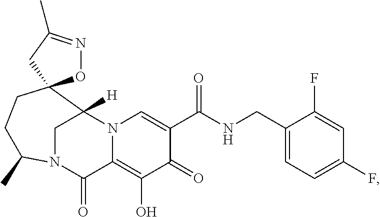 [SMILES: CC1=NO[C@@]2(CC[C@H](C)N3C[C@H]2n2cc(C(=O)NCc4ccc(F)cc4F)c(=O)c(O)c2C3=O)C1]

or a pharmaceutically acceptable salt thereof.

In some embodiments of combination, the compound of Formula II is:

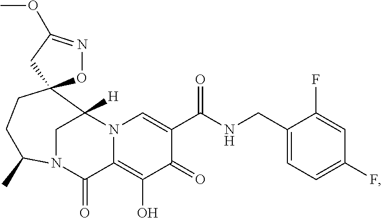 [SMILES: COC1=NO[C@@]2(CC[C@H](C)N3C[C@H]2n2cc(C(=O)NCc4ccc(F)cc4F)c(=O)c(O)c2C3=O)C1]

or a pharmaceutically acceptable salt thereof.

In some embodiments, the combination comprises the compound of Formula II. In some embodiments, the compound of Formula II is

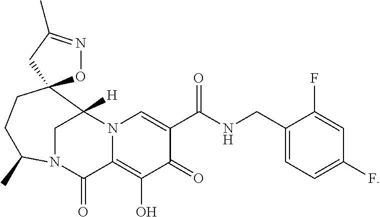 [SMILES: CC1=NO[C@@]2(CC[C@H](C)N3C[C@H]2n2cc(C(=O)NCc4ccc(F)cc4F)c(=O)c(O)c2C3=O)C1]

In some embodiments, the compound of Formula II is

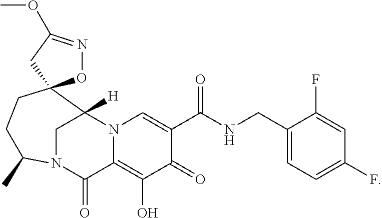 [SMILES: COC1=NO[C@@]2(CC[C@H](C)N3C[C@H]2n2cc(C(=O)NCc4ccc(F)cc4F)c(=O)c(O)c2C3=O)C1]

In some embodiments, the P-glycoprotein inhibitor in the combination is selected from the group consisting of verapamil, dexverapamil, cyclosporine, zosuquidar, laniquidar, elacridar, tariquidar, encequidar, and pharmaceutically acceptable salts thereof. In some embodiments, the P-glycoprotein inhibitor is encequidar, or a pharmaceutically acceptable salt thereof. In some embodiments, the P-glycoprotein inhibitor is a salt of encequidar. In some embodiments, the P-glycoprotein inhibitor is a mesylate salt of encequidar.

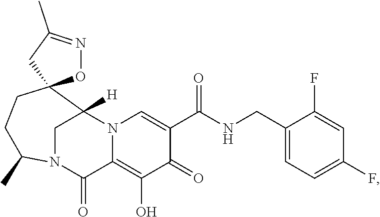 [SMILES: CC1=NO[C@@]2(CC[C@H](C)N3C[C@H]2n2cc(C(=O)NCc4ccc(F)cc4F)c(=O)c(O)c2C3=O)C1]

or a pharmaceutically acceptable salt thereof, and encequidar, or a pharmaceutically acceptable salt thereof.

In some embodiments, the combination comprises

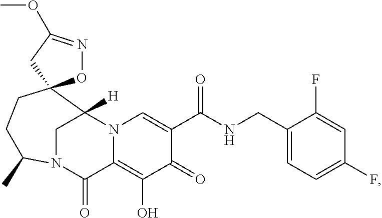 [SMILES: COC1=NO[C@@]2(CC[C@H](C)N3C[C@H]2n2cc(C(=O)NCc4ccc(F)cc4F)c(=O)c(O)c2C3=O)C1]

or a pharmaceutically acceptable salt thereof, and encequidar, or a pharmaceutically acceptable salt thereof.

In some embodiments, the combination comprises

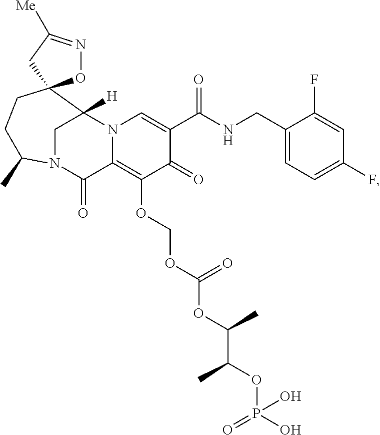 [SMILES: CC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1]

or a pharmaceutically acceptable salt thereof, and encequidar, or a pharmaceutically acceptable salt thereof.

In some embodiments, the combination comprises

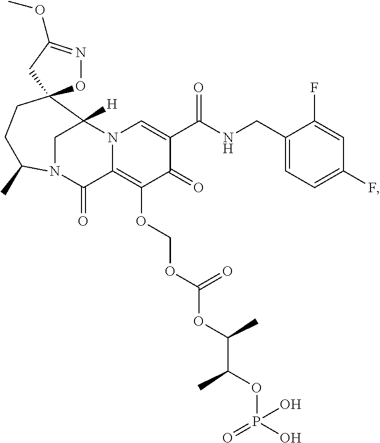 [SMILES: COC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1]

or a pharmaceutically acceptable salt thereof, and encequidar, or a pharmaceutically acceptable salt thereof.

In some embodiments, the combination comprises

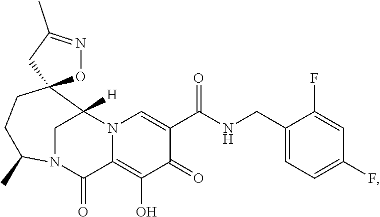 [SMILES: CC1=NO[C@@]2(CC[C@H](C)N3C[C@H]2n2cc(C(=O)NCc4ccc(F)cc4F)c(=O)c(O)c2C3=O)C1]

or a pharmaceutically acceptable salt thereof, and a salt of encequidar.

In some embodiments, the combination comprises

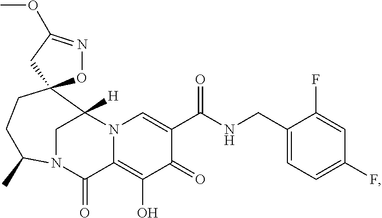 [SMILES: COC1=NO[C@@]2(CC[C@H](C)N3C[C@H]2n2cc(C(=O)NCc4ccc(F)cc4F)c(=O)c(O)c2C3=O)C1]

or a pharmaceutically acceptable salt thereof, and a salt of encequidar.

In some embodiments, the combination comprises

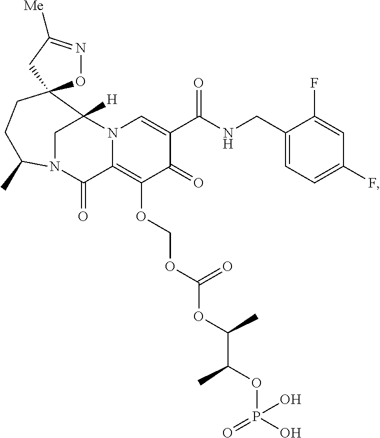 [SMILES: CC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1]

or a pharmaceutically acceptable salt thereof, and a salt of encequidar.

In some embodiments, the combination comprises

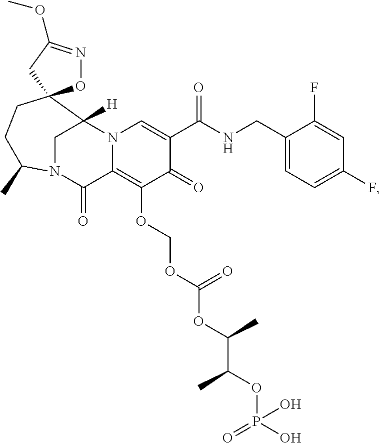 [SMILES: COC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1]

or a pharmaceutically acceptable salt thereof, and a salt of encequidar.

In some embodiments, the combination comprises

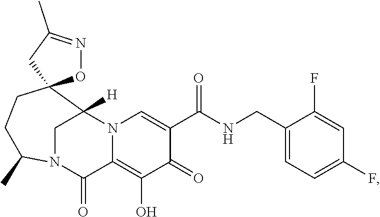 [SMILES: CC1=NO[C@@]2(CC[C@H](C)N3C[C@H]2n2cc(C(=O)NCc4ccc(F)cc4F)c(=O)c(O)c2C3=O)C1]

and a salt of encequidar.

In some embodiments, the combination comprises

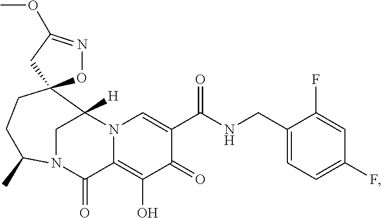 [SMILES: COC1=NO[C@@]2(CC[C@H](C)N3C[C@H]2n2cc(C(=O)NCc4ccc(F)cc4F)c(=O)c(O)c2C3=O)C1]

and a salt of encequidar.

In some embodiments, the combination comprises

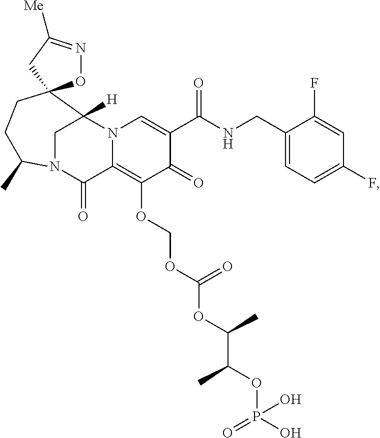 [SMILES: CC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1]

and a salt of encequidar.

In some embodiments, the combination comprises

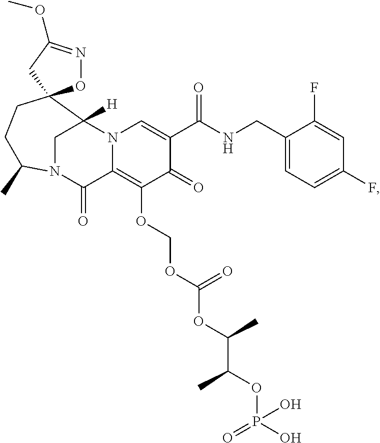 [SMILES: COC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1]

and a salt of encequidar.

In some embodiments, the combination comprises

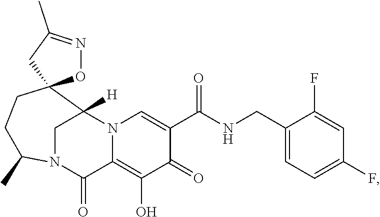 [SMILES: CC1=NO[C@@]2(CC[C@H](C)N3C[C@H]2n2cc(C(=O)NCc4ccc(F)cc4F)c(=O)c(O)c2C3=O)C1]

and a mesylate salt of encequidar.

In some embodiments, the combination comprises

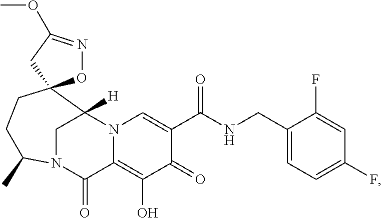 [SMILES: COC1=NO[C@@]2(CC[C@H](C)N3C[C@H]2n2cc(C(=O)NCc4ccc(F)cc4F)c(=O)c(O)c2C3=O)C1]

and a mesylate salt of encequidar.

In some embodiments, the combination comprises

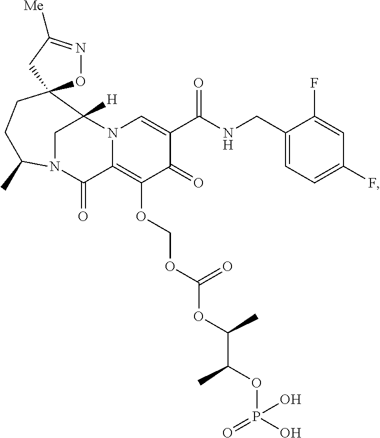 [SMILES: CC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1]

and a mesylate salt of encequidar.

In some embodiments, the combination comprises

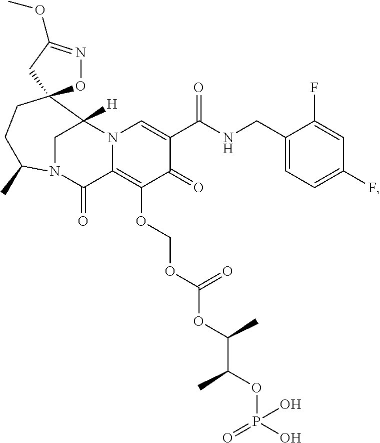 [SMILES: COC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1]

and a mesylate salt of encequidar.

In some embodiments, the disclosure provides method of treating an HIV infection in a human having or at risk of having the infection, comprising administering to the human a combination comprising (i) the compound of Formula I, or the pharmaceutically acceptable salt thereof, or the compound of Formula II, or the pharmaceutically acceptable salt thereof and (ii) the P-glycoprotein inhibitor. In some embodiments, (i) the compound of Formula I, or the pharmaceutically acceptable salt thereof, or the compound of Formula II, or the pharmaceutically acceptable salt thereof and (ii) the P-glycoprotein inhibitor are administered together. In some embodiments, (i) the compound of Formula I, or the pharmaceutically acceptable salt thereof, or the compound of Formula II, or the pharmaceutically acceptable salt thereof and (ii) the P-glycoprotein inhibitor are administered together. In some embodiments. (i) the compound of Formula I, or the pharmaceutically acceptable salt thereof, or the compound of Formula II, or the pharmaceutically acceptable salt thereof and (ii) the P-glycoprotein inhibitor are administered as a co-formulation (i.e., the compound of Formula I or Formula II, or a pharmaceutically acceptable salt thereof, are formulated in the same pharmaceutical composition as the PGP inhibitor). In some embodiments, (i) the compound of Formula I, or the pharmaceutically acceptable salt thereof, or the compound of Formula II, or the pharmaceutically acceptable salt thereof and (ii) the P-glycoprotein inhibitor are administered separately. In some embodiments, (i) the compound of Formula I, or the pharmaceutically acceptable salt thereof, or the compound of Formula II, or the pharmaceutically acceptable salt thereof and (ii) the P-glycoprotein inhibitor are administered orally.

In some embodiments, the methods disclosed herein comprise further comprising administering to the human a therapeutically effective amount of one, two, three, or four additional therapeutic agents. In some embodiments, the additional therapeutic agent or agents are anti-HIV agents. In some embodiments, the additional therapeutic agent or agents are HIV protease inhibitors, HIV non-nucleoside or non-nucleotide inhibitors of reverse transcriptase, HIV nucleoside or nucleotide inhibitors of reverse transcriptase, HIV capsid inhibitors, gp41 inhibitors, CXCR4 inhibitors, gp120 inhibitors, CCR5 inhibitors, latency reversing agents, capsid polymerization inhibitors, HIV bNAbs, TLR7 agonists, pharmacokinetic enhancers, other drugs for treating HIV, or combinations thereof. In some embodiments, the additional therapeutic agent or agents are abacavir, tenofovir alafenamide, tenofovir disoproxil, N—((S)-1-(3-(4-chloro-3-(methylsulfonamido)-1-(2,2,2-trifluoroethyl)-1H-indazol-7-yl)-6-(3-methyl-3-(methylsulfonyl)but-1-yn-1-yl)-pyridin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide, or a pharmaceutically acceptable salt thereof.

In certain embodiments, a method for treating an HIV infection is provided, comprising administering to the human (i) a therapeutically effective amount of a compound of Formula I or Formula II disclosed herein, or a pharmaceutically acceptable salt thereof, and (ii) a PGP inhibitor, in combination with a therapeutically effective amount of one, two, three, or four additional therapeutic agents. In one embodiment, a method for treating an HIV infection is provided, comprising administering to the human (i) a therapeutically effective amount of a compound of Formula I or Formula II disclosed herein, or a pharmaceutically acceptable salt thereof, and (ii) a PGP inhibitor, in combination with a therapeutically effective amount of one, two, three, or four additional therapeutic agents.

In one embodiment, pharmaceutical compositions comprising (i) a compound of Formula I or Formula II disclosed herein, or a pharmaceutically acceptable salt thereof, and (ii) a P-glycoprotein inhibitor in combination with one, two, three, or four additional therapeutic agents, and a pharmaceutically acceptable carrier, diluent, or excipient are provided.

In certain embodiments, the present disclosure provides a method for treating an HIV infection, comprising administering to a subject in need thereof (i) a therapeutically effective amount of a compound of Formula I or Formula II disclosed herein, or a pharmaceutically acceptable salt thereof, and (ii) a P-glycoprotein inhibitor, in combination with a therapeutically effective amount of one, two, three, or four additional therapeutic agents which are suitable for treating an HIV infection.

In certain embodiments, (i) a compound of Formula I or Formula II disclosed herein, or a pharmaceutically acceptable salt thereof, and (ii) a PGP inhibitor, is combined with one, two, three, four, or more additional therapeutic agents. In certain embodiments. (i) a compound of Formula I or Formula II disclosed herein, or a pharmaceutically acceptable salt thereof, and (ii) a PGP inhibitor, is combined with one, two, three, or four additional therapeutic agents. In certain embodiments, (i) a compound of Formula I or Formula II disclosed herein, or a pharmaceutically acceptable salt thereof, and (ii) a PGP inhibitor, is combined with two additional therapeutic agents. In other embodiments, (i) a compound of Formula I or Formula II disclosed herein, or a pharmaceutically acceptable salt thereof, and (ii) a PGP inhibitor, is combined with three additional therapeutic agents. In further embodiments, a compound of Formula I or Formula II disclosed herein, or a pharmaceutically acceptable salt thereof, and (ii) a PGP inhibitor, is combined with four additional therapeutic agents. The one, two, three, four, or more additional therapeutic agents can be different therapeutic agents selected from the same class of therapeutic agents, and/or they can be selected from different classes of therapeutic agents.

In some embodiments, (i) the compound of Formula I, or the pharmaceutically acceptable salt thereof, or the compound of Formula II, or the pharmaceutically acceptable salt thereof, (ii) the P-glycoprotein inhibitor, and (iii) additional therapeutic agent or agents are administered as a co-formulation.

In some embodiments, the disclosure provides a pharmaceutical composition comprising (i) the compound of Formula I, or the pharmaceutically acceptable salt thereof, or the compound of Formula II, or the pharmaceutically acceptable salt thereof, (ii) the P-glycoprotein inhibitor, and (iii) a pharmaceutically acceptable excipient. In some embodiments, the pharmaceutical composition further comprises (iv) additional therapeutic agent or agents.

In some embodiments, the disclosure provides a kit comprising the combination of (i) the compound of Formula I, or the pharmaceutically acceptable salt thereof, or the compound of Formula II, or the pharmaceutically acceptable salt thereof, (ii) the P-glycoprotein inhibitor. In some embodiments, the kit comprises (i) a pharmaceutical composition comprising the compound of Formula I, or a pharmaceutically acceptable salt thereof, or the compound of Formula II, or a pharmaceutically acceptable salt thereof, (ii) a pharmaceutical composition comprising the P-glycoprotein inhibitor, and (iii) instructions for use.

In some embodiments, the kit comprises (i) a pharmaceutical composition comprising the compound of Formula I, or a pharmaceutically acceptable salt thereof, or the compound of Formula II, or a pharmaceutically acceptable salt thereof, and the P-glycoprotein inhibitor, and (ii) instructions for use. In some embodiments, the kit further comprising one, two, three, or four additional therapeutic agent or agents. In some embodiments, the additional therapeutic agent or agents are anti-HIV agents. In some embodiments, the additional therapeutic agent or agents are HIV protease inhibitors. HIV non-nucleoside or non-nucleotide inhibitors of reverse transcriptase, HIV nucleoside or nucleotide inhibitors of reverse transcriptase, HIV capsid inhibitors, gp4l inhibitors, CXCR4 inhibitors, gp120 inhibitors, CCR5 inhibitors, latency reversing agents, capsid polymerization inhibitors, HIV bNAbs, TLR7 agonists, pharmacokinetic enhancers, other drugs for treating HIV, or combinations thereof. In some embodiments, the additional therapeutic agent or agents are abacavir, tenofovir alafenamide, tenofovir disoproxil. N—((S)-1-(3-(4-chloro-3-(methylsulfonamido)-1-(2,2,2-trifluoroethyl)-1H-indazol-7-yl)-6-(3-methyl-3-(methylsulfonyl)but-1-yn-1-yl)-pyridin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide, or a pharmaceutically acceptable salt thereof.

In some embodiments, the disclosure provides a compound of Formula I, or a pharmaceutically acceptable salt thereof, or a compound of Formula II, or a pharmaceutically acceptable salt thereof, for use in combination therapy for treating an HIV infection with a P-glycoprotein inhibitor.

In some embodiments, the disclosure provides a P-glycoprotein inhibitor for use in combination therapy for treating an HIV infection with a compound of Formula I, or a pharmaceutically acceptable salt thereof, or a compound of Formula II, or a pharmaceutically acceptable salt thereof, as defined in any of the preceding claims.

In some embodiments, the disclosure provides use of a combination of (i) a compound of Formula I, or a pharmaceutically acceptable salt thereof, or a compound of Formula II, or a pharmaceutically acceptable salt thereof, and (ii) a P-glycoprotein inhibitor or a pharmaceutically acceptable salt thereof, in the manufacture of a medicament for treating an HIV infection in a human having or at risk of having the infection.

In some embodiments, the exposure of a metabolite of the compound of Formula I, or a pharmaceutically acceptable salt thereof, is increased when the compound of Formula I, or a pharmaceutically acceptable salt thereof, is coadministered with a P-glycoprotein inhibitor, relative to the exposure of the metabolite of the compound of Formula I, or a pharmaceutically acceptable salt thereof, when the compound of Formula I, or a pharmaceutically acceptable salt therefore, is dosed in the absence of the P-glycoprotein inhibitor. In some embodiments, the C_{MAX} of a metabolite of the compound of Formula I, or a pharmaceutically acceptable salt thereof, is increased when the compound of Formula I, or a pharmaceutically acceptable salt thereof, is coadministered with the P-glycoprotein inhibitor, relative to the C_{MAX} of the metabolite of the compound of Formula I, or a pharmaceutically acceptable salt thereof, when the compound of Formula I, or a pharmaceutically acceptable salt therefore, is dosed in the absence of the P-glycoprotein inhibitor. In some embodiments, the AUC_{INF} of a metabolite of the compound of Formula I, or a pharmaceutically acceptable salt thereof, is increased when the compound of Formula I, or a pharmaceutically acceptable salt thereof, is coadministered with the P-glycoprotein inhibitor, relative to the AUC_{INF} of the metabolite of the compound of Formula I, or a pharmaceutically acceptable salt thereof, when the compound of Formula I, or a pharmaceutically acceptable salt therefore, is dosed in the absence of the P-glycoprotein inhibitor. In some embodiments, the oral bioavailability of a metabolite of the compound of Formula I, or a pharmaceutically acceptable salt thereof, is increased when the compound of Formula I, or a pharmaceutically acceptable salt thereof, is coadministered with the P-glycoprotein inhibitor, relative to the oral bioavailability of the metabolite of the compound of Formula I, or a pharmaceutically acceptable salt thereof, when the compound of Formula I, or a pharmaceutically acceptable salt therefore, is dosed in the absence of the P-glycoprotein inhibitor. In some embodiments, the metabolite is a compound of Formula II, or a pharmaceutically acceptable salt thereof.

In some embodiments, the exposure of the compound of Formula II, or a pharmaceutically acceptable salt thereof, in the patient is increased when coadministered with the P-glycoprotein inhibitor, relative to the exposure of the compound of Formula II, or a pharmaceutically acceptable salt thereof, when dosed in the absence of the P-glycoprotein inhibitor. In some embodiments, the C_{MAX} of the compound of Formula II, or a pharmaceutically acceptable salt thereof, in the patient is increased when coadministered with the P-glycoprotein inhibitor, relative to the C_{MAX} of the compound of Formula II, or a pharmaceutically acceptable salt thereof, when dosed in the absence of the P-glycoprotein inhibitor. In some embodiments, the AUC_{INF} of the compound of Formula II, or a pharmaceutically acceptable salt thereof, when dosed in the absence of the P-glycoprotein inhibitor. In some embodiments, the oral bioavailability of the compound of Formula II, or a pharmaceutically acceptable salt thereof, in the patient is increased when coadministered with the P-glycoprotein inhibitor, relative to the oral bioavailability of the compound of Formula II, or a pharmaceutically acceptable salt thereof, when dosed in the absence of the P-glycoprotein inhibitor.

In the above embodiments, (i) a compound of Formula I, or a pharmaceutically acceptable salt thereof, or a compound of Formula II, or a pharmaceutically acceptable salt thereof and (ii) a P-glycoprotein inhibitor may be provided in pharmaceutical compositions described herein. For example, the compound of Formula I or Formula II, or a pharmaceutically acceptable salt thereof, and the PGP inhibitor may be present in the same pharmaceutical composition. Alternatively, the compound of Formula I or Formula II, or a pharmaceutically acceptable salt thereof, may be present in a different pharmaceutical composition to the PGP inhibitor.

In some embodiments, the current disclosure provides a method of treating an HIV infection in a human having or at risk of having the infection, comprising administering to the human (i) a therapeutically effective amount of a compound of Formula I, or a pharmaceutically acceptable salt thereof and (ii) a P-glycoprotein inhibitor. In some embodiments, a compound of Formula I is administered. In some embodiments, a pharmaceutically acceptable salt of a compound of Formula I disclosed herein is administered.

In some embodiments, the current disclosure provides a method of treating an HIV infection in a human having or at risk of having the infection, comprising administering to the human (i) a therapeutically effective amount of a compound of Formula II, or a pharmaceutically acceptable salt thereof and (ii) a P-glycoprotein inhibitor. In some embodiments, a compound of Formula II is administered. In some embodiments, a pharmaceutically acceptable salt of a compound of Formula II disclosed herein is administered.

In some embodiments, the P-glycoprotein inhibitor is selected from the group consisting of verapamil, dexverapamil, cyclosporine, zosuquidar, laniquidar, elacridar, tariquidar, encequidar, and pharmaceutically acceptable salts thereof. In some embodiments, the P-glycoprotein inhibitor is encequidar, or a pharmaceutically acceptable salt thereof. In some embodiments, the P-glycoprotein inhibitor is a salt of encequidar. In some embodiments, the P-glycoprotein inhibitor is a mesylate salt of encequidar.

In some embodiments of the methods disclosed herein, (i) the compound of Formula I, or the pharmaceutically acceptable salt thereof, or the compound of Formula II, or the pharmaceutically acceptable salt thereof and (ii) the P-glycoprotein inhibitor are administered together. In some embodiments, (i) the compound of Formula I, or the pharmaceutically acceptable salt thereof, or the compound of Formula II, or the pharmaceutically acceptable salt thereof and (ii) the P-glycoprotein inhibitor are administered as a co-formulation.

In some embodiments, (i) the compound of Formula I, or the pharmaceutically acceptable salt thereof, or the compound of Formula I, or the pharmaceutically acceptable salt thereof and (ii) the P-glycoprotein inhibitor are administered separately.

In some embodiments, (i) the compound of Formula I, or the pharmaceutically acceptable salt thereof, or the compound of Formula I, or the pharmaceutically acceptable salt thereof and (ii) the P-glycoprotein inhibitor are administered orally.

In some embodiments, (i) the compound of Formula I, or the pharmaceutically acceptable salt thereof, or the compound of Formula II, or the pharmaceutically acceptable salt thereof and (ii) the P-glycoprotein inhibitor are administered to the human along with a therapeutically effective amount of one, two, three, or four additional therapeutic agents. In some embodiments, the additional therapeutic agent or agents are anti-HIV agents. In some embodiments, the additional therapeutic agent or agents are HIV protease inhibitors, HIV non-nucleoside or non-nucleotide inhibitors of reverse transcriptase, HIV nucleoside or nucleotide inhibitors of reverse transcriptase, HIV capsid inhibitors, gp41 inhibitors, CXCR4 inhibitors, gp120 inhibitors, CCR5 inhibitors, latency reversing agents, capsid polymerization inhibitors, HIV bNAbs, TLR7 agonists, pharmacokinetic enhancers, other drugs for treating HIV, or combinations thereof. In some embodiments, the additional therapeutic agent or agents are abacavir, tenofovir alafenamide, tenofovir disoproxil, N—((S)-1-(3-(4-chloro-3-(methylsulfonamido)-1-(2,2,2-trifluoroethyl)-1H-indazol-7-yl)-6-(3-methyl-3-(methylsulfonyl)but-1-yn-1-yl)-pyridin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide, or a pharmaceutically acceptable salt thereof.

In some embodiments, the additional therapeutic agent or agents are HIV protease inhibitors, HIV non-nucleoside or non-nucleotide inhibitors of reverse transcriptase, HIV nucleoside or nucleotide inhibitors of reverse transcriptase and/or translocation, HIV capsid inhibitors, gp41 inhibitors, CXCR4 inhibitors, gp120 inhibitors, CCR5 inhibitors, latency reversing agents, capsid polymerization inhibitors, HIV bNAbs, TLR7 agonists, pharmacokinetic enhancers, other drugs for treating HIV, or combinations thereof.

In another embodiment, the current disclosure provides a use of (i) a compound of Formula I, or a pharmaceutically acceptable salt thereof, or a compound of Formula II, or a pharmaceutically acceptable salt thereof, and (ii) a P-glycoprotein inhibitor, for treating an HIV infection in a human having or at risk of having the infection.

In another embodiment, the current disclosure provides (i) a compound of Formula I, or a pharmaceutically acceptable salt thereof, or a compound of Formula II, or a pharmaceutically acceptable salt thereof, and (ii) a P-glycoprotein inhibitor, for use in medical therapy.

In another embodiment, the current disclosure provides (i) a compound of Formula I, or a pharmaceutically acceptable salt thereof, or a compound of Formula II, or a pharmaceutically acceptable salt thereof, and (ii) a P-glycoprotein inhibitor, for use in treating an HIV infection.

In another embodiment, the current disclosure provides use of (i) a compound of Formula I, or a pharmaceutically acceptable salt thereof, or a compound of Formula II, or a pharmaceutically acceptable salt thereof, and (ii) a P-glycoprotein inhibitor, in the manufacture of a medicament for treating an HIV infection in a human having or at risk of having the infection.

An advantage of the combinations disclosed herein is that the use of a PGP inhibitor in combination with a compound of Formula I or Formula II, or a pharmaceutically acceptable salt thereof, disclosed herein can modulate (e.g. increase) the PK properties of the compound, such as AUC, Cmax, and oral bioavailability. Thus, in some embodiments, (i) the compound of Formula I, or the pharmaceutically acceptable salt thereof, or the compound of Formula II, or the pharmaceutically acceptable salt thereof and (ii) the P-glycoprotein inhibitor, are administered orally.

The compound of Formula I or Formula II, or a pharmaceutically acceptable salt thereof, and the PGP inhibitor disclosed herein may be administered simultaneously or sequentially, such that effective amounts of the compound of Formula I or Formula II, or a pharmaceutically acceptable salt thereof, and the PGP inhibitor are both present in the body of the patient. When administered sequentially, the combination may be administered in two or more administrations.

Unit dosages of the compounds of Formula I or Formula II, or a pharmaceutically acceptable salt thereof, disclosed herein may be administered before or after administration of unit dosages the PGP inhibitor. For example, the compound of Formula I or Formula II, or a pharmaceutically acceptable salt thereof, disclosed herein may be administered within seconds, minutes, or hours of the administration of the PGP inhibitor. In some embodiments, a unit dose of a compound of Formula I or Formula II, or a pharmaceutically acceptable salt thereof, disclosed herein is administered first, followed within seconds or minutes by administration of a PGP inhibitor. Alternatively, a unit dose of a PGP inhibitor is administered first, followed by administration of a unit dose of a compound of Formula I or Formula II, or a pharmaceutically acceptable salt thereof, disclosed herein within seconds or minutes. In other embodiments, a unit dose of a compound of Formula I or Formula II, or a pharmaceutically acceptable salt thereof, disclosed herein is administered first, followed, after a period of hours (e.g., 0.1-12 hours), by administration of a unit dose of a PGP inhibitor. In yet other embodiments, a unit dose of a PGP inhibitor is administered first, followed, after a period of hours (e.g., 0.1-12 hours), by administration of a unit dose of a compound of Formula I or Formula II, or a pharmaceutically acceptable salt thereof, disclosed herein. In some embodiments, a unit dose of a PGP inhibitor is administered first, followed, after a period of 10-40 mins, by administration of a unit dose of a compound of Formula I or Formula II, or a pharmaceutically acceptable salt thereof, disclosed herein. In some embodiments, a unit dose of a PGP inhibitor is administered first, followed, after a period of 30 mins, by administration of a unit dose of a compound of Formula I or Formula II, or a pharmaceutically acceptable salt thereof, disclosed herein. In some embodiments, a unit dose of a PGP inhibitor is administered at the same time as the administration of a unit dose of a compound of Formula I or Formula II, or a pharmaceutically acceptable salt thereof, disclosed herein.

### VII. Examples

### Intermediate A: (3S,7S)-12-(benzyloxy)-N-(2,4-difluorobenzyl)-3-methyl-6-methylene-1,11-dioxo-1,4,5,6,7,11-hexahydro-3H-2,7-methanopyrido[1,2-a][1,4]diazonine-10-carboxamide (A)

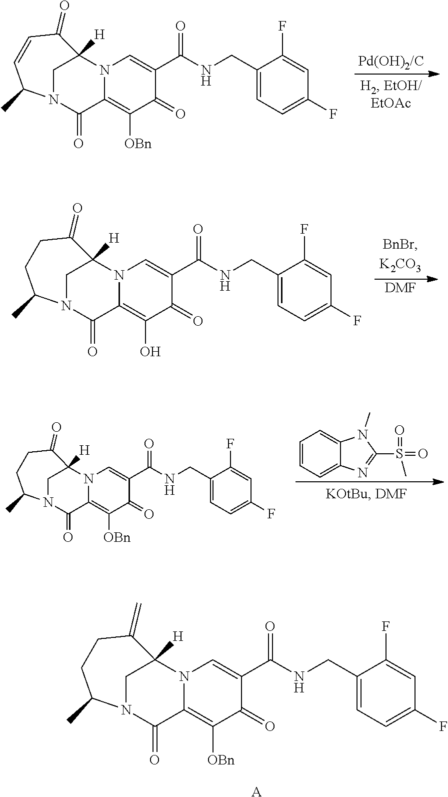

### Step 1: Synthesis of (3S,7R)—N-(2,4-difluorobenzyl)-12-hydroxy-3-methyl-1,6,11-trioxo-1,4,5,6,7,11-hexahydro-3H-2,7-methanopyrido[1,2-a][1,4]diazonine-10-carboxamide

To a solution of (3S,7R)-12-(benzyloxy)-N-(2,4-difluorobenzyl)-3-methyl-1,6,11-trioxo-1,6,7,11-tetrahydro-3H-2,7-methanopyrido[1,2-a][1,4]diazonine-10-carboxamide (17 g, 32.7 mmol) in EtOAc (200 mL) at room temperature was added EtOH (400 mL) followed by 20% Pd(OH)₂/C (50 wt % water, 7.6 g). The resulting mixture was degassed and flushed with nitrogen three times and then degassed and flushed with hydrogen three times before it was hydrogenated under hydrogen balloon for 4 hours. The reaction was then degassed and flushed with nitrogen, diluted with DCM, filtered through Celite®, concentrated and used directly in next step. MS (m/z) 432.124 [M+H]⁺.

### Step 2: Synthesis of (3S,7R)-12-(benzyloxy)-N-(2,4-difluorobenzyl-3-methyl-1,6,11-trioxo-1,4,5,6,7,11-hexahydro-3H-2,7-methanopyrido[1,2-a][1,4]diazonine-10-carboxamide

(3S,7R)—N-(2,4-difluorobenzyl)-12-hydroxy-3-methyl-1,6,11-trioxo-1,4,5,6,7,11-hexahydro-3H-2,7-methanopyrido[1,2-a][1,4]diazonine-10-carboxamide (33.1 g, 76.7 mmol) from Step 1 was dissolved in DMF (300 mL) at room temperature and K₂CO₃ (16.0 g, 115.0 mmol) and benzyl bromide (13.1 g, 76.7 mmol) were added. The resulting mixture was then heated to 50° C. for 4.5 hours and then cooled to room temperature. The mixture was filtered through a pad of Celite® and the filter cake was rinsed with DMF (100 mL). Combined filtrate was carried directly into the next step. MS (m/z) 554.086 [M+H+MeOH]⁺.

### Step 3: Synthesis of (35.71)-12-(benzyloxy-N-(2,4-difluorobenzyl)-3-methyl-6-methylene-1,11-dioxo-1,4,5,6,7,11-hexahydro-3H-2,7-methanopyrido[1,2-a][1,4]diazonine-10-carboxamide

The solution of (3S,7R)-12-(benzyloxy)-N-(2,4-difluorobenzyl)-3-methyl-1,6,11-trioxo-1,4,5,6,7,11-hexahydro-3H-2,7-methanopyrido[1,2-a][1,4]diazonine-10-carboxamide (39.7 g, 76.1 mmol) in DMF (350 mL) was immersed into a room temperature water bath. 1-methyl-2-(methylsulfonyl)-1H-benzo[d]imidazole (20.81 g, 99.0 mmol) was added in one portion followed by potassium tert-butoxide (21.36 g, 190 mmol) in 5 portions. The reaction was removed from the water bath and stirred at room temperature for 1.5 hours. The reaction was then quenched slowly with 0.5N HCl in water (180 mL) and extracted with EtOAc three times. The combined organic layer was washed with water and brine, dried over sodium sulfate, filtered and concentrated. The residue was purified by normal phase silica gel chromatography, eluting with 0-100% EtOAc/hexane, to afford intermediate A. MS (m/z) 520.060 [M+H].

### Intermediate B: Preparation of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (B)

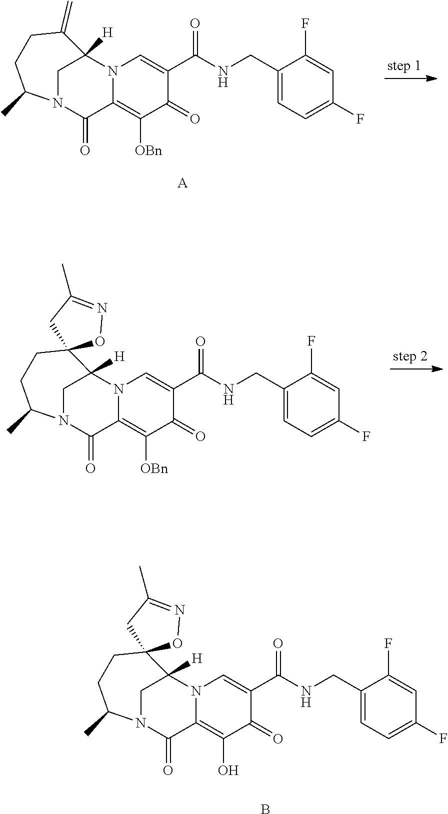

### Step 1: Preparation of (3′S,5S,7′R)-12′-(benzyloxy)-N-(2,4-difluorobenzyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide

Into the solution of acetaldehyde oxime (666 mg, 11.3 mmol) in DMF (50 ml) was added N-chlorosuccinimide (1.51 g, 11.3 mmol) at room temperature, then heated to 60° C. for 1 h. After cooling to room temperature, (3S,7S)-12-(benzyloxy)-N-(2,4-difluorobenzyl)-3-methyl-6-methylene-1,11-dioxo-1,4,5,6,7,11-hexahydro-3H-2,7-methanopyrido[1,2-a][1,4]diazonine-10-carboxamide (Intermediate A) (1.58 g, 3.04 mmol) and triethylamine (1.539 g, 15.2 mmol) were added at room temperature. After stirring at room temperature overnight, the reaction was quenched by adding sat. NaHCO₃ solution. The mixture was extracted with EtOAc, the organic phase was separated and dried over MgSO₄, filtered, concentrated down and purified by silica gel chromatography column (eluting with 0-100% EtOAc/hexane). MS (m/z) 577.135 [M+H]+

### Step 2: Preparation of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (B)

To a solution of (3′S,5S,7′R)-12′-(benzyloxy)-N-(2,4-difluorobenzyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (64.7 mg, 0.112 mmol) in toluene (2 mL) was added TFA (0.5 mL). The reaction mixture was stirred at room temperature overnight. The reaction mixture was concentrated down, and the residue was purified by reverse phase HPLC, eluting with 10-90% acetonitrile in water to give intermediate B. MS (m/z) 487.12 [M+H]⁺. ¹H NMR (400 MHz, Chloroform-d) δ 10.53 (s, 1H), 8.43 (s, 1H), 7.37 (td, J=8.6, 6.3 Hz, 1H), 6.92-6.78 (m, 2H), 4.82-4.70 (m, 1H), 4.67 (t, J=4.8 Hz, 2H), 4.18 (d, J=2.2 Hz, 1H), 3.86 (dd, J=14.9, 1.9 Hz, 1H), 3.72 (dd, J=14.9, 2.7 Hz, 1H), 2.94 (d, J=17.8 Hz, 1H), 2.53 (d, J=17.7 Hz, 1H), 2.06 (s, 3H), 2.04-1.88 (m, 3H), 1.56 (dd, J=14.3, 11.2 Hz, 1H), 1.32 (d, J=6.6 Hz, 3H).

### Intermediate C: Synthesis of (3′S,5S,7′R)—N-(2,4-difluorobenzyl)-12′-hydroxy-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide

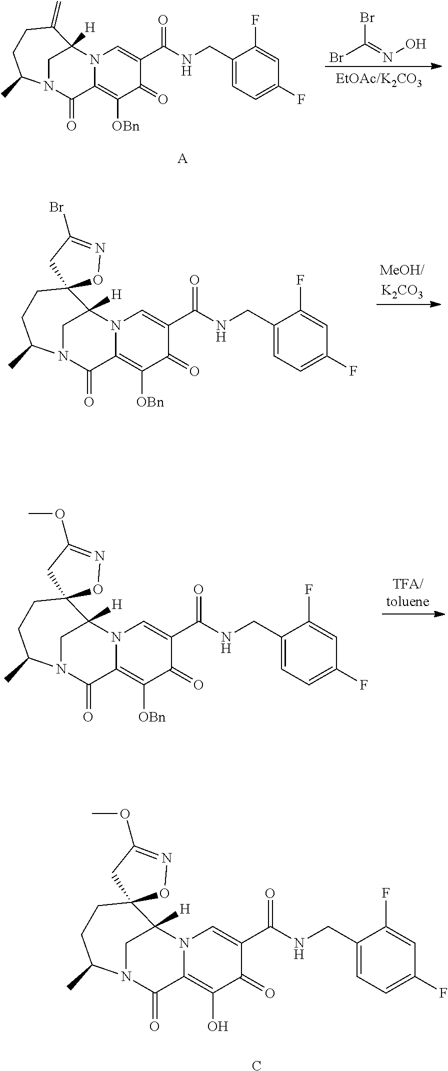

### Step 1: Synthesis of (3′S,5S,7′R)-12′-(benzyloxy)-3-bromo-N-(2,4-difluorobenzyl)-3′-methyl-1′,11′-dioxo-1′,4′,5′,11′-tetrahydro-3′H,4H,7′H-spiro[isoxazole-5′,6′-[2,7]methanopyrido[1,2-a][1,4]diazonine]-10′-carboxamide

To a solution of (3S,7S)-12-(benzyloxy)-N-(2,4-difluorobenzyl)-3-methyl-6-methylene-1,11-dioxo-1,4,5,6,7,11-hexahydro-3H-2,7-methanopyrido[1,2-a][1,4]diazonine-10-carboxamide (Intermediate A) (18.1 g, 34.8 mmol) in EtOAc (350 mL) at 0° C. was added dibromomethanone oxime (21.199 g, 105 mmol) followed by potassium carbonate (28.846 g, 209 mmol). The resulting mixture was stirred overnight before it was diluted with water (300 mL). The layers were separated. The aqueous layer was extracted with EtOAc (200 mL) and the combined organic phase was washed with brine, dried over sodium sulfate, filtered and concentrated. The residue was purified by normal phase silica gel chromatography, eluting with 0-80% EtOAc/Hexane to afford the title compound. MS (m/z) 640.904 [M+H]⁺. 1H NMR (400 MHz, DMSO) δ 10.41 (t, J=6.0 Hz, 1H), 8.89 (s, 1H), 7.54-7.21 (m, 7H), 7.13-7.05 (m, 1H), 5.30 (d, J=10.5 Hz, 1H), 5.04 (d, J=10.5 Hz, 1H), 4.78 (s, 1H), 4.72-4.54 (m, 3H), 3.75-3.62 (m, 2H), 3.39 (d, J=17.6 Hz, 1H), 3.07 (d, J=17.7 Hz, 1H), 1.92-1.73 (m, 3H), 1.41 (t, J=12.9 Hz, 1H), 1.15 (d, J=6.7 Hz, 3H).

### Step 2: Synthesis of (3′S,5S,7′R)-12′-(benzyloxy)-N-(2,4-difluorobenzyl-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide

To a mixture of (3′S,5S,7′R)-12′-(benzyloxy)-3-bromo-N-(2,4-difluorobenzyl)-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (23.2 g, 36.2 mmol) in MeOH (450 mL) and DMF (100 mL) at 55° C. was added potassium carbonate (12.496 g, 90.4 mmol). The reaction was stirred for 3 hrs and then cooled to room temperature. To this stirred mixture was added water (1100 mL) and stirring continued for 30 minutes. The mixture was filtered and the filter cake was rinsed with water, collected, and dried to give the title compound. MS (m/z) 593.207 [M+H]⁺. 1H NMR (400 MHz, DMSO) δ 10.41 (t, J=6.0 Hz, 1H), 8.73 (s, 1H), 7.53-7.48 (m, 2H), 7.47-7.30 (m, 4H), 7.25 (ddd, J=10.5, 9.3, 2.6 Hz, 1H), 7.08 (tdd, J=8.5, 2.6, 1.0 Hz, 1H), 5.30 (d, J=10.5 Hz, 1H), 5.03 (d, J=10.5 Hz, 1H), 4.73-4.63 (m, 2H), 4.63-4.49 (m, 2H), 3.82 (s, 3H), 3.65 (d, J=2.0 Hz, 2H), 3.08 (d, J=16.8 Hz, 1H), 2.77 (d, J=16.9 Hz, 1H), 1.96-1.71 (m, 3H), 1.38 (dd, J=15.5, 10.2 Hz, 1H), 1.15 (d, J=6.6 Hz, 3H).

### Step 3: Synthesis of (3′S,5S,7′R)—N-(2,4-difluorobenzyl)-12′-hydroxy-3-methoxy-3′-methyl-1′,11′-dioxo-1′,4′,5′,11′-tetrahydro-3′H,4H,7′H-spiro[isoxazole-5,6′-[2, 7]methanopyrido[1,2-a][1,4]diazonine]-10′-carboxamide (C)

To a solution of (3′S,5S,7′R)-12′-(benzyloxy)-N-(2,4-difluorobenzyl)-3-methoxy-3′-methyl-1′,1′-dioxo-1′,4′,5′,11′-tetrahydro-3′H,4H,7′H-spiro[isoxazole-5,6′-[2,7]methanopyrido[1,2-a][1,4]diazonine]-10′-carboxamide (19 g, 32.1 mmol) in toluene (64 mL) at room temperature was added TFA (32 mL) and stirred for 16 hrs. The solvents were removed by rotatory evaporator. The resulting residue was treated with EtOAc (60 mL) and concentrated, repeating three times. The residue was stirred with a mixture of toluene (42 mL)/MeOH (42 mL)/EtOAc (42 mL) at 70° C. for 1 hr and room temperature for 2 hours. The mixture was then filtered and the filter cake was rinsed with EtOAc, collected and dried to give title compound. MS (m/z) 503.263 [M+H]⁺. 1H NMR (400 MHz, DMSO) δ 10.99 (s, 1H), 10.34 (t, J=5.9 Hz, 1H), 8.64 (s, 1H), 7.42 (td, J=8.7, 6.6 Hz, 1H), 7.25 (ddd, J=10.5, 9.3, 2.6 Hz, 1H), 7.08 (tdd, J=8.6, 2.6, 1.0 Hz, 1H), 4.74 (s, 1H), 4.63-4.47 (m, 3H), 3.81 (s, 3H), 3.80-3.67 (m, 2H), 2.93 (d, J=16.9 Hz, 1H), 2.70 (d, J=16.9 Hz, 1H), 1.95-1.76 (m, 3H), 1.41-1.31 (m, 1H), 1.19 (d, J=6.8 Hz, 3H).

### Intermediate D: Synthesis of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-nitrophenyl) carbonate

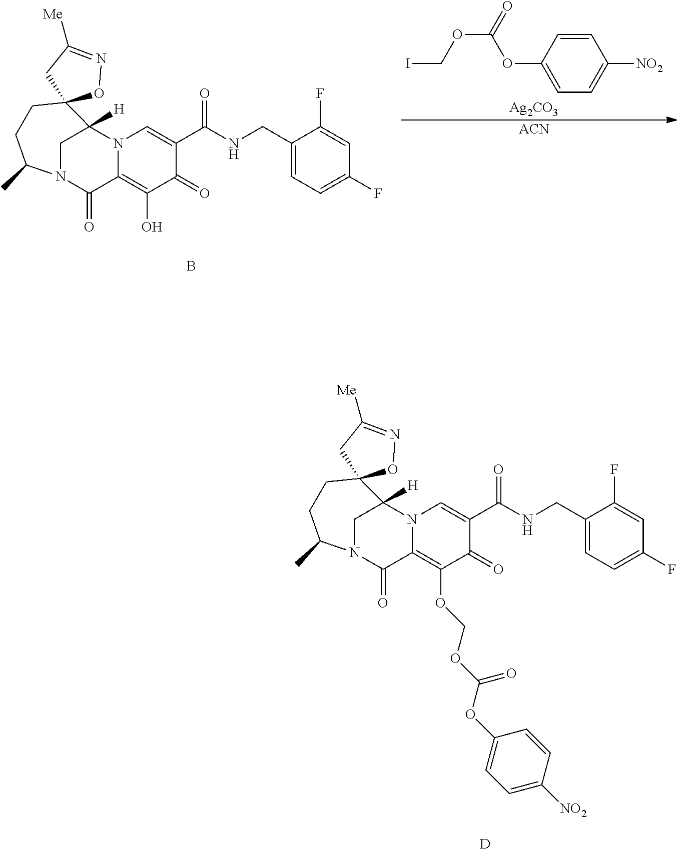

(3′S,5S,7′R)—N-(2,4-difluorobenzyl)-12′-hydroxy-3,3-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 3.06 g, 6.29 mol) and iodomethyl (4-nitrophenyl) carbonate (4.23 g, 13.1 mmol), prepared according to WO2010011812, were mixed with acetonitrile (73 mL). Ag₂CO₃ (5.29 g, 19.2 mmol) was added in one portion. The slurry was stirred at room temperature overnight and filtered through celite. The filtrate was collected and concentrated to dryness. The residue was purified on silica gel column with 0-20% MeOH in DCM to afford the crude material upon concentration. The crude product was then dissolved in EtOAc (200 mL) and was treated with water (200 mL) with agitation. Organic phase was separated, dried over Na₂SO₄ and filtered. The filtrate was concentrated to afford Intermediate D. Calculated for C₃₂H₂₉F₂N₅O₁₀: 681.19, Found MS (ESI+): 682.01 [M+H]+.

### Intermediate E: Synthesis of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-nitrophenyl) carbonate

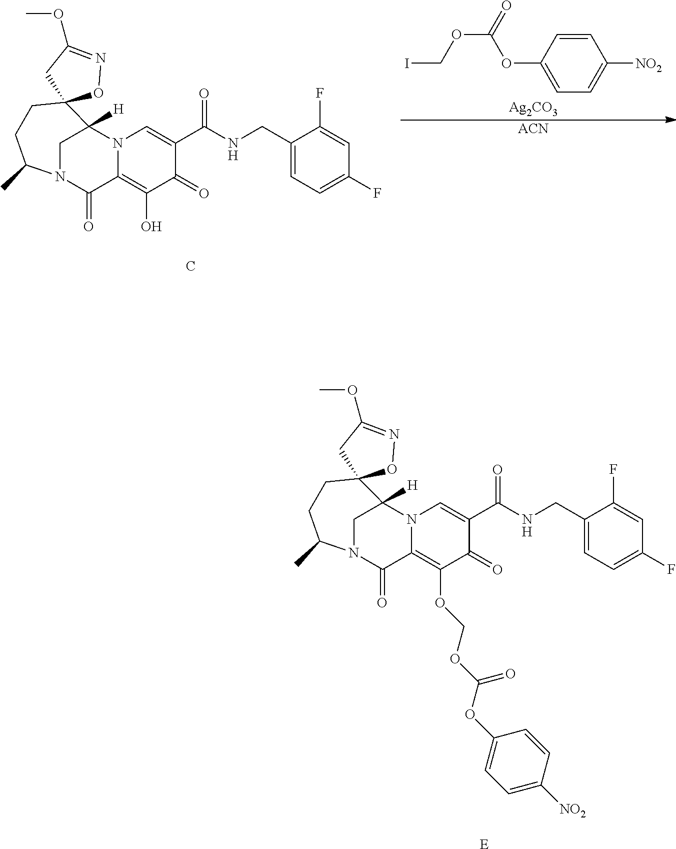

The title compound was prepared following a similar method for Intermediate D, except using Intermediate C instead of Intermediate B. LCMS-ESI+ (m/z): calcd H+ for C32H29F2N5011, Theoretical: 697.18, Found: 697.845.

### Example 1: Synthesis of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl dihydrogen phosphate (1)

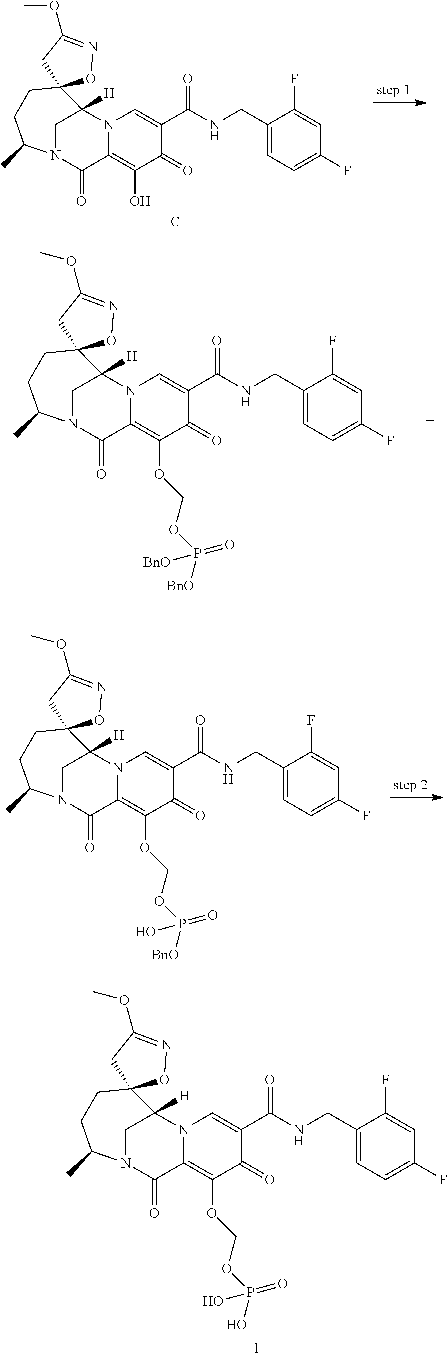

### Step 1: Synthesis of dibenzyl ((((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) phosphate and benzyl ((((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) hydrogen phosphate

To a solution of (3′S,5S,7′R)—N-(2,4-difluorobenzyl)-12′-hydroxy-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate C, 30 mg, 0.0597 mmol) in DMF (0.5 mL) at room temperature was added tetrabutylammonium iodide (14.7 mg, 0.0398 mmol) and potassium carbonate (11.0 mg, 0.0796 mmol). The resulting mixture was heated at 63° C. Dibenzyl chloromethyl phosphate (19.5 mg, 0.0597 mmol) was added to the hot mixture and the newly formed mixture was stirred for 30 minutes. The reaction was then cooled to room temperature and filtered. The mother liquor was purified by reverse phase preparative chromatography, fractions with desired mass were pooled and lyophilized. LCMS-ESI+ (m/z): calcd H+ for C39H39F2N4O10P, Theoretical: 792.24, Found: 792.697. LCMS-ESI+ (m/z): calcd H+ for C32H33F2N4O10P, Theoretical: 702.19, Found: 702.722.

### Step 2: Synthesis of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl dihydrogen phosphate (1)

The mixture of dibenzyl ((((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) phosphate and benzyl ((((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) hydrogen phosphate was dissolved in THF (10 mL) at room temperature. Platinum oxide (6.0 mg) was added and the resulting mixture was degassed and flushed with nitrogen three times. The reaction mixture was then degassed and flushed with hydrogen three times before it was hydrogenated under hydrogen balloon for 45 minutes. The reaction was then degassed and flushed with nitrogen, filtered, and concentrated. The residue was redissolved in DMF, filtered and purified by reverse phase preparative chromatography (5-100% MeCN/H₂O w/ 0.1% TFA). 1H NMR (400 MHz, Acetonitrile-d3) δ 9.85 (t, J=6.0 Hz, 1H), 8.68 (s, 1H), 7.50-7.38 (m, 1H), 7.03-6.92 (m, 2H), 5.70-5.57 (m, 2H), 4.86-4.72 (m, 1H), 4.61 (d, J=5.5 Hz, 2H), 4.49 (s, 1H), 3.88 (s, 3H), 3.80-3.69 (m, 2H), 2.98 (d, J=17.3 Hz, 1H), 2.70 (d, J=17.3 Hz, 1H), 2.07-1.81 (m, 3H), 1.60-1.48 (m, 1H), 1.23 (d, J=6.7 Hz, 3H). LCMS-ESI+(m/z): calcd H+ for C25H27F2N4O10P, Theoretical: 612.14, Found: 612.662.

### Example 2: Synthesis of N-(4-(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)-3,3-dimethyl-4-oxobutyl)-N-(((phosphonooxy)methoxy)carbonyl)glycine (2)

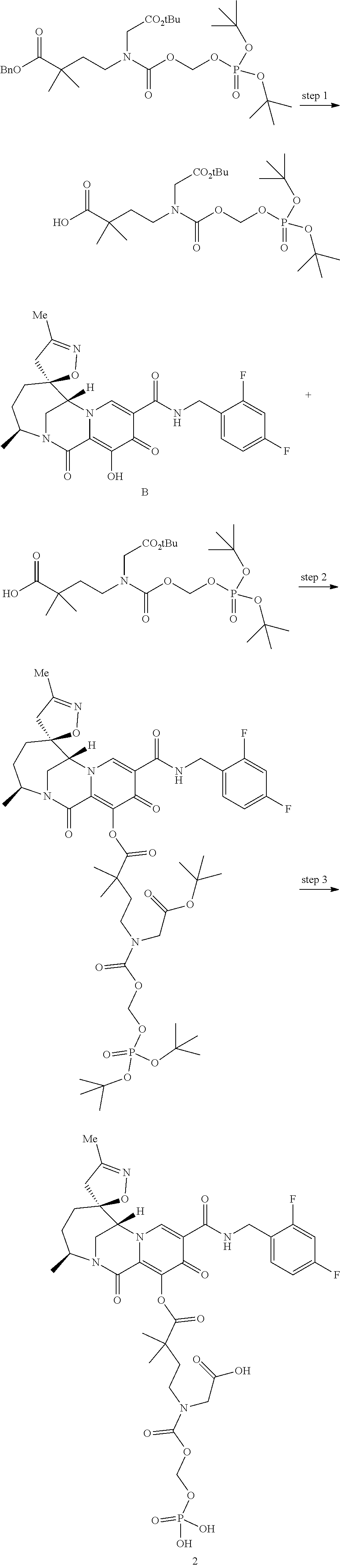

### Step 1: Synthesis of 4-((2-(tert-butoxy)-2-oxoethyl)((((di-tert-butoxyphosphoryl)oxy)methoxy)carbonyl)amino)-2,2-dimethylbutanoic acid

The solution of benzyl 4-((2-(tert-butoxy)-2-oxoethyl)((((di-tert-butoxyphosphoryl)oxy)methoxy)carbonyl)amino)-2,2-dimethylbutanoate (prepared according to WO2023102239) (200 mg, 0.332 mmol) in MeOH (20.0 mL) at room temperature was treated with platinum oxide (20.0 mg, 0.088 mmol). The resulting mixture was degassed and flushed with nitrogen three times and then degassed and flushed with hydrogen three times before it was hydrogenated under hydrogen balloon for 4 hours. The reaction was then degassed and flushed with nitrogen, diluted with DCM, filtered and concentrated and used directly in next step. LCMS-ESI+ (m/z): calcd H+ for C22H42NO10P, Theoretical: 511.25, Found: 511.512.

### Step 2: Synthesis of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 4-((2-(tert-butoxy)-2-oxoethyl)((((di-tert-butoxyphosphoryl)oxy)methoxy)carbonyl)amino)-22-dimethylbutanoate

To a mixture of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 60 mg, 0.123 mmol) and 4-((2-(tert-butoxy)-2-oxoethyl)((((di-tert-butoxyphosphoryl)oxV)methoxy)carbonyl)amino)-2,2-dimethylbutanoic acid (75.7 mg, 0.148 mmol) in DMF (0.5 mL) at room temperature was added 1-ethyl-3-(3-dimethylaminopropyl)carbodiimide hydrochloride (30.6 mg, 0.16 mmol), DMAP (19.6 mg, 0.16 mmol), and triethylamine (25.0 mg, 0.247 mmol). After stirring for 16 hours, the reaction was diluted with EtOAc, washed with 0.5N aq HCl, water, brine, dried over sodium sulfate, filtered, concentrated and purified by normal phase chromatography. MS (m/z) 979.529 [M]+.

### Step 3: Synthesis of N-(4-((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)-3,3-dimethyl-4-oxobutyl)-N-(((phosphonooxy)methoxy)carbonyl)glycine (2)

(3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 4-((2-(tert-butoxy)-2-oxoethyl)((((di-tert-butoxyphosphoryl)oxy)methoxy)carbonyl)amino)-2,2-dimethylbutanoate (60 mg) was dissolved in DCM (3.0 mL) at room temperature and treated with TFA (1.0 mL) slowly. After stirred for 30 minutes, the reaction was then concentrated, dissolved in DMF, filtered and purified by reverse phase preparative HPLC (5-100% MeCN/H₂O w/ 0.1% TFA) to afford the title compound. 1H NMR (400 MHz, Acetone-d6) δ 10.23 (s, 1H), 8.68 (d, J=7.1 Hz, 1H), 7.48 (td, J=8.6, 6.2 Hz, 1H), 7.09-6.89 (m, 2H), 5.72-5.53 (m, 2H), 4.86-4.72 (m, 1H), 4.72-4.55 (m, 3H), 4.36-4.13 (m, 2H), 4.07-3.77 (m, 2H), 3.74-3.44 (m, 2H), 3.29-3.16 (m, 1H), 2.85-2.69 (m, 1H), 2.01 (s, 5H), 1.97-1.83 (m, 3H), 1.67-1.43 (m, 1H), 1.36 (s, 6H), 1.26 (d, J=6.6 Hz, 3H). LCMS-ESI+ (m/z): calcd H+ for C34H40F2N5P14P, Theoretical: 811.23, Found: 811.769.

### Example 3: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (2-(phosphonooxy)ethyl) carbonate (3)

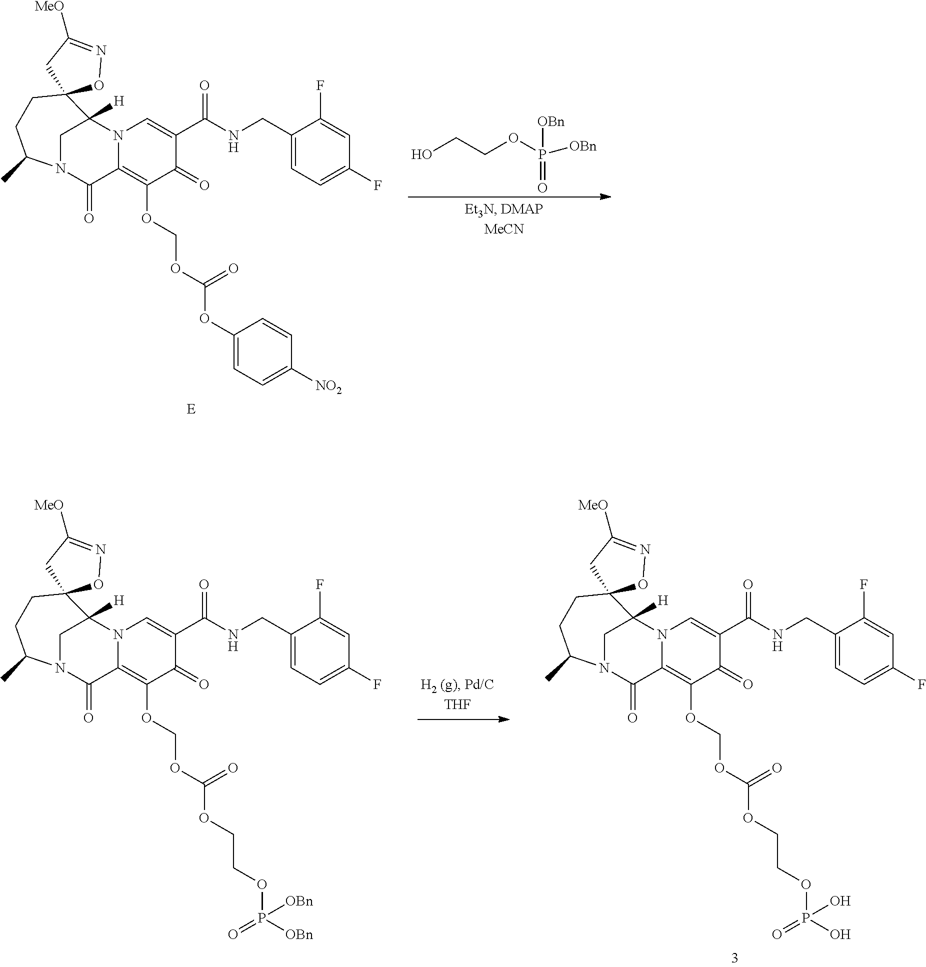

### Step 1: Preparation of 2-((bis(benzyloxy)phosphoryl)oxy)ethyl ((((3′S 5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate

To a solution of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-nitrophenyl) carbonate (Intermediate E) (11.9 g, 17.1 mmol) in MeCN (250 mL) was added Et₃N (7.13 mL, 51.2 mmol) and DMAP (0.208 g, 1.71 mmol), followed by dibenzyl (2-hydroxyethyl) phosphate (16.5 g, 51.2 mmol), prepared according to WO2010039474. The reaction mixture was left to stir at room temperature for 16 h and concentrated. The residue was purified by silica gel column chromatography (0-100% EtOAc/hexane then 0-20% MeOH/DCM, and again using 50-100% EtOAc/hexane) to afford the title compound. MS (m/z) 880.82 [M+H]+.

### Step 2: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (2-(phosphonooxy)ethyl) carbonate (3)

To a solution of 2-((bis(benzyloxy)phosphoryl)oxy)ethyl ((((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate (6.90 g, 7.83 mmol) in THF (500 mL) was added 10% Pd/C (0.834 g, 0.783 mmol). The flask was evacuated and backfilled with hydrogen gas (2×), then sparged with hydrogen for 2 min. The reaction mixture was left to stir under a hydrogen balloon atmosphere for 4 h. The reaction mixture was filtered through Celite and concentrated. The residue was dissolved in 1:1 MeCN/water and lyophilized to afford the title compound. MS (m/z) 700.90 [M+H]+. 1H NMR (400 MHz, DMSO-d6) δ 10.26 (t, J=6.0 Hz, 1H), 8.74 (s, 1H), 7.41 (td, J=8.7, 6.7 Hz, 1H), 7.24 (td, J=9.9, 2.6 Hz, 1H), 7.07 (td, J=8.5, 2.5 Hz, 1H), 5.80 (d, J=6.3 Hz, 1H), 5.64 (d, J=6.4 Hz, 1H), 4.71 (s, 1H), 4.62 (dq, J=14.5, 6.9 Hz, 1H), 4.55 (t, J=5.7 Hz, 2H), 4.23 (q, J=4.3 Hz, 2H), 4.00 (dt, J=7.1, 4.7 Hz, 2H), 3.81 (s, 3H), 3.75-3.59 (m, 2H), 3.05 (d, J=16.8 Hz, 1H), 2.75 (d, J=16.9 Hz, 1H), 1.98-1.65 (m, 3H), 1.37-1.29 (m, 1H), 1.15 (d, J=6.7 Hz, 3H).

### Example 4: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (2-(phosphonooxy)ethyl) carbonate (4)

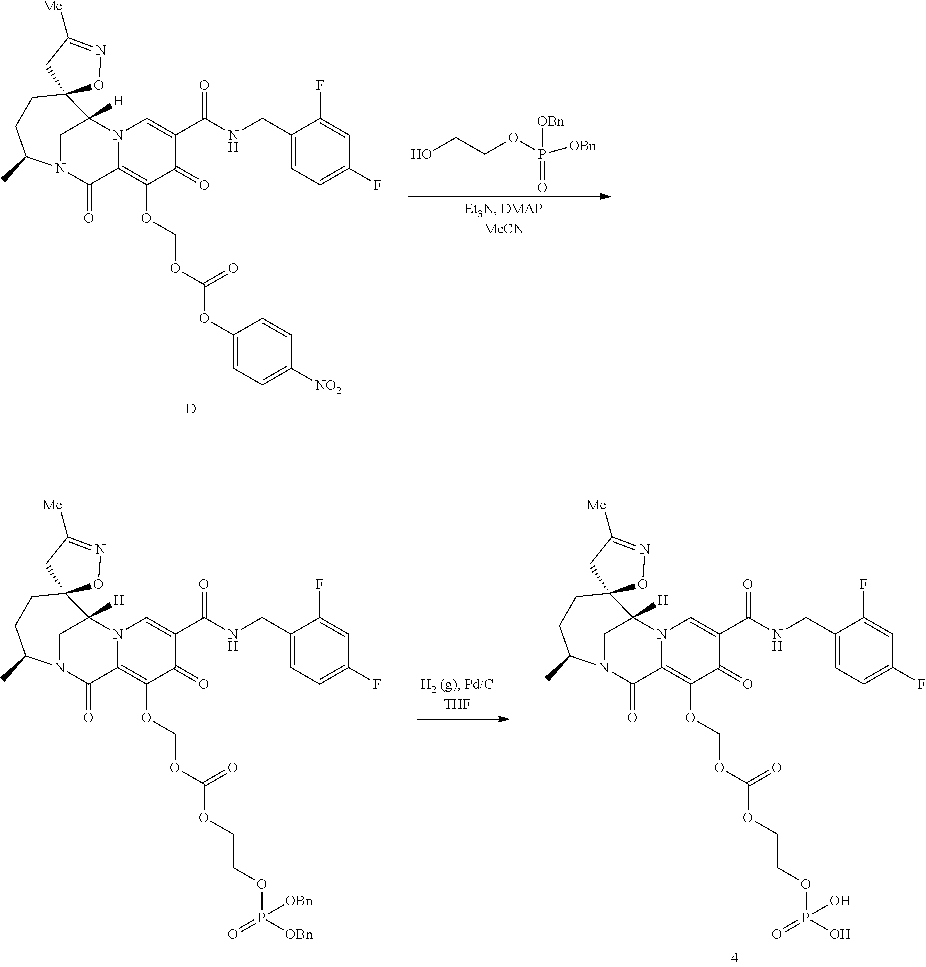

### Step 1: Preparation of 2-((bis(benzyloxy)phosphoryl)oxy)ethyl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate

To a solution of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-nitrophenyl) carbonate (Intermediate D) (2.50 g, 3.67 mmol) in MeCN (95 mL) was added Et₃N (2.56 mL, 18.3 mmol), DMAP (0.045 g, 0.367 mmol), and dibenzyl (2-hydroxyethyl) phosphate (2.36 g, 7.34 mmol), prepared according to WO2010039474. The reaction mixture was left to stir at room temperature for 16 h then diluted with EtOAc and quenched with aqueous NaHCO₃ solution. The phases were separated, and the aqueous phase was extracted with EtOAc. The combined organic phase was washed with brine, dried over Na₂SO₄, filtered, and concentrated. The residue was purified by silica gel column chromatography (20-100% EtOAc/hexane) and reverse phase prep HPLC (5-100% MeCN/H2O) to afford the title compound. MS (m/z) 864.87 [M+H]+.

### Step 2: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5′,6′-[2,7]methanopyrido[1,2-a][1,4]diazonin]-12′-yl)oxy)methyl (2-(phosphonooxy)ethyl) carbonate (4)

To a solution of 2-((bis(benzyloxy)phosphoryl)oxy)ethyl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate (0.336 g, 0.389 mmol) in THF (42 mL) was added 5% Pd/C (0.083 g, 0.039 mmol). The flask was evacuated and backfilled with hydrogen gas (2×), then sparged with hydrogen for 2 min. The reaction mixture was left to stir under a hydrogen balloon atmosphere for 2 h. The reaction mixture was filtered through Celite and concentrated. The residue was dissolved in 1:1 MeCN/water and lyophilized to afford the title compound. MS (m/z) 684.82 [M+H]. 1H NMR (400 MHz, DMSO-d6) δ 10.27 (t, J=6.0 Hz, 1H), 8.70 (s, 1H), 7.42 (td, J=8.7, 6.6 Hz, 1H), 7.24 (ddd, J=10.5, 9.3, 2.6 Hz, 1H), 7.07 (ddd, J=10.3, 8.2, 2.6 Hz, 1H), 5.81 (d, J=6.4 Hz, 1H), 5.65 (d, J=6.4 Hz, 1H), 4.64 (d, J=11.0 Hz, 2H), 4.59-4.46 (m, 2H), 4.22 (q, J=4.3 Hz, 2H), 3.99 (dd, J=7.2, 4.5 Hz, 2H), 3.76-3.61 (m, 2H), 3.00 (d, J=17.5 Hz, 1H), 2.63 (d, J=17.6 Hz, 1H), 1.94 (s, 3H), 1.89-1.67 (m, 3H), 1.35-1.22 (m, 1H), 1.15 (d, J=6.7 Hz, 3H).

### Example 5: Preparation of 1-(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)ethyl (2-(phosphonooxy)ethyl) carbonate (5)

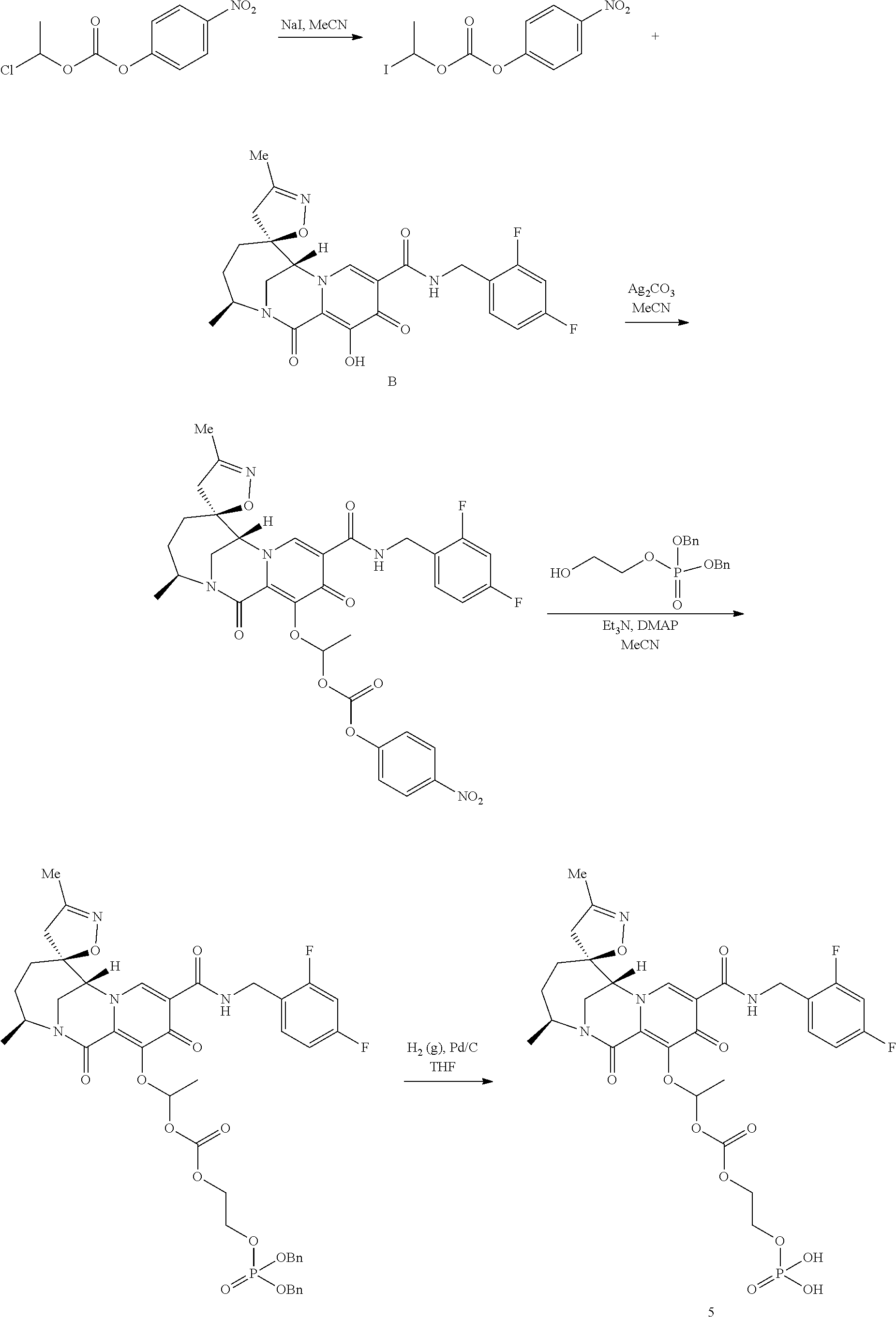

### Step 1: Preparation of 1-iodoethyl (4-nitrophenyl) carbonate

To a solution of 1-chloroethyl (4-nitrophenyl) carbonate (2.00 g, 8.14 mmol) in MeCN (16 mL) was added sodium iodide (8.54 g, 57.0 mmol) and heated to 50° C. After 16 h, the mixture was concentrated and suspended in EtOAc. The organic phase was washed with brine, Na₂S₂O₃, NaHCO₃ (2×), and brine. The organic phase was dried over Na₂SO₄, filtered, and concentrated to yield a residue, which was purified by silica gel column chromatography (0-100% EtOAc/hexane) to afford the title compound. MS (m/z) 336.02 [M]+.

### Step 2: Preparation of 1-(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)ethyl (4-nitrophenyl) carbonate

To a solution of 1-iodoethyl (4-nitrophenyl) carbonate (0.831 g, 2.47 mmol) in MeCN (10 mL) was added (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 0.400 g, 0.822 mmol) and silver carbonate (0.680 g, 2.47 mmol). The mixture was stirred at 50° C. for 16 h and concentrated. The residue was purified by reverse phase prep HPLC (10-100% MeCN/H₂O containing 0.1% TFA) to afford the title compound. MS (m/z) 695.39 [M]+.

### Step 3: Preparation of 2-((bis(benzyloxy)phosphoryl)oxy)ethyl (1-(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)ethyl) carbonate

To a solution of 1-(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)ethyl (4-nitrophenyl) carbonate (0.171 g, 0.246 mmol) in MeCN (10 mL) was added sequentially dibenzyl (2-hydroxyethyl) phosphate (0.238 g, 0.737 mmol), prepared according to WO2010039474, Et₃N (0.10 mL, 0.737 mmol), and DMAP (0.003 g, 0.025 mmol). The mixture was stirred at room temperature for 16 h and concentrated. The residue was redissolved in MeCN and purified by reverse phase prep HPLC (10-100% MeCN/water w/ 0.1% TFA) to afford the title compound. MS (m/z) 878.71 [M+H]+.

### Step 4: Preparation of 1-(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)ethyl (2-(phosphonooxy)ethyl) carbonate (5)

To a solution of 2-(bis(benzyloxy)phosphoryl)oxy)ethyl (1-(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)ethyl) carbonate (0.070 g, 0.080 mmol) in THF (5 mL) was added 10% Pd/C (0.008 g, 0.008 mmol). The flask was evacuated and backfilled with hydrogen gas (2×), then sparged with hydrogen for 2 min. The reaction mixture was left to stir under a hydrogen balloon atmosphere for 4 h. The reaction mixture was filtered through Celite and concentrated. The residue was dissolved in 1:1 MeCN/water and lyophilized to afford the title compound. MS (m/z) 698.80 [M+H]⁺. 1H NMR (400 MHz, DMSO-d6) δ 10.28 (t, J=6.0 Hz, 1H), 8.70 (s, 1H), 7.42 (td, J=8.6, 6.5 Hz, 1H), 7.29-7.18 (m, 1H), 7.07 (td, J=8.6, 2.7 Hz, 1H), 6.50 (q, J=5.2 Hz, 1H), 4.71-4.60 (m, 2H), 4.60-4.48 (m, 2H), 4.11 (dt, J=10.6, 5.1 Hz, 1H), 4.07-4.01 (m, 1H), 3.94 (dd, J=7.1, 4.6 Hz, 2H), 3.81 (dt, J=7.4, 5.4 Hz, 1H), 3.53 (d, J=10.8 Hz, 1H), 2.97 (d, J=17.5 Hz, 1H), 2.58 (d, J=17.3 Hz, 1H), 1.95 (d, J=3.4 Hz, 3H), 1.87-1.73 (m, 3H), 1.60 (d, J=5.3 Hz, 3H), 1.19 (d, J=7.2 Hz, 1H), 1.14 (d, J=6.6 Hz, 3H).

### Example 6: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (2-(phosphonooxy)ethyl) carbonate (6)

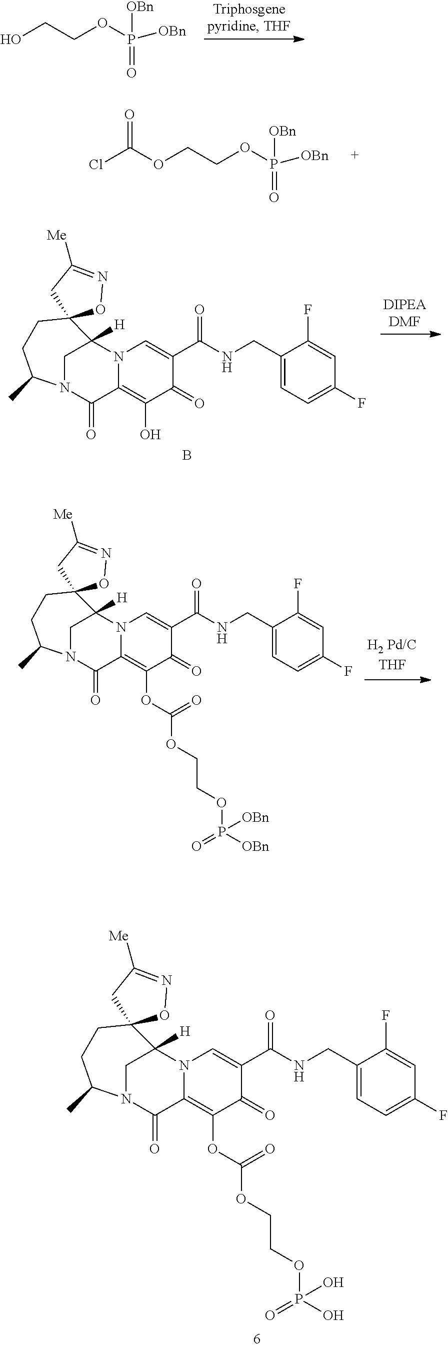

### Step 1: Preparation of 2-((bis(benzyloxy)phosphoryl)oxy)ethyl carbonochloridate

To a solution of triphosgene (0.034 g, 0.116 mmol) in THF (1.5 mL) was added pyridine (0.024 mL, 0.303 mmol) and the suspension was cooled to 0° C. To this mixture was added a solution of dibenzyl (2-hydroxyethyl) phosphate (0.075 g, 0.233 mmol), prepared according to WO2010039474, in THF (1.5 mL). The mixture was allowed to warm to room temperature and stir for 4 h. The reaction mixture was filtered through Celite, rinsing with DCM. The organic filtrate was washed with 1 N HCl, dried over Na₂SO₄, filtered, and concentrated to afford the title compound. MS (m/z) 366.80 [M+H2O—Cl]+.

### Step 2: Preparation of 2-((bis(benzyloxy)phosphoryl)oxy)ethyl ((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl) carbonate

To a mixture of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 0.075 g, 0.154 mmol) and DIPEA (0.081 mL, 0.463 mmol) in DMF (1 mL) was added 2-((bis(benzyloxy)phosphoryl)oxy)ethyl carbonochloridate (0.089 g, 0.231 mmol) at 0° C. The reaction mixture was warmed to room temperature and stirred for 16 h. Additional portions of 2-((bis(benzyloxy)phosphoryl)oxy)ethyl carbonochloridate (0.089 g, 0.231 mmol) and DIPEA (0.081 mL, 0.463 mmol) were added and stirred for 16 h. The reaction mixture was diluted with EtOAc, washed with 5% LiCl (aq), water, and brine. The organic phase was dried over Na₂SO₄, filtered, and concentrated to afford a residue, which was purified by column chromatography (0-100% EtOAc/hexane) and reverse phase prep HPLC (5-100% MeCN/water) to afford the title compound. MS (m/z) 834.89 [M+H]+.

### Step 3: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (2-(phosphonooxyethyl) carbonate (6)

To a solution of 2-((bis(benzyloxy)phosphoryl)oxy)ethyl ((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl) carbonate (0.014 g, 0.017 mmol) in THF (2 mL) was added 5% Pd/C (0.004 g, 0.002 mmol). The vial was evacuated and backfilled with hydrogen gas (2×), then sparged with hydrogen gas for 2 min. The reaction mixture was left to stir under a hydrogen balloon atmosphere for 2 h. The reaction mixture was filtered through Celite, rinsing with THF, and concentrated. The residue was dissolved in 1:1 MeCN/water and lyophilized to afford the title compound. MS (m/z) 654.99 [M+H]⁺. 1H NMR (400 MHz, DMSO-d6) δ 10.12 (t, J=6.0 Hz, 1H), 8.85 (s, 1H), 7.47-7.37 (m, 1H), 7.25 (td, J=10.0, 2.6 Hz, 1H), 7.14-7.02 (m, 1H), 4.72 (s, 1H), 4.56 (dq, J=15.3, 9.0, 7.7 Hz, 3H), 4.39-4.26 (m, 2H), 4.05 (d, J=6.2 Hz, 2H), 3.83 (d, J=15.8 Hz, 1H), 3.71 (d, J=15.2 Hz, 1H), 3.03 (d, J=17.4 Hz, 1H), 2.67 (d, J=17.7 Hz, 1H), 1.95 (s, 3H), 1.78 (d, J=19.4 Hz, 3H), 1.30-1.24 (m, 1H), 1.17 (d, J=6.6 Hz, 3H).

### Example 7: Preparation of N-(2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)oxy)ethyl)-N-(((phosphonooxy)methoxy)carbonyl)glycine (7)

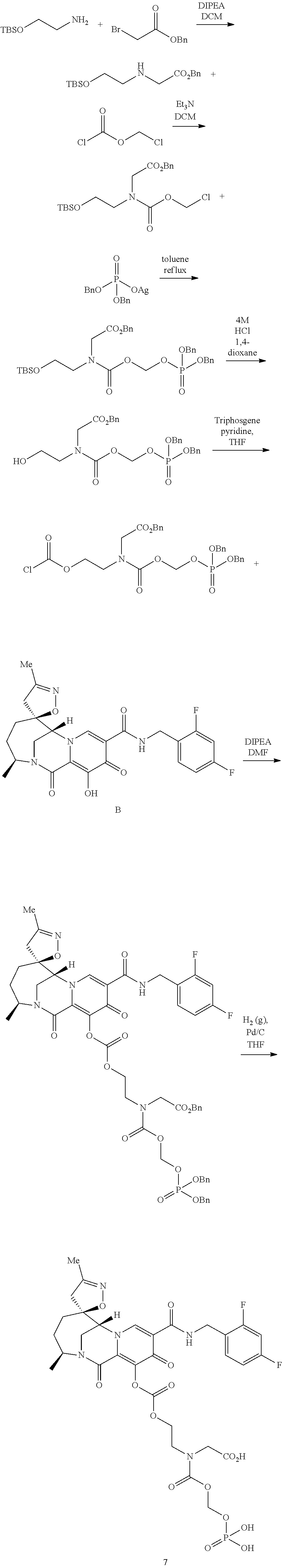

### Step 1: Preparation of benzyl (2-((tert-butyldimethylsilyl)oxy)ethyl)glycinate

To a stirred solution of 2-((tert-butyldimethylsilyl)oxy)ethan-1-amine (100 g, 571.4 mmol) in DCM (3.0 L) at 0° C. under argon was added benzyl 2-bromoacetate (64.85 g, 285.7 mmol) followed by DIPEA (81.0 g, 628.1 mmol). The reaction mixture was stirred for 16 h at room temperature. The reaction mixture was diluted with DCM and washed with water. The organic layer was dried over anhydrous Na₂SO₄, filtered, and concentrated under reduced pressure. The residue was purified by silica gel column chromatography (10-15% EtOAc in pet ether) to afford the title compound. MS (m/z) 324.25 [M+H]+.

### Step 2: Preparation of benzyl N-(2-((tert-butyldimethylsilyl)oxy)ethyl)-N-((chloromethoxy)carbonyl)glycinate

To a stirred solution of benzyl (2-((tert-butyldimethylsilyl)oxy)ethyl)glycinate (80 g, 247.6 mmol) in DCM (2.4 L) at 0° C. under argon was added chloromethyl chloroformate (41.5 g, 321.9 mmol) followed by Et₃N (62.5 g, 619 mmol). The mixture was stirred for 16 h at room temperature. The reaction mixture was diluted with DCM and washed with water. The organic layer was dried over anhydrous Na₂SO₄, filtered, and concentrated under reduced pressure. The crude residue was purified by silica gel column chromatography (20-30% EtOAc in pet ether) to afford the title compound. MS (m/z) 416.56 [M+H]+.

### Step 3: Preparation of benzyl N-((((bis(benzyloxy)phosphoryl)oxy)methoxy)carbonyl-N-(2-((tert-butyldimethylsilyl)oxy)ethyl)glycinate

To a stirred solution of benzyl N-(2-((tert-butyldimethylsilyl)oxy)ethyl)-N-((chloromethoxy)carbonyl)glycinate (20 g, 48.08 mmol) in toluene (200 mL) at room temperature under argon was added silver dibenzylphosphate (37 g, 96.08 mmol) under argon. The mixture was stirred at reflux for 16 h. The reaction mixture was allowed to cool to rt and was filtered, rinsing the solids with toluene (5V). The filtrate was concentrated under reduced pressure and purified by silica gel column chromatography (20-30% EtOAc in pet ether) to afford the title compound. MS (m/z) 658.21 [M+H]J.

### Step 4: Preparation of benzyl N-((((bis(benzyloxy)phosphoryl)oxy)methoxy)carbonyl)-N-(2-hydroxyethyl)glycinate

To a stirred solution of benzyl N-((((bis(benzyloxy)phosphoryl)oxy)methoxy)carbonyl)-N-(2-((tert-butyldimethylsilyl)oxy)ethyl)glycinate (13 g, 19.76 mmol) in 1,4-dioxane (390 mL) at 0° C. under argon was added 4 M HCl in 1,4-dioxane (9.8 mL, 39.2 mmol) under argon. The mixture was stirred for 4 h at room temperature and concentrated under reduced pressure. The crude residue was purified by silica gel column chromatography (50% EtOAc in pet-ether). The pure fractions were collected and concentrated under reduced pressure. The material was triturated with n-pentane, filtered, and dried under vacuum to obtain the title compound. MS (m/z) 544.25 [M+H]+.

### Step 5: Preparation of benzyl N-((((bis(benzyloxy)phosphoryl)oxy)methoxy)carbonyl)-N-(2-((chlorocarbonyloxy)ethyl)glycinate

To a solution of triphosgene (0.409 g, 1.38 mmol) in THF (12 mL) was added pyridine (0.289 mL, 3.59 mmol) and the suspension was cooled to 0° C. To this was added a solution of benzyl N-((((bis(benzyloxy)phosphoryl)oxy)methoxy)carbonyl)-N-(2-hydroxyethyl)glycinate (1.50 g, 2.76 mmol) in THF (12 ML). The suspension was allowed to warm to room temperature and stir for 4 h. The reaction mixture was filtered through Celite and rinsed with DCM. The organic filtrate was washed with 1 N HCl, dried over Na₂SO₄, filtered, and concentrated. MS (m/z) 605.70 [M+H]+.

### Step 6: Preparation of benzyl N-((((bis(benzyloxy)phosphoryl)oxy)methoxy)carbonyl)N-(2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)oxy)ethyl)glycinate

To a suspension of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 0.895 g, 1.84 mmol) and DIPEA (0.96 mL, 5.52 mmol) in DMF (8 mL) was added benzyl N-((((bis(benzyloxy)phosphoryl)oxy)methoxy)carbonyl)-N-(2-((chlorocarbonyl)oxy)ethyl)glycinate (1.67 g, 2.76 mmol) at 0° C. The reaction mixture was warmed to room temperature and stirred for 16 h. The reaction mixture was diluted with EtOAc and washed with 5% LiCl (aq), water, and brine. The organic phase was dried over Na₂SO₄, filtered, and concentrated to afford a residue, which was purified by silica gel column chromatography (0-100% EtOAc/hexane). The residue was then purified by reverse phase prep HPLC (5-100% MeCN/water) to afford the title compound. MS (m/z) 1055.96 [M+H]+.

### Step 7: Preparation of N-(2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyloxy)ethyl-N-(((phosphonooxy)methoxy)carbonyl)glycine

To a solution of benzyl N-((((bis(benzyloxy)phosphoryl)oxy)methoxy)carbonyl)-N-(2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)oxy)ethyl)glycinate (1.32 g, 1.25 mmol) in THF (100 mL) was added 10% Pd/C (0.133 g, 0.125 mmol). The flask was evacuated and backfilled with hydrogen gas (2×), then sparged with hydrogen for 2 min. The reaction mixture was left to stir under a hydrogen balloon atmosphere for 4 h. The reaction mixture was filtered, rinsed with THF, and concentrated to afford the title compound. MS (m/z) 785.88 [M+H]+. 1H NMR (400 MHz, DMSO-d6) δ 10.12 (t, J=6.0 Hz, 1H), 8.85 (s, 1H), 7.42 (td, J=8.7, 6.5 Hz, 1H), 7.25 (ddd, J=10.4, 9.3, 2.6 Hz, 1H), 7.08 (td, J=8.7, 2.7 Hz, 1H), 5.54-5.38 (m, 2H), 4.72 (s, 1H), 4.56 (qt, J=14.9, 6.7 Hz, 3H), 4.32 (dt, J=14.7, 5.8 Hz, 2H), 4.04 (d, J=25.5 Hz, 2H), 3.82 (d, J=16.5 Hz, 1H), 3.71 (d, J=14.8 Hz, 1H), 3.67-3.51 (m, 2H), 3.03 (d, J=17.5 Hz, 1H), 2.66 (d, J=17.5 Hz, 1H), 1.95 (s, 3H), 1.87-1.72 (m, 3H), 1.25 (dd, J=15.8, 9.3 Hz, 1H), 1.17 (d, J=6.7 Hz, 3H).

### Example 8: Preparation of N-(2-((((((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)ethyl)-N-(((phosphonooxy)methoxy)carbonyl)glycine (8)

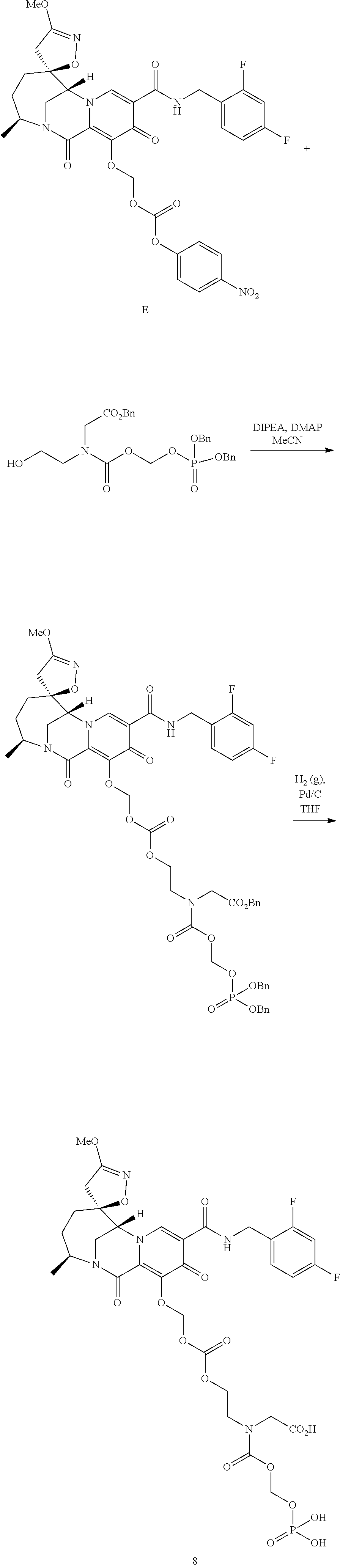

### Step 1: Preparation of benzyl N-((((bis(benzyloxy)phosphoryl)oxy)methoxy)carbonyl)N-(2-((((((3′S,5S,7′R)-10′-((2,4-difluorobenzyl)carbamoyl)-3-methyl-3′-methyl-1′,11′-dioxo-1′,4′,5′,11′-tetrahydro-3′H,4H,7′H-spiro[isoxazole-5,6′-[2,7]methanopyrido[1,2-a][1.4]diazonin]-12′-yl)oxy)methoxy)carbonyl)oxy)ethyl)glycinate

To a solution of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-nitrophenyl) carbonate (Intermediate E, 0.100 g, 0.143 mmol) in MeCN (2 mL) was added Et₃N (0.060 mL, 0.430 mmol) and DMAP (0.002 g, 0.014 mmol), followed by benzyl N-((((bis(benzyloxy)phosphoryl)oxy)methoxy)carbonyl)-N-(2-hydroxyethyl)glycinate (0.234 g, 0.430 mmol), prepared according to Step 4 of Example 7. The reaction mixture was left to stir at room temperature for 16 h and was concentrated. The residue was purified by silica gel column chromatography (0-100% EtOAc/hexane, then 0-20% MeOH/DCM) to afford the title compound. MS (m/z) 1101.92 [M+H]+.

### Step 2: Preparation of N-(2-((((((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)ethyl)-N-(((phosphonooxy)methoxy)carbonyl)glycine (8)

To a solution of benzyl N-((((bis(benzyloxy)phosphoryl)oxy)methoxy)carbonyl)-N-(2-((((((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)ethyl)glycinate (0.063 g, 0.057 mmol) in THF (5 mL) was added 5% Pd/C (0.012 g, 0.006 mmol). The vial was evacuated and backfilled with hydrogen gas (2×), then sparged with hydrogen for 2 min. The reaction mixture was left to stir under a hydrogen balloon atmosphere for 2 h. The reaction mixture was filtered and concentrated to yield a crude residue, which was dissolved in 1:1 MeCN/water and purified by reverse phase prep HPLC (10-100% MeCN/water w/ 0.1% TFA) to afford the title compound after lyophilization. MS (m/z) 831.82 [M+H]+. 1H NMR (400 MHz, DMSO-d6) δ 10.26 (t, J=6.0 Hz, 1H), 8.75 (s, 1H), 7.42 (q, J=8.3 Hz, 1H), 7.24 (td, J=9.9, 2.7 Hz, 1H), 7.07 (td, J=8.5, 2.5 Hz, 1H), 5.82 (d, J=6.5 Hz, 1H), 5.64 (dd, J=6.5, 2.7 Hz, 1H), 5.44 (dd, J=14.8, 13.0 Hz, 2H), 4.71 (s, 1H), 4.58 (dt, J=20.7, 7.7 Hz, 3H), 4.20 (t, J=6.5 Hz, 2H), 4.04 (s, 1H), 3.98 (s, 1H), 3.82 (s, 3H), 3.68 (d, J=6.5 Hz, 2H), 3.54 (t, J=5.2 Hz, 2H), 3.05 (d, J=16.8 Hz, 1H), 2.75 (d, J=16.9 Hz, 1H), 1.98-1.69 (m, 3H), 1.32 (s, 1H), 1.15 (d, J=6.6 Hz, 3H).

### Example 9: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((phosphonooxy)methyl) ethane-1,2-diylbis(methylcarbamate) (9)

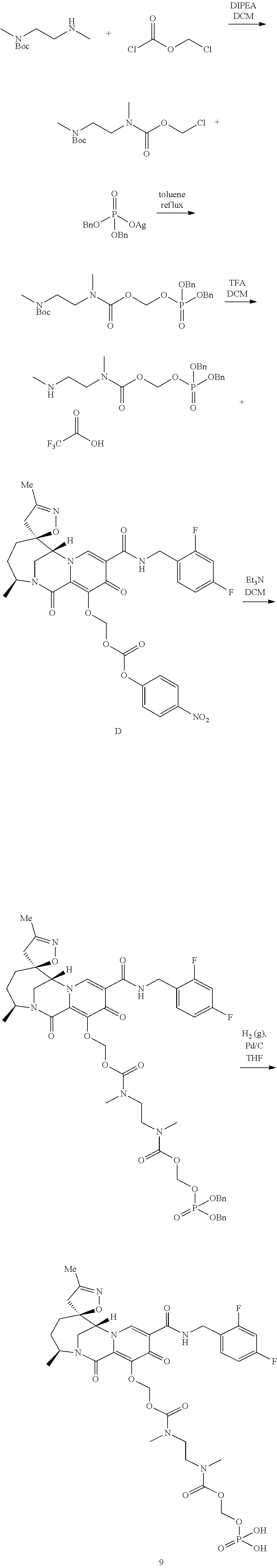

### Step 1: Preparation of tert-butyl (chloromethyl) ethane-1,2-diylbis(methylcarbamate)

To a solution of tert-butyl methyl(2-(methylamino)ethyl)carbamate (0.500 g, 2.66 mmol) in DCM (3 mL) was added DIPEA (0.694 mL, 3.98 mmol) and chloromethyl chloroformate (0.283 mL, 3.19 mmol) at 0° C. The reaction mixture was stirred at 0° C. for 2 h and concentrated. The residue was purified by silica gel column chromatography (10-100% EtOAc/hexane) to afford the title compound. MS (m/z) 180.99 [M-Boc+H]+.

### Step 2: Preparation of ((bis(benzyloxy)phosphoryl)oxy)methyl tert-butyl ethane-1,2-diylbis(methylcarbamate)

To a solution of tert-butyl (chloromethyl) ethane-1,2-diylbis(methylcarbamate) (0.100 g, 0.356 mmol) in toluene (12 mL) was added silver dibenzylphosphate (0.412 g, 1.07 mmol) and the reaction mixture was heated to reflux for 2 h. The reaction mixture was cooled to rt and concentrated. The residue was purified by silica gel column chromatography (10-100% EtOAc/hexane) to afford the title compound. MS (m/z) 545.03 [M+Na]+.

### Step 3: Preparation of ((bis(benzyloxy)phosphoryl)oxy)methyl methyl(2-(methylamino)ethylcarbamate; trifluoroacetic acid adduct

To a solution of ((bis(benzyloxy)phosphoryl)oxy)methyl tert-butyl ethane-1,2-diylbis(methylcarbamate) (0.186 g, 0.356 mmol) in DCM (2.5 mL) at 0° C. was added a solution of TFA (0.872 mL, 11.4 mmol) in DCM (1 mL). The reaction mixture was stirred at 0° C. for 1 h. The reaction mixture was concentrated to dryness, redissolved in 1 mL of DCM, and concentrated again. The residue was dried under high vacuum to afford the title compound as a 2,2,2-trifluoroacetic acid adduct. MS (m/z) 423.03 [M+H]+.

### Step 4: Preparation of ((bis(benzyloxy)phosphoryl)oxy)methyl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yloxy)methyl) ethane-1,2-diylbis(methylcarbamate)

To a solution of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-nitrophenyl) carbonate (Intermediate D) (0.130 g, 0.191 mmol) and ((bis(benzyloxy)phosphoryl)oxy)methyl methyl(2-(methylamino)ethyl)carbamate trifluoroacetic acid adduct (0.153 g, 0.286 mmol) in DCM (8 mL) at room temperature was added Et₃N (0.798 mL, 5.72 mmol). The reaction mixture was concentrated and purified by reverse phase prep HPLC (5-100% MeCN/water w/ 0.1% TFA) and lyophilized to afford the title compound. MS (m/z) 964.76 [M+H]+.

### Step 5: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl oxy)methyl ((phosphonooxy)methyl) ethane-1,2-diylbis(methylcarbamate) (9)

To a solution of ((bis(benzyloxy)phosphoryl)oxy)methyl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) ethane-1,2-diylbis(methylcarbamate) (0.084 g, 0.087 mmol) in THF (8 mL) was added 5% Pd/C (0.018 g, 0.009 mmol). The vial was evacuated and backfilled with hydrogen gas (2×), then sparged with hydrogen for 2 min. The reaction mixture was left to stir under a hydrogen balloon atmosphere for 2 h. The reaction mixture was filtered through Celite, rinsed with THF, and concentrated. The residue was dissolved in 1:1 MeCN/water and lyophilized to afford the title compound. MS (m/z) 784.80 [M+H]+. 1H NMR (400 MHz, DMSO-d6) δ 10.32 (t, J=6.0 Hz, 1H), 8.67 (d, J=1.8 Hz, 1H), 7.43 (td, J=8.6, 6.6 Hz, 1H), 7.25 (ddd, J=10.5, 9.3, 2.6 Hz, 1H), 7.08 (td, J=8.6, 2.6 Hz, 1H), 5.76-5.56 (m, 2H), 5.48-5.33 (m, 2H), 4.64 (d, J=10.9 Hz, 2H), 4.59-4.50 (m, 2H), 3.69 (d, J=13.9 Hz, 1H), 3.65 (s, 1H), 3.33 (s, 4H), 3.03-2.93 (m, 1H), 2.88-2.74 (m, 6H), 2.63 (dd, J=17.9, 4.8 Hz, 1H), 1.95 (s, 3H), 1.77 (dd, J=16.9, 9.8 Hz, 3H), 1.37-1.20 (m, 1H), 1.15 (d, J=6.6 Hz, 3H).

### Example 10: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (3-(phosphonooxy)propyl) carbonate (10)

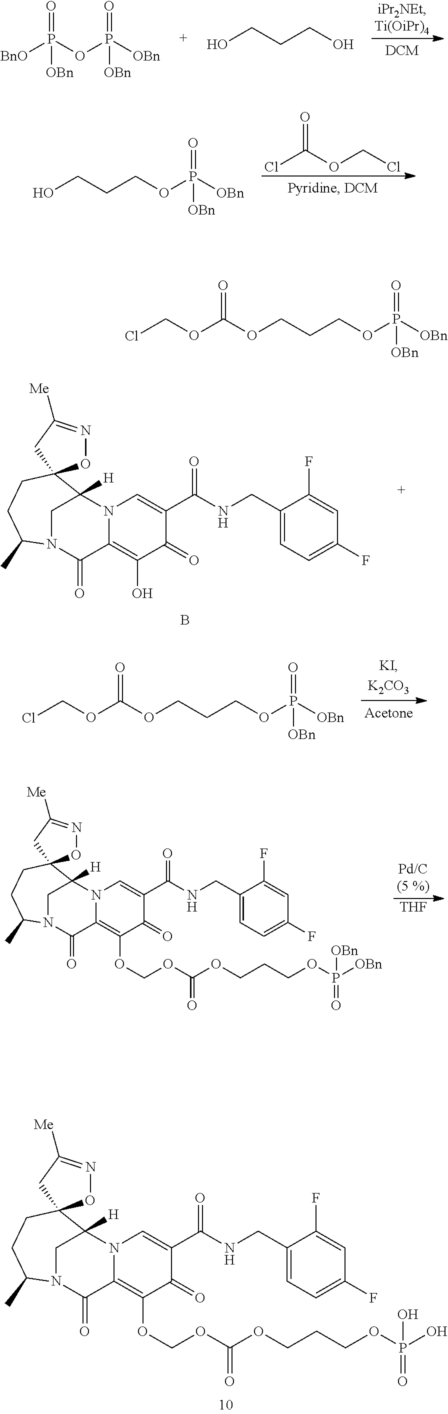

### Step 1: Preparation of dibenzyl (3-hydroxypropyl) phosphate

To the solution of propane-1,3-diol (0.22 g, 2.89 mmol) was added N,N-diisopropylethylamine (0.79 ml, 4.4 mmol) followed by tetrabenzylpyrophosphate (0.95 g, 1.76 mmol) and titanium(IV) isopropoxide (0.085 g, 0.3 mmol) sequentially. The resulting mixture was stirred at room temperature for 4 hrs. Then the reaction mixture was filtered through a pad of silica gel/magnesium sulfate mixture (20:1), rinsing the solids with ethyl acetate/hexane. The filtrate was dried over sodium sulfate, filtered, concentrated under reduced pressure. The residue was purified by normal phase chromatography on a silica gel to afford the title compound. MS (m/z) 337.2 [M+H]+.

### Step 2: Preparation of 3-((bis(benzyloxy)phosphoryl)oxy)propyl(chloromethyl) carbonate

Dibenzyl 3-hydroxypropyl phosphate (0.5 g, 1.5 mmol) was added to a stirred mixture of chloromethyl chloroformate (0.145 ml, 1.64 mmol) in CH2Cl2 (10 mL) then followed by the addition of pyridine (0.14 mL, 1.87 mmol) dropwise at room temperature. The reaction was stirred for overnight before it was diluted with DCM and washed sequentially with HCl (0.5M) and water. The organic layer was dried over magnesium sulfate, filtered, and evaporated to obtain the title compound. MS (m/z) 428.9 [M+H]+.

### Step 3: Preparation of 3-((bis(benzyloxy)phosphoryl)oxy)propyl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate

To the solution of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 0.08 g, 0.164 mmol) in acetone (20 mL) was added KI (0.036 mg, 0.21 mmol) and K₂CO₃ (0.046 g, 0.329 mmol) followed by 3-((bis(benzyloxy)phosphoryl)oxy)propyl (chloromethyl) carbonate (0.085 g, 0.197 mmol). The resulting mixture was left to stir at room temperature for overnight and then was concentrated. The residue was purified by silica gel column chromatography (0-100% EtOAc/Hexane) to afford the title compound. MS (m/z) 878.9[M+H]+.

### Step 4: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-2′-yl)oxy)methyl (3-(phosphonooxy)propyl) carbonate (10)

To the solution of 3-((bis(benzyloxy)phosphoryl)oxy)propyl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate (0.08 g, 0.009 mmol) in THF (20 ml) was added Pd/C (5 wt %) (0.020 g, 0.0009 mmol). The resulting mixture was purged with hydrogen three times before it was stirred under hydrogen for 1 h. The reaction was then filtered through Celite® and concentrated. The resulting residue was purified by reverse phase preparative HPLC to afford the title compound. MS (m/z) 698.88 [M+H]+. 1H NMR (400 MHz, DMSO-d6) δ 10.28 (t, J=6.0 Hz, 1H), 8.71 (s, 1H), 7.43 (td, J=8.7, 6.6 Hz, 1H), 7.25 (ddd, J=10.6, 9.3, 2.6 Hz, 1H), 7.13-7.03 (m, 1H), 5.83 (d, J=6.5 Hz, 1H), 5.64 (d, J=6.5 Hz, 1H), 4.71-4.47 (m, 5H), 4.14 (ddt, J=10.9, 6.3, 4.4 Hz, 2H), 3.88 (dt, J=7.5, 6.3 Hz, 2H), 3.68 (t, J=2.3 Hz, 2H), 3.01 (d, J=17.5 Hz, 1H), 2.63 (d, J=17.6 Hz, 1H), 1.97-1.86 (m, 4H), 1.91-1.75 (m, 2H), 1.79-1.69 (m, 1H), 1.35-1.23 (m, 1H), 1.16 (d, J=6.7 Hz, 3H).

### Example 11: Preparation (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (3-(phosphonooxy)propyl) carbonate (11)

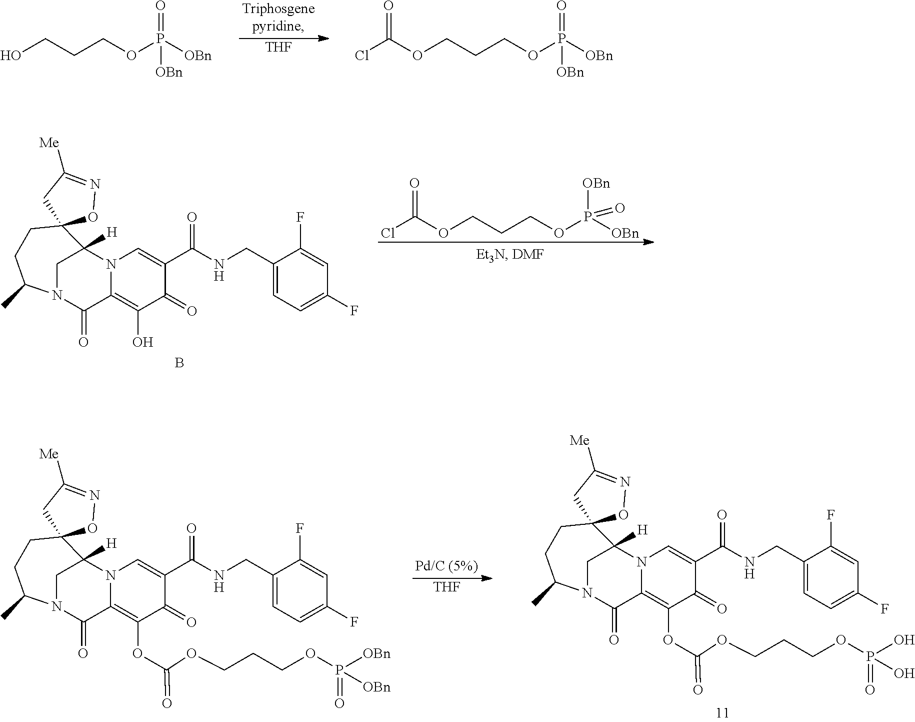

### Step 1: Preparation 3-((bis(benzyloxy)phosphoryl)oxy)propyl carbonochloridate

To a solution of triphosgene (0.088 g, 0.297 mmol) in THF (1 mL) was added pyridine (90.062 mL, 0.773 mmol) and the resulting suspension was cooled to 0° C. To this mixture was added a solution of dibenzyl (3-hydroxypropyl) phosphate (0.2 g, 0.595 mmol) in THF (1 mL). The suspension was allowed to warm to room temperature and stir for 4 hours. The mixture was filtered through Celite and rinsed with DCM. The filtrate was washed with 1 N HCl, dried over Na₂SO₄, filtered, concentrated, and used directly in next step.

### Step 2: Preparation 3-((bis(benzyloxy)phosphoryl)oxy)propyl ((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl) carbonate

To a suspension of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 0.1 g, 0.21 mmol) and DIPEA (0.11 mL, 0.62 mmol) in DMF (1 mL) was added 3-((bis(benzyloxy)phosphoryl)oxy)propyl carbonochloridate (0.123 g, 0.31 mmol) at 0° C. The reaction mixture was then warmed to rt and stirred overnight. The reaction mixture was diluted with EtOAc, washed with 5% LiCl (aq), water, and brine. The organic phase was dried over MgSO₄, filtered, and concentrated to afford a residue, which was purified by silica gel column chromatography (0-100% EtOAc/hexane) to afford the title compound. MS (m/z) 849.40 [M+H]+.

### Step 3: Preparation 3′S, 5S, 7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (3-(phosphonooxy)propyl) carbonate (11)

The title compound was prepared following the same method as Example 10 step 4, except using 3-((bis(benzyloxy)phosphoryl)oxy)propyl ((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl) carbonate instead of 3-((bis(benzyloxy)phosphoryl)oxy)propyl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate. MS (m/z) 668.88 [M+H]+. 1H NMR (400 MHz, Chloroform-d) δ 10.27 (s, 1H), 8.59 (s, 1H), 7.42-7.32 (m, 1H), 6.94-6.75 (m, 2H), 4.74 (d, J=8.5 Hz, 1H), 4.61 (d, J=5.7 Hz, 2H), 4.43 (s, 2H), 4.22 (d, J=34.6 Hz, 3H), 3.81 (s, 2H), 3.09 (d, J=17.8 Hz, 1H), 2.63 (d, J=17.9 Hz, 1H), 2.08 (m, 5H), 2.01-1.81 (m, 3H), 1.57 (t, J=10.3 Hz, 1H), 1.25 (d, J=6.5 Hz, 3H).

### Example 12: Preparation (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (2,2-dimethyl-3-(phosphonooxy)propyl) carbonate (12)

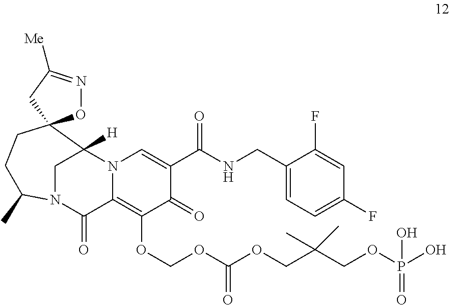 [SMILES: CC1=NO[C@@]2(CC[C@H](C)N3C[C@H]2n2cc(C(=O)NCc4ccc(F)cc4F)c(=O)c(OCOC(=O)OCC(C)(C)COP(=O)(O)O)c2C3=O)C1]

The title compound was made following the same method as Example 10, except in step 1, propane-1,3-diol was replaced by 2,2-dimethylpropane-1,3-diol. MS (m/z) 726.91 [M+H]+. 1H NMR (400 MHz, Chloroform-d) δ 10.28 (s, 1H), 8.43 (s, 1H), 7.38 (q, J=7.9 Hz, 1H), 6.82 (q, J=9.3 Hz, 2H), 5.93 (d, J=6.6 Hz, 1H), 5.79 (d, J=6.7 Hz, 1H), 4.85 (d, J=8.9 Hz, 1H), 4.62 (q, J=14.2, 12.8 Hz, 2H), 4.22 (s, 1H), 3.98 (d, J=10.4 Hz, 1H), 3.90 (d, J=10.3 Hz, 1H), 3.77 (m, 4H), 3.03 (d, J=17.8 Hz, 1H), 2.60 (d, J=17.8 Hz, 1H), 2.08 (s, 3H), 1.94 (s, 3H), 1.52 (m, 1H), 1.30-1.23 (m, 3H), 0.97 (d, J=7.3 Hz, 6H).

### Example 13: Preparation (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (2,2-dimethyl-3-(phosphonooxy)propyl) carbonate (13)

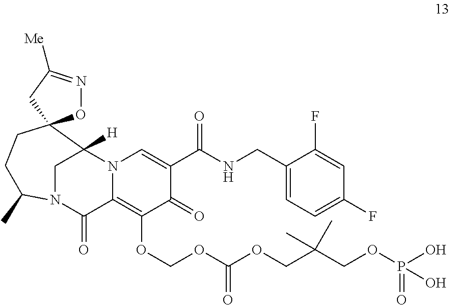 [SMILES: CC1=NO[C@@]2(CC[C@H](C)N3C[C@H]2n2cc(C(=O)NCc4ccc(F)cc4F)c(=O)c(OCOC(=O)OCC(C)(C)COP(=O)(O)O)c2C3=O)C1]

The title compound was made following the same method as Example 11, except in step 1, propane-1,3-diol was replaced by 2,2-dimethylpropane-1,3-diol. MS (m/z) 696.9 [M+H]+. 1H NMR (400 MHz, Chloroform-d) δ 10.23 (t, J=6.0 Hz, 1H), 8.56 (s, 1H), 7.35 (td, J=8.5, 6.3 Hz, 1H), 6.90-6.76 (m, 2H), 4.79-4.68 (m, 1H), 4.62 (d, J=5.9 Hz, 2H), 4.25 (d, J=2.2 Hz, 1H), 4.12 (s, 2H), 3.88-3.76 (m, 4H), 3.08 (d, J=17.9 Hz, 1H), 2.62 (d, J=17.9 Hz, 1H), 2.07 (s, 3H), 1.99-1.85 (m, 3H), 1.59-1.50 (m, 1H), 1.25 (d, J=6.6 Hz, 3H), 1.00 (d, J=4.5 Hz, 6H).

### Example 14: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (2-(dimethylamino)ethyl) carbonate (14)

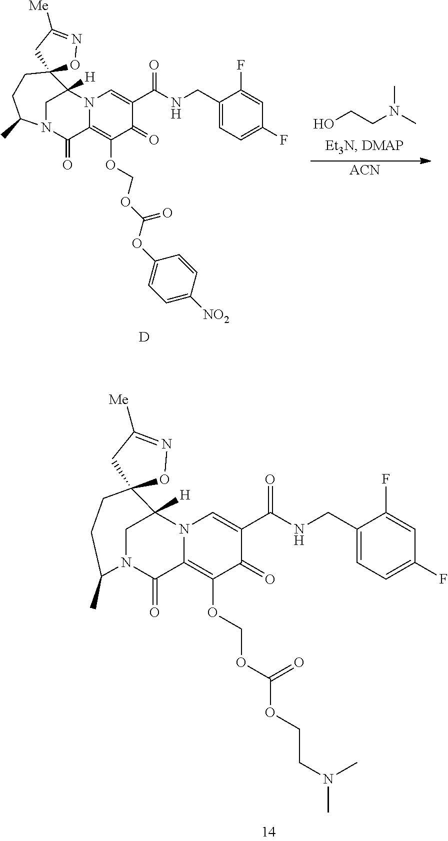

(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-nitrophenyl) carbonate (Intermediate D, 32 mg, 0.0467 mmol) and 2-(dimethylamino)ethanol (21 mg, 0.233 mmol) were mixed with acetonitrile (1 ml) at room temperature. Then Et₃N (23.6 mg, 0.233 mmol) and DMAP (0.854 mg, 0.007 mmol) were added. The resulting reaction mixture was then stirred at rt for 17 h. Reaction mixture was purified with reverse phase prep-HPLC with 0-100% acetonitrile in water with 0.1% TFA to give the title compound as TFA salt after lyophilization. Calculated for C₃₀H₃₅F₂N₅O₈: 631.25, Found MS (ESI+): 632.41 [M+H]+; ¹H NMR (400 MHz, Acetonitrile-d₃) δ 10.23 (s, 1H), 8.52 (s, 1H), 7.58-7.30 (m, 1H), 6.98 (ddt, J=13.5, 8.5, 3.1 Hz, 2H), 5.97 (d, J=6.5 Hz, 1H), 5.76 (d, J=6.6 Hz, 1H), 4.86-4.68 (m, 1H), 4.61 (d, J=5.9 Hz, 2H), 4.51 (qt, J=13.3, 5.0 Hz, 2H), 4.31 (s, 1H), 3.70 (d, J=2.3 Hz, 2H), 3.43 (t, J=5.0 Hz, 2H), 3.06 (d, J=17.9 Hz, 1H), 2.85 (s, 6H), 2.61 (d, J=17.8 Hz, 1H), 1.99 (s, 3H), 1.94-1.78 (m, 3H), 1.43 (dd, J=15.4, 10.8 Hz, 1H), 1.21 (d, J=6.7 Hz, 3H).

### Example 15: Preparation of 2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)-N,N,Ntrimethylethan-1-aminium trifluoroacetic acid (15)

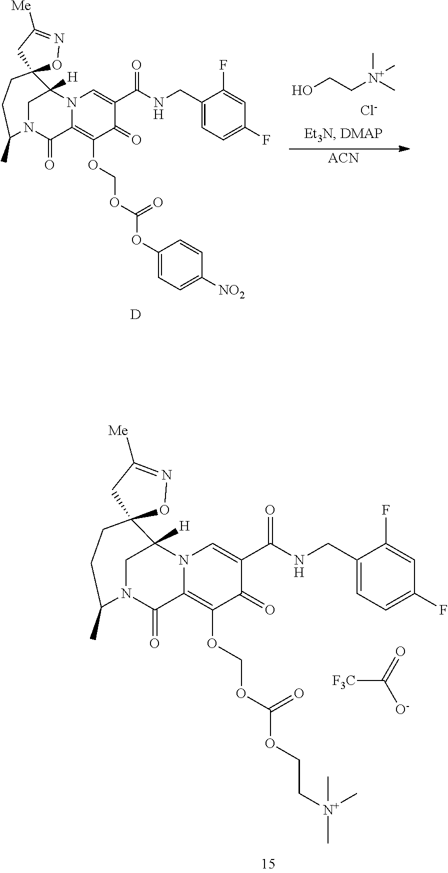

(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-nitrophenyl) carbonate (Intermediate D, 33 mg, 0.0484 mmol) and 2-hydroxy-N,N,N-trimethylethan-1-aminium chloride (33.8 mg, 0.242 mmol) were mixed with acetonitrile (0 ml) at rt. Then Et₃N (24.5 mg, 0.242 mmol) and DMAP (0.854 mg, 0.007 mmol) were added. The resulting reaction mixture was then stirred at rt for 17 h. Reaction mixture was purified with reverse phase prep-HPLC with 0-100% acetonitrile in water with 0.1% TFA to afford the title compound as TFA salt after lyophilization. Calculated for C₃₁H₃₈F₂N₅O₈⁺: 646.27, Found MS (ESI+): 646.14 [M]⁺. 1H NMR (400 MH z, CD3CN) δ 10.25 (t, J=5.8 Hz, 1H), 8.54 (s, 1H), 7.44 (td, J=8.8, 6.4 Hz, 1H), 7.04-6.93 (m, 2H), 6.03 (d, J=6.6 Hz, 1H), 5.79 (d, J=6.6 Hz, 1H), 4.72 (dt, J=9.9, 6.9 Hz, 1H), 4.61 (d, J=6.0 Hz, 2H), 4.67-4.48 (m, 2H), 4.32 (s, 1H), 3.70 (t, J=2.0 Hz, 2H), 3.67 (t, J=4.7 Hz, 2H), 3.14 (s, 9H), 3.07-3.03 (dd, J=17.8, 1.1 Hz, 1H), 2.61 (dd, J=17.8, 1.1 Hz, 1H), 1.99 (d, J=1.1 Hz, 3H), 1.94-1.79 (m, 3H), 1.45-1.34 (m, 1H), 1.22 (d, J=6.7 Hz, 3H).

### Example 16: Preparation of 2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)oxy)-N,N,N-trimethylethan-1-aminium trifluoroacetic acid (16)

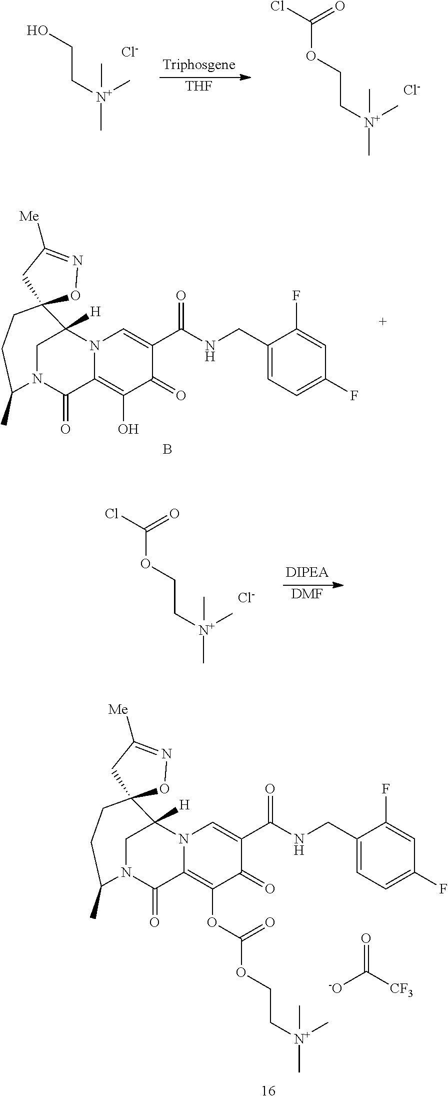

### Step 1: Synthesis of 2-((chlorocarbonyl)oxy)-N,N,N-trimethylethan-1-aminiun chloride

2-hydroxy-N,N,N-trimethylethan-1-aminium chloride (1 g, 7.16 mmol) was mixed with THF (50 mL) with stirring at room temperature to afford a slurry. Triphosgene (2.34 g, 7.88 mmol) was added in 4 portions at 0° C. The reaction mixture was then warmed up to rt and stirred for 24 hrs. The reaction mixture was filtered and the solid was collected. Hexane (3×30 mL) was used to wash the solid. The solid product 2-((chlorocarbonyl)oxy)-N,N,N-trimethylethan-1-aminium chloride was put under high vacuum for 3 h and used directly for next step. MS (m/z) 166.10 [M].

### Step 2. Synthesis of 2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)oxy)-N,N,N-trimethylethan-1-aminium trifluoroacetic acid salt (16)

(3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 103 mg, 0.212 mmol) was dissolved in DMF (3 mL) at rt. DIPEA (137 mg, 1.06 mmol) and 2-((chlorocarbonyl)oxy)-N,N,N-trimethylethan-1-aminium chloride (66 mg, 0.327 mmol) were added sequentially at room temperature. The reaction mixture was stirred for 17 h. Purification directly on reverse phase preparative HPLC with ACN/water (containing 0.1% TFA) gave title compound (16). Calculated for C₃₀H₃₆F₂N₅O₇⁺: 616.26. Found MS (M+): 616.37; 1H NMR (400 MHz, CD3CN) δ 10.11 (t, J=5.9 Hz, 1H), 8.63 (s, 1H), 7.44 (td, J=8.8, 6.5 Hz, 1H), 7.04-6.93 (m, 2H), 4.76-4.66 (m, 3H), 4.61 (d, J=6.0 Hz, 2H), 4.40 (d, J=2.5 Hz, 1H), 3.75 (d, J=2.3 Hz, 2H), 3.74-3.70 (m, 2H), 3.17 (s, 9H), 3.06 (d, J=17.8 Hz, 1H), 2.62 (d, J=17.8 Hz, 1H), 1.99 (s, 3H), 1.95-1.82 (m, 3H), 1.38 (ddt, J=13.2, 10.0, 5.0 Hz, 1H), 1.22 (d, J=6.7 Hz, 3H).

### Example 17: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((S)-2-(phosphonooxy)propyl) carbonate (17)

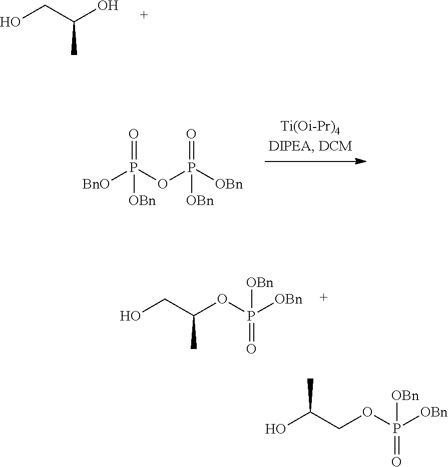

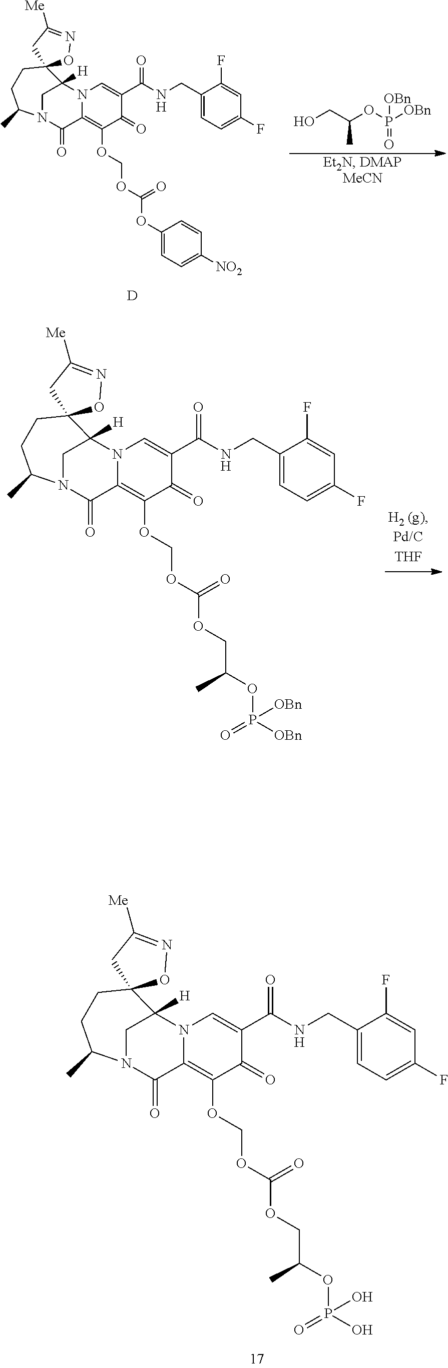

### Step 1: Preparation of (S)-dibenzyl (1-hydroxypropan-2-yl) phosphate and (S)-dibenzyl (2-hydroxypropyl) phosphate

To a mixture of (S)-propane-1,2-diol (0.44 mL, 4.16 mmol) in DCM (2.7 mL) was added DIPEA (1.66 mL, 9.25 mmol) followed by tetrabenzylpyrophosphate (2.00 g, 3.71 mmol) and Ti(i-PrO)₄ (0.19 mL, 0.631 mmol). The mixture was stirred at room temperature for 16 h. MgSO₄ and silica gel were added to reaction, stirred for 10 min, and filtered, rinsing with 4:3 EtOAc/hexane. The filtrate was concentrated and purified by reverse phase prep HPLC (10-100% MeCN/water) to afford the title compounds as an inseparable mixture of regioisomers (rr=1.1:1) by NMR MS (m/z) 336.99 [M+H]+.

### Step 2: Preparation of (S)-2-((bis(benzyloxy)phosphoryl)oxy)propyl (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate

To a solution of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-nitrophenyl) carbonate (Intermediate D) (0.250 g, 0.367 mmol) in MeCN (5 mL) was added Et₃N (0.153 mL, 1.10 mmol) and DMAP (0.004 g, 0.037 mmol), followed by the mixture of (S)-dibenzyl (1-hydroxypropan-2-yl) phosphate and (S)-dibenzyl (2-hydroxypropyl) phosphate (0.370 g, 1.10 mmol) prepared in Step 1. The reaction mixture was left to stir at room temperature for 16 h and concentrated. The residue was purified by silica gel column chromatography (0-100% EtOAc/hexane, then 0-20% MeOH/DCM) to afford the title compound. MS (m/z) 878.88 [M+H]⁺.

### Step 3: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((S)-2-(phosphonooxy)propyl) carbonate (17)

To a solution of (S)-2-((bis(benzyloxy)phosphoryl)oxy)propyl ((((3′S,5S,7′R)-10′-((2,4-di fluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate (0.068 g, 0.077 mmol) in THF (5 mL) was added 10% Pd/C (0.008 g, 0.008 mmol). The vial was evacuated and backfilled with hydrogen gas (2×), then sparged with hydrogen for 2 min. The reaction mixture was left to stir under a hydrogen balloon atmosphere for 2 h. The reaction mixture was filtered and concentrated. The crude residue was dissolved in 1:1 MeCN/water and purified by reverse phase prep HPLC (10-100% MeCN w/ 0.1% TFA) to afford the title compound after lyophilization. MS (m/z) 698.91 [M+H]⁺. 1H NMR (400 MHz, DMSO-d6) δ 10.28 (t, J=5.9 Hz, 1H), 8.71 (s, 1H), 7.42 (td, J=8.6, 6.7 Hz, 1H), 7.29-7.20 (m, 1H), 7.08 (td, J=8.5, 2.7 Hz, 1H), 5.84 (d, J=6.4 Hz, 1H), 5.64 (d, J=6.4 Hz, 1H), 4.63 (d, J=3.2 Hz, 2H), 4.61-4.48 (m, 2H), 4.42 (dq, J=11.9, 5.5 Hz, 1H), 4.13 (dd, J=11.2, 5.2 Hz, 1H), 4.04 (dd, J=11.2, 4.7 Hz, 1H), 3.78-3.58 (m, 2H), 3.00 (d, J=17.5 Hz, 1H), 2.64 (d, J=17.6 Hz, 1H), 1.95 (s, 3H), 1.88-1.66 (m, 3H), 1.36-1.24 (m, 1H), 1.22 (d, J=6.3 Hz, 3H), 1.16 (d, J=6.7 Hz, 3H).

### Example 18: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((R)-1-(phosphonooxy)propan-2-yl) carbonate (18)

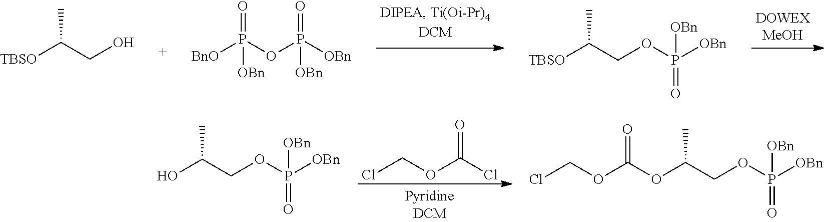

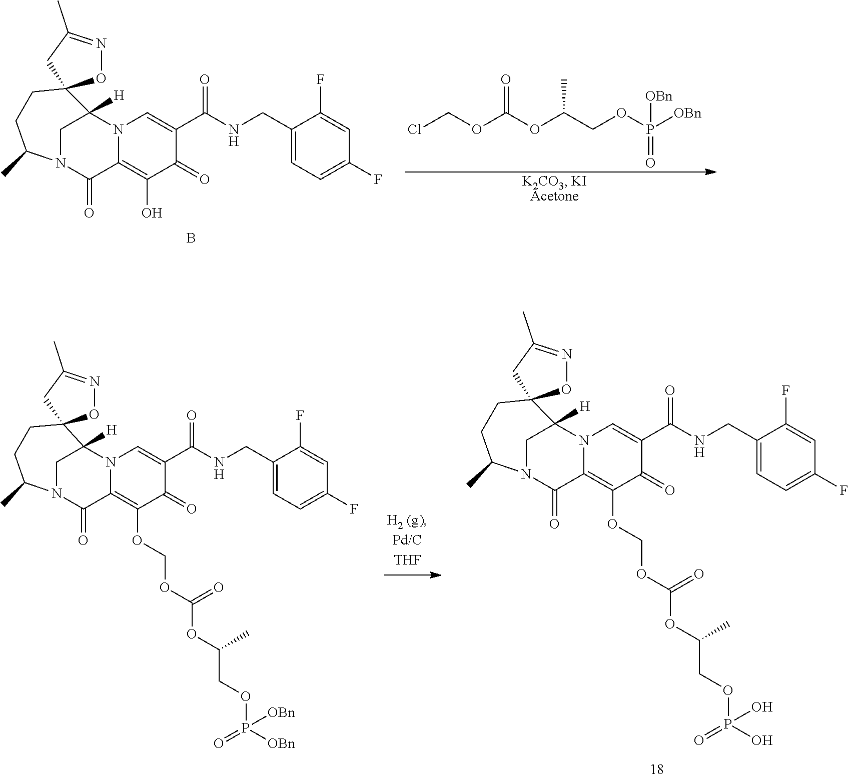

### Step 1: Preparation of (R)-dibenzyl (2-((tert-butyldimethyl)silyl)oxy)propyl) phosphate

To a mixture of (R)-2-((tert-butyldimethylsilyl)oxy)propan-1-ol (0.396 g, 2.08 mmol) in DCM (1.35 mL) was added DIPEA (0.83 mL, 4.62 mmol) followed by tetrabenzylpyrophosphate (1.00 g, 1.86 mmol) and Ti(i-PrO)₄ (0.094 mL, 0.316 mmol). The mixture was stirred at room temperature for 16 h. MgSO₄ and silica gel were added to reaction, stirred for 10 min. and filtered, rinsing with 4:3 EtOAc/hexanes. The filtrate was concentrated and purified by reverse phase prep HPLC (10-100% MeCN/water) to afford the title compound. MS (m/z) 451.66 [M+H]+.

### Step 2: Preparation of (R)-dibenzyl (2-hydroxypropyl) phosphate

To a solution of (R)-dibenzyl (2-((tert-butyldimethylsilyl)oxy)propyl) phosphate (0.344 g, 0.763 mmol) in MeOH (3.4 mL) was added Dowex resin 50Wx8 (hydrogen form) (3.40 g) at rt. After 6 h, the mixture was filtered, concentrated, and purified by silica gel column chromatography (0-100% EtOAc/hexanes) to afford the title compound. MS (m/z) 336.74 [M+H]+.

### Step 3: Preparation of (R)-1-((bis(benzyloxy)phosphoryl)oxy)propan-2-yl (chloromethyl) carbonate

To a solution of (R)-dibenzyl (2-hydroxypropyl) phosphate (0.316 g, 0.940 mmol) in DCM (22 mL) at 0° C. was added chloromethyl chloroformate (0.184 mL, 2.07 mmol) followed by pyridine (0.190 mL, 2.35 mmol). The reaction mixture was allowed to warm to rt and stir 1 h. The reaction mixture was quenched with water and 1 N HCl. The phases were separated and aqueous phase was extracted with DCM (3×). The combined organic phase was washed with brine, dried over Na₂SO₄, filtered, and concentrated. The residue was purified by column chromatography (0-100% EtOAc/hexanes) to afford the title compound. MS (m/z) 428.78 [M+H]+.

### Step 4: Preparation of (R)-1-((bis(benzyloxy)phosphoryl)oxy)propan-2-yl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate

To a solution of (R)-1-((bis(benzyloxy)phosphoryl)oxy)propan-2-yl (chloromethyl) carbonate (0.15 g, 0.35 mmol) in acetone (3.4 mL) was added Intermediate B (0.142 g, 0.292 mmol), KI (0.063 g, 0.379 mmol), and K₂CO₃ (0.081 g, 0.584 mmol). The reaction mixture was left to stir overnight at rt. The reaction mixture was partitioned between EtOAc and water and the phases separated. The aqueous phase was extracted with EtOAc and the combined organic phase was washed with brine, dried over Na₂SO₄, filtered, and concentrated. The residue was suspended in MeCN and purified by reverse phase prep HPLC (10-100% MeCN/water). The pooled fractions were concentrated to remove MeCN and extracted with EtOAc (3×). The combined organic phase was dried over Na₂SO₄, filtered, and concentrated. The residue was purified by silica gel column chromatography (0-100% EtOAc/hexanes) to afford the title compound. MS (m/z) 878.77 [M+H]+.

### Step 5: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((R)-1-(phosphonooxy)propan-2-v0) carbonate (18)

To a solution of (R)-1-((bis(benzyloxy)phosphoryl)oxy)propan-2-yl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate (0.035 g, 0.040 mmol) in THF (4 mL) was added 10% Pd/C (0.004 g, 0.004 mmol). The flask was evacuated and backfilled with hydrogen gas (2×), then sparged with hydrogen for 2 min. The reaction mixture was left to stir under a hydrogen balloon atmosphere for 4 h. The reaction mixture was filtered through Celite and concentrated. The residue was dissolved in 1:1 MeCN/water and lyophilized to afford the title compound. MS (m/z) 698.96 [M+H]+. 1H NMR (400 MHz, DMSO-d6) δ 10.28 (t, J=5.9 Hz, 1H), 8.71 (s, 1H), 7.43 (td, J=8.6, 6.5 Hz, 1H), 7.25 (ddd, J=10.5, 9.3, 2.6 Hz, 1H), 7.15-6.99 (m, 1H), 5.86 (d, J=6.4 Hz, 1H), 5.59 (d, J=6.4 Hz, 1H), 4.89-4.73 (m, 1H), 4.73-4.60 (m, 2H), 4.60-4.45 (m, 2H), 3.87 (dt, J=6.4, 4.6 Hz, 2H), 3.69 (dd, J=4.7, 2.3 Hz, 2H), 3.00 (d, J=17.6 Hz, 1H), 2.63 (d, J=17.6 Hz, 1H), 1.95 (s, 3H), 1.88-1.67 (m, 3H), 1.37-1.22 (m, 2H), 1.20 (d, J=6.4 Hz, 3H), 1.16 (d, J=6.7 Hz, 3H).

### Example 19: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((S)-1-(phosphonooxy)propan-2-yl) carbonate (19)

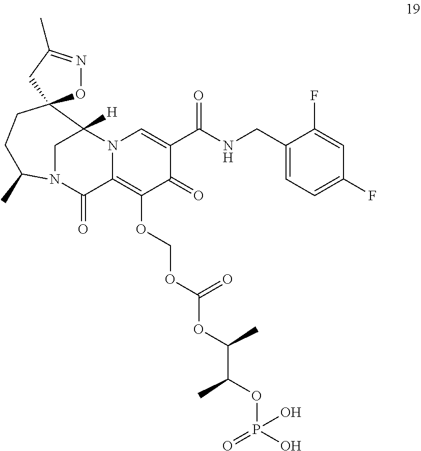 [SMILES: CC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1]

The title compound was prepared in a manner similar to Example 18, except using (S)-2-((tert-butyldimethylsilyl)oxy)propan-1-ol instead of (R)-2-((tert-butyldimethylsilyl)oxy)propan-1-ol in Step 1. MS (m/z) 698.83 [M+H]⁺. 1H NMR (400 MHz, DMSO-d6) δ 10.27 (t, J=5.9 Hz, 1H), 8.70 (s, 1H), 7.42 (td, J=8.6, 6.5 Hz, 1H), 7.29-7.20 (m, 1H), 7.07 (td, J=8.6, 2.6 Hz, 1H), 5.88-5.73 (m, 1H), 5.72-5.55 (m, 1H), 4.79 (dq, J=11.7, 6.2, 5.6 Hz, 1H), 4.61 (d, J=12.2 Hz, 2H), 4.59-4.47 (m, 2H), 3.85 (t, J=5.9 Hz, 2H), 3.68 (d, J=2.3 Hz, 2H), 3.00 (d, J=17.5 Hz, 1H), 2.64 (d, J=17.7 Hz, 1H), 1.94 (s, 3H), 1.85-1.70 (m, 3H), 1.33-1.22 (m, 1H), 1.20 (d, J=6.4 Hz, 3H), 1.15 (d, J=6.7 Hz, 3H).

### Example 20: Preparation (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 5-(phosphonooxy)pentanoate (20)

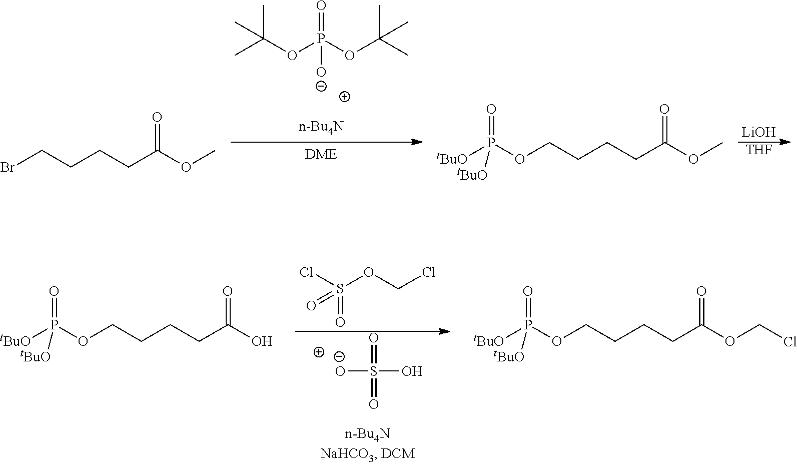

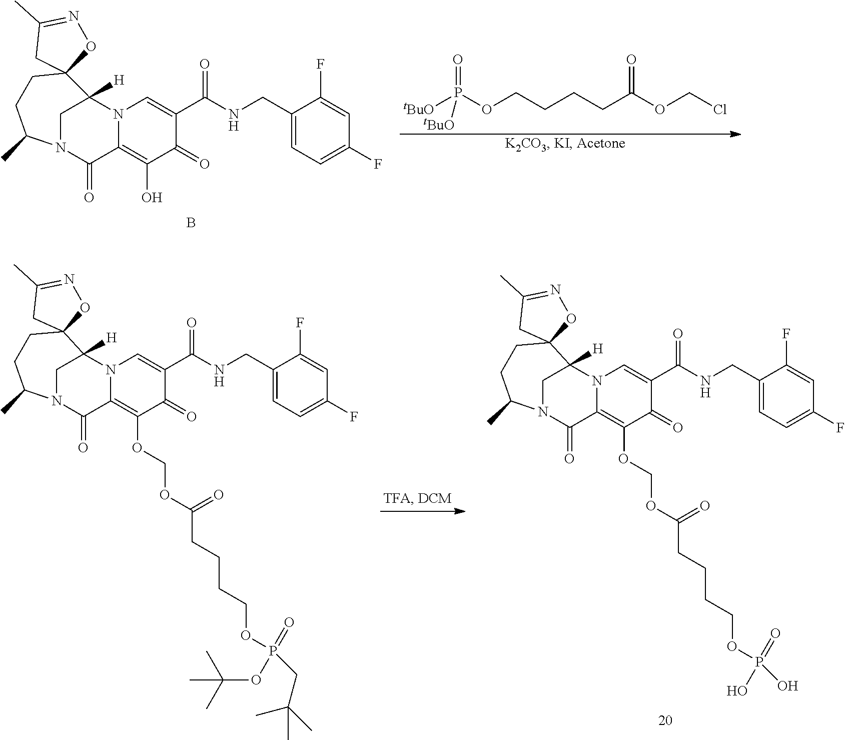

### Step 1: Preparation methyl 5-((di-tert-butoxyphosphoryl)oxy)pentanoate

A mixture of methyl 5-bromopentanoate (1 g, 5.13 mmol) and tetra-n-butylammonium di-tert-butylphosphate (2.78 g, 6.15 mmol) in dimethoxyethane (10 mL) was stirred at 80° C. for 3 hours. The reaction was cooled to ambient temperature, quenched with brine, and extracted with ethyl acetate (2×). The combined organic phase was dried over anhydrous MgSO₄, filtered, and concentrated under reduced pressure. The residue was purified by silica gel column chromatography to provide the title compound. MS (m/z) 324.7 [M+H]+.

### Step 2: Preparation 5-((di-tert-butoxyphosphoryl)oxy)pentanoic acid

A solution of 5-((di-tert-butoxyphosphoryl)oxy)pentanoate (1.32 g, 4.07 mmol) in tetrahydrofuran (15 mL) and methanol (10 mL) was treated with a solution of lithium hydroxide monohydrate (2.5 N) (3.58 ml, 8.95 mmol). The mixture was stirred for 3 hours, and then most of the solvent was evaporated under reduced pressure. The residue was diluted with 2 mL of water and acidified with 5% citric acid until pH 5. The resulting emulsion was extracted with ethyl acetate (2×). The combined organic phase was washed with brine, dried over anhydrous MgSO₄, filtered, and concentrated under reduced pressure to provide the title compound. MS (m/z) 311.1 [M+H]+.

### Step 3: Preparation chloromethyl S-((di-tert-butoxyphosphoryl)oxy)pentanoate

A biphasic mixture of 5-di-tert-butoxyphosphoryloxypentanoic acid (1.8 g, 5.8 mmol), tetrabutylammonium hydrogen sulfate (0.197 g, 0.58 mmol), and sodium bicarbonate (2.92 g, 34.8 mmol) in water (9 mL) and DCM (18 mL) was cooled to 0° C. While stirring, chloromethyl chlorosulfate (1.9 g, 11.6 mmol) was added dropwise. The reaction mixture was allowed to stir 4 h, slowly warming to rt. Water was added and the aqueous phase was extracted with DCM (3×). The combined organic phase was washed with sodium bicarbonate and brine, dried over Na₂SO₄, filtered, and concentrated. The crude product was purified by column chromatography (0-100% EtOAc/hexanes) to afford title compound. MS (m/z) 358.8 [M+H]+.

### Step 4: Preparation (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 5-((di-tert-butoxyphosphoryl)oxy)pentanoate

To a mixture of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 0.1 g, 0.26 mmol) and chloromethyl 5-di-tert-butoxyphosphoryloxypentanoate (0.147 g, 0.41 mmol) in acetone (1 mL) was added K₂CO₃ (0.0568 g, 0.411 mmol) and KI (0.044 g, 0.267 mmol). The resulting mixture was stirred at room temperature overnight. The mixture was diluted with EtOAc and was washed with H2O and brine. The organic phase was dried with MgSO₄ and the solvent was removed in vacuo. The residue was purified by silica gel column chromatography to provide the title compound. MS (m/z) 808.85 [M+H]+.

### Step 5: Preparation (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 5-(phosphonooxy)pentanoate (20)

To a mixture of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 5-((di-tert-butoxyphosphoryl)oxy)pentanoate (0.14 g, 0.173 mmol) in DCM (10 mL) was added trifluoroacetic acid (1 mL) at 0° C. The mixture was stirred at room temperature for 3 h and the solvent was removed in vacuo. The resulting residue was purified by prep HPLC to provide the title compound. MS (m/z) 696.9 [M+H]+. 1H NMR (400 MHz, DMSO-d6) δ 10.30 (t, J=6.0 Hz, 1H), 8.69 (s, 1H), 7.43 (td, J=8.7, 6.6 Hz, 1H), 7.25 (ddd, J=10.4, 9.3, 2.6 Hz, 1H), 7.08 (td, J=8.5, 2.6 Hz, 1H), 5.81 (d, J=6.3 Hz, 1H), 5.59 (d, J=6.2 Hz, 1H), 4.71-4.48 (m, 4H), 3.79 (q, J=6.3 Hz, 2H), 3.68 (d, J=2.2 Hz, 2H), 3.00 (d, J=17.5 Hz, 1H), 2.64 (d, J=17.6 Hz, 1H), 2.37-2.28 (m, 2H), 1.95 (s, 3H), 1.79 (dtd, J=19.1, 14.6, 8.7 Hz, 3H), 1.56 (m, 4H), 1.35-1.22 (m, 1H), 1.16 (d, J=6.6 Hz, 3H).

### Example 21: Preparation of N-(2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)ethyl)-N-(((phosphonooxy)methoxy)carbonyl)glycine (21)

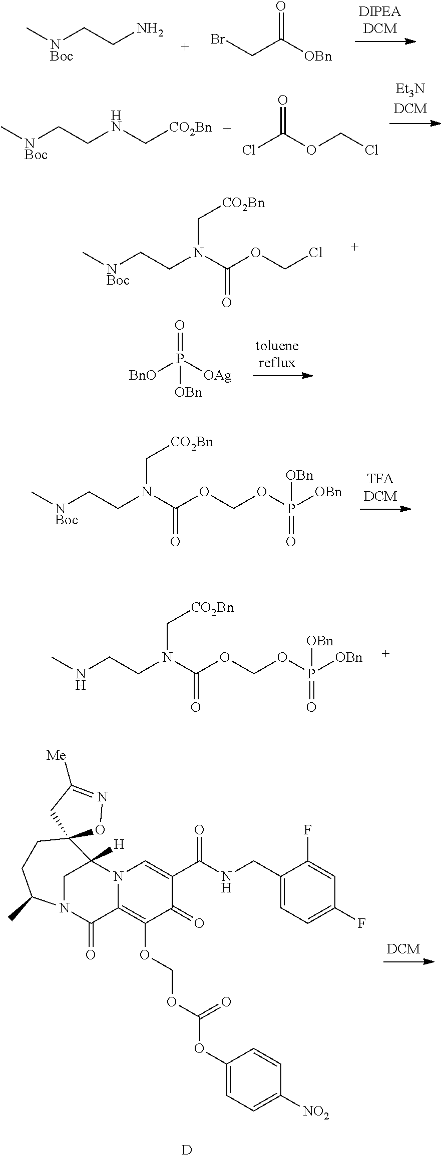

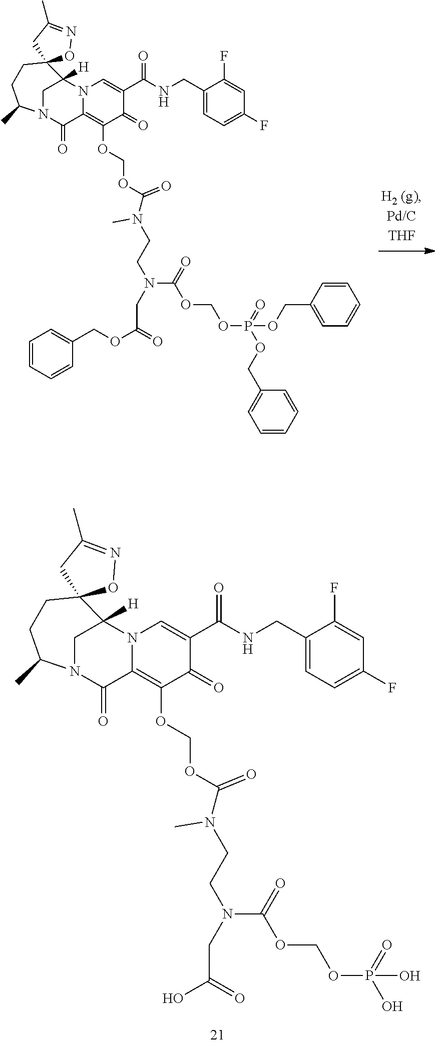 [SMILES: CC1=NO[C@@]2(CC[C@H](C)N3C[C@H]2n2cc(C(=O)NCc4ccc(F)cc4F)c(=O)c(OCOC(=O)N(C)CCN(CC(=O)OCc4ccccc4)C(=O)OCOP(=O)(OCc4ccccc4)OCc4ccccc4)c2C3=O)C1 ; CC1=NO[C@@]2(CC[C@H](C)N3C[C@H]2n2cc(C(=O)NCc4ccc(F)cc4F)c(=O)c(OCOC(=O)N(C)CCN(CC(=O)O)C(=O)OCOP(=O)(O)O)c2C3=O)C1]

### Step 1: Preparation of benzyl (2-((tert-butoxycarbonyl)amino)ethyl)glycinate

To a stirred solution of tert-butyl (2-aminoethyl)carbamate (3.0 g, 17.2 mmol) in DCM (90 mL) at 0° C. under argon was added benzyl 2-bromoacetate (1.97 g, 8.6 mmol) followed by DIPEA (2.45 g, 18.9 mmol). The reaction mixture was stirred for 16 h at room temperature. The reaction mixture was diluted with DCM and washed with water. The organic layer was dried over anhydrous Na₂SO₄, filtered, and concentrated under reduced pressure. The residue was purified by silica gel column chromatography (20-50% EtOAc/Hexanes) to afford the title compound. MS (m/z) 323.2 [M+H]+.

### Step 2: Preparation of benzyl N-(2-((tert-butoxycarbonyl)amino)ethyl)-N-((chloromethoxy)carbonyl)glycinate

To a stirred solution of benzyl (2-((tert-butoxycarbonyl)amino)ethyl)glycinate (2.8 g, 8.68 mmol) in DCM (45 mL) at 0° C. under argon was added chloromethyl chloroformate (1.46 g, 11.3 mmol) followed by Et₃N (2.2 g, 21.7 mmol). The mixture was stirred for 16 h at room temperature. The reaction mixture was diluted with DCM and washed with water. The organic layer was dried over anhydrous Na₂SO₄, filtered, and concentrated under reduced pressure. The crude residue was purified by silica gel column chromatography (20-50% EtOAc/Hexanes) to afford the title compound. MS (m/z) 415.6 [M+H]+.

### Step 3: Preparation of benzyl N-((((bis(benzyloxy)phosphoryl)oxy)methoxy)carbonyl)-N-(2-((tert-butoxycarbonyl)amino)ethyl)glycinate

To a stirred solution of benzyl N-(2-((tert-butoxycarbonyl)amino)ethyl)-N-((chloromethoxy)carbonyl)glycinate (3.25 g, 7.83 mmol) in toluene (20 mL) at room temperature under argon was added silver dibenzylphosphate (3.92 g, 10.2 mmol) under argon. The mixture was stirred at reflux for 16 h. The reaction mixture was allowed to cool to rt and was filtered, rinsing the solids with toluene (5V). The filtrate was concentrated under reduced pressure and purified by silica gel column chromatography (30-70% EtOAc/Hexanes) to afford the title compound. MS (m/z) 657.6 [M+H]+.

### Step 4: Preparation of benz N-(2-aminoethyl)-N-(((bis(benzyloxy)phosphoryl)oxy)methoxy)carbonyl)glycinate

To a stirred solution of benzyl N-((((bis(benzyloxy)phosphoryl)oxy)methoxy)carbonyl)-N-(2-((tert-butoxycarbonyl)amino)ethyl)glycinate (100 mg, 0.15 mmol) in DCM (1 mL) at 0° C. under argon was added 2,2,2-trifluoroacetic acid (0.375 mL) in 0.5 mL of DCM under argon. The mixture was stirred for 40 minutes at room temperature and concentrated under reduced pressure. The crude residue was washed with NaHCO₃(aq) twice. The organic layer was dried with Na₂SO₄ and filtered. The mixture was concentrated and dried under vacuum to obtain the title compound. MS (m/z) 557.6 [M+H]+.

### Step 5: benzyl N-((((bis(benzyloxy)phosphoryl)oxy)methoxy)carbonyl)-N-(2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)ethyl)glycinate

To a solution of benzyl N-(2-aminoethyl)-N-((((bis(benzyloxy)phosphoryl)oxy)methoxy)carbonyl)glycinate (200 mg, 0.36 mmol) in DCM (2 mL) was added (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-nitrophenyl) carbonate (Intermediate D, 100 mg, 0.15 mmol) at 0° C. The reaction mixture was warmed to room temperature and stirred for 4 h. The reaction mixture was concentrated to afford a residue, which was purified by reverse phase prep HPLC (5-100% MeCN/water) to afford the title compound. MS (m/z) 1099.2 [M+H]+.

### Step 6: Preparation of N-(2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methylaminoethyl)-N-(((phosphonooxy)methoxy)carbonyl)glycine (21)

To a solution of benzyl N-((((bis(benzyloxy)phosphoryl)oxy)methoxy)carbonyl)-N-(2-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)(methyl)amino)ethyl)glycinate (70 mg, 0.064 mmol) in THF (5 mL) was added 10% Pd/C (6.8 mg, 0.064 mmol). The flask was evacuated and backfilled with hydrogen gas (2×), then sparged with hydrogen for 2 min. The reaction mixture was left to stir under a hydrogen balloon atmosphere for 6 h. The reaction mixture was filtered, rinsed with THF, and concentrated to afford a residue, which was purified by reverse phase prep HPLC (5-100% MeCN/water) to afford the title compound. MS (m/z) 829.1 [M+H]+. 1H NMR (400 MHz, Methanol-d4) δ 8.56 (s, 1H), 7.46 (td, J=8.5, 6.3 Hz, 1H), 7.10-6.80 (m, 2H), 5.93-5.80 (m, 1H), 5.78-5.48 (m, 3H), 4.85-4.75 (m, 2H), 4.73-4.55 (m, 2H), 4.48 (d, J=2.2 Hz, 1H), 4.12 (s, 1H), 4.06 (d, J=4.5 Hz, 1H), 3.92-3.70 (m, 2H), 3.62-3.43 (m, 3H), 3.17 (d, J=17.9 Hz, 1H), 3.05-2.87 (m, 3H), 2.71 (d, J=18.1 Hz, 1H), 2.06 (s, 3H), 2.00-1.86 (m, 3H), 1.61-1.46 (m, 1H), 1.26 (dd, J=6.7, 1.3 Hz, 3H).

### Example 22: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((R)-2-(phosphonooxy)propyl) carbonate (22)

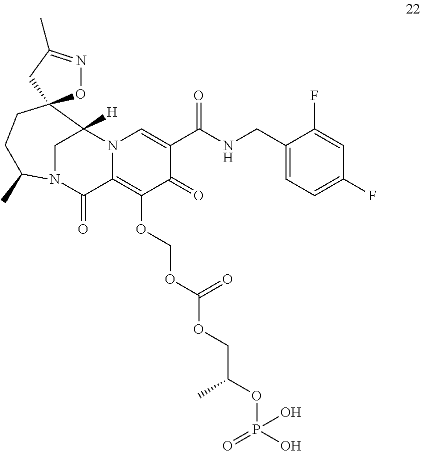 [SMILES: CC1=NO[C@@]2(CC[C@H](C)N3C[C@H]2n2cc(C(=O)NCc4ccc(F)cc4F)c(=O)c(OCOC(=O)OC[C@@H](C)OP(=O)(O)O)c2C3=O)C1]

The title compound was prepared in a manner similar to Example 18, except using (R)-1-((tert-butyldimethylsilyl)oxy)propan-2-ol instead of (R)-2-((tert-butyldimethylsilyl)oxy)propan-1-ol in Step 1. MS (m/z) 697.2 [M−H]⁻. 1H NMR (400 MHz, Chloroform-d) δ 10.36 (t, J=6.1 Hz, 1H), 8.49 (s, 1H), 7.36 (td, J=8.5, 6.3 Hz, 1H), 6.90-6.77 (m, 2H), 5.90 (d, J=6.5 Hz, 1H), 5.75 (d, J=6.4 Hz, 1H), 4.83 (q, J=7.9, 7.4 Hz, 1H), 4.62 (d, J=5.9 Hz, 2H), 4.22 (s, 1H), 4.23-4.06 (m, 2H), 3.85-3.77 (m, 1H), 3.73 (dd, J=15.1, 2.7 Hz, 1H), 3.08 (d, J=17.9 Hz, 1H), 2.62 (d, J=17.9 Hz, 1H), 2.08 (s, 3H), 1.94 (d, J=7.0 Hz, 4H), 1.63-1.52 (m, 1H), 1.34-1.22 (m, 6H).

### Example 23: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((S)-2-(phosphonooxy)propyl) carbonate (23)

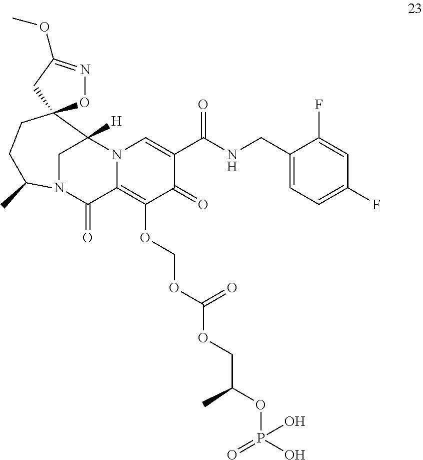 [SMILES: COC1=NO[C@@]2(CC[C@H](C)N3C[C@H]2n2cc(C(=O)NCc4ccc(F)cc4F)c(=O)c(OCOC(=O)OC[C@H](C)OP(=O)(O)O)c2C3=O)C1]

The title compound was prepared in a manner similar to Example 18, except using (S)-1-((tert-butyldimethylsilyl)oxy)propan-2-ol instead of (R)-2-((tert-butyldimethylsilyl)oxy)propan-1-ol in Step 1 and Intermediate C instead of Intermediate B in Step 4. MS (m/z) 714.96 [M+H]+. ¹H NMR (400 MHz, DMSO-d₆) δ 10.27 (t, J=6.0 Hz, 1H), 8.75 (s, 1H), 7.42 (td, J=8.6, 6.6 Hz, 1H), 7.25 (td, J=9.9, 2.5 Hz, 1H), 7.08 (td, J=8.6, 2.6 Hz, 1H), 5.82 (d, J=6.4 Hz, 1H), 5.64 (d, J=6.4 Hz, 1H), 4.75-4.48 (m, 4H), 4.41 (dq, J=11.8, 5.7 Hz, 1H), 4.13 (dd, J=11.1, 5.2 Hz, 1H), 4.04 (dd, J=11.1, 4.7 Hz, 1H), 3.82 (s, 3H), 3.75-3.59 (m, 2H), 3.05 (d, J=16.8 Hz, 1H), 2.76 (d, J=16.9 Hz, 1H), 1.97-1.68 (m, 3H), 1.35-1.29 (m, 1H), 1.21 (d, J=6.4 Hz, 3H), 1.16 (d, J=6.7 Hz, 3H).

### Example 24: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((S)-1-(phosphonooxy)propan-2-yl) carbonate (24)

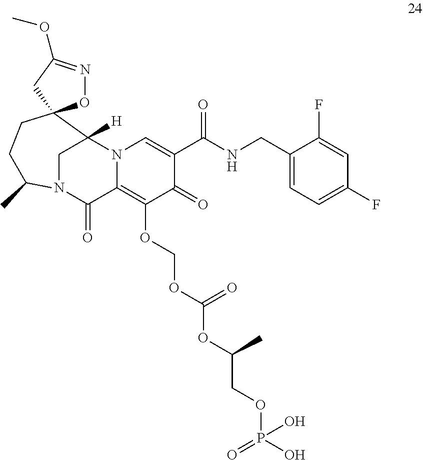 [SMILES: COC1=NO[C@@]2(CC[C@H](C)N3C[C@H]2n2cc(C(=O)NCc4ccc(F)cc4F)c(=O)c(OCOC(=O)O[C@@H](C)COP(=O)(O)O)c2C3=O)C1]

The title compound was prepared in a manner similar to Example 18, except using (S)-2-((tert-butyldimethylsilyl)oxy)propan-1-ol instead of (R)-2-((tert-butyldimethylsilyl)oxy)propan-1-ol in Step 1 and Intermediate C instead of Intermediate B in Step 4. MS (m/z) 714.95 [M+H]⁺. 1H NMR (400 MHz, DMSO-d6) δ 10.27 (t, J=5.9 Hz, 1H), 8.75 (s, 1H), 7.42 (td, J=8.7, 6.7 Hz, 1H), 7.25 (ddd, J=10.5, 9.3, 2.6 Hz, 1H), 7.13-7.00 (m, 1H), 5.89-5.73 (m, 1H), 5.73-5.53 (m, 1H), 4.88-4.75 (m, 1H), 4.71 (d, J=2.7 Hz, 1H), 4.69-4.58 (m, 1H), 4.54 (q, J=9.1, 7.7 Hz, 2H), 3.87 (s, 2H), 3.82 (s, 3H), 3.75-3.60 (m, 2H), 3.05 (d, J=16.8 Hz, 1H), 2.77 (d, J=16.9 Hz, 1H), 1.91 (dd, J=15.3.5.8 Hz, 1H), 1.88-1.69 (m, 2H), 1.40-1.26 (m, 1H), 1.21 (d, J=6.5 Hz, 3H), 1.15 (d, J=6.6 Hz, 3H).

### Example 25: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((R)-1-(phosphonooxy)butan-2-yl) carbonate (25)

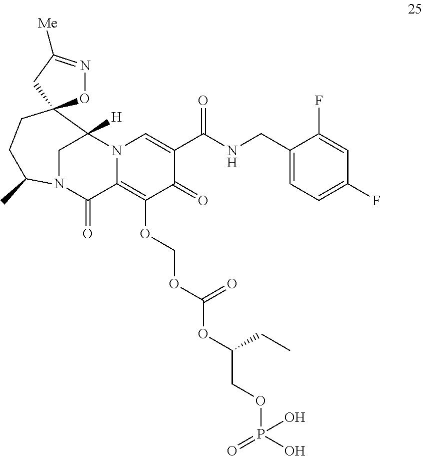 [SMILES: CC[C@H](COP(=O)(O)O)OC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a manner similar to Example 10, except using (2R)-butane-1,2-diol instead of propane-1,3-diol in Step 1. MS (m/z) 713.8 [M+H]+. ¹H NMR (400 MHz, DMSO) δ 10.40-10.15 (m, 1H), 8.78-8.62 (m, 1H), 7.52-7.33 (m, 1H), 7.33-7.17 (m, 1H), 7.15-6.94 (m, 1H), 5.90 (d, J=6.5 Hz, 1H), 5.61 (d, J=6.5 Hz, 1H), 4.72-4.47 (m, 5H), 4.32-4.07 (m, 1H), 3.99-3.81 (m, 1H), 3.81-3.58 (m, 2H), 3.07-2.92 (m, 1H), 2.72-2.58 (m, 1H), 1.95 (s, 3H), 1.89-1.69 (m, 3H), 1.69-1.50 (m, 2H), 1.35-1.20 (m, 1H), 1.20-1.09 (m, 3H), 0.94-0.80 (m, 3H).

### Example 26: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((S)-1-(phosphonooxy)butan-2-yl) carbonate (26)

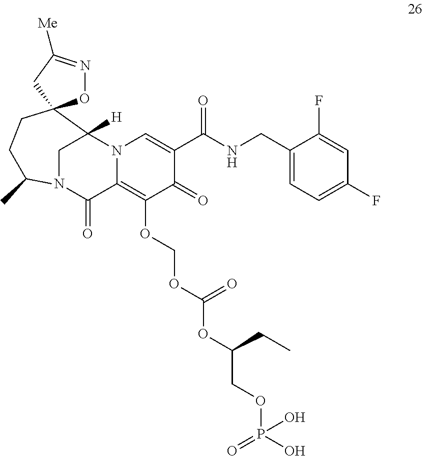 [SMILES: CC[C@@H](COP(=O)(O)O)OC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a manner similar to Example 10, except using (2S)-butane-1,2-diol instead of propane-1,3-diol in Step 1. MS (m/z) 713.4 [M+H]+. ¹H NMR (400 MHz, DMSO) δ 10.34-10.15 (m, 1H), 8.71 (s, 1H), 7.50-7.36 (m, 1H), 7.32-7.17 (m, 1H), 7.17-6.92 (m, 1H), 5.78 (d, J=6.5 Hz, 1H), 5.73 (d, J=6.5 Hz, 1H), 4.75-4.45 (m, 5H), 4.31-4.06 (m, 1H), 3.99-3.82 (m, 1H), 3.78-3.54 (m, 2H), 3.01 (d, J=17.6 Hz, 1H), 2.64 (d, J=17.7 Hz, 1H), 1.95 (s, 3H), 1.90-1.70 (m, 3H), 1.70-1.49 (m, 2H), 1.41-1.22 (m, 1H), 1.22-1.08 (m, 3H), 0.96-0.78 (m, 3H).

### Example 27: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2R,3R)-3-(phosphonooxy)butan-2-yl) carbonate (27)

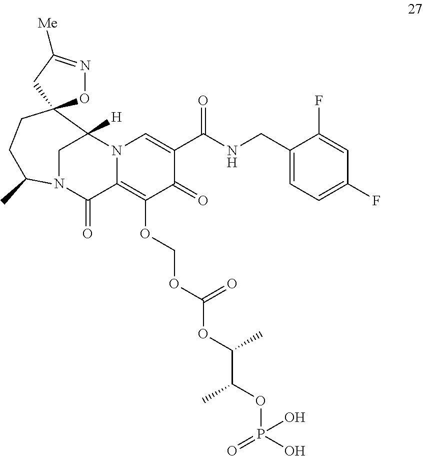 [SMILES: CC1=NO[C@@]2(CC[C@H](C)N3C[C@H]2n2cc(C(=O)NCc4ccc(F)cc4F)c(=O)c(OCOC(=O)O[C@H](C)[C@@H](C)OP(=O)(O)O)c2C3=O)C1]

The title compound was prepared in a manner similar to Example 28, except using (2R,3R)-butane-2,3-diol instead of (2S,3S)-butane-2,3-diol in Step 1. MS (m/z) 713.1 [M+H]+. ¹H NMR (400 MHz, DMSO) δ 10.28 (t, J=6.0 Hz, 1H), 8.72 (s, 1H), 7.50-7.34 (m, 1H), 7.34-7.16 (m, 1H), 7.16-7.01 (m, 1H), 5.84 (d, J=6.6 Hz, 1H), 5.65 (d, J=6.5 Hz, 1H), 4.80-4.45 (m, 5H), 4.42-4.25 (m, 1H), 3.78-3.62 (m, 2H), 3.15-2.91 (m, 1H), 2.73-2.56 (m, 1H), 1.95 (s, 3H), 1.87-1.66 (m, 3H), 1.39-1.23 (m, 1H), 1.23-1.09 (m, 9H).

### Example 28: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2S,3S)-3-(phosphonooxy)butan-2-yl) carbonate (28)

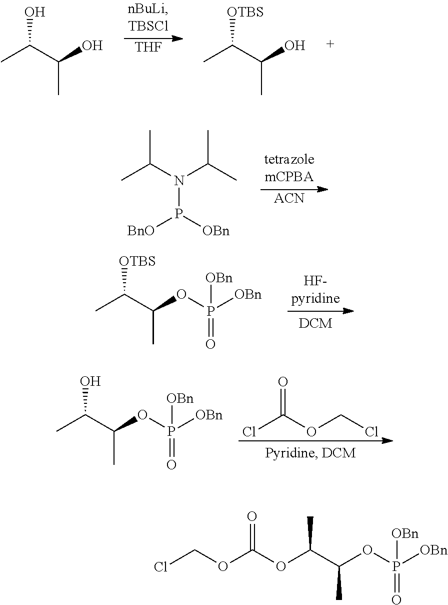

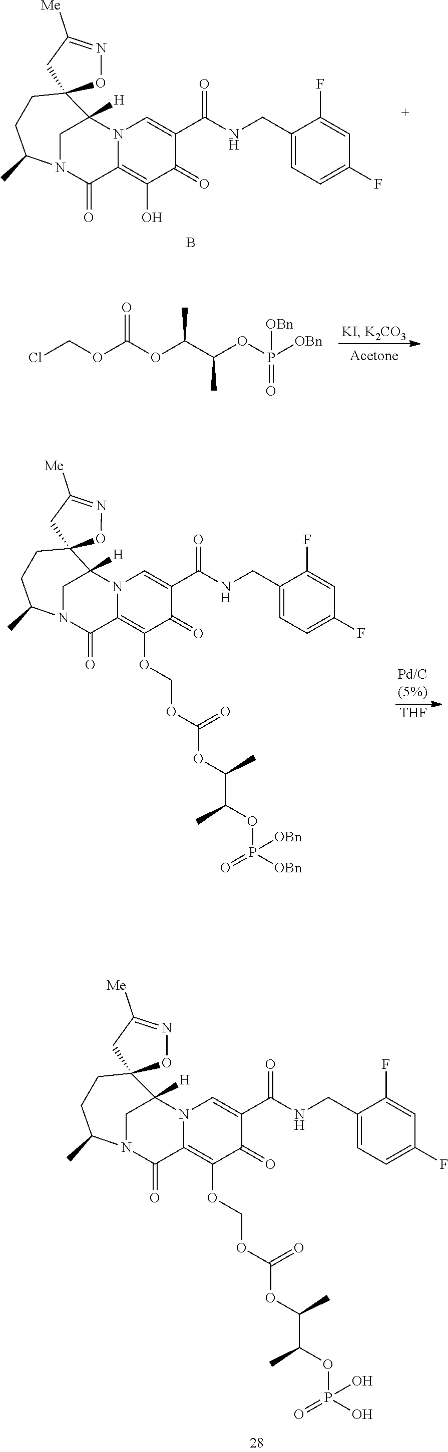

### Step 1: Preparation of (2S,3S)-3-((tert-butyldimethylsilyl)oxy)butan-2-ol

To a stirred solution of (2S,3S)-butane-2,3-diol (2.0 g, 22.2 mmol) in anhydrous THF (40 mL) at −78° C., was added n-butyllithium (1.7 M, 9.7 mL, 24.4 mmol) and stirred at −78° C. for 30 min, followed by TBDMS-Cl (3.68 g, 24.4 mmol). After being stirred at room temperature for 24 h, the reaction mixture was cooled to 0° C., quenched with water (10 mL) slowly, washed with ammonium chloride solution (10 mL) and extracted with ethyl acetate (20 mL). The organic layer was washed with water (2×10 mL), and saturated NaCl solution (2×10 mL), dried over anhydrous Na₂SO₄, filtered and concentrated under reduced pressure and purified by silica gel chromatography (0-40% EtOAc/Hexanes) to afford the title compound. ¹H NMR (400 MHz, CDCl₃) δ 3.57 (p, J=6.1 Hz, 1H), 3.49 (p, J=6.2 Hz, 1H), 1.15 (m, 6H), 0.93 (s, 9H), 0.12 (s, 6H).

### Step 2: Preparation of dibenzyl ((2S,3S)-3-((tert-butyldimethylsilyl)oxy)butan-2-yl) phosphate

A solution of (2S,3S)-3-((tert-butyldimethylsilyl)oxy)butan-2-ol (2.5 g, 12.2 mmol) and dibenzyl diisopropylphosphoramidite (6.47 g, 18.3 mmol), and 1H-tetrazole (1.29 g, 18.3 mmol) in DCM (80 mL) was stirred at room temperature. After 1 h, the reaction mixture was cooled to 0° C. to which mCPBA (5.48 g, 24.5 mmol) was added portion wise. After being stirred for 10 min at 0° C., the reaction mixture was washed thoroughly with saturated sodium bicarbonate (3×30 mL) and saturated sodium thiosulfate (3×30 mL), and extracted with DCM (2×50 mL). The organic layer was washed with water and saturated NaCl (25 mL) solution, dried over anhydrous Na₂SO₄, filtered and concentrated under reduced pressure. The residue was purified using silica gel column chromatography (0-60% EtOAc/Hexanes) to provide the title compound. MS (m/z) 464.9 [M+H]+

### Step 3: Preparation of dibenzyl ((2S,3S)-3-hydroxybutan-2-yl) phosphate

To a stirred solution of dibenzyl ((2S,3S)-3-((tert-butyldimethylsilyl)oxy)butan-2-yl) phosphate (5.0 g, 10.8 mmol) in anhydrous DCM (50 mL) at 0° C., was added dropwise 70% HF-pyridine (70%, 1.67 mL). The reaction mixture was warmed to room temperature and stirred for 2 h. The reaction mixture was cooled to 0° C., partitioned between sodium bicarbonate solution (20 mL) and DCM, and extracted with DCM (2×50 mL). The organic layer was washed with H₂O (2×20 mL) and saturated NaCl solution (20 mL), dried over anhydrous Na₂SO₄, filtered and concentrated under reduced pressure. The residue was purified by silica gel column chromatography (0-80% EtOAc/Hexanes) to afford the title compound. MS (m/z) 350.9 [M+H]+

### Step 4: Preparation of (2S,3S)-3-((bis(benzyloxy)phosphoryl)oxy)butan-2-yl (chloromethyl) carbonate

Dibenzyl ((2S,3S)-3-hydroxybutan-2-yl) phosphate (1.77 g, 5.1 mmol) was added to a stirred mixture of chloromethyl chloroformate (0.9 mL, 10.1 mmol) in DCM (25 mL) then followed by the addition of pyridine (0.72 mL, 8.8 mmol) dropwise at room temperature. The reaction was stirred for 30 minutes before it was diluted with DCM (10 mL) and washed sequentially with 0.5 M HCl (5 mL) and water (10 mL). The organic layer was dried over magnesium sulfate, filtered, and evaporated to obtain the title compound. MS (m/z) 444.2 [M+H]+.

### Step 5: Preparation of (2S,3S)-3-((bis(benzyloxy)phosphoryl)oxy)butan-2-yl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate

To the solution of Intermediate B (1.1 g, 2.3 mmol) in acetone (15 mL) was added KI (938 mg, 5.7 mmol) and K₂CO₃ (625 mg, 4.5 mmol) followed by (2S,3S)-3-((bis(benzyloxy)phosphoryl)oxy)butan-2-yl (chloromethyl) carbonate (2.0 g, 4.5 mmol). The resulting mixture was left to stir at room temperature for 48 h and then was concentrated. The residue was dissolved in DCM (30 mL), washed with water (2×20 mL), separated, dried and concentrated under reduced pressure. The residue was purified by silica gel column chromatography (0-100% EtOAc/Hexanes) to afford the title compound. MS (m/z) 893.3 [M+H]+.

### Step 6: Preparation of ((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2S,3S)-3-(phosphonooxy)butan-2-yl) carbonate (28)

To a solution (2S,3S)-3-((bis(benzyloxy)phosphoryl)oxy)butan-2-yl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate (1.0 g, 1.12 mmol) in THF (20 mL) was added 5 wt % Pd/C (119 mg, 0.11 mmol). The resulting mixture was purged with hydrogen three times before it was stirred under hydrogen for 1 h. The reaction was then filtered through Celite® and concentrated. The resulting residue was purified by reverse phase preparative HPLC (10-100% acetonitrile 0.1% TFA/water 0.1% TFA to afford the title compound. MS (m/z) 713.7 [M+H]+. ¹H NMR (400 MHz, DMSO) δ 10.28 (t, J=6.0 Hz, 1H), 8.71 (s, 1H), 7.52-7.34 (m, 1H), 7.34-7.19 (m, 1H), 7.19-7.02 (m, 1H), 5.84 (d, J=6.5 Hz, 1H), 5.66 (d, J=6.6 Hz, 1H), 4.75-4.47 (m, 5H), 4.41-4.15 (m, 1H), 3.79-3.59 (m, 2H), 3.00 (d, J=17.5 Hz, 1H), 2.74-2.58 (m, 1H), 1.95 (s, 3H), 1.89-1.67 (m, 3H), 1.41-1.22 (m, 1H), 1.22-1.05 (m, 9H).

### Example 29: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2S,3S)-3-(phosphonooxy)butan-2-yl) carbonate (29)

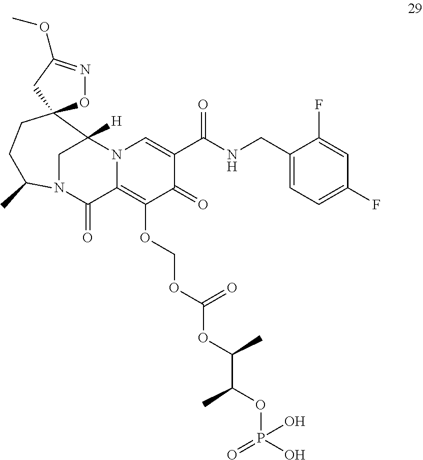 [SMILES: COC1=NO[C@@]2(CC[C@H](C)N3C[C@H]2n2cc(C(=O)NCc4ccc(F)cc4F)c(=O)c(OCOC(=O)O[C@@H](C)[C@H](C)OP(=O)(O)O)c2C3=O)C1]

The title compound was prepared following a similar method as Example 28, except using Intermediate C instead of Intermediate B in Step 5. MS (m/z) 728.80 [M+H]⁺. 1H NMR (400 MHz, Methanol-d4) δ 8.76 (s, 1H), 7.44 (td, J=8.5, 6.3 Hz, 1H), 7.08-6.81 (m, 2H), 5.90 (d, J=6.7 Hz, 1H), 5.71 (d, J=6.6 Hz, 1H), 4.79 (qd, J=7.1, 6.6, 2.8 Hz, 2H), 4.71 (d, J=2.5 Hz, 1H), 4.60 (s, 2H), 4.51-4.34 (m, 1H), 3.85 (s, 3H), 3.83-3.74 (m, 2H), 3.19 (d, J=17.0 Hz, 1H), 2.76 (d, J=17.1 Hz, 1H), 2.04 (dd, J=13.8, 4.4 Hz, 1H), 1.94 (td, J=10.4, 9.5, 3.7 Hz, 2H), 1.63-1.39 (m, 1H), 1.38-1.18 (m, 9H).

### Example 30: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2S,4S)-4-(phosphonooxy)pentan-2-yl) carbonate (30)

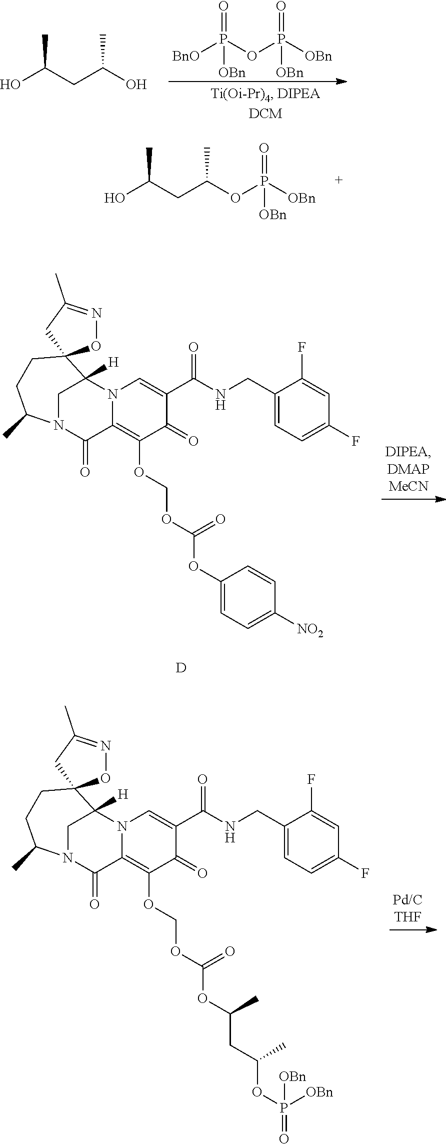

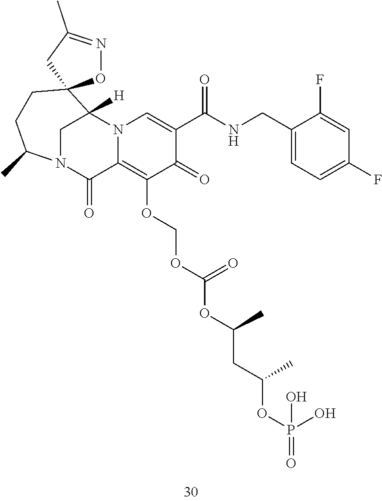 [SMILES: CC1=NO[C@@]2(CC[C@H](C)N3C[C@H]2n2cc(C(=O)NCc4ccc(F)cc4F)c(=O)c(OCOC(=O)O[C@@H](C)C[C@H](C)OP(=O)(O)O)c2C3=O)C1]

### Step 1: Preparation of dibenzyl ((2S,4S)-4-hydroxypentan-2-v1) phosphate

To a solution of (2S,4S)-pentane-2,4-diol (0.7 g, 6.72 mmol) in DCM (7 mL) was added N,N-diisopropylethylamine (1.76 mL, 10.1 mmol). To the resulting mixture was added dibenzyl dibenzyloxyphosphoryl phosphate (2.17 g, 4.03 mmol) and titanium(IV) isopropoxide (0.19 g, 0.67 mmol). The mixture was stirred at room temperature for 4 h then filtered through a column containing of silica gel and magnesium sulfate (20:1) and washed with ethyl acetate/hexanes (75%, 50 mL). The solution was dried over sodium sulfate, filtered, and concentrated under reduced pressure. The residue was purified by column chromatography on a silica gel to afford the title compound. MS (m/z) 364.9 [M+H]+.

### Step 2: Preparation of (2S,4S)-4-((bis(benzyloxyl)phosphoryl)oxy)pentan-2-yl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate

To the solution of Intermediate D (0.2 g, 0.29 mmol) in acetonitrile (2 mL) was added dibenzyl ((2S,4S)-4-hydroxypentan-2-yl) phosphate (0.21 g, 0.59 mmol), 4-dimethylaminopyridine (7.17 mg, 0.059 mmol) and N,N-diisopropylethylamine (0.076 g, 0.59 mmol). The resulting mixture was stirred at room temperature overnight. EtOAc was added to dilute and the mixture was washed with 0.5 N HCl, H₂O and brine. The organic phase was dried with MgSO₄, filtered, and concentrated. The resulting crude material was purified by silica gel column chromatography to obtain the title compound. MS (m/z) 906.7[M+H]+.

### Step 3: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2S,4S)-4-(phosphonooxy)pentan-2-yl) carbonate (30)

To the solution of (2S,4S)-4-((bis(benzyloxy)phosphoryl)oxy)pentan-2-yl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate (45.8 mg, 0.0505 mmol)) in THF (10 mL) was added 5% wt Pd/C (5.38 mg, 0.0051 mmol). The flask was purged with hydrogen three times and left to stir under hydrogen for 1 h. The reaction mixture was filtered through Celite and the filtrate was concentrated. The resulting residue was purified by prep HPLC (5-100% MeCN/water w/ 0.1% TFA) to afford the title compound. MS (m/z) 726.8 [M+H]+. 1H NMR (400 MHz, Chloroform-d) δ 10.19 (s, 1H), 8.67 (s, 1H), 7.39 (q, J=8.3 Hz, 1H), 6.95-6.66 (m, 2H), 5.97 (s, 1H), 5.53 (d, J=6.6 Hz, 1H), 5.12-4.75 (m, 2H), 4.63 (qd, J=15.0, 6.0 Hz, 4H), 4.34 (s, 1H), 3.96-3.56 (m, 2H), 2.64 (d, J=17.7 Hz, 1H), 2.08 (d, J=12.9 Hz, 3H), 2.02-1.85 (m, 3H), 1.79 (d, J=8.4 Hz, 2H), 1.67-1.45 (m, 1H), 1.30 (dd, J=24.7, 6.4 Hz, 9H).

### Example 31: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2R,4R)-4-(phosphonooxy)pentan-2-yl) carbonate (31)

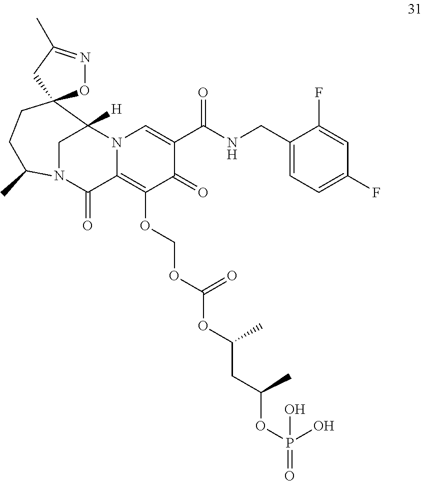 [SMILES: CC1=NO[C@@]2(CC[C@H](C)N3C[C@H]2n2cc(C(=O)NCc4ccc(F)cc4F)c(=O)c(OCOC(=O)O[C@H](C)C[C@@H](C)OP(=O)(O)O)c2C3=O)C1]

The title compound was prepared following a similar method as Example 30, except using (2R,4R)-pentane-2,4-diol instead of (2S,4S)-pentane-2,4-diol in Step 1. MS (m/z) 726.8 [M+H]+. 1H NMR (400 MHz, DMSO-d6) δ 10.29 (t, J=6.0 Hz, 1H), 8.71 (s, 1H), 7.43 (td, J=8.7, 6.6 Hz, 1H), 7.25 (ddd, J=10.5, 9.3, 2.6 Hz, 1H), 7.13-7.03 (m, 1H), 5.84 (d, J=6.4 Hz, 1H), 5.54 (d, J=6.4 Hz, 1H), 4.78-4.47 (m, 5H), 4.31-4.21 (m, 1H), 3.79-3.62 (m, 1H), 3.00 (d, J=17.6 Hz, 1H), 2.63 (d, J=17.5 Hz, 1H), 1.95 (s, 3H), 1.89-1.66 (m, 6H), 1.36-1.24 (m, 1H), 1.29-1.13 (m, 9H).

### Example 32: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (2,2-dimethyl-3-(phosphonooxy)propyl) carbonate (32)

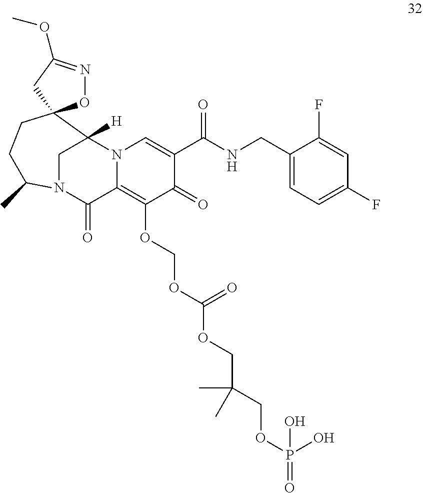 [SMILES: COC1=NO[C@@]2(CC[C@H](C)N3C[C@H]2n2cc(C(=O)NCc4ccc(F)cc4F)c(=O)c(OCOC(=O)OCC(C)(C)COP(=O)(O)O)c2C3=O)C1]

The title compound was prepared following a similar method as Example 10, except using 2,2-dimethylpropane-1,3-diol instead of propane-1,3-diol in Step 1 and Intermediate C instead of Intermediate B in Step 3. MS (m/z) 742.9 [M+H]+. 1H NMR (400 MHz, DMSO-d6) δ10.28 (t, J=6.0 Hz, 1H), 8.75 (s, 1H), 7.42 (q, J=8.2 Hz, 1H), 7.25 (td, J=9.9, 2.5 Hz, 1H), 7.08 (td, J=8.5, 2.5 Hz, 1H), 5.79 (d, J=6.4 Hz, 1H), 5.67 (d, J=6.4 Hz, 1H), 4.71 (s, 1H), 4.63 (dt, J=10.4, 6.9 Hz, 1H), 4.61-4.47 (m, 2H), 3.88 (m, 2H), 3.82 (m, 2H), 3.75-3.61 (m, 2H), 3.59 (d, J=5.2 Hz, 2H), 3.05 (d, J=16.8 Hz, 1H), 2.75 (d, J=16.9 Hz, 1H), 1.91 (dd. J=15.4, 5.9 Hz, 1H), 1.88-1.69 (m, 2H), 1.35 (d, J=12.1 Hz, 2H), 1.15 (d, J=6.7 Hz, 3H), 0.88 (s, 6H).

### Example 33: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((1-((phosphonooxy)methyl)cyclopropyl)methyl) carbonate (33)

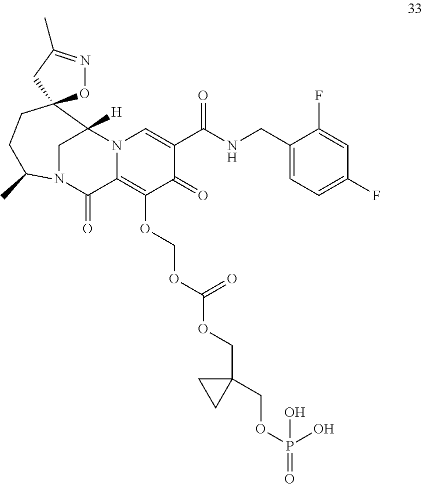 [SMILES: CC1=NO[C@@]2(CC[C@H](C)N3C[C@H]2n2cc(C(=O)NCc4ccc(F)cc4F)c(=O)c(OCOC(=O)OCC4(COP(=O)(O)O)CC4)c2C3=O)C1]

The title compound was prepared in a manner similar to Example 17, except using cyclopropane-1,1-diyldimethanol instead of (S)-propane-1,2-diol in Step 1. MS (m/z) 725.0 [M+H]⁺. 1H NMR (400 MHz, Methanol-d4) δ 8.58 (s, 1H), 7.46 (td, J=8.5, 6.3 Hz, 1H), 7.07-6.90 (m, 2H), 5.91 (d, J=6.5 Hz, 1H), 5.72 (d, J=6.6 Hz, 1H), 4.86-4.76 (m, 1H), 4.74-4.59 (m, 2H), 4.48 (d, J=2.5 Hz, 1H), 4.13 (s, 2H), 3.99-3.73 (m, 4H), 3.16 (d, J=17.8 Hz, 1H), 2.78-2.66 (m, 1H), 2.06 (s, 3H), 2.03-1.87 (m, 3H), 1.63-1.47 (m, 1H), 1.26 (d, J=6.7 Hz, 3H), 0.68 (d, J=2.6 Hz, 4H).

### Example 34: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-(phosphonooxy)butyl) carbonate (34)

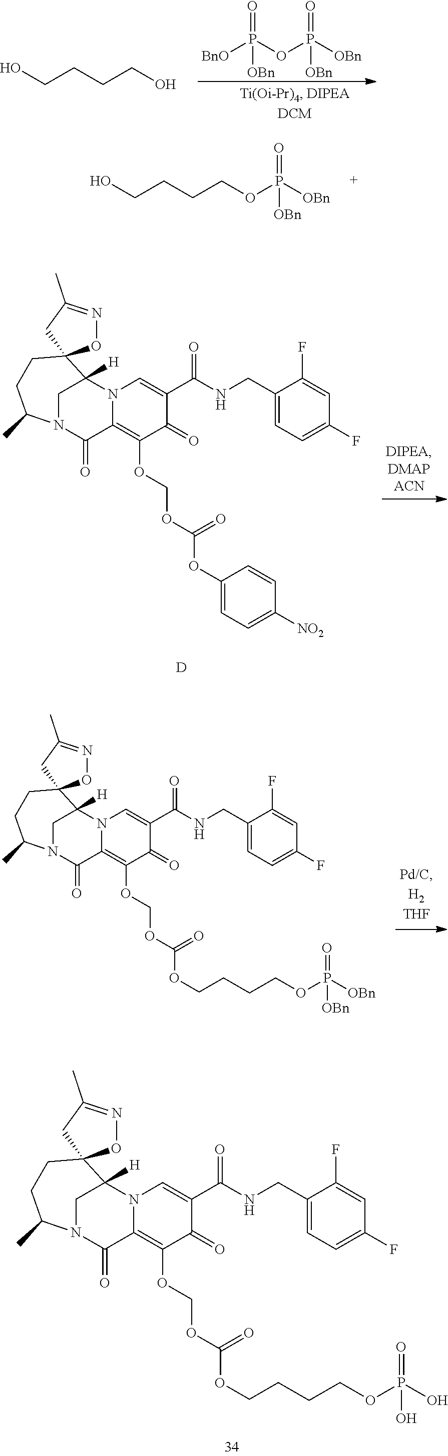

The title compound was prepared following a similar method as Example 30, except using butane-1,4-diol instead of (2S,4S)-pentane-2,4-diol. MS (m/z) 712.93 [M+H]+. 1H NMR (400 MHz, Chloroform-d) δ 10.51 (t, J=6.0 Hz, 1H), 8.43 (s, 1H), 7.42-7.30 (m, 1H), 6.96-6.78 (m, 2H), 5.90 (dd, J=49.9, 6.5 Hz, 2H), 4.84 (q, J=8.2, 7.2 Hz, 1H), 4.71-4.52 (m, 2H), 4.29-4.11 (m, 3H), 4.00 (d, J=6.6 Hz, 2H), 3.85-3.59 (m, 2H), 3.08 (d, J=17.9 Hz, 1H), 2.62 (d, J=17.9 Hz, 1H), 2.08 (s, 3H), 2.05-1.85 (m, 3H), 1.72 (m, 4H), 1.58 (dd, J=15.8, 9.6 Hz, 1H), 1.37-1.13 (m, 3H).

### Example 35: Preparation of (phosphonooxy)methyl ((2S)-1-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)propan-2-yl)(methyl)carbamate (35)

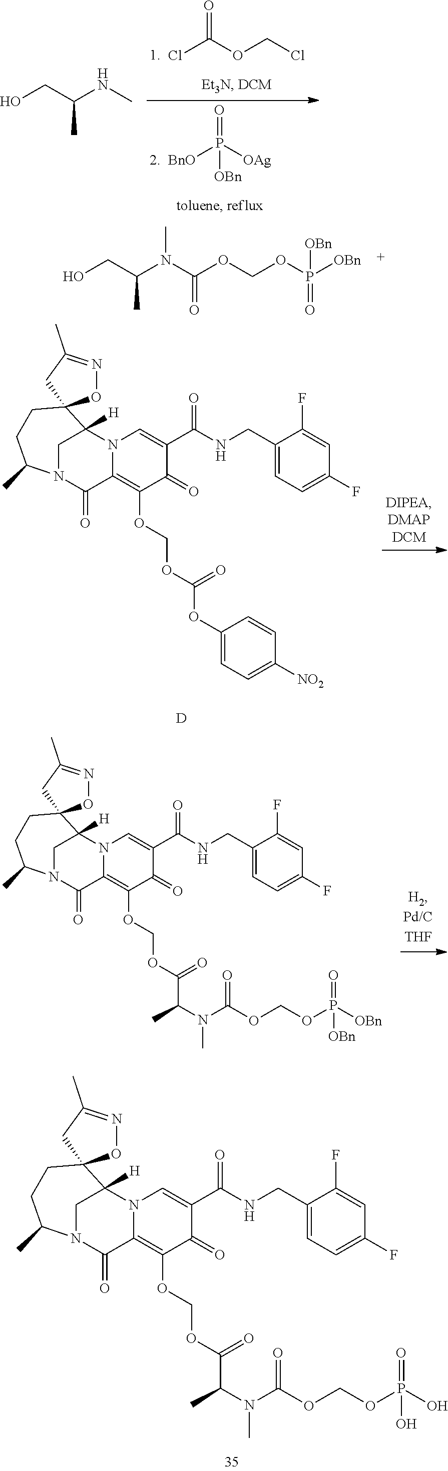

### Step 1: Preparation of ((bis(benzyloxy)phosphoryl)oxy)methyl (S)-(1-hydroxypropan-2-yl)(methyl)carbamate

To a stirred solution of (2S)-2-(methylamino)propan-1-ol (1.2 g, 13.5 mmol) in DCM (80) mL) at 0° C. under argon was added chloromethyl chloroformate (2.08 g, 16.2 mmol) followed by methylamine (3.5 g, 33.7 mmol). The mixture was stirred for 16 h at room temperature. The reaction mixture was diluted with DCM and washed with water. The organic layer was dried over anhydrous Na₂SO₄, filtered, and concentrated under reduced pressure. To the crude residue in toluene (60 mL) at room temperature under argon was added silver dibenzylphosphate (5.6 g, 15 mmol) under argon. The mixture was stirred at reflux for 16 h. The reaction mixture was allowed to cool to rt and was filtered, rinsing the solids with toluene (5V). The filtrate was concentrated under reduced pressure and purified by silica gel column chromatography (30-70% EtOAc/Hexanes) to afford the title compound. MS (m/z) 424.3 [M+H]+.

### Steps 2-3: Preparation of (phosphonooxy)methyl ((2S)-1-((((((3′S,5S,7′R)-10′-((2,4-difluorobenzyl)carbamoyl)-3,3′-dimethyl-1′,11′-oxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)propan-2-yl)(methyl)carbamate (35)

The title compound was prepared in a manner similar to Example 4, except using ((bis(benzyloxy)phosphoryl)oxy)methyl (S)-(1-hydroxypropan-2-yl)(methyl)carbamate, N,N-diisopropylethylamine, and dichloromethane instead of dibenzyl (2-hydroxyethyl) phosphate, triethylamine, and acetonitrile in Step 1. MS (m/z) 786.1 [M+H]. 1H NMR (400 MHz, Methanol-d4) δ 8.58 (s, 1H), 7.45 (td, J=8.5, 6.3 Hz, 1H), 7.04-6.88 (m, 2H), 5.92 (t, J=6.6 Hz, 1H), 5.73-5.50 (m, 3H), 4.86-4.75 (m, 1H), 4.73-4.56 (m, 2H), 4.56-4.41 (m, 2H), 4.25-4.05 (m, 2H), 3.89-3.71 (m, 2H), 3.17 (d, J=18.0 Hz, 1H), 2.86 (d, J=9.8 Hz, 3H), 2.72 (dd, J=18.0, 4.5 Hz, 1H), 2.06 (q, J=2.1, 1.5 Hz, 3H), 1.94 (q, J=11.7, 11.1 Hz, 3H), 1.55 (dd, J=10.0, 7.5 Hz, 1H), 1.26 (d, J=6.7 Hz, 3H), 1.20 (dd, J=9.4, 7.0 Hz, 3H).

### Example 36: Preparation of (phosphonooxy)methyl (1-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)-2-methylpropan-2-yl)(methyl)carbamate (36)

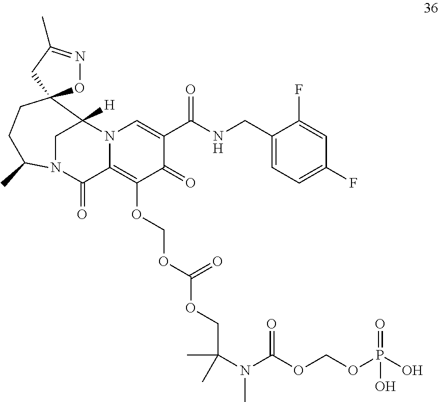 [SMILES: CC1=NO[C@@]2(CC[C@H](C)N3C[C@H]2n2cc(C(=O)NCc4ccc(F)cc4F)c(=O)c(OCOC(=O)OCC(C)(C)N(C)C(=O)OCOP(=O)(O)O)c2C3=O)C1]

The title compound was prepared in a manner similar to Example 35, except using 2-methyl-2-(methylamino)propan-1-ol instead of (2S)-2-(methylamino)propan-1-ol in Step 1. MS (m/z) 800.0 [M+H]⁺. 1H NMR (400 MHz, Methanol-d4) δ 8.58 (s, 1H), 7.54-7.37 (m, 1H), 7.05-6.93 (m, 2H), 5.89 (d, J=6.7 Hz, 1H), 5.74 (d, J=6.7 Hz, 1H), 5.57 (d, J=13.6 Hz, 2H), 4.85-4.75 (m, 1H), 4.71-4.59 (m, 2H), 4.47 (s, 1H), 4.41 (s, 2H), 3.79 (qd, J=15.2, 2.3 Hz. 2H), 3.16 (d, J=17.8 Hz, 1H), 3.01 (s, 3H), 2.76-2.66 (m, 1H), 2.06 (s, 3H), 1.95 (q, J=11.9, 10.4 Hz, 3H), 1.55 (dd, J=10.3, 7.8 Hz, 1H), 1.43 (s, 6H), 1.27 (d, J=6.7 Hz, 3H).

### Example 37: Preparation of N-(3-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)-2,2-dimethylpropyl)-N-(((phosphonooxy)methoxy)carbonyl)glycine (37)

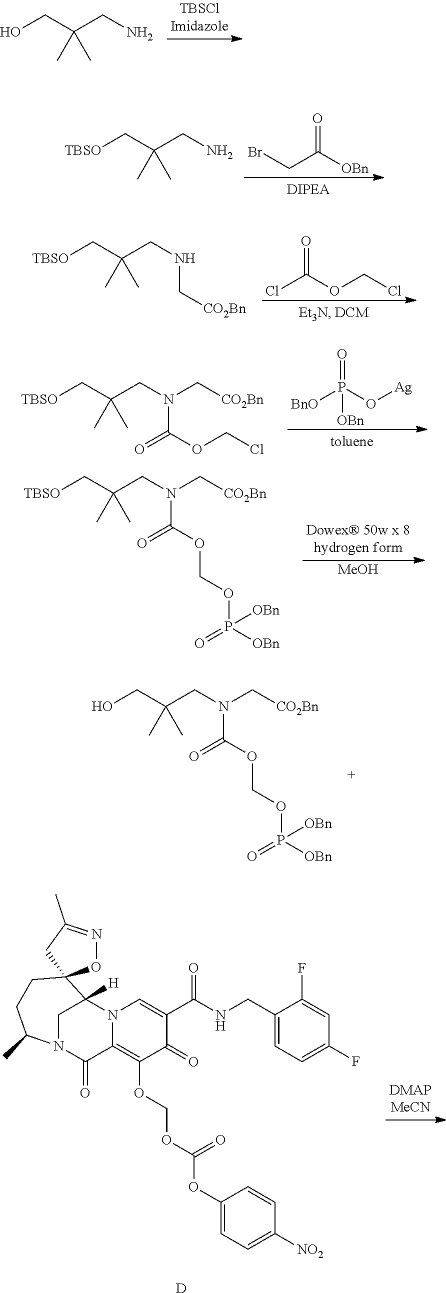

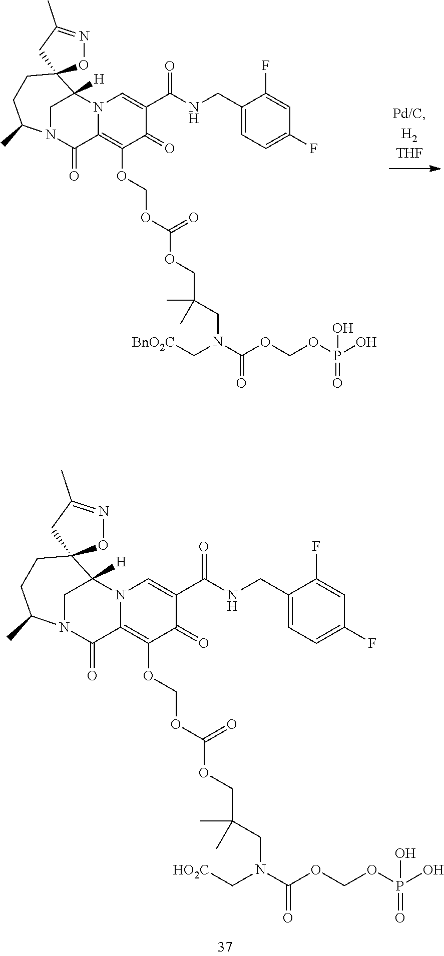

### Step 1: Preparation of 3-((tert-butyldimethylsilyl)oxy)-2,2-dimethylpropan-1-amine

To solution of 3-amino-2,2-dimethyl-propan-1-ol (4.5 g, 43.6 mmol) in DCM (40 mL) was added imidazole (5.94 g, 87.2 mmol), then followed by the addition of tert-butylchlorodimethylsilane (6.57 g, 43.6 mmol) dropwise at 0° C. The resulting solution was warmed up to room temperature and stirred of overnight. The solution was washed with H₂O, brine and dried with MgSO₄. The solvent was removed under vacuo to afford the title compound. MS (m/z) 217.9 [M+H]+.

### Step 2: Preparation of benzyl (3-((tert-butyldimethylsilyl)oxy)-2,2-dimethylprop 1)glycinate

To a stirred solution of 3-((tert-butyldimethylsilyl)oxy)-2,2-dimethylpropan-1-amine (4.7 g, 22 mmol) in CH₂Cl₂ (40 mL) at 0° C. was added DIPEA (8.4 g, 65 mmol) followed by the addition of benzyl 2-bromoacetate (2.5 g, 11 mmol) in CH₂Cl₂ (10 mL) dropwise. The reaction was stirred at 0° C. for 2 h. The solution was diluted with DCM and washed with H₂O and brine, then dried with MgSO₄. The solvent was removed under vacuo and the crude material was purified by silica gel column chromatography (0-10% DCM/MeOH) to obtain the title compound. MS (m/z) 366.23 [M+H]+.

### Step 3: Preparation of benzyl N-(3-((tert-butyldimethylsilyl)oxy)-2,2-dimethylpropyl)-N-((chloromethoxy)carbonyl)glycinate

To a stirred solution of benzyl (3-((tert-butyldimethylsilyl)oxy)-2,2-dimethylpropyl)glycinate (3 g, 8.21 mol) in DCM (30 mL) at −5° C. was added triethylamine (1.73 mL, 12.3 mmol) followed by the addition of chloromethyl carbonochloridate (0.98 g, 7.63 mmol) slowly. The mixture was stirred at −5° C. for 1 h. Saturated aqueous NaHCO₃ (30 mL) was added at 0° C. and the mixture was stirred for 10 minutes. EtOAc (100 mL) was added and the organic phase was washed with H₂O and brine, and dried over MgSO₄. The solvent was removed under vacuum to afford the title compound, which was used without further purification.

### Step 4: Preparation of benzyl N-((((bis(benzyloxy)phosphoryl)oxy)methoxycarbonyl)-N-(3-((tert-butyldimethylsilyl)oxy)-2,2-dimethylpropyl)glycinate

To a stirred solution of benzyl N-(3-((tert-butyldimethylsilyl)oxy)-2,2-dimethylpropyl)-N-((chloromethoxy)carbonyl)glycinate (3.5 g, 7.64 mmol) in toluene (35 mL) was added silver dibenzyl phosphate (5.9 g, 15.3 mmol). The resulting mixture was stirred at reflux for overnight. The mixture was filtered through celite and washed with toluene. The solvent was removed under vacuum and the resulting crude residue was purified by silica gel column chromatography (0-100% EtOAc/hexane) to obtain the title compound. MS (m/z) 700.4 [M+H]+.

### Step 5: Preparation of benzyl N-((((bis(benzyloxy)phosphoryl)oxy)methoxy)carbonyl)-N-(3-hydroxy-2,2-dimethylpropyl)glycinate

To a stirred solution of benzyl N-((((bis(benzyloxy)phosphoryl)oxy)methoxy)carbonyl)-N-(3-((tert-butyldimethylsilyl)oxy)-2,2-dimethylpropyl)glycinate (0.49 g, 0.7 mmol) in MeOH (15 mL) was added Dowex® 50W x8 hydrogen form (0.5 g). The resulting mixture was stirred at room temperature for overnight. The mixture was filtered through Celite and the solvent was removed under vacuum. The resulting crude material was purified by silica gel column chromatography (0-100% EtOAc/hexane) to obtain the title compound. MS (m/z) 585.9 [M+H]+.

### Step 6: Preparation of benzyl N-((((bis(benzyloxy)phosphoryl)oxy)methoxy)carbonyl)-N-(3-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5′,6′-[2,7]methanopyrido[1,2-a][1,4]diazonin]-12′-yl)oxy)methoxy)carbonyl)oxy)-2,2-dimethylpropyl)glycinate

To a stirred solution of Intermediate D (0.28 g, 0.41 mmol) in acetonitrile (5 mL) was added benzyl N-((((bis(benzyloxy)phosphoryl)oxy)methoxy)carbonyl)-N-(3-hydroxy-2,2-dimethylpropyl)glycinate (0.29 g, 0.49 mmol) and 4-dimethylaminopyridine (0.05 g, 0.41 mmol). The resulting mixture was stirred at room temperature for overnight. The mixture was diluted with EtOAc and washed with H₂O, brine and dried by MgSO₄. The solvent was removed under vacuum and the resulting crude material was purified by silica gel column chromatography (0-100% EtOAc/hexane) to obtain the title compound. MS (m/z) 1128.9 [M+H]+.

### Step 7: Preparation of N-(3-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)-2,2-dimethylpropyl)-N-(((phosphonooxy)methoxy)carbonyl)glycine (37)

A solution of benzyl N-((((bis(benzyloxy)phosphoryl)oxy)methoxy)carbonyl)-N-(3-((((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)oxy)-2,2-dimethylpropyl)glycinate (0.26 g, 0.23 mmol) in THF (25 mL) was added 10% Pd/C (0.0245 g, 0.023 mmol). The mixture was purged with H₂ and stirred at room temperature for 1 hour. The mixture was filtered through Celite and washed with THF. The solvent was removed under vacuum and the residue was purified by prep-HPLC (5-100% MeCN/water containing 0.1% TFA) to obtain the title compound. MS (m/z) 857.96 [M+H]+. 1H NMR (400 MHz, Chloroform-d) δ 10.78-10.45 (m, 1H), 8.58 (d, J=4.2 Hz, 1H), 7.35 (td, J=8.4, 6.3 Hz, 1H), 6.90-6.79 (m, 2H), 6.00-5.69 (m, 2H), 5.53 (dd, J=37.4, 10.3 Hz, 2H), 4.83 (d, J=7.9 Hz, 1H), 4.62 (d, J=5.6 Hz, 2H), 4.30 (s, 1H), 3.93 (ddd, J=30.2, 25.1, 8.8 Hz, 4H), 3.74 (d, J=14.5 Hz, 2H), 3.31 (q, J=13.5, 11.7 Hz, 1H), 3.26-3.01 (m, 2H), 2.61 (d, J=18.0 Hz, 1H), 2.05 (s, 3H), 1.90 (d, J=18.2 Hz, 3H), 1.53 (s, 1H), 1.24 (d, J=6.4 Hz, 3H), 0.96 (dd, J=6.3, 3.2 Hz, 6H).

### Example 38: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl ((S)-2-(phosphonooxy)propyl) carbonate (38)

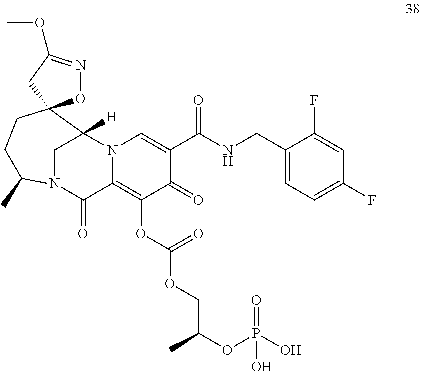 [SMILES: COC1=NO[C@@]2(CC[C@H](C)N3C[C@H]2n2cc(C(=O)NCc4ccc(F)cc4F)c(=O)c(OC(=O)OC[C@H](C)OP(=O)(O)O)c2C3=O)C1]

The title compound was prepared in a manner similar to Example 6 except using (S)-dibenzyl (1-hydroxypropan-2-yl) phosphate, prepared according to WO2019136112, instead of dibenzyl (2-hydroxyethyl) phosphate in Step 1 and Intermediate C instead of Intermediate B in Step 2. MS (m/z) 685.04 [M+H]+. 1H NMR (400 MHz, DMSO-d6) δ 10.12 (t, J=6.0 Hz, 1H), 8.90 (s, 1H), 7.42 (td, J=8.7, 6.6 Hz, 1H), 7.24 (ddd, J=10.4, 9.3, 2.6 Hz, 1H), 7.14-7.02 (m, 1H), 4.80 (s, 1H), 4.66-4.41 (m, 4H), 4.25 (dd, J=11.2, 4.4 Hz, 1H), 4.17 (dd, J=11.2, 4.4 Hz, 1H), 3.88-3.76 (m, 4H), 3.70 (dd, J=15.2, 1.9 Hz, 1H), 3.09 (d, J=16.7 Hz, 1H), 2.79 (d, J=16.9 Hz, 1H), 1.98-1.74 (m, 3H), 1.33-1.22 (m, 4H), 1.17 (d, J=6.7 Hz, 3H).

### Example 39: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl ((S)-2-(phosphonooxy)propyl) carbonate (39)

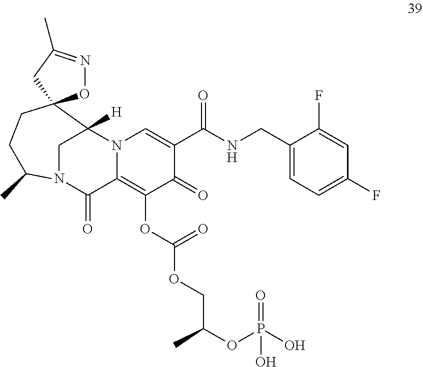 [SMILES: CC1=NO[C@@]2(CC[C@H](C)N3C[C@H]2n2cc(C(=O)NCc4ccc(F)cc4F)c(=O)c(OC(=O)OC[C@H](C)OP(=O)(O)O)c2C3=O)C1]

The title compound was prepared in a manner similar to Example 11, except using (S)-dibenzyl (1-hydroxypropan-2-yl) phosphate, prepared according to WO2019136112, instead of dibenzyl (3-hydroxypropyl) phosphate. MS (m/z) 669.1 [M+H]+. 1H NMR (400 MHz, DMSO-d6) δ 10.12 (t, J=6.0 Hz, 1H), 8.86 (s, 1H), 7.42 (td, J=8.7, 6.6 Hz, 1H), 7.25 (ddd, J=10.5, 9.3, 2.6 Hz, 1H), 7.08 (tdd, J=8.5, 2.6, 1.0 Hz, 1H), 4.72 (d, J=4.7 Hz, 1H), 4.63-4.52 (m, 3H), 4.48 (dtd, J=8.7, 6.8, 4.8 Hz, 1H), 4.26 (dd, J=11.1, 4.5 Hz, 1H), 4.18 (dd, J=11.1, 4.4 Hz, 1H), 3.83 (dd, J=15.2, 2.7 Hz, 1H), 3.71 (dt, J=13.9, 2.8 Hz, 1H), 3.03 (d, J=17.5 Hz, 1H), 2.67 (d, J=17.5 Hz, 1H), 1.95 (s, 3H), 1.84-1.73 (m, 3H), 1.28 (d, J=6.4 Hz, 3H), 1.26-1.21 (m, 1H), 1.17 (d, J=6.6 Hz, 3H).

### Example 40: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 4-(phosphonooxy)butanoate (40)

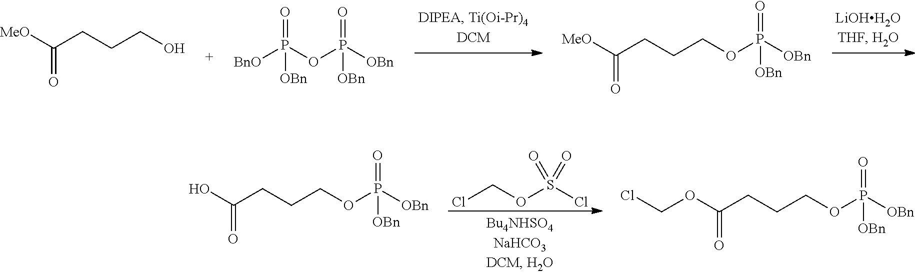 [SMILES: COC(=O)CCCO ; O=P(OCc1ccccc1)(OCc1ccccc1)OP(=O)(OCc1ccccc1)OCc1ccccc1 ; COC(=O)CCCOP(=O)(OCc1ccccc1)OCc1ccccc1 ; O=C(O)CCCOP(=O)(OCc1ccccc1)OCc1ccccc1 ; O=S(=O)(Cl)OCCl ; O=C(CCCOP(=O)(OCc1ccccc1)OCc1ccccc1)OCCl]

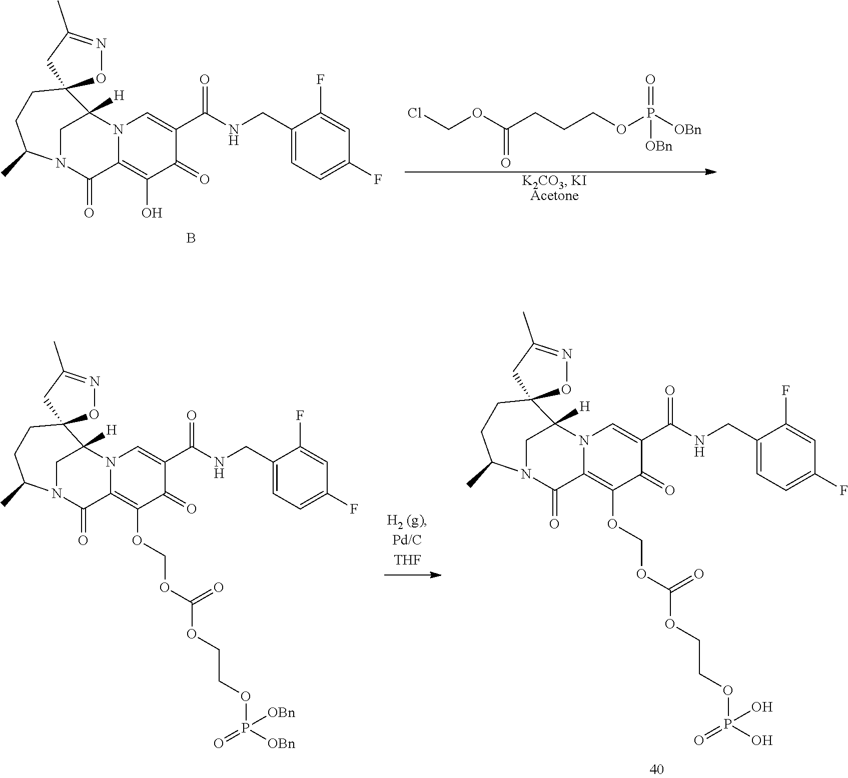

### Step 1: Preparation of methyl 4-((bis(benzyloxy) phosphoryl)oxy)butanoate

To a mixture of methyl 4-hydroxybutanoate (0.491 g, 4.16 mmol) in DCM (4 mL) was added DIPEA (1.66 mL, 9.25 mmol) followed by tetrabenzylpyrophosphate (2.00 g, 3.71 mmol) and Ti(i-PrO)₄ (0.187 mL, 0.631 mmol). The mixture was stirred at rt overnight. MgSO₄ and silica gel (4:1) were added to reaction, stirred for 10 min, then filtered and washed with 4:3 EtOAc/hexanes. The filtrate was concentrated and purified by reverse phase prep HPLC (10-100% MeCN/water) and lyophilized to afford the title compound. MS (m/z) 378.73 [M+H]+.

### Step 2: Preparation of 4-((bis(benzyloxy)phosphoryl)oxy)butanoic acid

To a solution of methyl 4-((bis(benzyloxy)phosphoryl)oxy)butanoate (0.224 g, 0.592 mmol) in THF (2.7 mL) and water (1.8 mL) at 0° C. was added LiOH—H₂O (0.0497 g, 1.18 mmol). The reaction mixture was stirred at 0° C. for 3 h and poured into 1 M HCl at 0° C. EtOAc was added and the phases were separated. The aqueous phase was extracted with EtOAc (2×) and the combined organic phase was washed with brine. The organic phase was dried over Na₂SO₄, filtered, and concentrated. The crude residue was dissolved in DCM/MeOH and purified by silica gel column chromatography (0-20% MeOH/DCM) to afford the title compound. MS (m/z) 364.83 [M+H]+.

### Step 3: Preparation of chloromethyl 4-((bis(benzyloxy)phosphoryl)oxy)butanoate

To a solution of 4-((bis(benzyloxy)phosphoryl)oxy)butanoic acid (0.154 g, 0.423 mmol) in DCM (1.5 mL) at 0° C. was added a suspension of NaHCO₃ (0.178 g, 2.11 mmol) in water (0.6 mL). Tetrabutylammonium hydrogen sulfate (0.0144 g, 0.042 mmol) was added to the biphasic mixture followed by chloromethyl chlorosulfate (0.107 mL, 1.06 mmol). The mixture was allowed to warm to rt and stir for 3 h. The mixture was partitioned between saturated aqueous NaHCO₃ and DCM. The phases were separated, and the aqueous phase was extracted with DCM (2×). The combined organic phase was washed with brine, dried over Na₂SO₄, filtered, and concentrated to afford the title compound. MS (m/z) 412.82 [M+H]+.

### Step 4: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 4-((bis(benzyloxy)phosphoryl)oxy)butanoate

To a solution of chloromethyl 4-((bis(benzyloxy)phosphoryl)oxy)butanoate (0.127 g, 0.308 mmol) in acetone (3 mL) was added Intermediate B (0.125 g, 0.257 mmol), potassium iodide (0.056 g, 0.334 mmol), and potassium carbonate (0.071 g, 0.514 mmol). The reaction mixture was left to stir overnight at rt. The mixture was partitioned between EtOAc and water and the phases were separated. The organic phase was washed with brine, dried over Na₂SO₄, filtered, and concentrated to afford the title compound. MS (m/z) 862.85 [M+H]+.

### Step 5: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 4-(phosphonooxy)butanoate (40)

To a solution of (((3′S,5S,7′R)-10′-((2,4-difluorobenzyl)carbamoyl)-3,3′-dimethyl-1′,1′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 4-((bis(benzyloxy)phosphoryl)oxy)butanoate (0.143 g, 0.166 mmol) in THF (17 mL) was added 10% Pd/C (0.018 g, 0.017 mmol). The flask was evacuated and backfilled with H₂ (g) (2×) then sparged with H₂ (g) for 2 min. The reaction mixture was left to stir under an atmosphere of hydrogen for 4 h then filtered, rinsing with THF. The filtrate was concentrated and the residue was purified by reverse phase prep HPLC (10-1000% MeCN/water w/ 0.1% TFA) and lyophilized to afford the title compound. MS (m/z) 682.82 [M+H]+. 1H NMR (400 MHz, DMSO-d6) δ 10.29 (t, J=6.0 Hz, 1H), 8.69 (s, 1H), 7.42 (td, J=8.7, 6.6 Hz, 1H), 7.24 (ddd, J=10.5, 9.3, 2.6 Hz, 1H), 7.07 (tdd, J=8.6, 2.6, 1.0 Hz, 1H), 5.80 (d, J=6.2 Hz, 1H), 5.60 (d, J=6.3 Hz, 1H), 4.70-4.60 (m, 2H), 4.60-4.48 (m, 2H), 3.81 (q, J=6.7 Hz, 2H), 3.70-3.64 (m, 2H), 2.99 (d, J=17.6 Hz, 1H), 2.63 (d, J=17.6 Hz, 1H), 2.37 (t, J=7.4 Hz, 2H), 1.94 (s, 3H), 1.78 (ddt, J=17.2, 10.4, 6.1 Hz, 5H), 1.36-1.21 (m, 1H), 1.15 (d, J=6.7 Hz, 3H).

### Example 41: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 3,3-dimethyl-4-(phosphonooxy)butanoate (41)

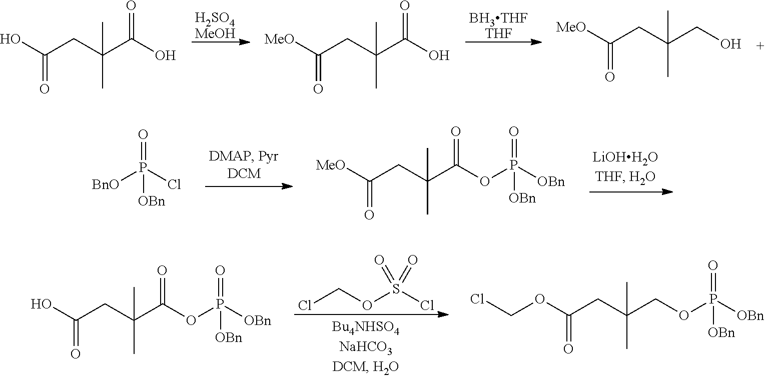

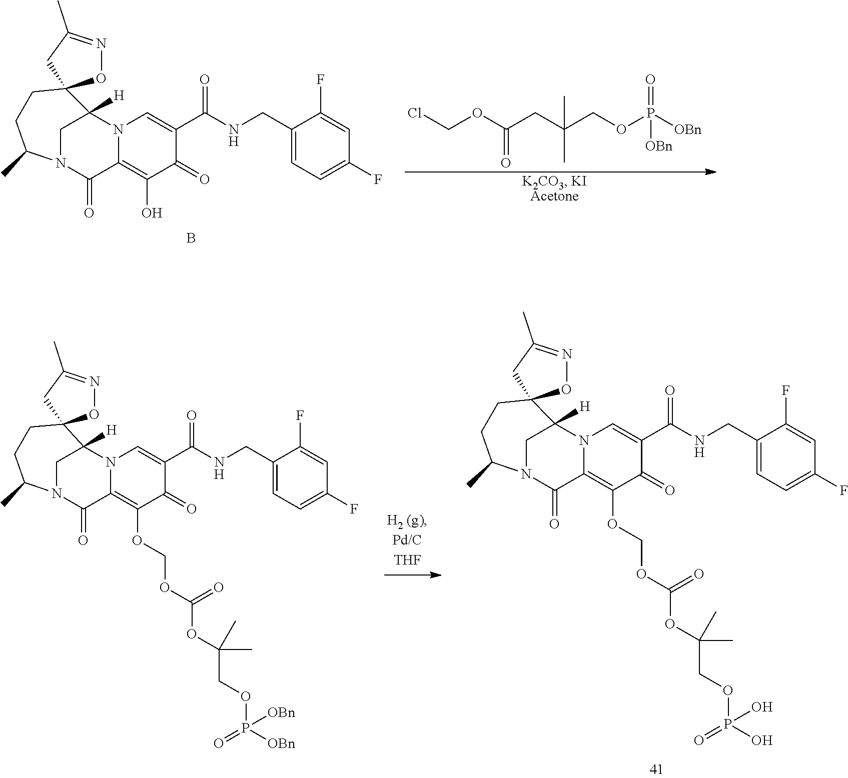

### Step 1: Preparation of 4-methoxy-2,2-dimethyl-4-oxobutanoic acid

To a solution of 2,2-dimethylsuccinic acid (10 g, 0.068 mol) in MeOH (50 mL) was added H2SO4 (conc, 0.6 g) at 0° C. It was stirred at r.t. overnight. After cooled to r.t., it was diluted with NaHCO₃ solution and washed with hexane. The aqueous phase was adjusted to pH=2 and extracted with EtOAc. The combined organic phase was dried and concentrated to give the title compound. 1H NMR (400 MHz, CDCl₃) δ 3.68 (s, 3H), 2.62 (s, 2H), 1.31 (s, 6H).

### Step 2: Preparation of methyl 4-hydroxy-3,3-dimethylbutanoate

To a solution of 4-methoxy-2,2-dimethyl-4-oxobutanoic acid (10 g, 62.4 mmol) in THF (150 mL) was added BH₃·THF (IM in THF, 62 mL) at 0° C. It was stirred at r.t. overnight. The reaction mixture was quenched with MeOH (20 mL) at 0° C. and concentrated. It was purified by silica gel chromatography (PE:EtOAc=5:1 to 2:1) to afford the title compound. 1H NMR (400 MHz, CDCl₃) δ 3.68 (s, 3H), 3.40 (s, 2H), 2.32 (s, 2H), 0.99 (s, 6H).

### Step 3: Preparation of methyl 4-((bis(benzyloxy)phosphoryl)oxy)-3,3-dimethylbutanoate

Dibenzylphosphite (8.64 g, 32.96 mmol) was added to a solution of N-chlorosuccinimide (4.84 g, 36.24 mmol) in toluene (160 mL) at room temperature for 2 h. The mixture was filtered and the filtrate was evaporated under vacuum to give dibenzyl phosphorochloridate.

To a stirred solution of crude dibenzyl phosphorochloridate (5.6 g, 13.8 mmol) in DCM (50 mL) under argon with an ice bath were sequentially added methyl 4-hydroxy-3,3-dimethylbutanoate (1.35 g, 9.2 mmol), pyridine (1.45 g, 18.4 mmol) and DMAP (122 mg, 1 mmol). The reaction mixture was stirred at r.t. for 4 hours. H₂O was added and extracted with DCM (2×100 mL). The organic phase was dried and concentrated, then purified by silica gel column chromatography (PE:EtOAc=5:1) to give the title compound. MS (m/z) 407.1 [M+H]+.

### Step 4: Preparation of 4-((bis(benzyloxy)phosphoryloxy)-3,3-dimethylbutanoic acid

methyl 4-((bis(benzyloxy)phosphoryl)oxy)-3,3-dimethylbutanoate (12.7 g, 31.3 mmol) was added to a solution of LiOH—H₂O (10.5 g, 250 mmol) in H₂O/THF (1:1, 150 mL). It was stirred at 40° C. for 8 hours. After cooled to r.t., H₂O (300 mL) was added and it was washed with EtOAc (2×200 mL). The pH of the aqueous phase was adjusted to 6 with 1M HCl. It was extracted with EtOAc (2×200 mL). The combined organic phase was washed with H₂O, brine, dried (Na₂SO₄ anhydrous) and filtered then concentrated. It was purified by silica gel column chromatography to give the title compound. MS (m/z) 393.1 [M+H]+.

### Step 5: Preparation of chloromethyl 4-((bis(benzyloxy)phosphoryl)oxy)-3,3-dimethylbutanoate

To a stirred solution of 4-((bis(benzyloxy)phosphoryl)oxy)-3,3-dimethylbutanoic acid (2.2 g, 5.5 mmol) in DCM (30 mL) with an ice bath was added a suspension of NaHCO3 (1.66 g, 26.8 mmol) in water (10 mL). The reaction mixture was stirred with an ice bath for 15 minutes. To the resulting white suspension were added Bu₄NHSO₄ (200 mg, 0.6 mmol) and chloromethyl chlorosulfate (1.76 g, 10.72 mmol) via syringe. The reaction mixture was warmed gradually to room temperature and stirred for 1-3 hours. The reaction mixture was purified by silica gel chromatography, eluting with PE:EtOAc (from I/O to 2/1) to afford the title compound. MS (m/z) 441.1 [M+H]+.

### Step 6: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 4-((bis(benzyloxy)phosphor yl)oxy)-3,3-dimethylbutanoate

To a solution of chloromethyl 4-((bis(benzyloxy)phosphoryl)oxy)-3,3-dimethylbutanoate (0.136 g, 0.308 mmol) in acetone (3 mL) was added Intermediate B (0.125 g, 0.257 mmol), potassium iodide (0.056 g, 0.334 mmol), and potassium carbonate (0.071 g, 0.514 mmol). The reaction mixture was left to stir overnight at rt. The reaction mixture was partitioned between EtOAc and water and the phases separated. The organic phase was washed with brine, dried over Na₂SO₄, filtered, and concentrated to afford the title compound. MS (m/z) 890.82 [M+H]+.

### Step 7: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 3,3-dimethyl-4-(phosphonooxy)butanoate (41)

To a solution of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 4-((bis(benzyloxy)phosphoryl)oxy)-3,3-dimethylbutanoate (0.196 g, 0.220 mmol) in THF (20 mL) was added 10% Pd/C (0.023 g, 0.022 mmol). The flask was evacuated and backfilled with H₂ (g) (2×) then sparged with H₂ (g) for 2 min. The reaction mixture was left to stir under an atmosphere of hydrogen for 4 h then filtered, rinsing with THF. The filtrate was concentrated and the residue was purified by reverse phase prep HPLC (10-100% MeCN/water w/ 0.1% TFA) and lyophilized to afford the title compound. MS (m/z) 710.79 [M+H]+. 1H NMR (400 MHz, DMSO-d6) δ 10.29 (t, J=6.0 Hz, 1H), 8.68 (s, 1H), 7.42 (td, J=8.7, 6.6 Hz, 1H), 7.24 (ddd, J=10.5, 9.3, 2.6 Hz, 1H), 7.13-7.01 (m, 1H), 5.78 (d, J=6.3 Hz, 1H), 5.59 (d, J=6.3 Hz, 1H), 4.68-4.58 (m, 2H), 4.54 (t, J=6.2 Hz, 2H), 3.67 (d, J=2.3 Hz, 2H), 3.61-3.54 (m, 2H), 2.99 (d, J=17.5 Hz, 1H), 2.63 (d, J=17.6 Hz, 1H), 2.25 (d, J=1.4 Hz, 2H), 1.94 (s, 3H), 1.88-1.68 (m, 3H), 1.30 (dd, J=15.5, 10.8 Hz, 1H), 1.15 (d, J=6.7 Hz, 3H), 0.92 (d, J=2.3 Hz, 6H).

### Example 42: Preparation of N-(4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)-2,2-dimethyl-4-oxobutyl)-N-(((phosphonooxy)methoxy)carbonyl)glycine (42)

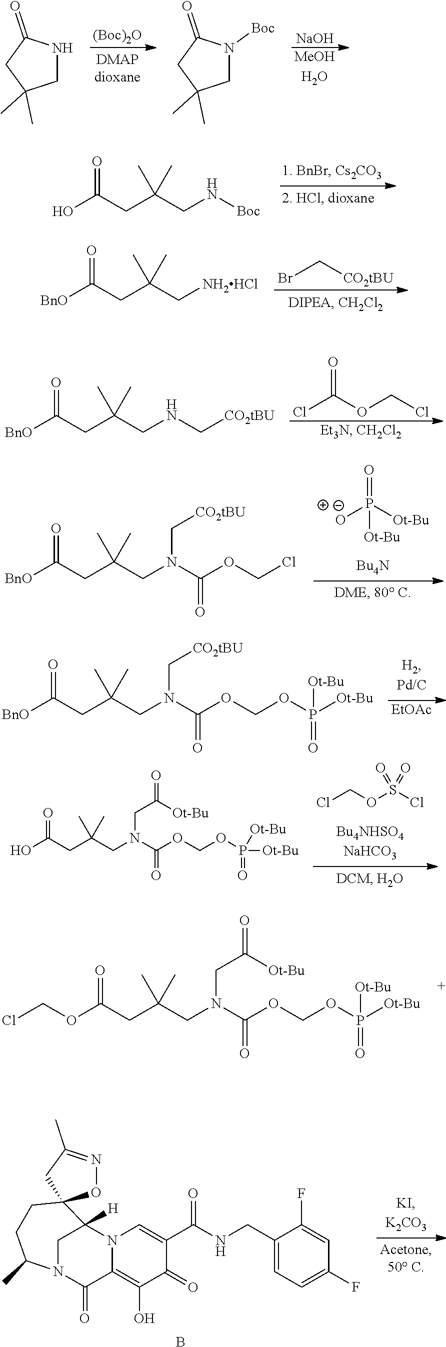

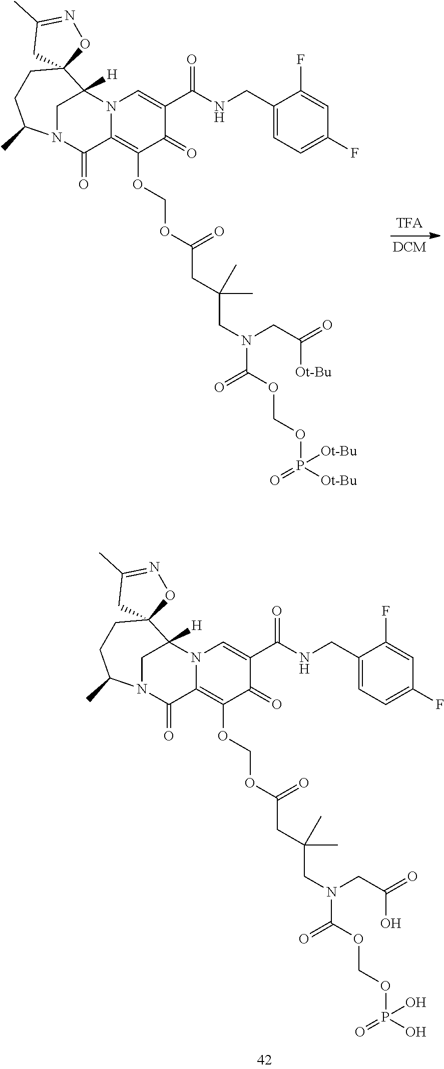 [SMILES: CC1=NO[C@@]2(CC[C@H](C)N3C[C@H]2n2cc(C(=O)NCc4ccc(F)cc4F)c(=O)c(OCOC(=O)CC(C)(C)CN(CC(=O)OC(C)(C)C)C(=O)OCOP(=O)(OC(C)(C)C)OC(C)(C)C)c2C3=O)C1 ; CC1=NO[C@@]2(CC[C@H](C)N3C[C@H]2n2cc(C(=O)NCc4ccc(F)cc4F)c(=O)c(OCOC(=O)CC(C)(C)CN(CC(=O)O)C(=O)OCOP(=O)(O)O)c2C3=O)C1]

Step 1: Preparation of tert-butyl 4,4-dimethyl-2-oxopyrrolidine-1-carboxylate

To a mixture of 4,4-dimethylpyrrolidin-2-one (67.8 g, 0.60 mol) and DMAP (87.9 g, 0.72 mol) in dioxane (1.5 L) was added (Boc)₂O (156.9 g, 0.72 mol) at rt. The mixture was stirred at 45° C. for 2 hi. The reaction mixture was concentrated and the residue was dissolved in EtOAc (1.5 L), washed with 0.2N HC (2×500 mL) and brine, dried over Na₂SO₄, concentrated in vacuum to give the title compound. MS (m/z) 158.2 [M-isobutene]⁺.

### Step 2: Preparation of 4-((tert-butoxycarbonyl)amino)-3,3-dimethylbutanoic acid

To a solution of tert-butyl 4,4-dimethyl-2-oxopyrrolidine-1-carboxylate (115 g, 540 mmol) in MeOH (1000 mL) was 2N NaOH (64.8 g, 1.62 mol) in H₂O (750 mL) at r.t. The mixture was stirred at r.t for 16 h. The reaction mixture was concentrated and the residue was adjusted to pH 3-4 with 2 N HCl, extracted with EtOAc (3×600 mL) and concentrated in vacuum to give the title compound. MS (m/z) 230.1 [M−H]⁺.

### Step 3: Preparation of benzyl 4-amino-3,3-dimethylbutanoate hydrochloride

To a solution of 4-((tert-butoxycarbonyl)amino)-3,3-dimethylbutanoic acid (118 g, 0.51 mol) in CH₃CN (1500 mL) was added Cs₂CO₃ (200 g, 0.61 mol) and BnBr (105 g, 0.61 mol) sequentially at r.t. The mixture was stirred at r.t for 16 h. The reaction mixture was filtered and concentrated, purified by chromatography on silica gel (0-10% EtOAc/PE) to give a residue. The residue was dissolved in dioxane (200 mL) and 4N HCl (200 mL, 797.5 mmol) was added at r.t. The mixture was stirred at r.t for 4 h and concentrated in vacuum to give the title compound as white solid. MS (m/z) 222.2 [M+H]⁺.

### Step 4: Preparation of benzyl 4-((2-(tert-butoxy)-2-oxoethyl)amino)-3,3-dimethylbutanoate

To a mixture of benzyl 4-amino-3,3-dimethylbutanoate hydrochloride (50 g, 199.2 mmol) and DIPEA (77.1 g, 597.6 mmol) in DCM (500 mL) was added t-butyl bromoacetate (42.7 g, 219.1 mmol) in DCM (50 mL) dropwise at 0° C. The mixture was stirred at 0° C. for 1.5 h. The reaction mixture was washed with 0.5 N HCl, water and brine to give the crude product in DCM. MS (m/z) 336.2 [M+H]⁺.

### Step 5: Preparation of benzyl 4-((2-(tert-butoxy)-2-oxoethyl)((chloromethoxy)carbonyl)amino)-3,3-di methylbutanoate

To a stirred solution of benzyl 4-((2-(tert-butoxy)-2-oxoethyl)amino)-3,3-dimethylbutanoate (250 g, 0.75 mol) in DCM (700 mL) at 0° C. was added Et₃N (188 g, 1.86 mol) and chloromethyl chloroformate (144 g, 1.12 mol). The reaction mixture was stirred at 0° C. for 2 h. The reaction was washed with sat. aq. solutions of NH₄C1 then brine. The organic layer was concentrated and purified by column chromatography (0-10% EtOAc/PE) to give the title product. MS (m/z) 426.2 [M−H].

### Step 6: Preparation of benzyl 4-(2-(tert-butoxy)-2-oxoethyl)((((di-tert-butoxyphosphoryl)oxy)methoxy)carbonyl)amino)-3,3-dimethylbutanoate

To a solution of benzyl 4-((2-(tert-butoxy)-2-oxoethyl)((chloromethoxy)carbonyl)amino)-3,3-dimethylbutanoate (28 g, 65.4 mmol) in DME (200 mL) was added di-tert-butyl phosphate tetrabutylammonium salt (41.3 g, 91.6 mmol). The reaction mixture was stirred at 80° C. for 1 h. The mixture was cooled to room temperature, concentrated and dissolved in EtOAc. The organic layer was washed with water (3×), brine, dried over Na₂SO₄, filtered, and concentrated. The crude mixture was purified by column chromatography (0-50% EtOAc/PE) to afford the title compound. 1H NMR (400 MHz, CDCl3) δ: 7.34-7.33 (m, 5H), 5.60 (d, J=11.6 Hz, 1H), 5.56 (d, J=12.0 Hz, 1H), 5.10 (s, 2H), 3.96 (d, J=5.2 Hz, 2H), 3.32 (d, J=5.6 Hz, 2H), 2.33 (d, J=3.6 Hz, 2H), 1.48 (s, 18H), 1.45 (s, 9H), 1.03 (s, 6H).

### Step 7: Preparation of 4-((2-(tert-butoxy)-2-oxoethyl)((((di-tert-butoxyphosphoryl)oxy)methoxy)carbonyl)amino)-3,3-dimethylbutanoic acid

To a solution of benzyl 4-((2-(tert-butoxy)-2-oxoethyl)((((di-tert-butoxyphosphoryl)oxy)methoxy)carbonyl)amino)-3,3-dimethylbutanoate (4000 mg, 6.6 mmol) in EtOAc (25 mL) was added 10% Pd/C (1.4 g, 1.3 mmol). The flask was evacuated and backfilled with hydrogen gas (2×), then sparged with hydrogen for 2 min. The reaction mixture was left to stir under a hydrogen balloon atmosphere overnight. The reaction mixture was filtered, rinsed with EtOAc, and concentrated to afford the crude title compound. MS (m/z) 510.2 [M−H]+.

### Step 8: Preparation of chloromethyl 4-((2-(tert-butoxy)-2-oxoethyl)((((di-tert-butoxyphosphoryl)oxy)methoxy)carbonyl)amino)-3,3-dimethylbutanoate

A biphasic mixture of 4-((2-(tert-butoxy)-2-oxoethyl)((((di-tert-butoxyphosphoryl)oxy)methoxy)carbonyl)amino)-3,3-dimethylbutanoic acid (3 g, 5.86 mmol), tetrabutylammonium hydrogen sulfate (199 mg, 0.58 mmol), and sodium bicarbonate (3.94 g, 46.9 mmol) in water (15 mL) and DCM (15 mL) was cooled to 0° C. While stirring, chloromethyl chlorosulfate (1.94 g, 11.7 mmol) was added dropwise. The reaction mixture was allowed to stir at rt overnight. Brine was added and the aqueous phase was extracted with DCM (3×). The combined organic phase was concentrated. The crude product was purified by column chromatography (0-100% EtOAc/hexanes) to afford the title compound. MS (m/z) 581.9 [M+Na]⁺.

### Step 9: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 4-((2-(tert-butoxy)-2-oxoethyl)((((di-tert-butoxyphosphoryl)oxy)methoxy)carbonyl)amino)-3,3-dimethylbutanoate

Intermediate B (107 mg, 0.22 mmol), K₂CO₃ (61 mg, 0.44 mmol) and KI (48 mg, 0.28 mmol) were mixed in acetone (3 mL) at room temperature. Then a solution of chloromethyl 4-((2-(tert-butoxy)-2-oxoethyl)((((di-tert-butoxyphosphoryl)oxy)methoxy)carbonyl)amino)-3,3-dimethylbutanoate (246 mg, 0.44 mmol) in acetone (1 mL) was added. The reaction mixture was heated at 50° C. for 3 h. The solvent was evaporated and the residue was dissolved in DCM and washed with saturated aqueous NH₄Cl (10 mL). The organic layer was separated and concentrated to dryness. The residue was purified by silica gel column chromatography (80% MeOH/DCM) to afford the title compound. MS (m/z) 1009.9 [M+H]⁺.

### Step 10: Preparation of N-(4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)-2,2-dimethyl-4-oxobutyl)-N-(((phosphonooxy)methoxy)carbonyl)glycine (42)

(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 4-((2-(tert-butoxy)-2-oxoethyl)((((di-tert-butoxyphosphoryl)oxy)methoxy)carbonyl)amino)-3,3-dimethylbutanoate (250 mg, 0.21 mmol) was dissolved in DCM (3 mL) and TFA (0.3 mL) was added. The reaction mixture was stirred at rt for 5 h. The solvent was evaporated and the residue was purified with C-18 reverse phase column chromatography to afford the title compound. MS (m/z) 841.8 [M+H]⁺. 1H NMR (400 MHz, Methanol-d4) δ 8.57 (s, 1H), 7.46 (td, J=8.5, 6.4 Hz, 1H), 7.10-6.84 (m, 2H), 5.90 (dd, J=6.4, 2.2 Hz, 1H), 5.67 (dd, J=6.4, 1.1 Hz, 1H), 5.58 (dd, J=17.8, 13.2 Hz, 2H), 4.86-4.80 (m, 2H), 4.73-4.55 (m, 2H), 4.48 (d, J=2.4 Hz, 1H), 4.10 (d, J=24.4 Hz, 2H), 3.92-3.69 (m, 2H), 3.39 (dd, J=13.0, 3.8 Hz, 1H), 3.16 (d, J=17.9 Hz, 1H), 2.70 (d, J=18.0 Hz, 1H), 2.37 (d, J=8.9 Hz, 2H), 2.06 (s, 3H), 2.00-1.85 (m, 3H), 1.68-1.42 (m, 1H), 1.27 (dd, J=6.8, 1.5 Hz, 3H), 1.08-0.91 (m, 6H).

### Example 43: Preparation of N-(4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)-3,3-dimethyl-4-oxobutyl)-N-(((phosphonooxy)methoxy)carbonyl)glycine (43)

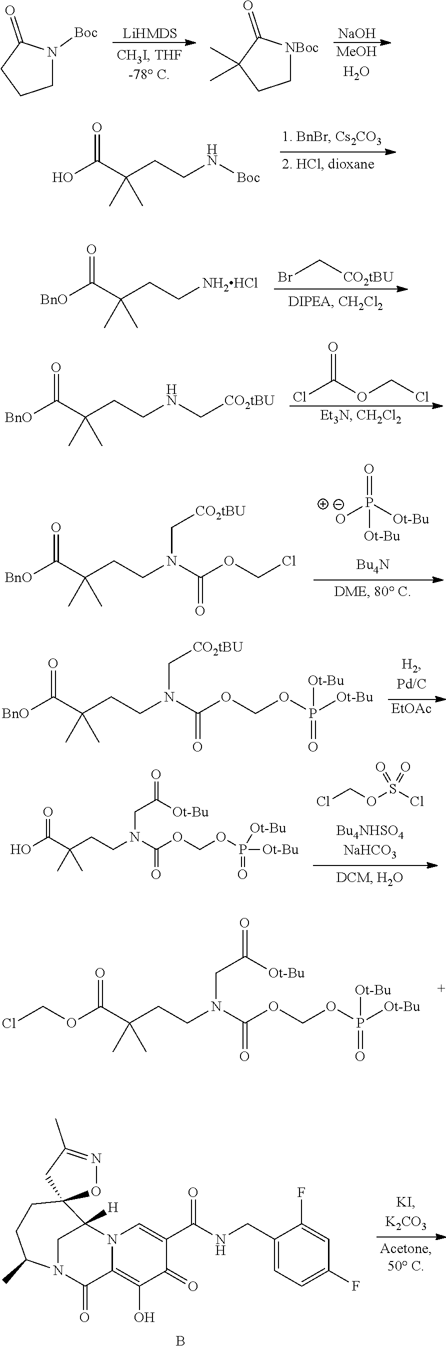

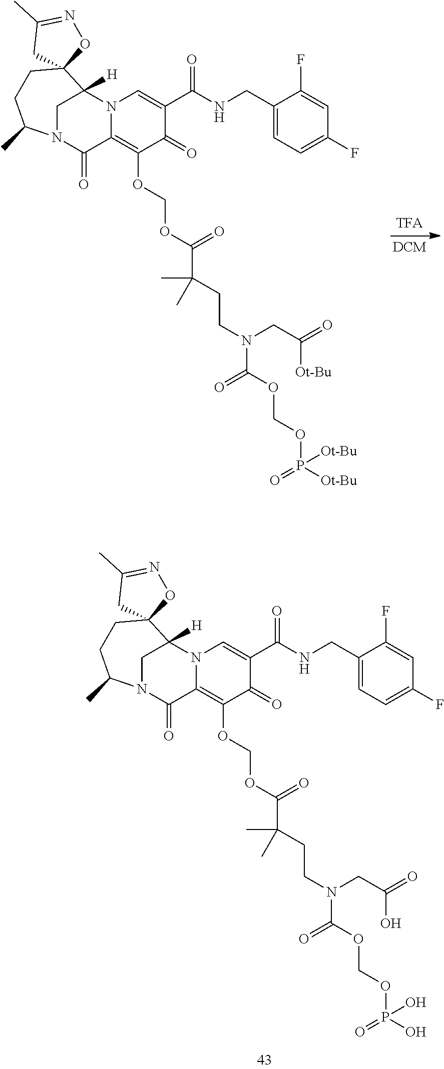 [SMILES: CC1=NO[C@@]2(CC[C@H](C)N3C[C@H]2n2cc(C(=O)NCc4ccc(F)cc4F)c(=O)c(OCOC(=O)C(C)(C)CCN(CC(=O)OC(C)(C)C)C(=O)OCOP(=O)(OC(C)(C)C)OC(C)(C)C)c2C3=O)C1 ; CC1=NO[C@@]2(CC[C@H](C)N3C[C@H]2n2cc(C(=O)NCc4ccc(F)cc4F)c(=O)c(OCOC(=O)C(C)(C)CCN(CC(=O)O)C(=O)OCOP(=O)(O)O)c2C3=O)C1]

### Step 1: Preparation of tert-butyl 3,3-dimethyl-2-oxopyrrolidine-1-carboxylate

To a mixture of tert-butyl 2-oxopyrrolidine-1-carboxylate (600 g, 3.24 mol) and CH₃I (2303 g, 16.22 mol) in THF (3200 mL) was added 1M LiHMDS (8760 mL, 8.76 mol) at −78° C. The mixture was stirred at −78° C. for 1 h. The reaction mixture was quenched by saturated ammonium chloride solution, extracted with EtOAc (3×1000 mL), washed with brine, dried over Na₂SO₄ and concentrated. The residue was purified by column chromatography on silica gel (0-10% EtOAc/PE) to give the title compound. MS (m/z) 158.2 [M-isobutene]⁺.

### Step 2: preparation of 4-((tert-butoxycarbonyl)amino)-2,2-dimethylbutanoic acid

To a solution of tert-butyl 3,3-dimethyl-2-oxopyrrolidine-1-carboxylate (370 g, 1.74 mol) in THF (1500 mL) and EtOH (1500 mL) was added NaOH (347 g, 5.0 mmol) in H₂O (700 mL) at r.t. The mixture was stirred at r.t for 16 h. The reaction mixture was adjusted pH to 3-4 with 2 N HCl, extracted with EtOAc (3×200 mL), washed with brine, dried over Na₂SO₄ and concentrated in vacuum to give the title compound. MS (m/z) 230.11 [M−H]⁺.

### Step 3: Preparation of benzyl 4-amino-2,2-dimethylbutanoate hydrochloride

To a solution of 4-((tert-butoxycarbonyl)amino)-2,2-dimethylbutanoic acid (350 g, 1.52 mol) in MeCN (4900 mL) was added Cs2CO3 (593 g, 1.82 mol) and BnBr (311 g, 1.82 mol) sequentially at r.t. The mixture was stirred at r.t for 16 h. The reaction mixture was filtered, concentrated, and purified by chromatography on silica gel (0-10% EtOAc/PE) to afford a residue. To a solution of the purified residue in dioxane (1300 mL) was added 4N HCl/dioxane (1270 mL, 1.27 mol) at r.t. and stirred at r.t for 4 h. The reaction mixture was concentrated in vacuo to give the title compound. MS (m/z) 222.2 [M+H].

### Step 4: Preparation of benzyl 4-((2-(tert-butoxy)-2-oxoethyl)amino)-2,2-dimethylbutanoate

To a mixture of benzyl 4-amino-2,2-dimethylbutanoate hydrochloride (328 g, 1.27 mol) and DIPEA (494 g, 3.83 mol) in DCM (11 L) was added dropwise t-butyl bromoacetate (124 g, 0.32 mol) in DCM (500 mL) at 0° C. The mixture was stirred at 0° C. for 1 h and then warmed to room temp for 16 h. The reaction mixture was washed with water and brine. The organic layer was concentrated and purified by column chromatography (0-10% MeOH/CH₂Cl₂) to give the desired product. MS (m/z) 336.2 [M+H]⁺.

### Step 5: Preparation of benzyl 4-((2-(tert-butoxy)-2-oxoethyl)((chloromethoxy)carbonyl)amino)-2,2-dimethylbutanoate

To a stirred solution of benzyl 4-((2-(tert-butoxy)-2-oxoethyl)amino)-2,2-dimethylbutanoate (250 g, 0.75 mol) and triethylamine (188 g, 1.86 mol) in DCM (7000 mL) was added chloromethyl chloroformate (144 g, 1.12 mol) at 0° C. and stirred at 0° C. for 1 h. The reaction mixture was quenched by sat. aq. NH₄Cl, washed with brine, concentrated and purified by column chromatography (0-10% EtOAc/PE) to give the desired product. MS (m/z) 426.2 [M−H]⁺.

### Step 6: Preparation of benzyl 4-(2-(tert-butoxy)-2-oxoethyl)((((di-tert-butoxyphosphoryl)oxy)methoxy)carbonyl)amino)-2,2-dimethylbutanoate

To a solution of benzyl 4-((2-(tert-butoxy)-2-oxoethyl)((chloromethoxy)carbonyl)amino)-2,2-dimethylbutanoate (21.2 g, 49.1 mmol) in DME (180 mL) was added di-tert-butyl phosphate tetrabutylammonium salt (31.5 g, 69.7 mmol). The reaction mixture was stirred at 80° C. for 1 h. The mixture was cooled to room temperature, concentrated and dissolved in EtOAc. The organic layer was washed with water (3×), brine, dried over Na₂SO₄, filtered, and concentrated. The crude mixture was purified by column chromatography (0-50% EtOAc/PE) to afford the title product. MS (m/z) 624.2 [M+Na]⁺. 1H NMR (400 MHz, CDCl3) δ: 7.36-7.31 (m, 5H), 5.58 (t, J=10.8 Hz, 2H), 5.10 (d, J=1.2 Hz, 2H), 3.79 (d, J=2.8 Hz, 2H), 3.31-3.26 (m, 2H), 1.85-1.80 (m, 2H), 1.48 (s, 9H), 1.47 (s, 9H), 1.45 (s, 9H), 1.23 (s, 3H), 1.22 (s, 3H).

### Step 7: Preparation of 4-((2-(tert-butoxy)-2-oxoethyl)((((di-tert-butoxyphosphoryl)oxy)methoxy)carbonyl)amino)-2,2-dimethylbutanoic acid

To a solution of benzyl 4-((2-(tert-butoxy)-2-oxoethyl)((((di-tert-butoxyphosphoryl)oxy)methoxy)carbonyl)amino)-2,2-dimethylbutanoate (1151 mg, 1.9 mmol) in EtOAc (25 mL) was added 10% Pd/C (407 mg, 0.38 mmol). The flask was evacuated and backfilled with hydrogen gas (2×), then sparged with hydrogen for 2 min. The reaction mixture was left to stir under a hydrogen balloon atmosphere overnight. The reaction mixture was filtered, rinsed with EtOAc, and concentrated to afford crude product. MS (m/z) 510.2 [M−H]+.

### Step 8: Preparation of chloromethyl 4-((2-(tert-butoxy-2-oxoethyl)((((di-tert-butoxyphosphoryl)oxy)methoxy)carbonyl)amino)-22-dimethylbutanoate

A biphasic mixture of 4-((2-(tert-butoxy)-2-oxoethyl)((((di-tert-butoxyphosphoryl)oxy)methoxy)carbonyl)amino)-2,2-dimethylbutanoic acid (0.98 g, 1.92 mmol), tetrabutylammonium hydrogen sulfate (65 mg, 0.19 mmol), and sodium bicarbonate (1.29 g, 15.3 mmol) in water and DCM was cooled to 0° C. While stirring, chloromethyl chlorosulfate (0.63 g, 3.83 mmol) was added dropwise. The reaction mixture was allowed to stir at rt overnight. Brine was added and the aqueous phase was extracted with DCM (3×). The combined organic phase was concentrated. The crude product was purified by column chromatography (0-100% EtOAc/hexanes) to afford the title compound. MS (m/z) 581.9 [M+Na]+.

### Step 9: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 4-((2-(tert-butoxy)-2-oxoethyl)((((di-tert-butoxyphosphoryl)oxy)methoxy)carbonyl)amino)-2,2-dimethylbutanoate

Intermediate B (115 mg, 0.24 mmol), K2CO3 (65 mg, 0.47 mmol) and KI (51 mg, 0.31 mmol) were mixed in acetone (3 mL) at room temperature. Then a solution of chloromethyl 4-((2-(tert-butoxy)-2-oxoethyl)((((di-tert-butoxyphosphoryl)oxy)methoxy)carbonyl)amino)-2,2-dimethylbutanoate (264 mg, 0.47 mmol) in acetone (1 mL) was added. The reaction mixture was heated at 50° C. for 3 h. The solvent was evaporated, dissolved in DCM, and washed with aqueous saturated NH₄Cl (10 mL). The organic layer was separated and concentrated to dryness. The residue was purified with silica gel column chromatography (80% MeOH/DCM) to afford the title compound. MS (m/z) 1009.81[M+H]+.

### Step 10: Preparation of N-(4-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxymethoxy)-33-dimethyl-4-oxobutyl)-N-(((phosphonooxy)methoxy)carbonyl)glycine (43)

(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 4-((2-(tert-butoxy)-2-oxoethyl)((((di-tert-butoxyphosphoryl)oxy)methoxy)carbonyl)amino)-2,2-dimethylbutanoate (155 mg, 0.15 mmol) was dissolved in DCM (3 mL) and TFA (0.3 mL) was added. The reaction mixture was stirred at rt for 5 h. The solvent was evaporated and the residue was purified by reverse phase C-18 column chromatography to afford the title compound. MS (m/z) 841.7 [M+H]+. 1H NMR (400 MHz, Methanol-d4) δ 8.57 (s, 1H), 7.59-7.27 (m, 1H), 7.07-6.86 (m, 2H), 5.86 (dd, J=11.1, 6.3 Hz, 1H), 5.70 (d, J=6.3 Hz, 1H), 5.65-5.43 (m, 2H), 4.81 (q, J=7.7 Hz, 1H), 4.72-4.54 (m, 2H), 4.52 (d, J=2.3 Hz, 1H), 4.12-4.04 (m, 1H), 4.02 (d, J=1.2 Hz, 1H), 3.89-3.68 (m, 2H), 3.39 (ddd, J=9.2, 5.7, 1.7 Hz, 1H), 3.30-3.25 (m, 1H), 3.24-3.05 (m, 1H), 2.70 (ddd, J=18.1, 2.9, 1.3 Hz, 1H), 2.05 (s, 3H), 1.99-1.71 (m, 5H), 1.64-1.46 (m, 1H), 1.26 (d, J=6.7 Hz, 3H), 1.22-1.12 (m, 6H).

### Example 44: Preparation of N-(2-(1-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)cyclopropyl)ethyl)-N-(((phosphonooxy)methoxy)carbonyl)glycine (44)

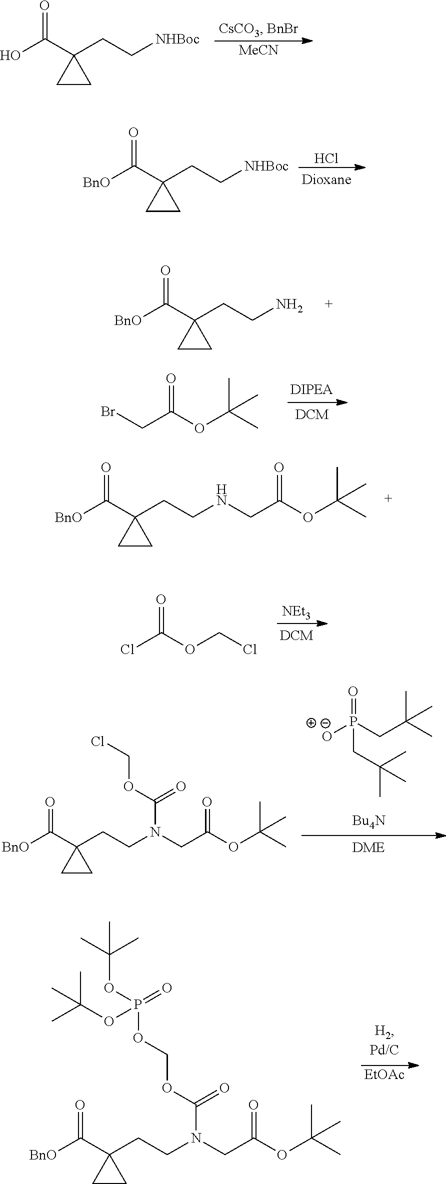

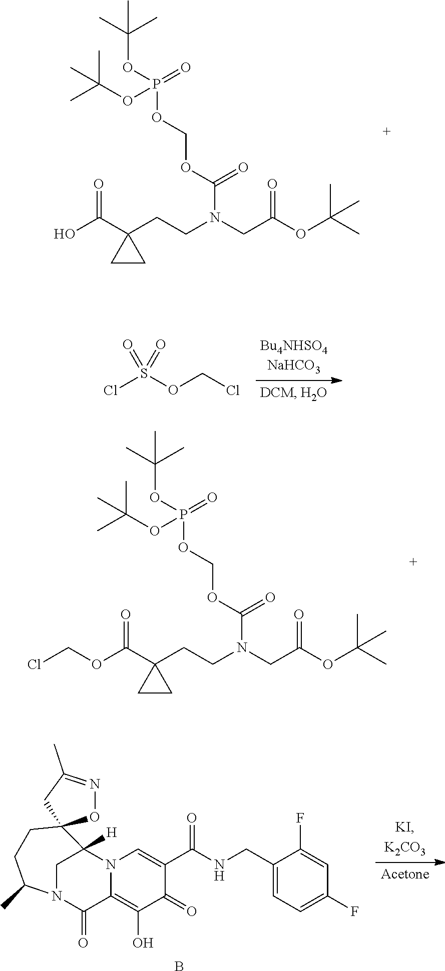

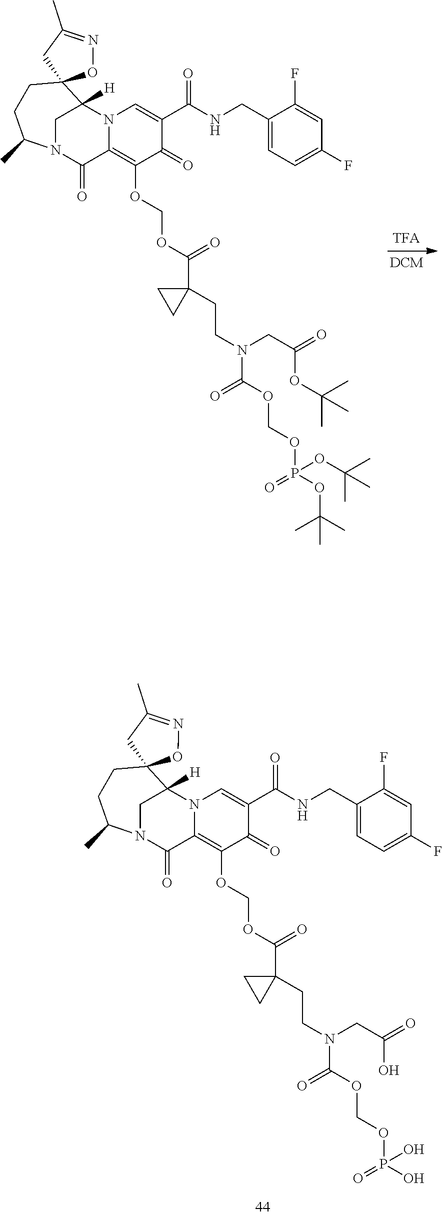

### Step 1: Preparation of benzyl 1-(2-((tert-butoxycarbonyl)aminoethyl)cyclopropane-1-carboxylate

1-(2-((tert-butoxycarbonyl)amino)ethyl)cyclopropane-1-carboxylic acid (25 g, 97 mmol) was dissolved in MeCN (200 mL), and Cs2CO3 and BnBr were added to the mixture. The mixture was stirred at RT overnight. The reaction mixture was then filtered and concentrated. The residue was purified by column chromatography to afford benzyl 1-(2-((tert-butoxycarbonyl)amino)ethyl)cyclopropane-1-carboxylate. MS (m/z) 264.0 [M-isobutene]⁺.

### Step 2: Preparation of benzyl 1-(2-aminoethyl)cyclopropane-1-carboxylate

Benzyl 1-(2-((tert-butoxycarbonyl)amino)ethyl)cyclopropane-1-carboxylate (5.1 g, 16 mmol) was treated with 4N HCl (64 mL) in dioxane (5 mL). After stirring for 1 h, the reaction mixture was concentrated to afford benzyl 1-(2-aminoethyl)cyclopropane-1-carboxylate. MS (m/z) 220.0 [M+H].

### Step 3: Preparation of benzyl 1-(2-((2-(tert-butoxy-2-oxoethyl)amino)ethyl)cyclopropane-1-carboxylate

To a stirred suspension of benzyl 1-(2-aminoethyl)cyclopropane-1-carboxylate (4.1 g, 16 mmol) in CH₂Cl₂ (100 mL) at 0° C. was added DIPEA (6.2 g, 48 mmol). t-Butyl bromoacetate (1.6 g, 8.0 mmol) in CH₂Cl₂ (100 mL) was added dropwise over 30 min, then warmed to rt. The reaction mixture was then washed with 0.5 M aq. HCl solution and brine. The organic layer was dried over Na₂SO₄, filtered, and concentrated. The residue was purified by column chromatography to afford benzyl 1-(2-((2-(tert-butoxy)-2-oxoethyl)amino)ethyl)cyclopropane-1-carboxylate. MS (m/z) 334.10 [M+H]⁺.

### Step 4: Preparation of benzyl 1-(2-((2-(tert-butoxy)-2-oxoethyl)((chloromethoxy)carbonyl)amino)ethyl)cyclopropane-1-carboxylate

To a solution of benzyl 1-(2-((2-(tert-butoxy)-2-oxoethyl)amino)ethyl)cyclopropane-1-carboxylate (1059 mg, 3.2 mmol) in DCM (15 mL) at 0° C. was added NEt₃ (0.48 g, 4.8 mmol), followed by addition of chloromethyl chloroformate (0.61 g, 4.8 mmol) dropwise. After stirring at 0° C. for 10 min. the reaction was washed with brine. The organic layer was dried over anhydrous MgSO₄, filtered, and concentrated. The reside was purified with flash column to afford benzyl 1-(2-(N-(2-(tert-butoxy)-2-oxoethyl)-2-chloroacetamido)ethyl)cyclopropane-1-carboxylate. MS (m/z) 369.90 [M-isobutene]⁺.

### Step 5: Preparation of benzyl 1-(2-((2-(tert-butoxy)-2-oxoethyl)((((di-tert-butoxyphosphoryl)oxy)methoxy)carbonyl)amino)ethyl)cyclopropane-1-carboxylate

To a solution of benzyl 1-(2-((2-(tert-butoxy)-2-oxoethyl)((chloromethoxy)carbonyl)amino)ethyl)cyclopropane-1-carboxylate (840 mg, 2.0 mmol) in DCM (10 mL) was added tetrabutylammonium di-tert-butyl phosphate (1.2 g, 2.8 mmol) and the reaction was heated to 80° C. for 2 h. The reaction was then washed with brine. The organic layer was dried over anhydrous MgSO₄, filtered, and concentrated. The reside was purified by column chromatography to afford benzyl 1-(2-((2-(tert-butoxy)-2-oxoethyl)((((di-tert-butoxyphosphoryl)oxy)methoxy)carbonyl)amino)ethyl)cyclopropane-1-carboxylate. MS (m/z) 621.9 [M+Na]*.

### Step 6: Preparation of 1-(2-((2-(tert-butoxy)-2-oxoethyl)((((di-tert-butoxyphosphoryl)oxy)methoxy)carbonyl)amino)ethyl)cyclopropane-1-carboxylic acid

To a solution of benzyl 1-(2-((2-(tert-butoxy)-2-oxoethyl)((((di-tert-butoxyphosphoryl)oxy)methoxy)carbonyl)amino)ethyl)cyclopropane-1-carboxylate (648 mg, 1.1 mmol) in EtOAc (5 mL) was added 10% Pd/C (0.2 g, 0.22 mmol). The flask was evacuated and backfilled with hydrogen gas (2×), then sparged with hydrogen for 2 min. The reaction mixture was left to stir under a hydrogen balloon atmosphere for 4 h. The reaction mixture was filtered, rinsed with EtOAc, and concentrated to afford 1-(2-((2-(tert-butoxy)-2-oxoethyl)((((di-tert-butoxyphosphoryl)oxy)methoxy)carbonyl)amino)ethyl)cyclopropane-1-carboxylic acid. MS (m/z) 531.9 [M+Na]*.

### Step 7: Preparation of chloromethyl 1-(2-((2-(tert-butoxy)-2-oxoethyl)((((di-tert-butoxyphosphoryl)oxy)methoxy)carbonyilamino)ethyQlcyclopropane-1-carboxylate

A biphasic mixture of 1-(2-((2-(tert-butoxy)-2-oxoethyl)((((di-tert-butoxyphosphoryl)oxy)methoxy)carbonyl)amino)ethyl)cyclopropane-1-carboxylic acid (0.55 g, 1.35 mmol), tetrabutylammonium hydrogen sulfate (46 mg, 0.14 mmol) and sodium bicarbonate (0.91 g, 10.8 mmol) in water and DCM was cooled to 0° C. While stirring, chloromethyl chlorosulfate (0.44 g, 2.7 mmol) was added dropwise. The reaction mixture was allowed to stir at rt overnight. Brine was added and the aqueous phase was extracted with DCM (3×). The combined organic phase was concentrated. The crude product was purified by column chromatography (0-100% EtOAc/hexanes) to afford chloromethyl 1-(2-((2-(tert-butoxy)-2-oxoethyl)((((di-tert-butoxyphosphoryl)oxy)methoxy)carbonyl)amino)ethyl)cyclopropane-1-carboxylate. MS (m/z) 580.1 [M+Na]⁺.

### Step 8: Preparation of (((3′S,5S,7′RJ-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl J-(2-((2-(tert-butoxy)-2-oxoethyl)((((di-tert-butoxyphosphoryl)oxy)methoxycarbonyl)amino)ethyl)cyclopropane-1-carboxylate

Intermediate B (250 mg, 0.51 mmol), K2C03 (142 mg, 1 mmol) and KI (111 mg, 0.67 mmol) were mixed in acetone (5 mL) at room temperature. Then a solution of chloromethyl 1-(2-((2-(tert-butoxy)-2-oxoethyl)((((di-tert-butoxyphosphoryl)oxy)methoxy)carbonyl)amino)ethyl)cyclopropane-1-carboxylate (459 mg, 0.82 mmol) in acetone (1 mL) was added. The reaction mixture was heated at 50° C. for 3 h. The solvent was evaporated and then dissolved in DCM. Washed with aqueous saturated NH₄Cl (10 mL). The organic layer was separated and concentrated to dryness. The residue was purified by silica gel column chromatography (80% MeOH/DCM) to afford the title compound. MS (m/z) 1007.8 [M+H]⁺.

### Step 9: N-(2-(1-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)cyclopropyl)ethyl)-N-(((phosphonooxy)methoxy)carbonyl)glycine (44)

(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 1-(2-((2-(tert-butoxy)-2-oxoethyl)((((di-tert-butoxyphosphoryl)oxy)methoxy)carbonyl)amino)ethyl)cyclopropane-1-carboxylate (51 mg, 0.05 mmol) was dissolved in DCM (2 mL) and TFA (0.3 mL) was added. The reaction mixture was stirred at rt for 5 h. The solvent was evaporated, the residue was purified reverse phase chromatography to afford the title compound. MS (m/z) 839.70 [M+H]⁺. 1H NMR (400 MHz, Methanol-d4) δ 8.58 (d, J=4.1 Hz, 1H), 7.45 (q, J=7.8 Hz, 1H), 7.11-6.85 (m, 2H), 5.86 (dd, J=12.8, 6.3 Hz, 1H), 5.68 (dd, J=6.4, 4.8 Hz, 1H), 5.53 (q, J=10.3, 6.1 Hz, 2H), 4.86-4.78 (m, 1H), 4.74-4.55 (m, 2H), 4.51 (d, J=2.4 Hz, 1H), 4.13-3.94 (m, 2H), 3.92-3.73 (m, 2H), 3.57-3.39 (m, 2H), 3.16 (d, J=17.9 Hz, 1H), 2.71 (dd, J=18.2, 3.3 Hz, 1H), 2.05 (s, 3H), 1.98-1.46 (m, 6H), 1.34-1.13 (m, 5H), 0.88 (dd, J=19.5, 3.0 Hz, 2H).

### Example 45: Preparation of N-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)-N-methylglycine (45)

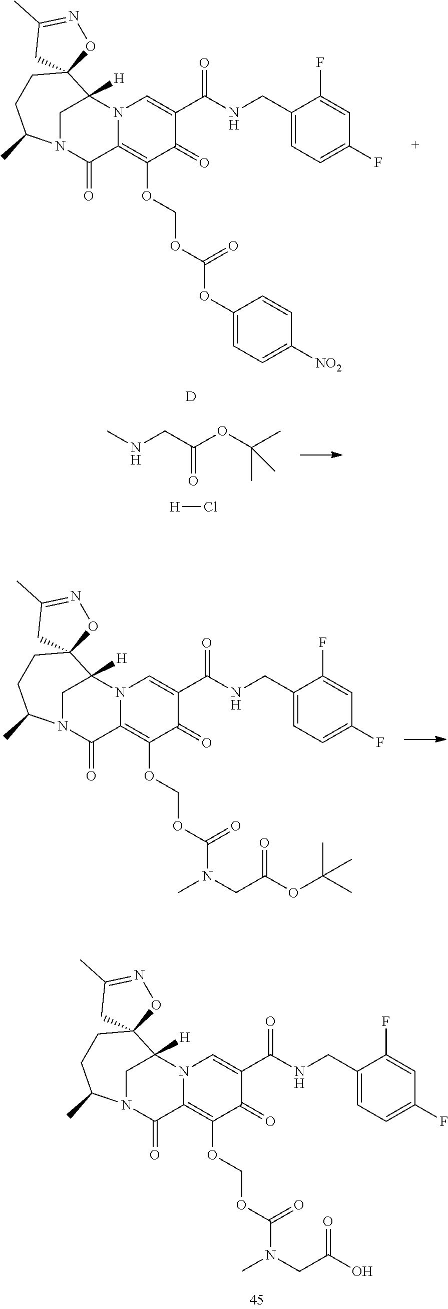

### Step 1: Preparation of tert-butyl N-(((((3′S,5S,7′J)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)-N-methylglycinate

To a mixture of Intermediate D (40.0 mg, 0.0587 mmol) in acetonitrile (1 mL) at room temperature was added tert-butyl methylglycinate hydrochloride HCl (21.3 mg, 0.117 mmol) and triethylamine (23.8 mg, 0.235 mmol). After being stirred for 30 minutes, the reaction was diluted with EtOAc, washed sequentially with water and brine, dried over sodium sulfate, filtered, and concentrated. The residue was purified by silica gel column chromatography (0-100% EtOAc/Hexane then 0-15% MeOH/EtOAc) to give title compound. MS (m/z) 687.86 [M]+.

### Step 2: Preparation of N-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)-N-methylglycine (45)

The residue from Step 1 was dissolved in DCM (2.0 mL) before it was cooled to 0° C. To this cold mixture was added TFA (0.4 mL). The mixture was stirred for 30 minutes and then removed from cooling bath and stirred at room temperature for 2 hours. The reaction was then concentrated, redissolved in DMF, filtered, and purified by reverse phase prep HPLC (10-100% MeCN/water containing 0.1% TFA) to give title compound. MS (m/z) 631.908 [M]+. 1H NMR (400 MHz, DMSO-d6) δ 10.32 (t, J=5.9 Hz, 1H), 8.66 (d, J=1.4 Hz, 1H), 7.43 (td, J=8.7, 6.6 Hz, 1H), 7.25 (ddd, J=10.5, 9.3, 2.6 Hz, 1H), 7.08 (td, J=8.6, 2.7 Hz, 1H), 5.72-5.59 (m, 2H), 4.71-4.50 (m, 4H), 3.94-3.85 (m, 2H), 3.62 (d, J=2.3 Hz, 2H), 3.00 (d, J=17.6 Hz, 1H), 2.87-2.79 (m, 3H), 2.63 (d, J=17.6 Hz, 1H), 1.95 (s, 3H), 1.86-1.70 (m, 3H), 1.39-1.27 (m, 1H), 1.16 (t, J=6.2 Hz, 3H).

### Example 46: Preparation of N-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)-N-(2-(phosphonooxy)ethyl)glycine (46)

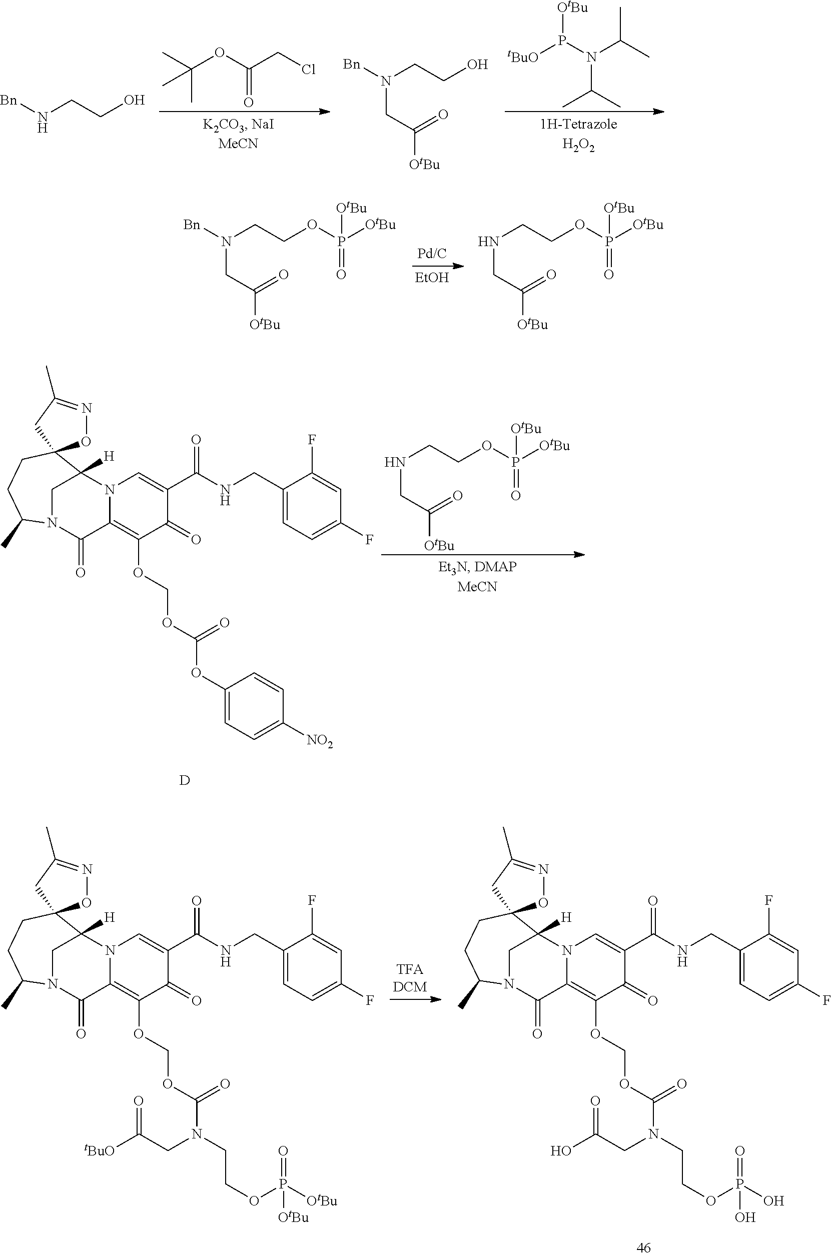

### Step 1: Preparation of tert-butyl N-benzyl-N-(2-hydroxyethyl)glycinate

To mixture of 2-(benzylamino)ethanol (2 g, 13.2 mmol) in acetonitrile (20 mL) was added tert-butyl 2-chloroacetate (2.2 g, 14.5 mmol), K2CO3 (2.19 g, 15.9 mmol) and NaI (0.40 g, 2.65 mmol). The resulting mixture was stirred at reflux for overnight. Ethyl acetate was added to dilute the mixture, and the organic phase was washed with H₂O and brine. The solvent was removed under reduced pressure to obtain the title compound, which was used without further purification. MS (m/z) 265.9 [M+H]+.

### Step 2: Preparation of tert-butyl N-benzyl-N-(2-((di-tert-butoxyphosphoryl)oxy)ethyl)glycinate

To a mixture of tert-butyl N-benzyl-N-(2-hydroxyethyl)glycinate (1 g, 3.77 mmol) in THF (10 mL) was added 1H-tetrazole (0.53 g, 7.54 mmol) and di-tert-butyl N,N-diisopropylphosphoramidite (1.57 g, 5.65 mmol). The resulting mixture was stirred at room temperature for 16 hours. The mixture was cooled to 0° C. and 30% H₂O₂ aqueous solution (4.70 g, 41.5 mmol) was added to the mixture. After 15 minutes, the cooling bath was removed and the mixture was stirred for an additional 6 h. Aqueous 10/u Na₂SO₃ (50 mL) was added to the mixture with water bath cooling. After 25 minutes, EtOAc was added and the phases separated. The organic phase was washed with H₂O, brine and dried with MgSO₄. The solvent was removed under reduced pressure and the residue was purified by silica gel column chromatography (0-100% EtOAc/hexane) to obtain the title compound. MS (m/z) 458.4 [M+H]+.

### Step 3: Preparation of tert-butyl (2-((di-tert-butoxyphosphoryl)oxy)ethyl)glycinate

To a mixture of tert-butyl N-benzyl-N-(2-((di-tert-butoxyphosphoryl)oxy)ethyl)glycinate (1.04 g, 2.27 mmol) in EtOH (20 mL) was added 10% wt. Pd/C (0.242 g, 0.27 mmol). The resulting mixture was purged with H₂. After 1 h, the reaction mixture was filtered through celite and the filter cake was washed with EtOH. The filtrate was concentrated under reduced pressure to provide the title compound, which was used without further purification. MS (m/z) 368.4 [M+H]+.

### Step 4: Preparation of N-(2-((di-tert-butoxyphosphoryl)oxy)ethyl)-N-(((((3′S,5S,7′R)-10′-((2,4-difluorobenzyl)carbamoyl)-3,3′-dimethyl-1′,11′-dioxo-1′,4′,55′,11′-tetrahydro-3′H,4H,7′H-spiro[isoxazole-5,6′-[2,7]methanopyrido[1,2-a][1,4]diazonin]-12′-yl)oxy)methoxy)carbonyl)glycine

To a mixture of Intermediate D (0.2 g, 0.29 mmol) in acetonitrile (5 mL) was added tert-butyl (2-((di-tert-butoxyphosphoryl)oxy)ethyl)glycinate (0.22 g, 0.587 mmol) followed by the addition of triethylamine (0.082 mL, 0.587 mmol) and DMAP (0.358 g, 0.029 mmol). The resulting mixture was stirred at room temperature overnight. The reaction mixture was diluted with EtOAc and washed with IN HCl, H₂O and brine. The solvent was removed under reduced pressure and the residue was purified by silica gel column chromatography (0-100% EtOAc/hexane) to obtain the title compound. MS (m/z) 910.7 [M+H]+.

### Step 5: Preparation of N-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5′,6′-[2,7]methanopyrido[1,2-a][1,4]diazonin]-12′-yl)oxy)methoxy)carbonyl)-N-(2-(phosphonooxy)ethyl)glycine (46)

To a mixture of N-(2-((di-tert-butoxyphosphoryl)oxy)ethyl)-N-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methoxy)carbonyl)glycine (0.18 g, 0.198 mmol) in DCM (5 mL) was added TFA (0.5 mL) at 0° C. The mixture was warmed to room temperature and stirred for 2 h. The solvent was removed under vacuo and the resulting residue was purified by prep-HPLC to obtain the title compound. MS (m/z) 741.9 [M+H]+. 1H NMR (400 MHz, DMSO-d6) δ 10.31 (td, J=6.0.3.5 Hz, 1H), 8.66 (d, J=5.8 Hz, 1H), 7.43 (td, J=8.7, 6.6 Hz, 1H), 7.25 (ddd, J=10.4, 9.3, 2.6 Hz, 1H), 7.08 (dddd, J=8.7, 7.6, 2.7, 1.3 Hz, 1H), 5.78-5.70 (m, 1H), 5.64 (d, J=6.1 Hz, 1H), 4.76-4.44 (m, 4H), 4.06-3.94 (m, 1H), 3.96-3.82 (m, 3H), 3.75-3.60 (m, 2H), 3.43 (dt, J=16.8, 6.4 Hz, 2H), 3.00 (d, J=17.5 Hz, 1H), 2.64 (dd, J=17.6, 4.3 Hz, 1H), 1.95 (s, 3H), 1.83-1.65 (m, 3H), 1.34 (dd, J=16.2, 8.3 Hz, 1H), 1.16 (dd, J=6.7, 2.7 Hz, 3H).

### Example 47: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl dimethylcarbamate (47)

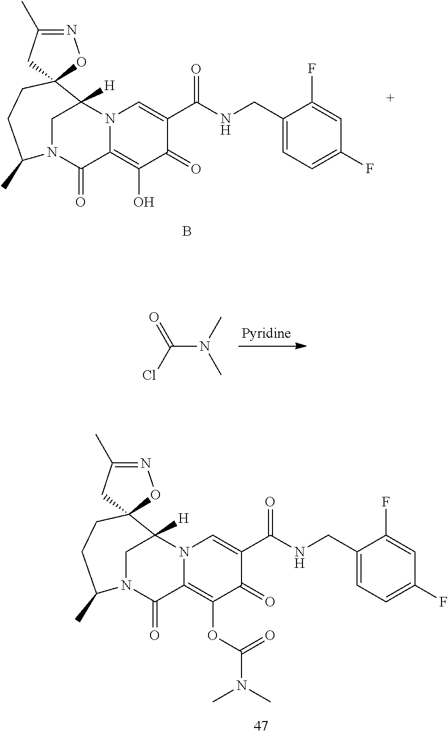

N,N-dimethyl carbamic chloride (221 mg, 2.06 mmol) was added to a suspension of Intermediate B (100 mg, 0.206 mmol) in pyridine (2 mL). The resulting suspension was heated at 50° C. for 2 h. The reaction mixture was then concentrated to dryness. The residue was placed under high vacuum overnight. The residue was dissolved in DMF and purified by reverse phase prep HPLC (10-100% MeCN/water) to afford the title compound. MS (m/z): calculated for C₂₇H₂₉F₂N₅O₆, 557.21; found, 557.99 [M+H]+. 1H NMR (400 MHz, DMSO) δ 10.22 (t, J=5.9 Hz, 1H), 8.76 (s, 1H), 7.42 (td, J=8.7, 6.6 Hz, 1H), 7.25 (ddd, J=10.5, 9.3, 2.6 Hz, 11H), 7.08 (tdd, J=8.6, 2.7, 1.0 Hz, 1H), 4.68 (s, 1H), 4.66-4.47 (m, 3H), 3.82-3.63 (m, 2H), 3.05-2.97 (m, 4H), 2.88 (s, 3H), 2.65 (d, J=17.5 Hz, 1H), 1.95 (s, 3H), 1.89-1.70 (m, 3H), 1.36-1.2 (m, 11H), 1.16 (d, J=6.7 Hz, 3H).

### Example 48: Preparation of N-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)-N-(2-(phosphonooxy)ethyl)glycine (48)

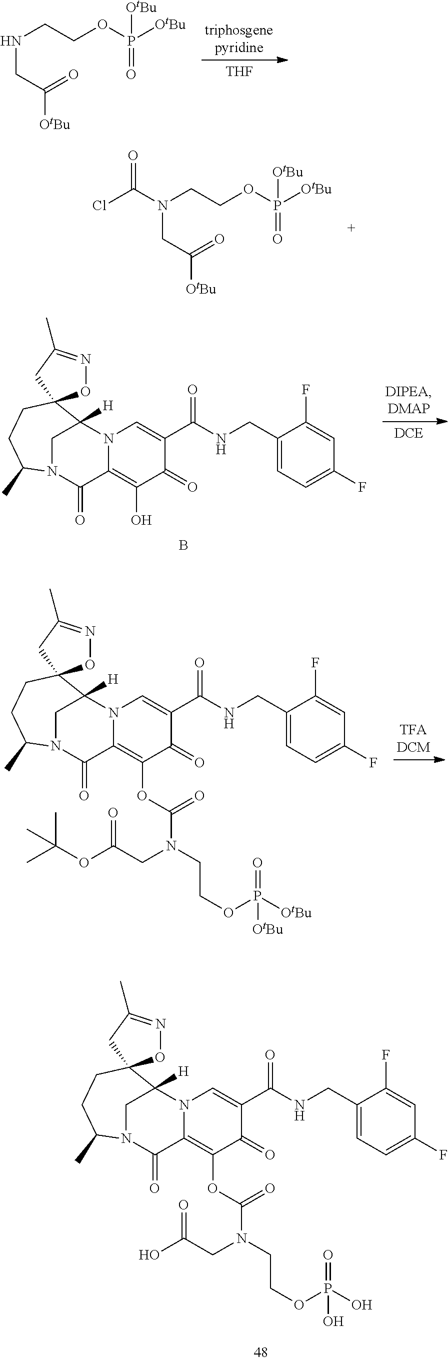

### Step 1: Preparation of tert-butyl N-(chlorocarbonyl)-N-(2-((di-tert-butoxyphosphoryl)oxy)ethyl)glycinate

To a solution of triphosgene (0.12 g, 0.41 mmol) in THF (6 mL) at 0° C. was added pyridine (0.09 ml, 1.22 mmol). To the resulting suspension was added a solution of tert-butyl 2-(2-ditert-butoxyphosphoryloxyethylamino)acetate (0.3 g, 0.817 mmol), prepared according to Example 46, dissolved in THF (3 mL). The suspension was allowed to warm to rt and stir for 3 h. The reaction mixture was filtered through Celite and rinsed with DCM/ethyl ether. The filtrate was concentrated and used directly in next step.

### Step 2: Preparation of tert-butyl N-(2-((di-tert-butoxyphosphoryl)oxy)ethyl)-N-((((3′S,5S,7′R)-10′-((2,4-difluorobenzyl)carbamoyl)-3,3′-dimethyl-1′,11′-dioxo-1′,4′,5,11′-tetrahydro-3′H,4H,7′H-spiro[isoxazole-5,6′-[2,7]methanopyrido[1,2-a][1,4]diazonin]-12′-yl)oxy)carbonyl)glycinate

To a suspension of Intermediate B (0.25 g, 0.514 mmol) in DCE (5 ml) was added tert-butyl N-(chlorocarbonyl)-N-(2-((di-tert-butoxyphosphoryl)oxy)ethyl)glycinate (0.33 g, 0.77 mmol), followed by the addition of DMAP (0.063 g, 0.514 mmol) and DIPEA (0.45 mL, 2.57 mmol) at 0° C. The reaction mixture was warmed to rt and stirred overnight. The reaction mixture was diluted with EtOAc, washed with HCl (IN), water, and brine. The organic phase was dried over MgSO₄, filtered, and concentrated to afford a residue, which was purified by column chromatography (0-100% EtOAc/hexanes) to afford the title compound. MS (m/z) 880.5 [M+H]+.

### Step 3: Preparation of N-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5′,6′-[2,7]methanopyrido[1,2-a][1,4]diazonin]-12′-yl)oxy)carbonyl)-N-(2-(phosphonooxy)ethyl)glycine (48)

To mixture of tert-butyl N-(2-((di-tert-butoxyphosphoryl)oxy)ethyl)-N-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)glycinate (0.094 g, 0.11 mmol) in DCM (3 mL) was added TFA (0.3 mL) at 0° C. The mixture was warmed to room temperature and stirred for 2 h. The solvent was removed under vacuo and the resulting residue was purified by prep-HPLC (5-95% MeCN/water containing 0.5% TFA) to obtain the title compound. MS (m/z) 711.96 [M+H]+. 1H NMR (400 MHz, DMSO-d6) δ 10.19 (q, J=6.0 Hz, 1H), 8.78 (d, J=30.7 Hz, 1H), 7.42 (td, J=8.6, 6.7 Hz, 1H), 7.30-7.21 (m, 1H), 7.10-6.98 (m, 1H), 4.91-4.35 (m, 5H), 4.28-3.88 (m, 4H), 3.77-3.35 (m, 3H), 3.00 (d, J=17.5 Hz, 1H), 2.74-2.59 (m, 1H), 1.95 (s, 3H), 1.86-1.71 (m, 3H), 1.33-1.02 (m, 4H).

### Example 49: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl ((phosphonooxy)methyl) ethane-1,2-diylbis(methylcarbamate) (49)

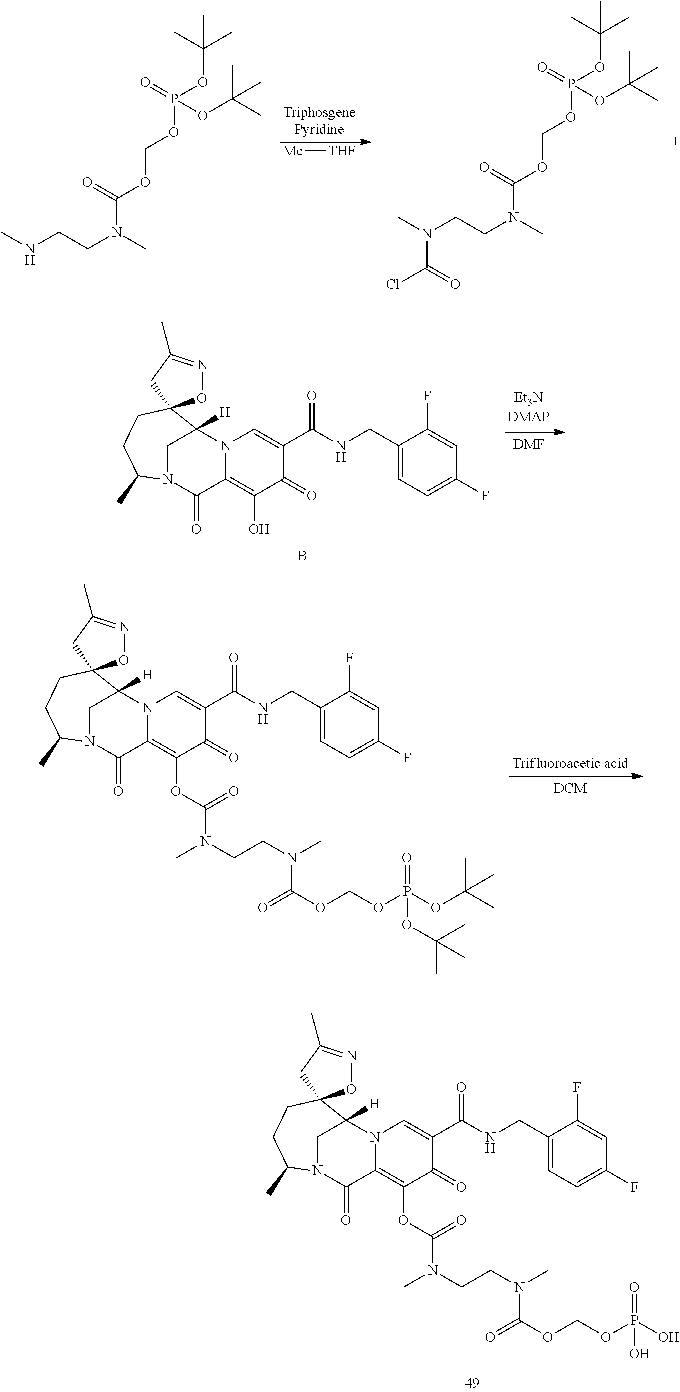

### Step 1: Preparation of ((di-tert-butoxyphosphoryl)oxy)methyl (2-((chlorocarbonyl)(methyl)amino)ethyl)(methyl)carbamate

((Di-tert-butoxyphosphoryl)oxy)methyl methyl(2-(methylamino)ethyl)carbamate (268 mg, 0.756 mmol), prepared according to WO2023102523, was dissolved in Me-THF (30 mL) at rt. Under argon balloon, the solution was cooled down to 0° C. Then pyridine (120 mg, 1.59 mmol) was added with stirring. After 10 min, triphosgene (202 mg, 0.681 mmol) was added in one portion as solid. After 3 h, the reaction mixture was diluted with EtOAc (10 mL) and was then treated with HCl (IN) (10 mL) and brine (20 mL). The organic phase was separated and dried over Na₂SO₄. The resulting solution was concentrated to afford the title compound. MS (m/z): calculated for C₁₅H₃₀ClN₂O₇P, 416.15; found, 416.90 [M+H]+.

### Step 2: Preparation of ((di-tert-butoxyphosphoryl)oxy)methyl ((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl) ethane-1,2-diylbis(methylcarbamate)

Intermediate B (72 mg, 0.148 mmol) and ((di-tert-butoxyphosphoryl)oxy)methyl (2-((chlorocarbonyl)(methyl)amino)ethyl)(methyl)carbamate (96 mg, 0.230 mmol) were dissolved in DMF (5 mL) at rt. Triethylamine (45 mg, 0.444 mmol) and DMAP (11 mg, 0.089 mmol) were added sequentially. The reaction mixture was stirred at rt for 2 hrs. The reaction mixture was then diluted with EtOAc (10 mL) and treated with saturated aqueous solution of NH₄Cl (10 mL). The organic phase was separated and the aqueous phase was extracted with EtOAc (1×10 mL). The combined organic phase was washed with water (20 mL) and brine (20 mL) and concentrated. The residue was purified by silica gel column chromatography (0-100% EtOAc/hexanes) to afford the title compound. MS (m/z): calculated for C₃₉H₅₃F₂N₆O₁₂P, 866.34; found, 866.70 [M+H]+.

### Step 3: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl ((phosphonooxy)methyl) ethane-1,2-diylbis(methylcarbamate) (49)

((Di-tert-butoxyphosphoryl)oxy)methyl ((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl) ethane-1,2-diylbis(methylcarbamate) (22 mg, 0.0254 mmol) was dissolved in DCM (7 mL) at rt. The solution was cooled down to 0° C. under argon atmosphere. Trifluoroacetic acid (1 g, 9.15 mmol) was added dropwise. The reaction was stirred at 0° C. for 70 min and concentrated under reduced pressure. The residue was purified by reversed phase prep HPLC (10-100% acetonitrile/water with 0.1% trifluoroacetic acid) to afford the title compound. MS (m/z): calculated for C₃₁H₃₇F₂N₆O₁₂P, 754.22; found, 754.92 [M+H]+. ¹H NMR (400 MHz, DMSO) δ 10.24-10.17 (m, 1H), 8.76 (d, J=36.3 Hz, 1H), 7.42 (q, J=8.4 Hz, 1H), 7.24 (ddd, J=10.4, 9.3, 2.6 Hz, 1H), 7.13-7.03 (m, 1H), 5.48 (t, J=11.8 Hz, 2H), 4.67-3.28 (m, 10H), 3.11-2.88 (m, 7H), 2.66 (d, J=17.4 Hz, 1H), 1.95 (s, 3H), 1.86-1.67 (m, 3H), 1.43-1.22 (m, 1H), 1.16 (d, J=6.7 Hz, 3H).

### Example 50: Preparation of N-(2-(((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)(methyl)amino)ethyl)-N-(((phosphonooxy)methoxy)carbonyl)glycine (50)

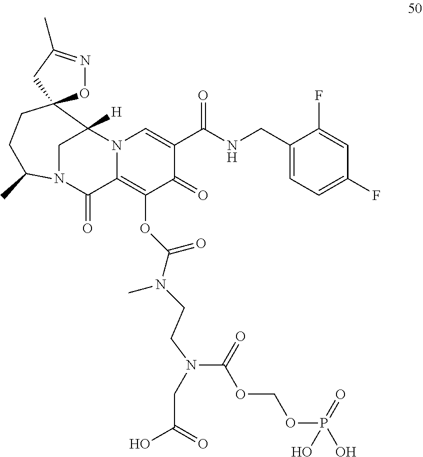 [SMILES: CC1=NO[C@@]2(CC[C@H](C)N3C[C@H]2n2cc(C(=O)NCc4ccc(F)cc4F)c(=O)c(OC(=O)N(C)CCN(CC(=O)O)C(=O)OCOP(=O)(O)O)c2C3=O)C1]

The title compound was prepared in a similar manner as Example 11, except using benzyl N-((((bis(benzyloxy)phosphoryl)oxy)methoxy)carbonyl)-N-(2-(methylamino)ethyl)glycinate, prepared according to Example 21, instead of dibenzyl (3-hydroxypropyl) phosphate in Step 1. MS (m/z) 799.1 [M+H]+. 1H NMR (400 MHz, Methanol-d4) δ 8.65 (d, J=3.0 Hz, 1H), 7.46 (q, J=7.9 Hz, 1H), 7.07-6.88 (m, 2H), 5.77-5.45 (m, 2H), 4.83-4.57 (m, 3H), 4.51 (s, 1H), 4.37-4.12 (m, 2H), 3.83 (s, 2H), 3.77-3.54 (m, 3H), 3.24-3.12 (m, 2H), 3.07 (s, 2H), 2.70 (d, J=17.9 Hz, 1H), 2.07 (s, 3H), 2.03-1.84 (m, 3H), 1.60 (s, 1H), 1.42 (s, 1H), 1.26 (d, J=6.8 Hz, 3H).

### Example 51: Preparation of N-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)-N-(2-(methyl(((phosphonooxy)methoxy)carbonyl)amino)ethyl)glycine (51)

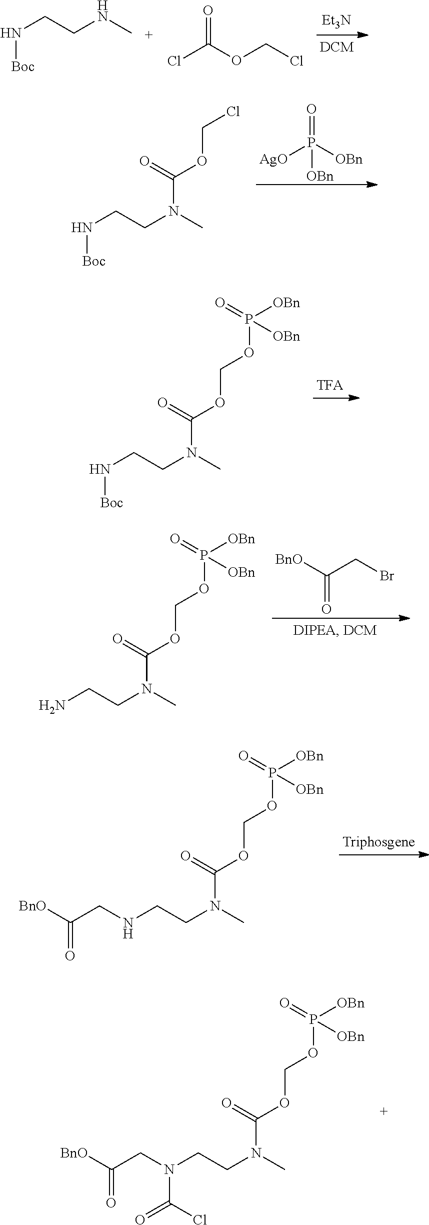

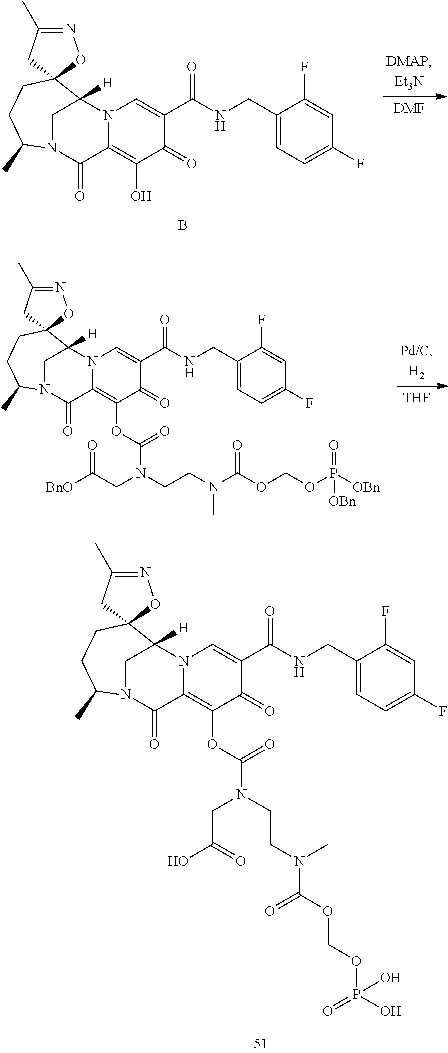

### Step 1: Synthesis of chloromethyl (2-((tert-butoxycarbonylamino)ethyl)(methyl)carbamate

To a solution of tert-butyl (2-(methylamino)ethyl)carbamate (1.02 g, 5.85 mmol) in DCM (60.0 mL) at 0° C. was added chloromethyl carbonochloridate (0.83 g, 6.44 mmol) followed by triethylamine (0.711 g, 7.02 mmol). The reaction was stirred at 0° C. for 30 minutes. The reaction was then diluted with DCM, washed sequentially with IN HCl, water, brine, dried over sodium sulfate, filtered, and concentrated to give the title compound.

### Step 2: Synthesis of ((bis(benzyloxy)phosphoryl)oxy)methyl (2-((tert-butoxycarbonyl)amino)ethyl)(methyl)carbamate

The mixture of chloromethyl (2-((tert-butoxycarbonyl)amino)ethyl)(methyl)carbamate (1.56 g, 5.85 mmol) and ((bis(benzyloxy)phosphoryl)oxy)silver (2.93 g, 7.6 mmol) in toluene (14.0 mL) was heated at 110° C. for 16 hours. The reaction was cooled to room temperature, filtered through a pad of Celite, concentrated and purified by normal phase silica gel chromatography (0-70% EtOAc/Hexane) to give the title compound. MS (m/z) 508.740 [M+H]⁺

### Step 3: Synthesis of ((bis(benzyl)oxy)phosphoryl) oxy)methyl (2-aminoethyl)(methyl)carbamate

A solution of ((bis(benzyloxy)phosphoryl)oxy)methyl (2-((tert-butoxycarbonyl)amino)ethyl)(methyl)carbamate (4.09 g, 8.04 mmol) in DCM (80 mL) at 0° C. was treated with TFA (23.2 mL). After one hour, the reaction was quenched with saturated sodium bicarbonate until pH-7. The mixture was concentrated to remove DCM and extracted with EtOAc (3×). The combined organic layer was washed with brine, dried over sodium sulfate, filtered and concentrated to give the title compound. MS (m/z) 409.038 [M+H]⁺

### Step 4: Synthesis of benzyl (2-(((((bis(benzyloxy)phosphoryl)oxy)methoxy)carbonyl)(methyl)amino)ethyl)glycinate

To a solution of ((bis(benzyloxy)phosphoryl)oxy)methyl (2-aminoethyl)(methyl)carbamate (118.3 mg, 0.29 mmol) in DCM (8.0 mL) at room temperature was added benzyl 2-bromoacetate (39.8 mg, 0.17 mmol) followed by DIPEA (22 mg, 0.17 mmol). The reaction was stirred for 1 hour before it was diluted with DCM, washed with saturated sodium bicarbonate, brine, dried over sodium sulfate, filtered and concentrated. The residue was purified by normal phase silica gel chromatography (20-100% EtOAc/Hexane then 0-15% MeOH/EtOAc) to afford the title compound. MS (m/z) 557.074 [M+H]

### Step 5: Synthesis of benzyl N-(2-(((((bis(benzyloxy)phosphoryl)oxy)methoxy)carbonyl)(methyl)amino)ethyl)-N-(chlorocarbonyl)glycinate

To a solution of benzyl (2-(((((bis(benzyloxy)phosphoryl)oxy)methoxy)carbonyl)(methyl)amino)ethyl)glycinate (50 mg, 0.09 mmol) in DCM (2.0 mL) at 0° C. was added pyridine (9.23 mg, 0.117 mmol) followed by bis(trichloromethyl) carbonate (24.0 mg, 0.081 mmol). After stirring for 30 minutes, the reaction was diluted with DCM, washed sequentially with IN HCl, water, brine, dried over sodium sulfate, filtered and concentrated to give the title compound. MS (m/z) 618.754 [M+H]⁺

### Step 6: Synthesis of benzyl N-(2-(((((bis(benzyloxy)phosphoryl)oxy)methoxy)carbonyl)(methyl)amino)ethyl)-N-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)glycinate

To a mixture of Intermediate B (20 mg, 0.04 mmol) in DMF (1.0 mL) at room temperature was added benzyl N-(2-(((((bis(benzyloxy)phosphoryl)oxy)methoxy)carbonyl)(methyl)amino)ethyl)-N-(chlorocarbonyl)glycinate (76.3 mg, 0.12 mmol) followed by triethylamine (16.6 mg, 0.164 mmol) and DMAP (0.5 mg, 0.004 mmol). After stirring overnight, the reaction was diluted with EtOAc, washed with water and brine, dried over sodium sulfate, filtered, and concentrated. The residue was purified by normal phase silica gel chromatography (0-100% EtOAc/Hexane then 0-15% MeOH/EtOAc) to give title compound. MS (m/z) 1068.713 [M]⁺.

### Step 7: Synthesis of N-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5′,6′-[2,7]methanopyrido[1,2-a][1,4]diazonin]-12′-yl)oxy)carbonyl)-N-(2-(methyl(((phosphonooxy)methoxy)carbonyl)amino)ethyl)glycine (51)

To a solution of benzyl N-(2-(((((bis(benzyloxy)phosphoryl)oxy)methoxy)carbonyl)(methyl)amino)ethyl)-N-((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)glycinate (36.0 mg, 0.034 mmol) in a mixture of MeOH (0.2 mL) and EtOAc (2.0 mL) at room temperature was added 5 wt % Pd/C (2.0 mg). The reaction was degassed and flushed with nitrogen then degassed and flushed with hydrogen three times before it was hydrogenated under hydrogen balloon overnight. The reaction was then degassed and flushed with nitrogen and filtered through a pad of Celite, rinsing the filter cake with EtOAc. The filtrate was concentrated and purified by reverse phase preparative HPLC (10-100% MeCN/water containing 0.1% TFA) to give the title compound. MS (m/z) 798.799 [M]⁺. 1H NMR (400 MHz, Methanol-d4) δ 8.67-8.59 (m, 1H), 7.46 (q, J=8.2 Hz, 1H), 7.04-6.89 (m, 2H), 5.71-5.53 (m, 2H), 4.83-4.72 (m, 1H), 4.72-4.59 (m, 2H), 4.54-4.46 (m, 1H), 4.31-4.09 (m, 2H), 3.89-3.54 (m, 6H), 3.16 (dd, J=17.7, 4.6 Hz, 1H), 3.10-2.98 (m, 3H), 2.70 (d, J=17.9 Hz, 1H), 2.07 (s, 3H), 2.01-1.85 (m, 3H), 1.69-1.49 (m, 1H), 1.27 (dd, J=6.8, 2.2 Hz, 3H).

### Example 52: Preparation of N-(2-((carboxymethyl)((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)carbonyl)amino)ethyl)-N-(((phosphonooxy)methoxy)carbonyl)glycine (52)

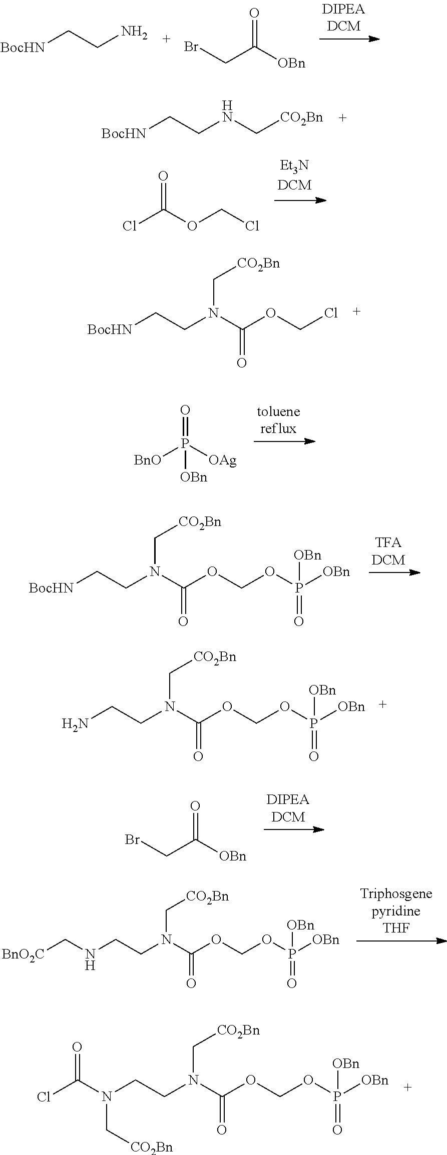

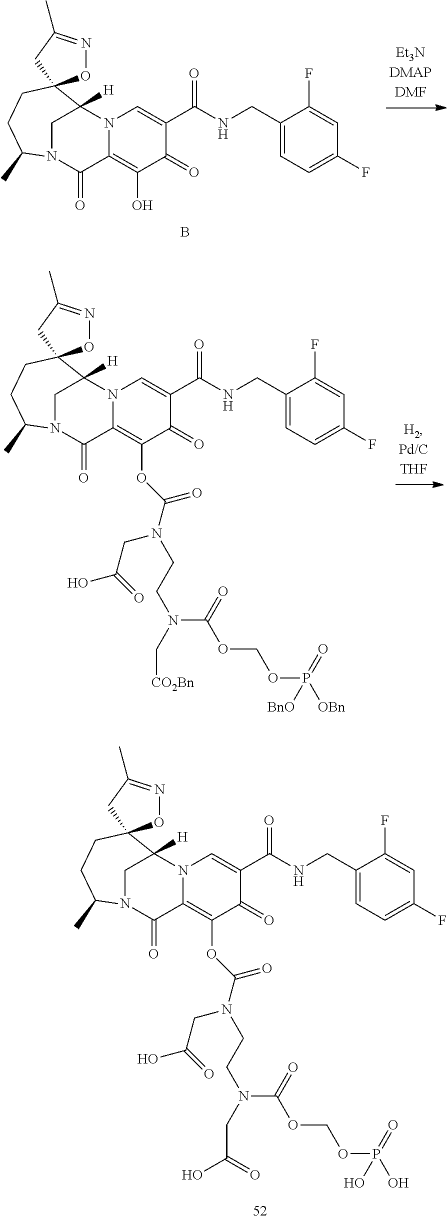

### Step 1: Preparation of benzyl (2-((tert-butoxycarbonyl)amino)ethyl)glycinate

To a stirred solution of tert-butyl N-(2-aminoethyl)carbamate (5.0 g, 31.2 mmol) in DCM (30 mL) at 0° C. under argon was added benzyl 2-bromoacetate (3.57 g, 15.6 mmol) followed by DIPEA (3.1 g, 24 mmol). The reaction mixture was stirred for 16 h at room temperature. The reaction mixture was diluted with DCM and washed with water. The organic layer was dried over anhydrous Na₂SO₄, filtered, and concentrated under reduced pressure. The residue was purified by silica gel column chromatography (20-50% EtOAc/Hexanes) to afford the title compound. MS (m/z) 309.1 [M+H]+.

### Step 2: Preparation of benzyl N-(2-((tert-butoxycarbonyl)amino)ethyl)-N-((chloromethoxy)carbonyl)glycinate

To a stirred solution of benzyl (2-((tert-butoxycarbonyl)amino)ethyl)glycinate (5.0 g, 16.2 mmol) in DCM (50 mL) at 0° C. under argon was added chloromethyl chloroformate (2.51 g, 19.5 mmol) followed by Et₃N (2.46 g, 24.3 mmol). The mixture was stirred for 16 h at room temperature. The reaction mixture was diluted with DCM and washed with water. The organic layer was dried over anhydrous Na₂SO₄, filtered, and concentrated under reduced pressure. The crude residue was purified by silica gel column chromatography (20-50% EtOAc/Hexanes) to afford the title compound. MS (m/z) 401.3 [M+H]+.

### Step 3: Preparation of benzyl N-((((bis(benzyloxy)phosphoryl)oxy)methoxy)carbonyl)-N-(2-((tert-butoxycarbonyl)amino)ethyl)glycinate

To a stirred solution of benzyl N-(2-((tert-butoxycarbonyl)amino)ethyl)-N-((chloromethoxy)carbonyl)glycinate (6.5 g, 16.2 mmol) in toluene (50 mL) at room temperature under argon was added silver dibenzylphosphate (8.12 g, 21.1 mmol) under argon. The mixture was stirred at reflux for 16 h. The reaction mixture was allowed to cool to rt and was filtered, rinsing the solids with toluene (5V). The filtrate was concentrated under reduced pressure and purified by silica gel column chromatography (30-70% EtOAc/Hexanes) to afford the title compound. MS (m/z) 643.2 [M+H]⁺.

### Steps 4-5: Preparation of benzyl (2-((2-(benzyloxy)-2-oxoethyl)((((bis(benzyloxy)phosphoryl)oxy)methoxy)carbonyl)amino)ethyl)etcinate

To a stirred solution of benzyl N-((((bis(benzyloxy)phosphoryl)oxy)methoxy)carbonyl)-N-(2-((tert-butoxycarbonyl)amino)ethyl)glycinate (0.5 g, 0.78 mmol) in DCM (3 mL) at 0° C. under argon was added 2,2,2-trifluoroacetic acid (0.6 mL) in 0.5 mL of DCM under argon. The mixture was stirred for 40 minutes at room temperature and concentrated under reduced pressure. To the crude mixture in DCM (10 mL) at 0° C. under argon was added benzyl 2-bromoacetate (107.0 mg, 0.47 mmol) followed by triethylamine (394 mg, 3.89 mmol). The reaction mixture was stirred for 16 h at room temperature. The reaction mixture was diluted with DCM and washed with water. The organic layer was dried over anhydrous Na₂SO₄, filtered, and concentrated under reduced pressure to afford the title compound. MS (m/z) 691.3 [M+H]+.

### Steps 6-8: Preparation of N-(2-((carboxymethyl)((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5′,6′-[2,7]methanopyrido[1,2-a][1,4]diazonin]-12′-yl)oxy)carbonyl)amino)ethyl)-N-(((phosphonooxy)methoxy)carbonyl)glycine (52)

The title compound was prepared in a manner similar to Steps 1-3 of Example 11, except using benzyl (2-((2-(benzyloxy)-2-oxoethyl)((((bis(benzyloxy)phosphoryl)oxy)methoxy)carbonyl)amino)ethyl)glycinate instead of dibenzyl (3-hydroxypropyl) phosphate in Step 1 and including 0.2 equiv of DMAP in Step 2. MS (m/z) 842.9 [M+H]+. 1H NMR (400 MHz, Methanol-d4) δ 8.63 (d, J=5.4 Hz, 1H), 7.46 (q, J=8.0 Hz, 1H), 7.07-6.89 (m, 2H), 5.73-5.45 (m, 2H), 4.83-4.42 (m, 4H), 4.45-4.03 (m, 4H), 3.95-3.50 (m, 6H), 3.25-3.09 (m, 1H), 2.69 (d, J=17.9 Hz, 1H), 2.06 (s, 3H), 2.01-1.79 (m, 3H), 1.62 (d, J=13.6 Hz, 1H), 1.30-1.17 (m, 3H).

### Example 53: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((R)-2-(phosphonooxy)propyl) carbonate (53)

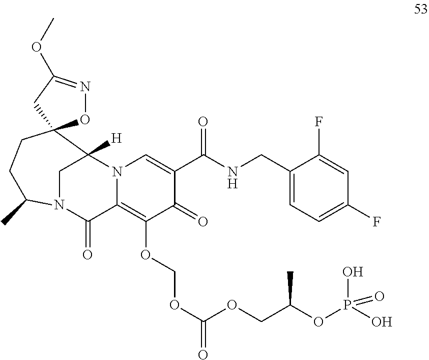 [SMILES: COC1=NO[C@@]2(CC[C@H](C)N3C[C@H]2n2cc(C(=O)NCc4ccc(F)cc4F)c(=O)c(OCOC(=O)OC[C@@H](C)OP(=O)(O)O)c2C3=O)C1]

The title compound was prepared in a manner similar to Example 18, except using (R)-1-((tert-butyldimethylsilyl)oxy)propan-2-ol instead of (R)-2-((tert-butyldimethylsilyl)oxy)propan-1-ol in Step 1, and Intermediate C is used instead of Intermediate B in Step 4. MS (m/z) 714.9[M−H]+. 1H NMR (400 MHz, DMSO-d6) δ 10.27 (t, J=5.9 Hz, 1H), 8.75 (s, 1H), 7.42 (td, J=8.7, 6.6 Hz, 1H), 7.25 (ddd, J=10.5, 9.3, 2.6 Hz, 1H), 7.08 (ddd, J=10.5, 7.9, 2.5 Hz, 1H), 5.79 (d, J=6.4 Hz, 1H), 5.68 (d, J=6.4 Hz, 1H), 4.72 (m, 2H), 4.64 (dt, J=11.1, 7.4 Hz, 1H), 4.55 (t, J=5.9 Hz, 2H), 4.53-4.39 (m, 1H), 4.15 (dd, J=11.2, 5.2 Hz, 1H), 4.04 (dd, J=11.3, 4.6 Hz, 1H), 3.82 (s, 3H), 3.75-3.61 (m, 2H), 3.06 (d, J=16.9 Hz, 1H), 2.76 (dd, J=16.9, 5.2 Hz, 1H), 1.96-1.85 (m, 1H), 1.76 (dd, J=23.5, 12.0 Hz, 2H), 1.39-1.28 (m, 1H), 1.24-1.12 (m, 6H).

### Example 54: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((R)-1-(phosphonooxy)propan-2-yl) carbonate (54)

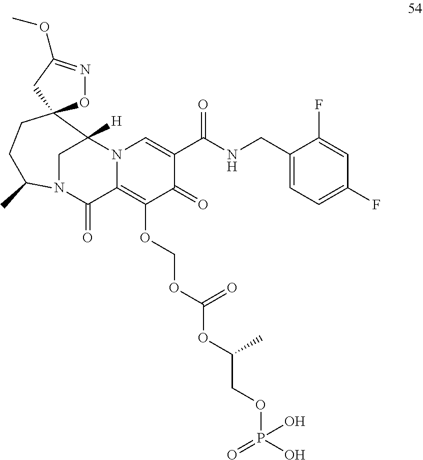 [SMILES: COC1=NO[C@@]2(CC[C@H](C)N3C[C@H]2n2cc(C(=O)NCc4ccc(F)cc4F)c(=O)c(OCOC(=O)O[C@H](C)COP(=O)(O)O)c2C3=O)C1]

The title compound was prepared in a manner similar to Example 18, except using Intermediate C instead of Intermediate B in Step 4. MS (m/z) 715.00 [M+H]+. ¹H NMR (400 MHz, DMSO) δ 10.28 (t, J=6.0 Hz, 1H), 8.75 (s, 1H), 7.54-7.33 (m, 1H), 7.33-7.16 (m, 1H), 7.16-6.96 (m, 1H), 5.85 (d, J=6.4 Hz, 1H), 5.59 (d, J=6.4 Hz, 1H), 4.93-4.76 (m, 1H), 4.74-4.47 (m, 5H), 3.98-3.78 (m, 5H), 3.78-3.59 (m, 2H), 3.05 (d, J=16.8 Hz, 1H), 2.75 (d, J=16.9 Hz, 1H), 1.95-1.69 (m, 4H), 1.44-1.26 (m, 1H), 1.26-1.10 (m, 6H).

### Intermediates F and G: Preparation of 3-((bis(benzyloxy)phosphoryl)oxy)-2-methylbutan-2-yl (chloromethyl) carbonate (Intermediate F) and 3-((bis(benzyloxy)phosphoryl)oxy)-3-methylbutan-2-yl (chloromethyl) carbonate (Intermediate G)

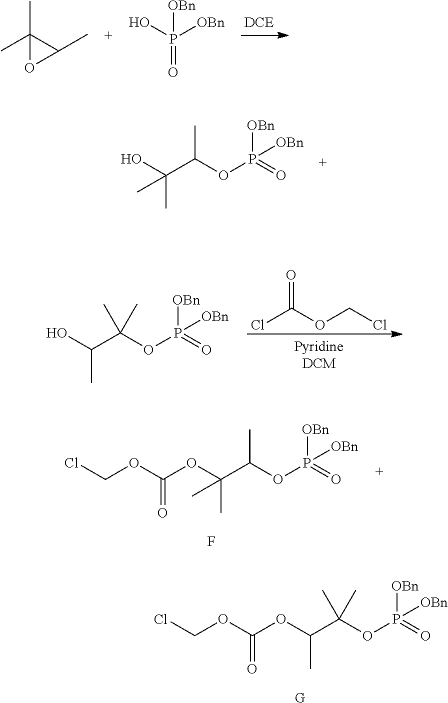

### Step 1: Synthesis of mixture of dibenzyl (3-hydroxy-3-methylbutan-2-yl) phosphate and dibenzyl (3-hydroxy-2-methylbutan-2-yl) phosphate

Into a solution of dibenzyl hydrogen phosphate (1.925 g, 6.92 mmol) in DCM (24 mL), 2,2,3-trimethyloxirane (0.596 g 6.92 mmol) was added at rt. After stirring for overnight, solvent was removed and the residue was purified by column chromatography (0-100% EtOAc/Hexanes) on silica gel to provide the mixture of title products. MS (m z) 364.9 [M+H]⁺.

### Step 2: Synthesis of 3-((bis(benzyloxy)phosphoryl)oxy)-2-meth ylbutan-2-yl (chloromethyl) carbonate (Intermediate D) and 3-((bis(benzyloxy)phosphoryl)oxy)-3-methylbutan-2-v (chloromethyl) carbonate (Intermediate E)

Into the mixture of dibenzyl (3-hydroxy-3-methylbutan-2-yl) phosphate in DCM (20 mL) at 0° C., was added, chloromethyl carbonochloridate (0.442 g, 3.43 mmol), and pyridine (326 mg, 4.12 mmol). After addition, the reaction was allowed to warm to rt for overnight. Then the reaction mixture was extracted with ethyl acetate and washed with brine. After drying with anhydrous MgSO₄, the solvent was removed and the residue was purified by silica gel column chromatography (0-100% EtOAc/Hexanes) to provide the two title products.

Intermediate F (Peak 1): 3-((bis(benzyloxy)phosphoryl)oxy)-2-methylbutan-2-yl (chloromethyl) carbonate: ¹H NMR (400 MHz, Chloroform-d) δ 7.37 (s, 10H), 5.64 (d, J=6.2 Hz, 1H), 5.55 (d, J=6.3 Hz, 1H), 5.13-5.06 (m, 3H), 5.05 (d, J=2.3 H z, 1H), 4.85-4.75 (m, 1H), 1.51 (d, J=8.7 Hz, 6H), 1.33 (d, J=6.5 Hz, 3H).

Intermediate G (Peak 2): 3-((bis(benzyloxy)phosphoryl)oxy)-3-methylbutan-2-yl (chloromethyl) carbonate: ¹H NMR (400 MHz, Chloroform-d) δ 7.43-7.3 (m, 10H), 5.68 (d, J=0.8 Hz, 2H), 5.03 (dd, J=7.8, 3.9 Hz, 4H), 4.9 (qd, J=6.5, 1.2 Hz, 1H, 1.54 (d, J=4.2 Hz, 6H), 1.3 (d, J=6.5 Hz, 3H)

### Example 55: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (2-methyl-3-(phosphonooxy)butan-2-yl) carbonate (55)

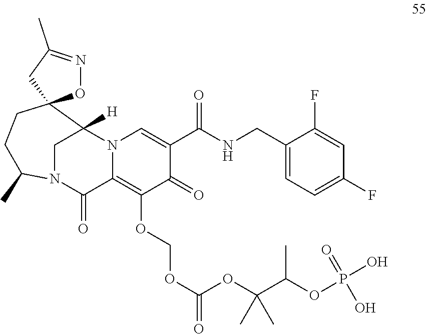 [SMILES: CC1=NO[C@@]2(CC[C@H](C)N3C[C@H]2n2cc(C(=O)NCc4ccc(F)cc4F)c(=O)c(OCOC(=O)OC(C)(C)C(C)OP(=O)(O)O)c2C3=O)C1]

The title compound was prepared in a manner similar to Example 18 except using 3-((bis(benzyloxy)phosphoryl)oxy)-2-methylbutan-2-yl (chloromethyl) carbonate (Intermediate F) instead of (R)-1-((bis(benzyloxy)phosphoryl)oxy)propan-2-yl (chloromethyl) carbonate in Step 4. MS (m/z) 727.97 [M+H]+. ¹H NMR (400 MHz, DMSO) δ 10.28 (t, J=6.1 Hz, 1H), 8.71 (s, 1H), 7.50-7.36 (m, 1H), 7.36-7.17 (m, 1H), 7.17-7.01 (m, 1H), 5.91-5.73 (m, 1H), 5.73-5.58 (m, 1H), 4.78-4.42 (m, 5H), 3.69 (s, 2H), 3.44-3.34 (m, 2H), 3.00 (d, J=17.5 Hz, 1H), 2.63 (d, J=17.6 Hz, 1H), 1.95 (s, 3H), 1.89-1.66 (m, 3H), 1.46-1.23 (m, 6H), 1.23-1.13 (m, 6H), 1.14-1.06 (m, 1H).

### Example 56: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (3-methyl-3-(phosphonooxy)butan-2-yl) carbonate (56)

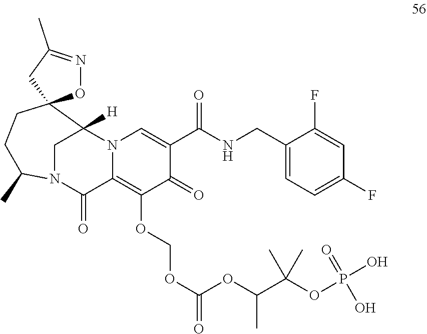 [SMILES: CC1=NO[C@@]2(CC[C@H](C)N3C[C@H]2n2cc(C(=O)NCc4ccc(F)cc4F)c(=O)c(OCOC(=O)OC(C)C(C)(C)OP(=O)(O)O)c2C3=O)C1]

The title compound was prepared in a manner similar to Example 18 except using 3-((bis(benzyloxy)phosphoryl)oxy)-3-methylbutan-2-yl (chloromethyl) carbonate (Intermediate G) instead of (R)-1-((bis(benzyloxy)phosphoryl)oxy)propan-2-yl (chloromethyl) carbonate in Step 4. MS (m/z) 727.619 [M+H]. ¹H NMR (400 MHz, DMSO) δ 10.40-10.23 (m, 11H), 8.77-8.62 (m, 1H), 7.56-7.34 (m, 11H), 7.34-7.18 (m, 1H), 7.18-7.02 (m, 1H), 5.86-5.66 (m, 1H), 5.66-5.49 (m, 1H), 4.77-4.69 (m, 1H), 4.69-4.47 (m, 4H), 4.47-4.29 (m, 1H), 3.77-3.62 (m, 2H), 3.46-3.34 (m, 1H), 3.00 (d, J=17.3 Hz, 1H), 2.75-2.59 (m, 1H), 1.95 (s, 3H), 1.87-1.68 (m, 3H), 1.50-1.23 (m, 7H), 1.23-0.98 (m, 6H).

### Intermediates H and I: Preparation (2R,3S)-3-((bis(benzyloxy)phosphoryl)oxy)butan-2-yl (chloromethyl) carbonate (Intermediate H) and (2S,3R)-3-((bis(benzyloxy)phosphoryl)oxy)butan-2-yl (chloromethyl) carbonate (Intermediate I)

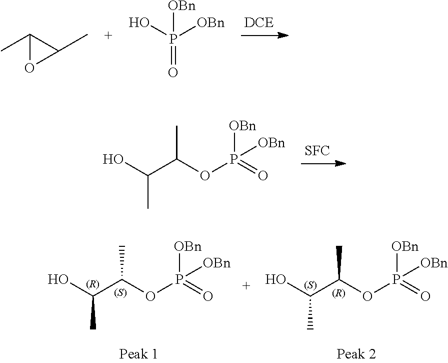

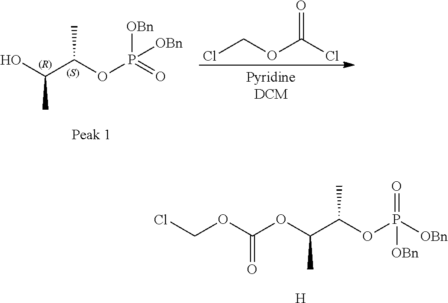

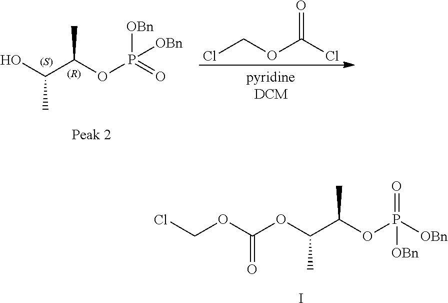

### Step 1: Synthesis of mixture of dibenzyl (3-hydroxybutan-2-yl) phosphate

Into a solution of dibenzyl hydrogen phosphate (2.42 g, 33.6 mmol) in DCM (24 mL), 2,3-dimethyloxirane (0.596 g 6.92 mmol) was added at rt. After stirring for overnight, solvent was removed and the residue was purified by column chromatography (0-100% EtOAc/Hexanes) on silica gel to provide the mixture of title products. MS (m/z) 350.8 [M+H]⁺.

### Step 2: Preparation of dibenzyl ((2S,3R)-3-hydroxybutan-2-yl) phosphate (Peak 1) and dibenzyl ((2R,3S)-3-hydroxybutan-2-yl) phosphate (Peak 2)

SFC separation of dibenzyl (3-hydroxybutan-2-yl) phosphate (Column: IG 4.6×100 mm 5 mic, 3 mL/min. 20% EtOH).

(Peak 1): dibenzyl ((2S,3R)-3-hydroxybutan-2-yl) phosphate: MS (m/z): 350.8 [M+H]+.

(Peak 2): dibenzyl ((2R,3S)-3-hydroxybutan-2-yl) phosphate: MS (m/z): 350.9 [M+H]+.

### Step 3: Preparation of (2R,3S)-3-((bis(benzyloxy)phosphoryl)oxy)butan-2-yl (chloromethyl) carbonate (Intermediate H) and (2S,3R)-3-((bis(benzyloxy)phosphoryl)oxy)butan-2-yl (chloromethyl) carbonate (Intermediate I)

Into the mixture of dibenzyl ((2S,3R)-3-hydroxybutan-2-yl) phosphate in DCM (20 mL) at 0° C., was added, chloromethyl carbonochloridate (0.237 g, 1.84 mmol), and pyridine (165 mg, 2.09 mmol). After addition, the reaction was allowed to warm to rt for overnight. Then the reaction mixture was extracted with ethyl acetate and washed with brine. After drying with anhydrous MgSO₄, the solvent was removed and the residue was purified by silica gel column chromatography (0-100% EtOAc/Hexanes) to provide the two title products.

(2R,3S)-3-((bis(benzyloxy)phosphoryl)oxy)butan-2-yl (chloromethyl) carbonate (Intermediate H): MS (m/z): 442.7 [M+H]+.

(2S,3R)-3-((bis(benzyloxy)phosphoryl)oxy)butan-2-yl (chloromethyl) carbonate (Intermediate 1): MS (m/z): 442.9[M+H]+.

### Example 57: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2S,3R)-3-(phosphonooxy)butan-2-yl) carbonate (57)

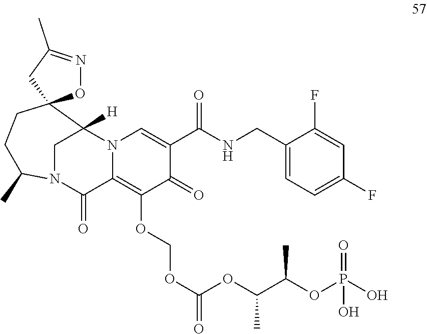 [SMILES: CC1=NO[C@@]2(CC[C@H](C)N3C[C@H]2n2cc(C(=O)NCc4ccc(F)cc4F)c(=O)c(OCOC(=O)O[C@@H](C)[C@@H](C)OP(=O)(O)O)c2C3=O)C1]

The title compound was prepared in a manner similar to Example 18 except using (2S,3R)-3-((bis(benzyloxy)phosphoryl)oxy)butan-2-yl (chloromethyl) carbonate (Intermediate I) instead of (R)-1-((bis(benzyloxy)phosphoryl)oxy)propan-2-yl (chloromethyl) carbonate in Step 4. MS (m/z) 712.98 [M+H]+. ¹H NMR (400 MHz, DMSO) δ 10.28 (t, J=5.9 Hz, 1H), 8.70 (s, 1H), 7.53-7.36 (m, 1H), 7.36-7.15 (m, 1H), 7.15-6.99 (m, 1H), 5.85 (d, J=6.5 Hz, 1H), 5.60 (d, J=6.5 Hz, 1H), 4.78-4.39 (m, 5H), 4.28-4.09 (m, 1H), 3.79-3.58 (m, 2H), 3.00 (d, J=17.5 Hz, 1H), 2.63 (d, J=17.6 Hz, 1H), 1.94 (s, 3H), 1.89-1.67 (m, 3H), 1.44-1.21 (m, 3H), 1.23-0.98 (m, 9H).

### Example 58: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2R,3S)-3-(phosphonooxy)butan-2-yl) carbonate (58)

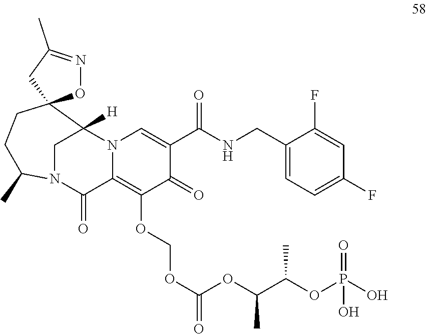 [SMILES: CC1=NO[C@@]2(CC[C@H](C)N3C[C@H]2n2cc(C(=O)NCc4ccc(F)cc4F)c(=O)c(OCOC(=O)O[C@H](C)[C@H](C)OP(=O)(O)O)c2C3=O)C1]

The title compound was prepared in a manner similar to Example 18 except using (2R,3S)-3-((bis(benzyloxy)phosphoryl)oxy)butan-2-yl (chloromethyl) carbonate (Intermediate H) instead of (R)-1-((bis(benzyloxy)phosphoryl)oxy)propan-2-yl (chloromethyl) carbonate in Step 4. MS (m/z) 712.9 [M+H]+. 1H NMR (400 MHz, DMSO-d6) δ 10.28 (t, J=5.9 Hz, 1H), 8.70 (s, 1H), 7.43 (td, J=8.6, 6.5 Hz, 1H), 7.35-7.18 (m, 1H), 7.08 (td, J=8.5, 2.6 Hz, 1H), 5.79-5.65 (m, 2H), 4.74-4.41 (m, 5H), 4.25-4.12 (m, 2H), 3.69 (s, 2H), 3.00 (d, J=17.5 Hz, 1H), 2.64 (d, J=17.5 Hz, 1H), 1.95 (s, 4H), 1.78 (p, J=6.7 Hz, 3H), 1.33-1.23 (m, 1H), 1.21-1.01 (m, 9H).

### Example 59: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2S,3R)-3-(phosphonooxy)butan-2-yl) carbonate (59)

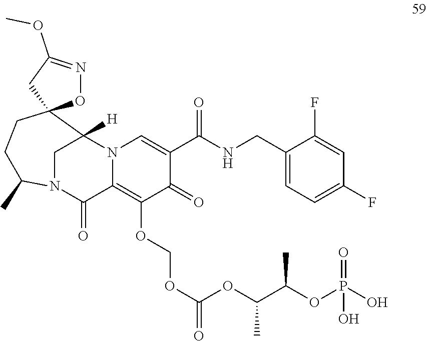 [SMILES: COC1=NO[C@@]2(CC[C@H](C)N3C[C@H]2n2cc(C(=O)NCc4ccc(F)cc4F)c(=O)c(OCOC(=O)O[C@@H](C)[C@@H](C)OP(=O)(O)O)c2C3=O)C1]

The title compound was prepared in a manner similar to Example 57 except using Intermediate C instead of Intermediate B in Step 4. MS (m/z) 728.98 [M+H]+. ¹H NMR (400 MHz, DMSO) δ 10.28 (t, J=5.9 Hz, 1H), 8.74 (s, 1H), 7.51-7.33 (m, 1H), 7.33-7.18 (m, 1H), 7.18-6.99 (m, 11H), 5.83 (d, J=6.4 Hz, 1H), 5.60 (d, J=6.4 Hz, 1H), 4.77-4.47 (m, 5H), 4.31-4.11 (m, 1H), 3.82 (s, 3H), 3.74-3.61 (m, 2H), 3.05 (d, J=16.9 Hz, 1H), 2.75 (d, J=16.9 Hz, 1H), 1.95-1.65 (m, 3H), 1.43-1.28 (m, 3H), 1.21-1.12 (m, 9H).

### Example 60: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2R,3S)-3-(phosphonooxy)butan-2-yl) carbonate (60)

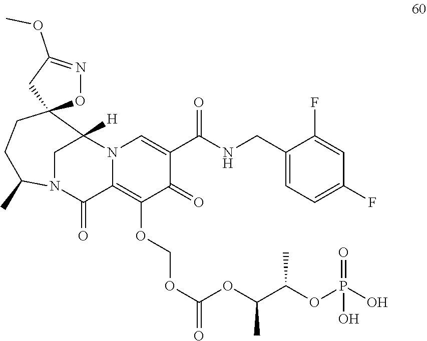 [SMILES: COC1=NO[C@@]2(CC[C@H](C)N3C[C@H]2n2cc(C(=O)NCc4ccc(F)cc4F)c(=O)c(OCOC(=O)O[C@H](C)[C@H](C)OP(=O)(O)O)c2C3=O)C1]

The title compound was prepared in a manner similar to Example 59 except using Intermediate C instead of Intermediate B in Step 4. MS (m/z) 728.8 [M+H]+. ¹H NMR (400 MHz, DMSO) δ 10.27 (t, J=5.9 Hz, 1H), 8.74 (s, 1H), 7.52-7.32 (m, 1H), 7.32-7.16 (m, 1H), 7.16-7.00 (m, 1H), 5.83-5.66 (m, 2H), 4.74-4.46 (m, 5H), 4.32-4.14 (m, 1H), 3.82 (s, 3H), 3.73-3.61 (m, 2H), 3.05 (d, J=16.8 Hz, 1H), 2.85-2.71 (m, 1H), 1.95-1.69 (m, 3H), 1.40-1.31 (m, 3H), 1.20-1.10 (m, 9H).

### Example 61: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2R,3R)-3-(phosphonooxy)butan-2-yl) carbonate (61)

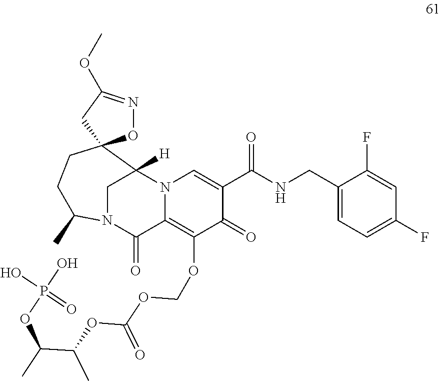 [SMILES: COC1=NO[C@@]2(CC[C@H](C)N3C[C@H]2n2cc(C(=O)NCc4ccc(F)cc4F)c(=O)c(OCOC(=O)O[C@H](C)[C@@H](C)OP(=O)(O)O)c2C3=O)C1]

The title compound was prepared in a manner similar to Example 29, except using dibenzyl [(1R,2R)-2-hydroxy-1-methyl-propyl]phosphate instead of dibenzyl [(1S,2S)-2-hydroxy-1-methyl-propyl]phosphate. MS (m/z) 728.70 [M+H]⁺. ¹H NMR (400 MHz, MeOD) δ 8.73 (d, J=2.2 Hz, 1H), 7.53-7.30 (m, 1H), 7.08-6.84 (m, 2H), 5.93 (d, J=6.6 Hz, 1H), 5.68 (d, J=6.6 Hz, 1H), 4.90-4.76 (m, 3H), 4.76-4.65 (m, 1H), 4.61 (s, 2H), 4.55-4.36 (m, 1H), 3.93-3.85 (m, 3H), 3.85-3.71 (m, 2H), 3.20 (d, J=17.0 Hz, 1H), 2.75 (d, J=17.0 Hz, 11H), 2.15-1.89 (m, 4H), 1.66-1.49 (m, 1H), 1.38-1.18 (m, 10H).

### Example 62: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (2-methyl-1-(phosphonooxy)propan-2-yl) carbonate (62)

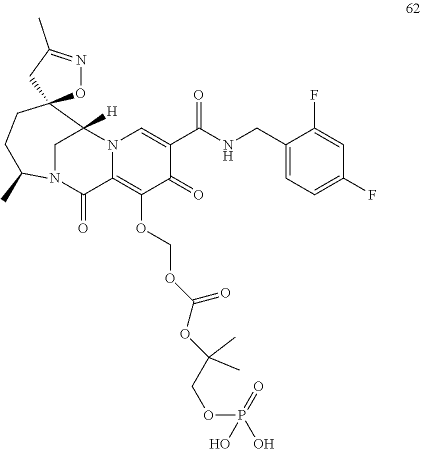 [SMILES: CC1=NO[C@@]2(CC[C@H](C)N3C[C@H]2n2cc(C(=O)NCc4ccc(F)cc4F)c(=O)c(OCOC(=O)OC(C)(C)COP(=O)(O)O)c2C3=O)C1]

The title compound was made following the same method as Example 10, except using 2-methylpropane-1,2-diol instead of propane-1,3-diol in Step 1. MS (m/z) 713.3 [M+H]⁺. 1H NMR (400 MHz, Methanol-d4) δ 8.58 (s, 1H), 7.45 (td, J=8.5, 6.4 Hz, 1H), 7.05-6.86 (m, 2H), 5.84 (d, J=6.6 Hz, 1H), 5.65 (d, J=6.6 Hz, 1H), 4.92-4.77 (m, 1H), 4.73-4.55 (m, 2H), 4.49 (d, J=2.2 Hz, 1H), 4.06 (h, J=5.2 Hz, 2H), 3.90-3.70 (m, 2H), 3.16 (d, J=17.8 Hz, 1H), 2.77-2.62 (m, 1H), 2.06 (s, 3H), 2.01-1.85 (m, 3H), 1.56 (dt, J=10.2, 6.4 Hz, 1H), 1.48 (d, J=3.5 Hz, 6H), 1.27 (d, J=6.7 Hz, 3H).

### Example 63: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (2-methyl-1-(phosphonooxy)propan-2-yl) carbonate (63)

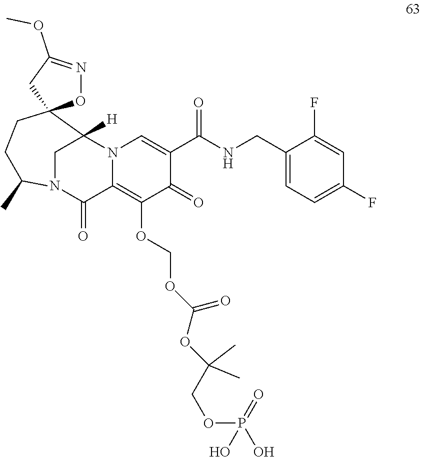 [SMILES: COC1=NO[C@@]2(CC[C@H](C)N3C[C@H]2n2cc(C(=O)NCc4ccc(F)cc4F)c(=O)c(OCOC(=O)OC(C)(C)COP(=O)(O)O)c2C3=O)C1]

The title compound was made following the same method as Example 10, except using 2-methylpropane-1,2-diol instead of propane-1,3-diol in Step 1 and Intermediate C instead of Intermediate B in Step 3. MS (m/z) 729.2 [M+H]⁺. 1H NMR (400 MHz, Methanol-d4) δ 8.64 (s, 1H), 7.46 (td, J=8.5, 6.3 Hz, 1H), 7.05-6.91 (m, 2H), 5.83 (d, J=6.6 Hz, 1H), 5.65 (d, J=6.6 Hz, 1H), 4.92-4.77 (m, 1H), 4.63 (d, J=8.8 Hz, 3H), 4.18-3.98 (m, 2H), 3.90 (s, 3H), 3.86-3.73 (m, 2H), 3.18 (d, J=17.1 Hz, 1H), 2.77 (d, J=17.1 Hz, 1H), 2.13-1.86 (m, 3H), 1.65-1.52 (m, 1H), 1.48 (d, J=3.5 Hz, 6H), 1.27 (d, J=6.7 Hz, 3H).

### Intermediate J: Preparation of (2R)-1-[tert-butyl(dimethyl)silyl]oxybutan-2-ol (Intermediate J)

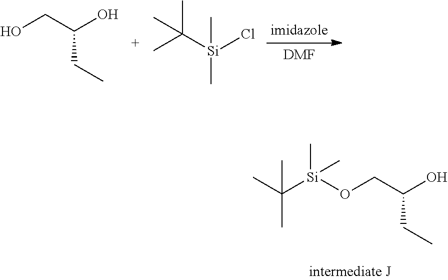

To a stirred solution of (2R)-butane-1,2-diol (1.0 g, 11.1 mmol), imidazole (755 mg, 11.1 mmol), and DMAP (136 mg, 1.11 mmol) in DMF (3 mL) at 70° C. under argon was added tert-butyl-chloro-dimethyl-silane (1.84 g, 12.2 mmol). The mixture was stirred for 3 hours. The reaction mixture was diluted with EtOAc (50 mL) and washed with saturated NH₄Cl and water. The organic layer was dried over anhydrous Na₂SO₄, filtered, and concentrated under reduced pressure and purified by silica gel column chromatography (20-70% EtOAc/Hexanes) to afford the title compound. MS (m/z) 205.1 [M+H]⁺.

### Example 64: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((R)-2-(phosphonooxy)butyl) carbonate (64)

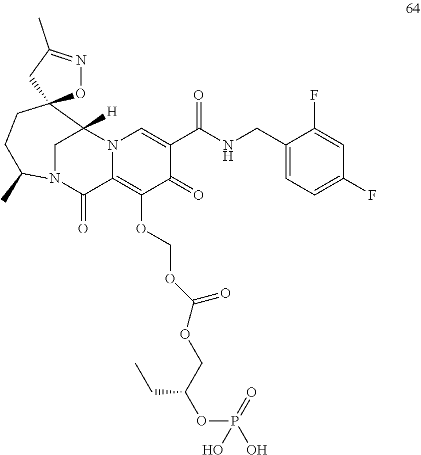 [SMILES: CC[C@H](COC(=O)OCOc1c2n(cc(C(=O)NCc3ccc(F)cc3F)c1=O)[C@@H]1CN(C2=O)[C@@H](C)CC[C@]12CC(C)=NO2)OP(=O)(O)O]

The title compound was made following the same method as Example 18, except using (2R)-1-[tert-butyl(dimethyl)silyl]oxybutan-2-ol instead of (R)-2-((tert-butyldimethylsilyl)oxy)propan-1-ol. MS (m/z) 713.1 [M+H]⁺. 1H NMR (400 MHz, DMSO-d6) δ 10.28 (t, J=6.0 Hz, 1H), 8.72 (s, 1H), 7.42 (td, J=8.7, 6.6 Hz, 1H), 7.25 (ddd, J=10.4, 9.3, 2.6 Hz, 1H), 7.16-7.03 (m, 1H), 5.81 (d, J=6.4 Hz, 1H), 5.69 (d, J=6.4 Hz, 1H), 4.75-4.48 (m, 4H), 4.32-4.04 (m, 3H), 3.79-3.63 (m, 2H), 3.01 (d, J=17.6 Hz, 1H), 2.65 (d, J=17.6 Hz, 1H), 1.95 (s, 3H), 1.91-1.69 (m, 3H), 1.69-1.49 (m, 2H), 1.38-1.20 (m, 1H), 1.16 (d, J=6.6 Hz, 3H), 0.87 (t, J=7.4 Hz, 3H).

### Example 65: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((R)-2-(phosphonooxy)butyl) carbonate (65)

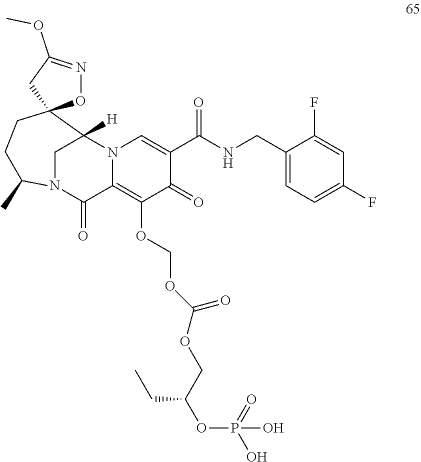 [SMILES: CC[C@H](COC(=O)OCOc1c2n(cc(C(=O)NCc3ccc(F)cc3F)c1=O)[C@@H]1CN(C2=O)[C@@H](C)CC[C@]12CC(OC)=NO2)OP(=O)(O)O]

The title compound was made following the same method as Example 1, except using (2R)-1-[tert-butyl(dimethyl)silyl]oxybutan-2-ol instead of (R)-2-((tert-butyldimethylsilyl)oxy)propan-1-ol in Step 1 and Intermediate C instead of Intermediate B in Step 4. MS (m/z) 729.1 [M+H]⁺. 1H NMR (400 MHz, DMSO-d6) δ 10.27 (t, J=6.0 Hz, 1H), 8.76 (s, 1H), 7.42 (td, J=8.7, 6.6 Hz, 1H), 7.25 (ddd, J=10.5, 9.3, 2.6 Hz, 1H), 7.14-6.92 (m, 1H), 5.80 (d, J=6.4 Hz, 1H), 5.69 (d, J=6.4 Hz, 1H), 4.77-4.49 (m, 4H), 4.34-4.18 (m, 2H), 4.11 (dd, J=11.0, 4.4 Hz, 1H), 3.82 (s, 3H), 3.77-3.61 (m, 2H), 3.06 (d, J=16.8 Hz, 1H), 2.77 (d, J=16.9 Hz, 1H), 2.01-1.86 (m, 1H), 1.86-1.69 (m, 2H), 1.69-1.48 (m, 2H), 1.40-1.24 (m, 1H), 1.16 (d, J=6.7 Hz, 3H), 0.87 (t, J=7.4 Hz, 3H).

### Intermediate K: Preparation of (2S)-1-[tert-butyl(dimethyl)silyl]oxybutan-2-ol (Intermediate K)

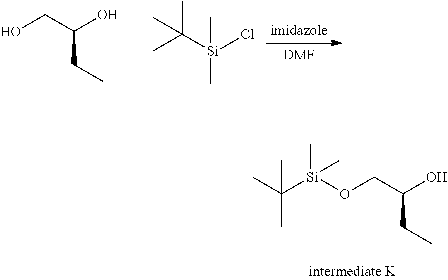

To a stirred solution of (2S)-butane-1,2-diol (1.0 g, 11.1 mmol), imidazole (755 mg, 11.1 mmol), and DMAP (136 mg, 1.11 mmol) in DMF (3 mL) at 70° C. under argon was added tert-butyl-chloro-dimethyl-silane (1.84 g, 12.2 mmol). The mixture was stirred for 3 hours. The reaction mixture was diluted with EtOAc (50 mL) and washed with saturated NH₄C1 and water. The organic layer was dried over anhydrous Na₂SO₄, filtered, concentrated under reduced pressure and purified by silica gel column chromatography (20-70% EtOAc/Hexanes) to afford the title compound. MS (m/z) 205.1 [M+H]⁺.

### Example 66: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((S)-2-(phosphonooxy)butyl) carbonate (66)

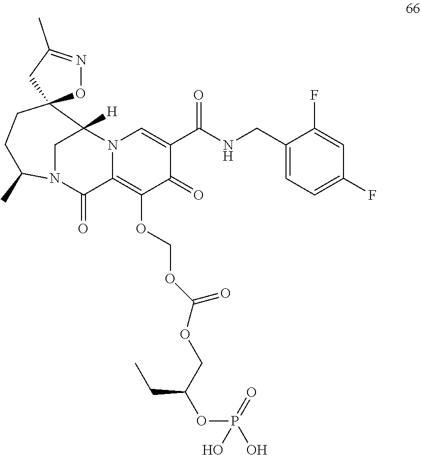 [SMILES: CC[C@@H](COC(=O)OCOc1c2n(cc(C(=O)NCc3ccc(F)cc3F)c1=O)[C@@H]1CN(C2=O)[C@@H](C)CC[C@]12CC(C)=NO2)OP(=O)(O)O]

The title compound was made following the same method as Example 18, except using (2S)-1-[tert-butyl(dimethyl)silyl]oxybutan-2-ol instead of (R)-2-((tert-butyldimethylsilyl)oxy)propan-1-ol. MS (m/z) 713.1 [M+H]⁺. 1H NMR (400 MHz, Methanol-d4) δ 8.58 (s, 1H), 7.57-7.37 (m, 1H), 7.09-6.88 (m, 2H), 5.93 (d, J=6.5 Hz, 1H), 5.71 (d, J=6.5 Hz, 1H), 4.91-4.76 (m, 1H), 4.72-4.59 (m, 2H), 4.47 (s, 1H), 4.44-4.35 (m, 1H), 4.33-4.21 (m, 2H), 3.86-3.70 (m, 2H), 3.23-3.11 (m, 1H), 2.71 (d, J=17.9 Hz, 1H), 2.06 (s, 3H), 2.00-1.86 (m, 3H), 1.84-1.65 (m, J=6.8 Hz, 2H), 1.64-1.46 (m, 1H), 1.27 (d, J=6.7 Hz, 3H), 1.00 (t, J=7.5 Hz, 3H).

### Example 67: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((S)-2-(phosphonooxy)butyl) carbonate (67)

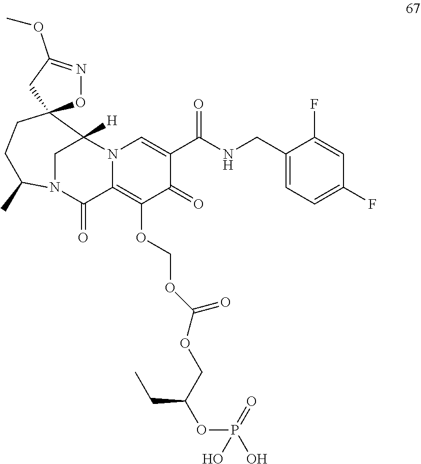 [SMILES: CC[C@@H](COC(=O)OCOc1c2n(cc(C(=O)NCc3ccc(F)cc3F)c1=O)[C@@H]1CN(C2=O)[C@@H](C)CC[C@]12CC(OC)=NO2)OP(=O)(O)O]

The title compound was made following the same method as Example 1, except using (2S)-1-[tert-butyl(dimethyl)silyl]oxybutan-2-ol instead of (R)-2-((tert-butyldimethylsilyl)oxy)propan-1-ol in Step 1 and Intermediate C instead of Intermediate B in Step 4. MS (m/z) 729.1 [M+H]⁺. 1H NMR (400 MHz, Methanol-d4) δ 8.66 (s, 1H), 7.45 (td, J=8.5, 6.4 Hz, 1H), 7.05-6.85 (m, 2H), 5.93 (d, J=6.5 Hz, 1H), 5.70 (d, J=6.6 Hz, 1H), 4.80 (q, J=8.2, 7.8 Hz, 1H), 4.63 (s, 3H), 4.44-4.35 (m, 1H), 4.35-4.17 (m, 2H), 3.90 (s, 3H), 3.80 (dd, J=4.0.2.3 Hz, 2H), 3.19 (d, J=17.0 Hz, 1H), 2.77 (d, J=17.1 Hz, 1H), 2.11-1.87 (m, 3H), 1.84-1.64 (m, 2H), 1.56 (ddd, J=15.5, 9.2, 3.8 Hz, 1H), 1.26 (d, J=6.7 Hz, 3H), 1.00 (t, J=7.5 Hz, 3H).

### Intermediates L and M: Preparation of (R)-3-((bis(benzyloxy)phosphoryl)oxy)-2-methylpropyl (chloromethyl) carbonate (Intermediate L) and (S)-3-((bis(benzyloxy)phosphoryl)oxy)-2-methylpropyl (chloromethyl) carbonate (Intermediate M)

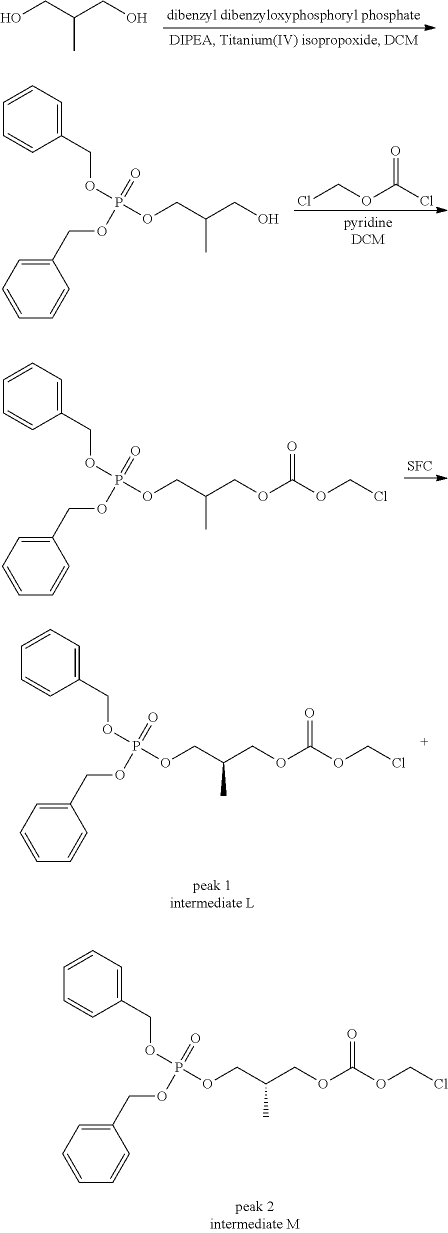

### Step 1: Preparation of mixture of dibenzyl (3-hydroxy-2-methylpropyl) phosphate

Into a solution of dibenzyl dibenzyloxyphosphoryl phosphate (3 g, 5.57 mmol) and 2-methylpropane-1,3-diol (0.8 g, 8.91 mmol) in DCM (30 mL), DIPEA (1.8 g, 13.9 mmol) and titanium(IV) isopropoxide (269 mg, 0.95 mmol) were added at rt. After stirring for overnight, solvent was removed and the residue was purified by column chromatography (0-100% EtOAc/Hexanes) on silica gel to provide the mixture of title products. MS (m/z) 350.9[M+H].

### Step 2: Preparation of mixture of 3-((bis(benzyloxy)phosphoryl)oxy)-2-methylpropyl (chloromethyl) carbonate

Into the mixture of dibenzyl (3-hydroxy-2-methyl-propyl) phosphate (1.4 g, 4 mmol) in DCM (50 mL) at 0° C. chloromethyl carbonochloridate (1.29 g, 0.89 mmol), and pyridine (948 mg, 12 mmol) were added. After addition, the reaction was allowed to warm to rt for overnight. Then the reaction mixture was extracted with ethyl acetate and washed with brine. After drying with anhydrous MgSO₄, the solvent was removed and the residue was submitted to SFC chiral separation.

### Step 3: Preparation of (R)-3-((bis(benzyloxy)phosphoryl)oxy)-2-methylpropyl (chloromethyl) carbonate (Intermediate L) and (S)-3-((bis(benzyloxy)phosphoryl)oxy)-2-methylpropyl (chloromethyl) carbonate (Intermediate M)

SFC separation of 3-((bis(benzyloxy)phosphoryl)oxy)-2-methylpropyl (chloromethyl) carbonate (Column: IG 4.6×100 mm 5mic, 3 mL/min, 20% EtOH).

(Peak 1): (R)-3-((bis(benzyloxy)phosphoryl)oxy)-2-methylpropyl (chloromethyl) carbonate (Intermediate L): MS (m/z): 442.9 [M+H]⁺.

(Peak 2): (S)-3-((bis(benzyloxy)phosphoryl)oxy)-2-methylpropyl (chloromethyl) carbonate (Intermediate M): MS (m/z): 442.9 [M+H]⁺.

### Example 68: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((S)-2-methyl-3-(phosphonooxy)propyl) carbonate (68)

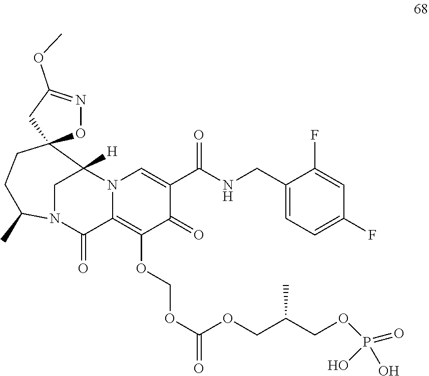 [SMILES: COC1=NO[C@@]2(CC[C@H](C)N3C[C@H]2n2cc(C(=O)NCc4ccc(F)cc4F)c(=O)c(OCOC(=O)OC[C@H](C)COP(=O)(O)O)c2C3=O)C1]

The title compound was prepared following a similar method as Example 57 except using Intermediate M instead of Intermediate B in Step 4. MS (m/z) 728.9 [M+H]⁺. 1H NMR (400 MHz, DMSO-d6) δ 10.27 (t, J=6.0 Hz, 1H), 8.74 (s, 1H), 7.42 (td, J=8.7, 6.7 Hz, 1H), 7.25 (ddd, J=12.9, 7.9, 2.7 Hz, 1H), 7.08 (td, J=8.6, 2.6 Hz, 1H), 5.80 (d, J=6.6 Hz, 1H), 5.65 (d, J=6.4 Hz, 1H), 4.71-4.52 (m, 4H), 4.05 (dd, J=10.4, 5.8 Hz, 1H), 3.98-3.87 (m, 1H), 3.82 (s, 3H), 3.71-3.59 (m, 4H), 3.07-2.90 (m, 1H), 2.76 (dd, J=22.1, 5.2 Hz, 1H), 2.09-1.74 (m, 4H), 1.36 (m, 1H), 1.15 (d, J=6.7 Hz, 3H), 0.88 (d, J=6.8 Hz, 3H).

### Example 69: Preparation (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((S)-2-methyl-3-(phosphonooxy)propyl) carbonate (69)

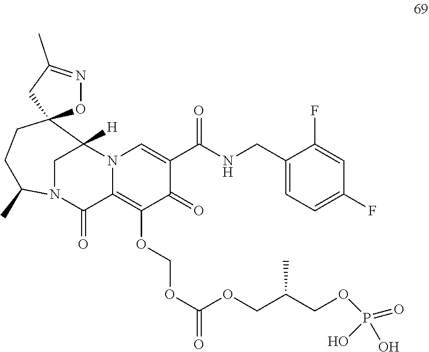 [SMILES: CC1=NO[C@@]2(CC[C@H](C)N3C[C@H]2n2cc(C(=O)NCc4ccc(F)cc4F)c(=O)c(OCOC(=O)OC[C@H](C)COP(=O)(O)O)c2C3=O)C1]

The title compound was prepared following a similar method as Example 18 except using (R)-3-((bis(benzyloxy)phosphoryl)oxy)-2-methylpropyl (chloromethyl) carbonate (Intermediate M) instead of (R)-1-((bis(benzyloxy)phosphoryl)oxy)propan-2-yl (chloromethyl) carbonate in Step 4. MS (m/z) 712.6 [M+H]⁺. ¹H NMR (400 MHz, DMSO) δ 10.28 (t, J=6.0 Hz, 1H), 8.71 (s, 1H), 7.42 (q, J=8.4 Hz, 1H), 7.36-7.18 (m, 1H), 7.10-7.03 (m, 1H), 5.83 (d, J=6.5 Hz, 1H), 5.65 (d, J=6.5 Hz, 1H), 4.66-4.53 (m, 4H), 4.05 (dd, J=10.6, 5.7 Hz, 1H), 3.94 (t, J=8.6 Hz, 1H), 3.71 (d, J=21.9 Hz, 4H), 2.96 (d, J=10.4 Hz, 1H), 2.69-2.60 (m, 1H), 2.05 (dd, J=12.4, 6.2 Hz, 1H), 1.95 (d, J=8.9 Hz, 3H), 1.82-1.73 (m, 3H), 1.29-1.13 (m, 5H), 0.89 (d, J=6.7 Hz, 3H).

### Example 70: Preparation (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((R)-2-methyl-3-(phosphonooxy)propyl) carbonate (70)

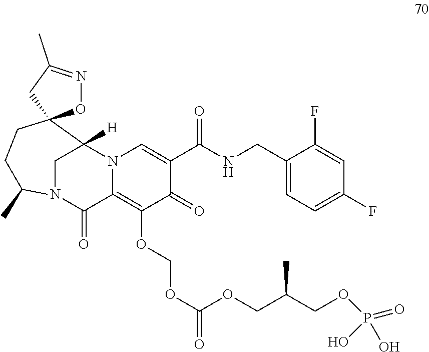 [SMILES: CC1=NO[C@@]2(CC[C@H](C)N3C[C@H]2n2cc(C(=O)NCc4ccc(F)cc4F)c(=O)c(OCOC(=O)OC[C@@H](C)COP(=O)(O)O)c2C3=O)C1]

The title compound was prepared following a similar method as Example 18 except using (S)-3-((bis(benzyloxy)phosphoryl)oxy)-2-methylpropyl (chloromethyl) carbonate (Intermediate L) instead of (R)-1-((bis(benzyloxy)phosphoryl)oxy)propan-2-yl (chloromethyl) carbonate in Step 4. MS (m/z) 712.6 [M+H]⁺. 1H NMR (400 MHz, DMSO-d6) δ 10.28 (t, J=6.0 Hz, 1H), 8.71 (s, 1H), 7.43 (td, J=8.7, 6.6 Hz, 1H), 7.25 (ddd, J=10.5, 9.3, 2.6 Hz, 1H), 7.13-7.03 (m, 1H), 5.82 (d, J=6.5 Hz, 1H), 5.66 (d, J=6.5 Hz, 1H), 4.63 (s, 2H), 4.62-4.47 (m, 2H), 4.06 (dd, J=10.6, 6.0 Hz, 1H), 3.95 (dd, J=10.6, 6.3 Hz, 1H), 3.79-3.65 (m, 4H), 3.01 (d, J=17.6 Hz, 1H), 2.63 (d, J=17.6 Hz, 1H), 2.07 (dq, J=12.7, 6.3 Hz, 1H), 1.95 (s, 3H), 1.78 (ddd, J=21.2, 10.7, 5.6 Hz, 3H), 1.29 (dd, J=15.0, 11.6 Hz, 1H), 1.16 (d, J=6.7 Hz, 3H), 0.90 (d, J=6.9 Hz, 3H).

### Example 71: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((R)-2-methyl-3-(phosphonooxy)propyl) carbonate (71)

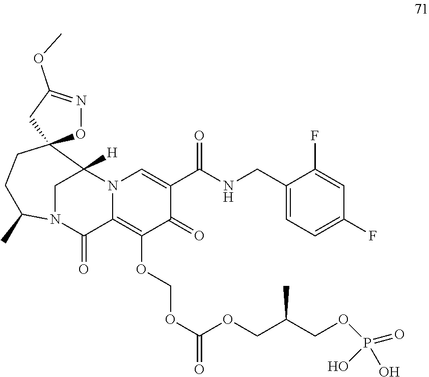 [SMILES: COC1=NO[C@@]2(CC[C@H](C)N3C[C@H]2n2cc(C(=O)NCc4ccc(F)cc4F)c(=O)c(OCOC(=O)OC[C@@H](C)COP(=O)(O)O)c2C3=O)C1]

The title compound was prepared following a similar method as Example 57 except using Intermediate L instead of Intermediate B in Step 4. MS (m/z) 728.9 [M+H]⁺. 1H NMR (400 MHz, DMSO-d6) δ 10.27 (t, J=5.9 Hz, 1H), 8.75 (s, 1H), 7.42 (td, J=8.7, 6.6 Hz, 1H), 7.25 (ddd, J=10.5, 9.3, 2.6 Hz, 1H), 7.08 (td, J=8.6, 2.8 Hz, 1H), 5.80 (d, J=6.4 Hz, 1H), 5.66 (d, J=6.4 Hz, 1H), 4.71 (s, 1H), 4.66-4.58 (m, 1H), 4.61-4.47 (m, 2H), 4.07 (dd, J=10.6, 6.0 Hz, 1H), 3.95 (dd, J=10.6, 6.3 Hz, 1H), 3.82 (s, 3H), 3.79-3.64 (m, 4H), 3.05 (d, J=16.8 Hz, 1H), 2.76 (d, J=16.9 Hz, 1H), 2.08 (h, J=6.3 Hz, 1H), 1.91 (dq, J=13.5, 7.6, 5.9 Hz, 1H), 1.87-1.69 (m, 2H), 1.33 (dd, J=15.5, 10.8 Hz, 1H), 1.15 (d, J=6.7 Hz, 3H), 0.90 (d, J=6.8 Hz, 3H).

### Intermediates N and O: Preparation of (2S,3S)-3-((bis(benzyloxy)phosphoryl)oxy)pentan-2-yl (chloromethyl) carbonate (Intermediate N) and (2S,3R)-3-((bis(benzyloxy)phosphoryl)oxy)pentan-2-yl (chloromethyl) carbonate (Intermediate O)

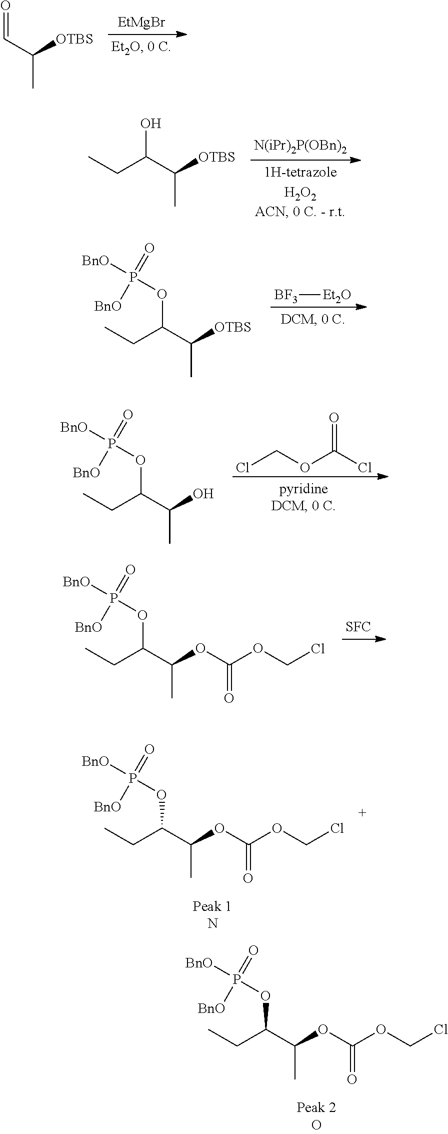

### Step 1: Synthesis of (2S)-2-((tert-butyldimethylsilyl)oxy)pentan-3-ol

To a solution of (S)-2-((tert-butyldimethylsilyl)oxy)propanal (2.0 g, 10.6 mmol) in diethyl ether (0.2 M) at 0° C. was added ethylmagnesium bromide (3.0 M in diethyl ether, 3.90 mL, 11.7 mmol) dropwise. The reaction was then warmed to room temperature and stirred at room temperature for 16 h. The reaction mixture was then quenched with saturated ammonium chloride, extracted with ethyl acetate, dried over magnesium sulfate, filtered, concentrated to afford the title compound as a mixture of diastereomers. The product was used directly in the next step without further purification.

### Step 2: Synthesis of dibenzyl ((2S)-2-((tert-butyldimethylsilyl)ox)pentan-3-yl) phosphate

To a solution of (2S)-2-((tert-butyldimethylsilyl)oxy)pentan-3-ol (2.2 g, 10.1 mmol) in acetonitrile (0.1 M) at 0° C. was added 1H-tetrazole (0.85 g, 12.1 mmol) followed by N-dibenzyloxyphosphanyl-N-isopropyl-propan-2-amine (4.26 g, 12.1 mmol). The reaction mixture was stirred at 0° C. for 1 h. Then hydrogen peroxide (30% in water, 1.48 mL, 20.1 mmol) was added dropwise, and the reaction mixture was stirred for an addition 30 min. The reaction was then quenched with saturated sodium thiosulfate and saturated sodium bicarbonate, extracted with DCM, dried over magnesium sulfate, filtered, dried, concentrated and purified by normal phase silica gel chromatography (0-100% EtOAc in Hexanes) to afford the title compound. ¹H NMR (400 MHz, CDCl₃) δ 7.42-7.31 (m, 1OH), 5.12-4.97 (m, 4H), 4.30-4.11 (m, 1H), 4.08-3.92 (m, 1H), 1.88-1.48 (m, 2H), 1.17-1.07 (m, 2H), 1.04-0.93 (m, 3H), 0.93-0.85 (m, 9H), 0.11-0.00 (m, 6H).

### Step 3: Synthesis of dibenzyl ((2S)-2-hydroxypentan-3-yl) phosphate

To a solution of dibenzyl ((2S)-2-((tert-butyldimethylsilyl)oxy)pentan-3-yl) phosphate (2.89 g, 5.74 mmol) in DCM (0.3 M) at 0° C. was added boron trifluoride diethyl etherate (1.81 mL, 6.88 mmol) dropwise. The reaction mixture was then stirred at 0° C. for 30 min. The reaction mixture was quenched with saturated sodium bicarbonate, extracted with DCM, dried, concentrated and purified by silica gel chromatography (0-100% EtOAc in Hexanes) to afford the title compound. MS (m/z): 365.0 [M+H]⁺.

### Step 4: Synthesis of (2S)-3-((bis(benzyloxy)phosphoryl)oxy)pentan-2-yl (chloromethyl) carbonate

To a solution of dibenzyl ((2S)-2-hydroxypentan-3-yl) phosphate (1.0 g, 2.74 mmol) in DCM at 0° C. (0.2 M) was added chloromethyl chloroformate (0.268 mL, 3.02 mmol) followed by pyridine (0.277 mL, 3.43 mmol) dropwise. The reaction mixture was stirred for 30 min, then quenched with water and 1 M HCl. The reaction was extracted with DCM, dried, filtered and concentrated to afford the title compound used directly in the next step without further purification. MS (m/s): 456.9 [M+H]⁺.

### Step 5: Synthesis of (2S,3S)-3-((bis(benzyloxy)phosphoryl)oxy)pentan-2-yl (chloromethyl) carbonate (Intermediate N) and (2S3R)-3-((bis(benzyloxy)phosphoryl)oxy)pentan-2-yl (chloromethyl) carbonate (Intermediate O)

SFC separation of (2S)-3-((bis(benzyloxy)phosphoryl)oxy)pentan-2-yl (chloromethyl) carbonate (Column: AS-H 5 μm 20×250 mm, 50 mL/min, 15% EtOH).

Peak 1: (2S,3S)-3-((bis(benzyloxy)phosphoryl)oxy)pentan-2-yl (chloromethyl) carbonate (Intermediate N): MS (m/z): 456.8 [M+H]⁺.

Peak 2: (2S,3R)-3-((bis(benzyloxy)phosphoryl)oxy)pentan-2-yl (chloromethyl) carbonate (Intermediate O): MS (m/z): 456.9 [M+H]⁺.

### Example 72: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2S,3S)-3-(phosphonooxy)pentan-2-yl) carbonate (72)

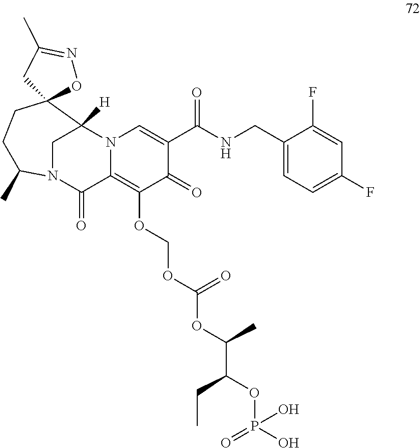 [SMILES: CC[C@H](OP(=O)(O)O)[C@H](C)OC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared following a similar method as Example 18 except using (2S,3S)-3-((bis(benzyloxy)phosphoryl)oxy)pentan-2-yl (chloromethyl) carbonate (Intermediate N) instead of (R)-1-((bis(benzyloxy)phosphoryl)oxy)propan-2-yl (chloromethyl) carbonate in Step 4. MS (m/z): 726.9 [M+H]⁺. ¹H NMR (400 MHz, MeOD) δ 8.58 (s, 1H), 7.55-7.32 (m, 1H), 7.06-6.83 (m, 2H), 6.02-5.86 (m, 1H), 5.85-5.59 (m, 1H), 5.00-4.90 (m, 1H), 4.69-4.60 (m, 3H), 4.47 (s, 1H), 4.22 (s, 1H), 3.93-3.73 (m, 2H), 3.21-3.08 (m, 1H), 2.95 (d, J=53.0 Hz, 2H), 2.78-2.64 (m, 1H), 2.07 (s, 3H), 1.98-1.88 (m, 3H), 1.75-1.64 (m, 2H), 1.64-1.46 (m, 1H), 1.37-1.16 (m, 7H), 0.99 (t, J=7.4 Hz, 3H).

### Example 73: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2S,3R)-3-(phosphonooxy)pentan-2-yl) carbonate (73)

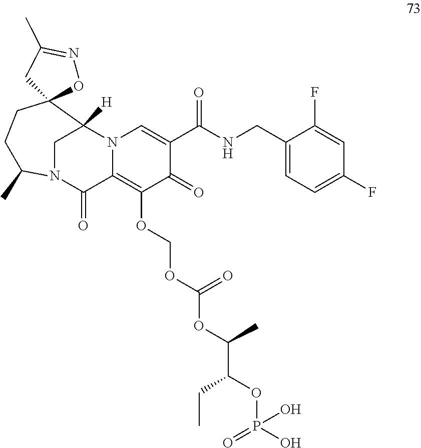 [SMILES: CC[C@@H](OP(=O)(O)O)[C@H](C)OC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared following a similar method as Example 18 except using (2S,3S)-3-((bis(benzyloxy)phosphoryl)oxy)pentan-2-yl (chloromethyl) carbonate (Intermediate O) instead of (R)-1-((bis(benzyloxy)phosphoryl)oxy)propan-2-yl (chloromethyl) carbonate in Step 4. MS (m/z): 726.9 [M+H]⁺. ¹H NMR (400 MHz, MeOD) δ 8.58 (s, 1H), 7.58-7.34 (m, 1H), 7.08-6.87 (m, 2H), 5.94 (d, J=6.5 Hz, 1H), 5.70 (d, J=6.5 Hz, 1H), 4.88-4.74 (m, 1H), 4.65 (s, 2H), 4.47 (s, 1H), 4.35-4.23 (m, 1H), 3.91-3.65 (m, 2H), 3.16 (d, J=17.9 Hz, 1H), 2.95 (d, J=53.0 Hz, 3H), 2.70 (d, J=17.9 Hz, 1H), 2.06 (s, 3H), 2.02-1.85 (m, 4H), 1.78-1.64 (m, 2H), 1.62-1.49 (m, 1H), 1.36-1.18 (m, 6H), 1.01 (t, J=7.4 Hz, 3H).

### Example 74: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2S,3S)-3-(phosphonooxy)pentan-2-yl) carbonate (74)

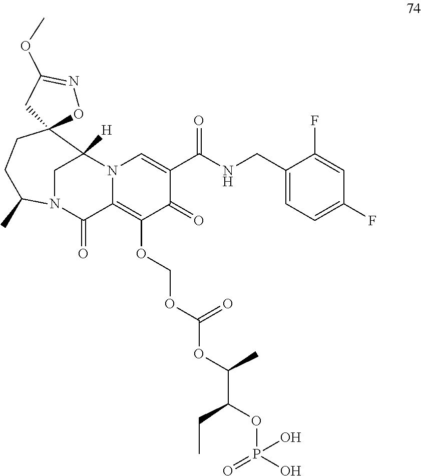 [SMILES: CC[C@H](OP(=O)(O)O)[C@H](C)OC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared following a similar method as Example 72 except using Intermediate C instead of Intermediate B in Step 4. MS (m/z): 742.9 [M+H]+. ¹H NMR (400 MHz, MeOD) δ 8.69-8.50 (m, 1H), 7.57-7.40 (m, 1H), 7.07-6.89 (m, 2H), 5.93 (d, J=6.6 Hz, 1H), 5.78-5.58 (m, 1H), 5.06-4.87 (m, 1H), 4.83 (t, J=5.6 Hz, 3H), 4.75-4.54 (m, 3H), 4.35-4.13 (m, 1H), 3.98-3.66 (m, 4H), 3.11-2.98 (m, 3H), 2.98-2.64 (m, 2H), 2.12-1.86 (m, 3H), 1.83-1.51 (m, 3H), 1.42 (s, 2H), 1.35-1.21 (m, 4H), 1.07-0.92 (m, 2H).

### Example 75: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2S,3R)-3-(phosphonooxy)pentan-2-yl) carbonate (75)

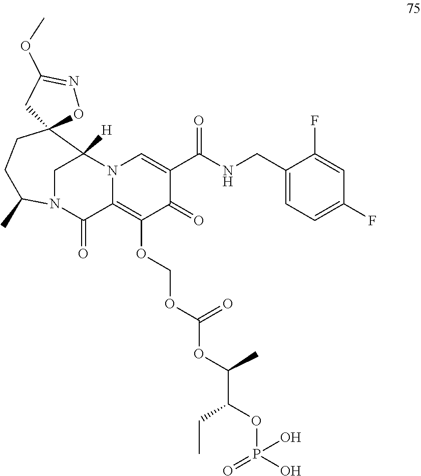 [SMILES: CC[C@@H](OP(=O)(O)O)[C@H](C)OC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared following a similar method as Example 73 except using Intermediate C instead of Intermediate B in Step 4. MS (m/z): 726.9 [M+H]. ¹H NMR (400 MHz, MeOD) δ 8.62 (s, 1H), 7.52-7.36 (m, 1H), 7.07-6.86 (m, 2H), 5.93 (d, J=6.5 Hz, 1H), 5.69 (d, J=6.5 Hz, 1H), 4.75-4.49 (m, 5H), 4.26 (s, 1H), 3.99-3.73 (m, 5H), 3.23-3.06 (m, 1H), 3.07-2.85 (m, 2H), 2.77 (d, J=17.1 Hz, 1H), 2.14-1.88 (m, 4H), 1.81-1.65 (m, 2H), 1.65-1.53 (m, 1H), 1.35-1.20 (m, 6H), 1.01 (t, J=7.4 Hz, 3H).

### Intermediates P and Q: Preparation of (2R,3R)-3-((bis(benzyloxy)phosphoryl)oxy)pentan-2-yl (chloromethyl) carbonate (Intermediate P) and (2R,3S)-3-((bis(benzyloxy)phosphoryl)oxy)pentan-2-yl (chloromethyl) carbonate (Intermediate Q)

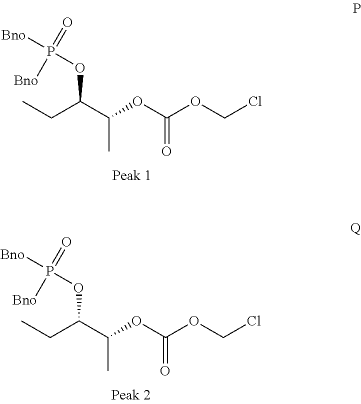

The title compounds were prepared in a manner similar to Intermediate N and Intermediate O, except using (R)-2-((tert-butyldimethylsilyl)oxy)propanal instead of (S)-2-((tert-butyldimethylsilyl)oxy)propanal in step 1.

SFC separation of (2R)-3-((bis(benzyloxy)phosphoryl)oxy)pentan-2-yl (chloromethyl) carbonate (Column: AD-H 5 μm 21×250 mm, 60 mL/min, 5% EtOH—NH₃).

Peak 1: (2R,3R)-3-((bis(benzyloxy)phosphoryl)oxy)pentan-2-yl (chloromethyl) carbonate (Intermediate P): MS (m/z): 456.8 [M+H]⁺.

Peak 2: (2R,3S)-3-((bis(benzyloxy)phosphoryl)oxy)pentan-2-yl (chloromethyl) carbonate (Intermediate Q): MS (m/z): 456.8 [M+H]⁺.

### Example 76: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2R,3R)-3-(phosphonooxy)pentan-2-yl) carbonate (76)

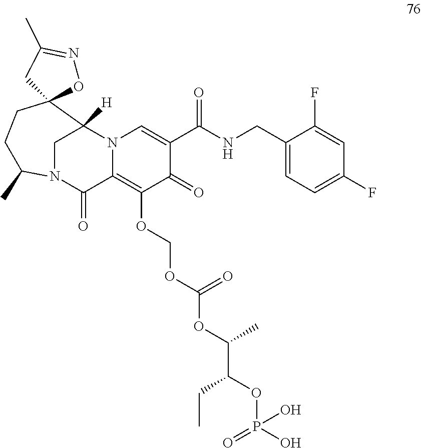 [SMILES: CC[C@@H](OP(=O)(O)O)[C@@H](C)OC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a manner similar to Example 18 except using (2R,3R)-3-((bis(benzyloxy)phosphoryl)oxy)pentan-2-yl (chloromethyl) carbonate (Intermediate P) instead of (R)-1-((bis(benzyloxy)phosphoryl)oxy)propan-2-yl (chloromethyl) carbonate in Step 4. MS (m/z): 726.9 [M+H]+. ¹H NMR (400 MHz, MeOD) δ 8.58 (s, 1H), 7.59-7.30 (m, 1H), 7.14-6.88 (m, 2H), 6.06-5.57 (m, 2H), 4.86-4.55 (m, 3H), 4.48 (s, 1H), 4.35-4.20 (m, 1H), 3.91-3.67 (m, 2H), 3.21-3.12 (m, 2H), 2.95 (d, J=53.0 Hz, 2H), 2.71 (d, J=17.9 Hz, 1H), 2.17-2.01 (m, 4H), 2.01-1.86 (m, 3H), 1.77-1.64 (m, 2H), 1.64-1.47 (m, 1H), 1.34-1.18 (m, 6H), 1.00 (t, J=7.4 Hz, 3H).

### Example 77: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2R,3S)-3-(phosphonooxy)pentan-2-yl) carbonate (77)

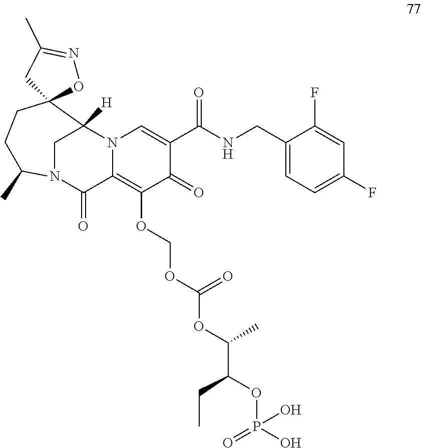 [SMILES: CC[C@H](OP(=O)(O)O)[C@@H](C)OC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a manner similar to Example 18 except using (2R,3S)-3-((bis(benzyloxy)phosphoryl)oxy)pentan-2-yl (chloromethyl) carbonate (Intermediate Q) instead of (R)-1-((bis(benzyloxy)phosphoryl)oxy)propan-2-yl (chloromethyl) carbonate in Step 4. MS (m/z): 726.9 [M+H]. ¹H NMR (400 MHz, MeOD) δ 8.58 (s, 1H), 7.63-7.33 (m, 1H), 7.24-6.82 (m, 2H), 5.89 (d, J=6.6 Hz, 1H), 5.75 (d, J=6.7 Hz, 1H), 5.00-4.90 (m, 1H), 4.75-4.45 (m, 4H), 4.20 (s, 1H), 3.87-3.57 (m, 3H), 3.22-3.09 (m, 1H), 2.95 (d, J=52.9 Hz, 1H), 2.72 (d, J=17.9 Hz, 1H), 2.09-2.03 (m, 4H), 2.00-1.85 (m, 3H), 1.85-1.47 (m, 2H), 1.37-1.22 (m, 7H), 1.07-0.94 (m, 3H).

### Example 78: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2R,3R)-3-(phosphonooxy)pentan-2-yl) carbonate (78)

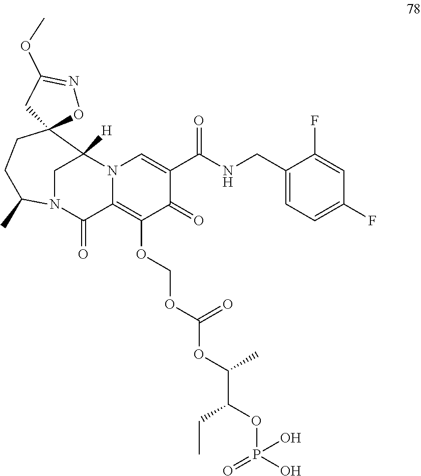 [SMILES: CC[C@@H](OP(=O)(O)O)[C@@H](C)OC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared following a similar method as Example 76 except using Intermediate C instead of Intermediate B in Step 4. MS (m/z): 742.9 [M+H]⁺. ¹H NMR (400 MHz, MeOD) δ 8.63 (s, 1H), 7.61-7.36 (m, 1H), 7.12-6.59 (m, 2H), 5.95-5.72 (m, 2H), 4.87-4.74 (m, 1H), 4.71-4.55 (m, 3H), 4.35-4.18 (m, 1H), 3.96-3.87 (m, 3H), 3.89-3.69 (m, 2H), 3.18 (d, J=17.1 Hz, 1H), 2.95 (d, J=53.0 H z, 2H), 2.78 (d, J=17.1 Hz, 1H), 2.13-1.90 (m, 4H), 1.79-1.50 (m, 3H), 1.36-1.21 (m, 7H), 1.00 (t, J=7.4 Hz, 3H).

### Example 79: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((2R,3S)-3-(phosphonooxy)pentan-2-yl) carbonate (79)

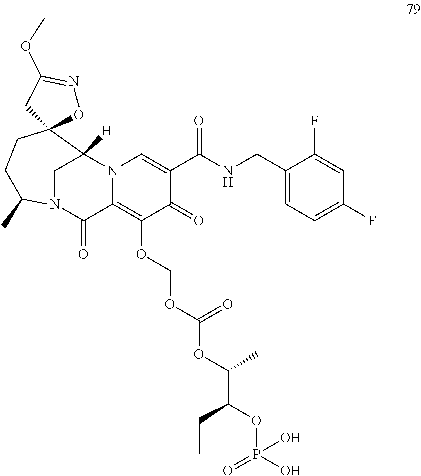 [SMILES: CC[C@H](OP(=O)(O)O)[C@@H](C)OC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared following a similar method as Example 77 except using Intermediate C instead of Intermediate B in Step 4. MS (m/z): 742.9 [M+H]⁺. ¹H NMR (400 MHz, MeOD) δ 8.62 (s, 1H), 7.49-7.40 (m, 1H), 7.09-6.82 (m, 2H), 5.89 (d, J=6.6 Hz, 1H), 5.74 (d, J=6.7 Hz, 1H), 5.03-4.89 (m, 1H), 4.85-4.75 (m, 1H), 4.70-4.57 (m, 3H), 4.28-4.17 (m, 1H), 3.91 (s, 3H), 3.90-3.73 (m, 2H), 3.24-3.12 (m, 1H), 2.95 (d, J=53.0 Hz, 2H), 2.78 (d, J=17.1 Hz, 1H), 2.16-1.84 (m, 3H), 1.84-1.48 (m, 3H), 1.36-1.18 (m, 7H), 1.05-0.92 (m, 3H).

### Intermediates R and S: (R)-2-((bis(benzyloxy)phosphoryl)oxy)-3-methylbutyl (chloromethyl) carbonate (Intermediate R) and (S)-2-((bis(benzyloxy)phosphoryl)oxy)-3-methylbutyl (chloromethyl) carbonate (Intermediate S)

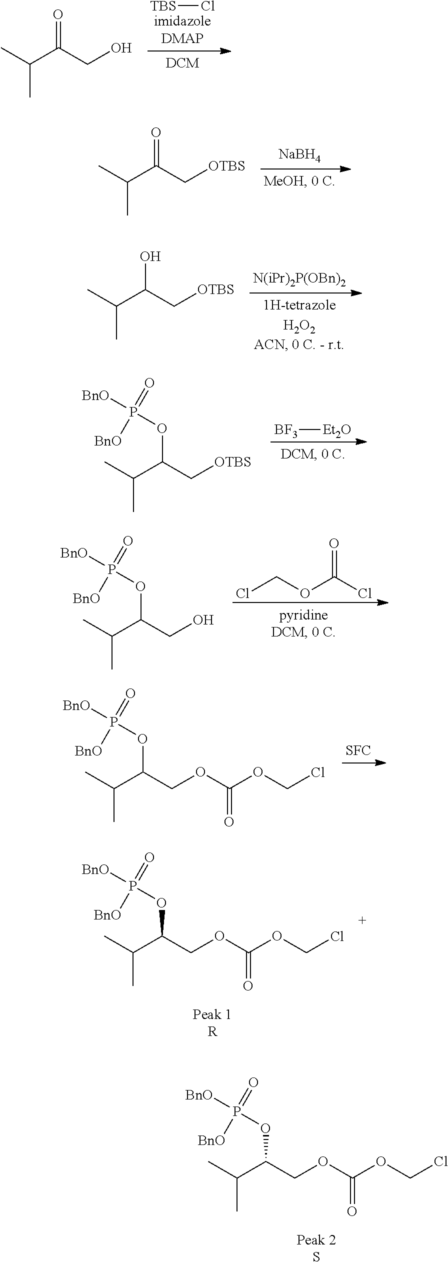

### Step 1: Synthesis of 1-((tert-butyldimethylsilyl)oxy)-3-methylbutan-2-one

To a solution of 1-hydroxy-3-methyl-butan-2-one (1.0 g, 9.79 mmol) in DCM (0.2 M) was added imidazole (800 mg, 11.7 mmol), 4-dimethylaminopyridine (120 mg, 0.97 mmol) and TBS-Cl (1.77 g, 11.7 mmol). The reaction was stirred for 16 h at room temperature. The reaction mixture was then concentrated and purified by silica gel chromatography to afford the title compound. ¹H NMR (400 MHz, CDCl₃) δ 4.28 (s, 2H), 2.92 (hept, J=6.9 Hz, 1H), 1.12 (d, J=6.9 Hz, 6H), 0.95 (s, 9H), 0.11 (s, 6H).

### Step 2: Synthesis of 1-((tert-butyldimethylsilyl)oxy)-3-methylbutan-2-ol

To a solution of 1-((tert-butyldimethylsilyl)oxy)-3-methylbutan-2-one (1.8 g, 8.3 mmol) was in MeOH (0.2 M) at 0° C. was added sodium borohydride (472 mg, 12 mmol) portionwise. The reaction mixture was stirred for 3 h and then quenched with water, extracted with EtOAc, dried, filtered, concentrated and purified by silica gel chromatography (0-100% EtOAc in Hexanes) to afford the title compound. ¹H NMR (400 MHz, CDCl₃) δ 3.75-3.67 (m, 1H), 3.54-3.45 (m, 1H), 3.43-3.31 (m, 1H), 1.76-1.62 (m, 1H), 1.00 (d, J=6.8 Hz, 3H), 0.97-0.86 (m, 9H), 0.15-0.03 (m, 6H).

### Step 3: Synthesis of dibenzyl (1-((tert-butyldimethylsilyl)oxy)-3-methylbutan-2-yl) phosphate

To a solution of 1-((tert-butyldimethylsilyl)oxy)-3-methylbutan-2-ol (900 mg, 4.12 mmol) in acetonitrile (0.1 M) at 0° C. was added 1H-tetrazole (0.35 g, 4.94 mmol) followed by N-dibenzyloxyphosphanyl-N-isopropyl-propan-2-amine (1.74 g, 4.94 mmol). The reaction mixture was stirred at 0° C. for 1 h. Then hydrogen peroxide (30% in water, 0.61 mL, 8.24 mmol) was added dropwise, and the reaction mixture was stirred for an addition 30 min. The reaction was then quenched with saturated sodium thiosulfate and saturated sodium bicarbonate, extracted with DCM, dried over magnesium sulfate, filtered, dried, concentrated and purified by normal phase silica gel chromatography (0-100% EtOAc in Hexanes) to afford the title compound. MS (m/z): 463 [M+(—CH₃)]⁺.

### Step 4: Synthesis of dibenzyl (1-hydroxy-3-methylbutan-2-yl) phosphate

To a solution of dibenzyl (1-((tert-butyldimethylsilyl)oxy)-3-methylbutan-2-yl) phosphate (1.6 g, 3.18 mmol) in DCM (0.3 M) at 0° C. was added boron trifluoride diethyl etherate (1.00 mL, 3.81 mmol) dropwise. The reaction mixture was then stirred at 0° C. for 30 min. The reaction mixture was quenched with saturated sodium bicarbonate, extracted with DCM, dried, concentrated and purified by silica gel chromatography (0-100% EtOAc in Hexanes) to afford the title compound. MS (m/z): 365.0 [M+H]⁺.

### Step 5: Synthesis of 2-((bis(benzyloxy)phosphoryl)oxy)-3-methylbutyl (chloromethyl) carbonate

To a solution of dibenzyl (1-hydroxy-3-methylbutan-2-yl) phosphate (1.3 g, 3.60 mmol) in DCM at 0° C. (0.2 M) was added chloromethyl chloroformate (0.799 mL, 8.99 mmol) followed by pyridine (0.798 mL, 9.89 mmol) dropwise. The reaction mixture was stirred for 30 min. then quenched with water and 1 M HCl. The reaction was extracted with DCM, dried, filtered and concentrated to afford the title compound used directly in the next step without further purification. MS (m/s): 456.9 [M+H].

### Step 6: Synthesis of (R)-2-((bis(benzyloxy)phosphoryl)oxy)-3-methylbutyl (chloromethyl) carbonate (Intermediate R) and (S)-2-((bis(benzyloxy)phosphoryl)oxy)-3-methylbutyl (chloromethyl) carbonate (Intermediate S)

SFC separation of 2-((bis(benzyloxy)phosphoryl)oxy)-3-methylbutyl (chloromethyl) carbonate (Column: AD-H 5 μm 21×250 mm, 60 mL/min, 10% EtOH—NH₃).

Peak 1: (R)-2-((bis(benzyloxy)phosphoryl)oxy)-3-methylbutyl (chloromethyl) carbonate (Intermediate R): MS (m/z): 456.7 [M+H]⁺.

Peak 2: (S)-2-((bis(benzyloxy)phosphoryl)oxy)-3-methylbutyl (chloromethyl) carbonate (Intermediate S): MS (m/z): 456.3 [M+H]⁺.

### Example 80: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((R)-3-methyl-2-(phosphonooxy)butyl) carbonate (80)

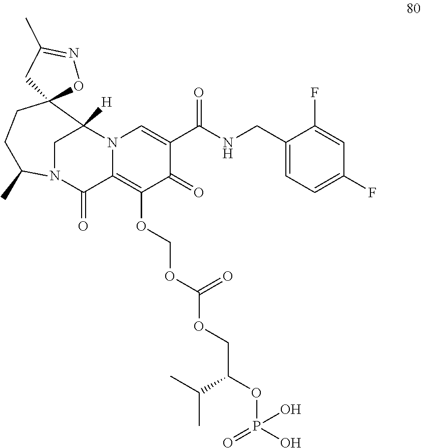 [SMILES: CC1=NO[C@@]2(CC[C@H](C)N3C[C@H]2n2cc(C(=O)NCc4ccc(F)cc4F)c(=O)c(OCOC(=O)OC[C@H](OP(=O)(O)O)C(C)C)c2C3=O)C1]

The title compound was prepared in a manner similar to Example 18 except using (R)-2-((bis(benzyloxy)phosphoryl)oxy)-3-methylbutyl (chloromethyl) carbonate (Intermediate R) instead of (R)-1-((bis(benzyloxy)phosphoryl)oxy)propan-2-yl (chloromethyl) carbonate in Step 4. MS (m/z): 726.8 [M+H]⁺. ¹H NMR (400 MHz, MeOD) δ 8.58 (d, J=2.0 Hz, 1H), 7.55-7.37 (m, 1H), 7.08-6.88 (m, 3H), 6.08-5.67 (m, 2H), 4.76-4.57 (m, 3H), 4.47 (s, 1H), 4.40-4.18 (m, 1H), 4.09 (s, 1H), 3.94-3.69 (m, 2H), 3.23-3.10 (m, 1H), 3.01 (s, 2H), 2.95-2.81 (m, 2H), 2.81-2.61 (m, 1H), 2.07 (s, 3H), 2.01-1.88 (m, 3H), 1.61-1.49 (m, 1H), 1.29-1.24 (m, 3H), 1.03-0.96 (m, 6H).

### Example 81: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((R)-3-methyl-2-(phosphonooxy)butyl) carbonate (81)

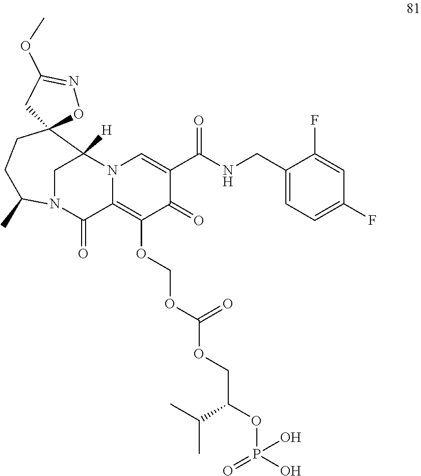 [SMILES: COC1=NO[C@@]2(CC[C@H](C)N3C[C@H]2n2cc(C(=O)NCc4ccc(F)cc4F)c(=O)c(OCOC(=O)OC[C@H](OP(=O)(O)O)C(C)C)c2C3=O)C1]

The title compound was prepared in a manner similar to Example 80 except using Intermediate C instead of Intermediate B, in Step 4. MS (m/z): 742.9 [M+H]⁺. ¹H NMR (400 MHz, MeOD) δ 8.67-8.46 (m, 1H), 7.55-7.36 (m, 1H), 7.04-6.92 (m, 2H), 6.03-5.68 (m, 1H), 4.73-4.55 (m, 3H), 4.37-3.97 (m, 1H), 3.97-3.70 (m, 3H), 3.21-3.12 (m, 1H), 3.11-2.84 (m, 4H), 2.84-2.65 (m, 1H), 2.26-1.83 (m, 7H), 1.62-1.54 (m, 2H), 1.42 (s, 1H), 1.38-1.15 (m, 3H), 1.09-0.90 (m, 6H).

### Example 82: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((S)-3-methyl-2-(phosphonooxy)butyl) carbonate (82)

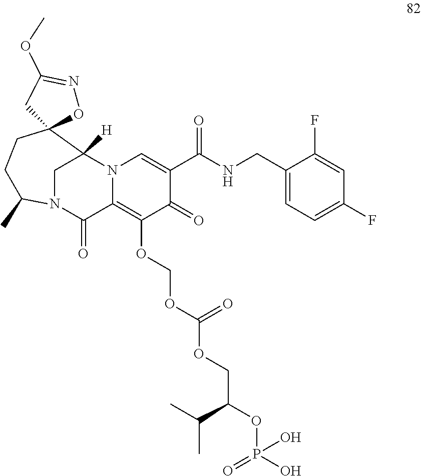 [SMILES: COC1=NO[C@@]2(CC[C@H](C)N3C[C@H]2n2cc(C(=O)NCc4ccc(F)cc4F)c(=O)c(OCOC(=O)OC[C@@H](OP(=O)(O)O)C(C)C)c2C3=O)C1]

The title compound was prepared in a manner similar to Example 80 except using Intermediate C instead of Intermediate B, in Step 4. MS (m/z): 742.8 [M+H]⁺. ¹H NMR (400 MHz, MeOD) δ 8.62 (s, 1H), 7.46 (s, 1H), 7.07-6.85 (m, 2H), 5.90 (d, J=6.2 Hz, 1H), 5.74 (s, 1H), 4.94-4.89 (m, 1H), 4.86-4.76 (m, 1H), 4.72-4.49 (m, 3H), 4.37-4.26 (m, 1H), 4.28-4.12 (m, 2H), 4.01-3.88 (m, 2H), 3.87-3.57 (m, 3H), 2.85-2.54 (m, 2H), 2.24 (s, 1H), 2.17-1.75 (m, 3H), 1.67-1.49 (m, 1H), 1.42 (s, 3H), 1.36-1.19 (m, 4H), 1.08-0.86 (m, 4H).

### Example A: HIV MT-4 Antiviral and Cytotoxicity Assay

### Antiviral Assay in MT-4 Cells

Compounds were tested in a high-throughput 384-well assay format for their ability to inhibit the replication of HIV-1 (IIIB) in MT-4 cells. Compounds were serially diluted (1:3) in DMSO on 384-well polypropylene plates and further diluted 200-fold into complete RPMI media (10% FBS, 1% P/S) using the Biotek Micro Flow and Labcyte ECHO acoustic dispenser. Each plate contained up to 8 test compounds, with negative (No Drug Control) and 5 M AZT positive controls. MT-4 cells were pre-infected with 10 μL of either RPMI (mock-infected) or a fresh 1:250 dilution of HIV-1 IIIB concentrated virus stock. Infected and uninfected MT-4 cells were further diluted in complete RPMI media and added to each plate using a Micro Flow dispenser. After 5 days incubation in a humidified and temperature controlled incubator (37° C.), Cell Titer Glo (Promega) was added to the assay plates and chemiluminescence read using an Envision plate-reader. EC₅₀ values were defined as the compound concentration that causes a 50% decrease in luminescence signal, and were calculated using a sigmoidal dose-response model to generate curve fits. The EC₅₀ data for exemplary compounds is shown in Table 1.

### Cytotoxicity Assay in MT-4 Cells

Assays were performed as above except uninfected MT-4 cells were added to each well containing test compound. In addition, 10 μM puromycin was added to the last column of each assay plate to assess a base level of cytotoxicity. The CC₅₀ data for exemplary compounds is shown in Table 1.

TABLE 1

| | MT4 EC₅₀ | MT4 CC₅₀ |
|---|---|---|
| Compound No. | (nM) | (nM) |
| 1 | 1.77 | 15429 |
| 2 | 1.37 | 12251 |
| 3 | 0.46 | 13012 |
| 4 | 0.95 | 11556 |
| 5 | 1.96 | 10608 |
| 6 | 2.09 | 15182 |
| 7 | 0.68 | 15305 |
| 8 | 2.04 | 17793 |
| 9 | 2.79 | 12251 |
| 10 | 2.08 | 12408 |
| 11 | 0.53 | 9751.9 |
| 12 | 2.01 | 13975 |
| 13 | 1.98 | 13953 |
| 14 | 1.27 | 17142 |
| 15 | 4.13 | 17332 |
| 16 | 1.25 | 14118 |
| 17 | 2.05 | 13695 |
| 18 | 2.27 | 12105 |
| 19 | 2.35 | 12228 |
| 20 | 2.37 | 20383 |
| 21 | 1.50 | 15152 |
| 22 | 1.93 | 9776 |
| 23 | 2.33 | 14825 |
| 24 | 2.29 | 17094 |
| 25 | 2.21 | 13939 |
| 26 | 2.71 | 13454 |
| 27 | 2.47 | 18747 |
| 28 | 4.85 | 28949 |
| 29 | 4.87 | 28956 |
| 30 | 2.72 | 10251 |
| 31 | 3.50 | 12919 |
| 32 | 2.92 | 17748 |
| 33 | 2.17 | 12319 |
| 34 | 10.58 | 50000 |
| 35 | 1.85 | 9633.8 |
| 36 | 1.22 | 9328.7 |
| 37 | 1.49 | 11097 |
| 38 | 1.45 | 17557 |
| 39 | 1.60 | 16135 |
| 40 | 1.26 | 12570 |
| 41 | 3.09 | 44340 |
| 42 | 1.06 | 10276 |
| 43 | 1.77 | 8455.3 |
| 44 | 1.41 | 11143 |
| 45 | 89.68 | 50000 |
| 46 | 33.66 | 50000 |
| 47 | 100.00 | 50000 |
| 48 | 1.94 | 15272 |
| 49 | 1.63 | 17498 |
| 50 | 1.75 | 17805 |
| 52 | 2.13 | 10580 |
| 53 | 1.96 | 15155 |
| 54 | 4.32 | 15475 |
| 57 | 2.32 | 19940 |
| 58 | 2.11 | 24201 |
| 59 | 2.04 | 18824 |
| 60 | 1.90 | 23786 |
| 61 | 2.35 | 20853 |
| 62 | 2.58 | 28088 |
| 63 | 5.12 | 35409 |
| 64 | 1.24 | 13574 |
| 65 | 1.02 | 14146 |
| 66 | 2.07 | 12766 |
| 67 | 2.41 | 14422 |
| 68 | 2.11 | 16071 |
| 69 | 2.90 | 17317 |
| 70 | 2.86 | 15284 |
| 71 | 5.04 | 21331 |
| 72 | | 27179 |
| 73 | | 30756 |
| 74 | | 26574 |
| 75 | | 34281 |
| 76 | | 24778 |
| 77 | | 36973 |
| 78 | | 28570 |
| 79 | | 45683 |
| 80 | | 27203 |
| 81 | | 36933 |
| 82 | | 39664 |

### Example B: Thermodynamic Solubility Assay

Approximately 7 mg of each test compound as a dry powder was placed in a vial. Aliquots were weighed out for each assay media at 2 hour and 24 hour time points, to be analyzed. The appropriate buffer (FaSSIF or PBS) was added to each vial such that the final dose concentration of 5 mg/mL was achieved. Samples were then vortexed for 5-10 seconds. Following a 2-hour or a 24-hour incubation on a rotary shaker (200 RPM) at ambient temperature (22.3-23.8° C.), the samples were vacuum filtered through a Millipore solubility filter plate with 0.45 μM polycarbonate filter membrane and the filtrates were collected in a 96 well polypropylene plate. The plate was sealed with a pierceable heat seal and analyzed by HPLC-UV.

An Agilent 1290 UHPLC equipped with a micro-well plate autosampler, quaternary HPLC pump, and diode array detector was used for analysis. Each filtrate (1.5 μL) was injected onto the column (AQUASIL C18, 5 μM, 50×2.1 mm) and eluted using a gradient of 10-100% MeCN/water containing 0.1% formic acid. A system QC (0.01 mg/mL Caffeine in DMSO) was injected every 12-15 injections. Data was collected at 214, 254 and 280 nm. Results are reported using data obtained at 280 nm. The resulting peak areas were plotted against the known concentrations from the calibration and the filtrates are quantified with respect to the linear regression using Agilent OpenLab Intelligent Reporting Software. The results are reported electronically in mg/mL.

Solubility data for exemplary compounds is shown in Table 2. Comparative solubility data for Intermediate B and Intermediate C is also included in Table 2.

TABLE 2

| | Thermodynamic FaSSIF |
|---|---|
| Example No. | Solubility @ 24 h (mg/mL) |
| 3 | 6.65 |
| 4 | 4.30 |
| 5 | 0.17 |
| 7 | 3.84 |
| 8 | 3.02 |
| 9 | 2.87 |
| 10 | 4.69 |
| 11 | 5.09 |
| 12 | 5.28 |
| 13 | 4.38 |
| 15 | 4.37 |
| 16 | 2.23 |
| 17 | 4.59 |
| 19 | 5.058 |
| 21 | 5.04 |
| 22 | 4.185 |
| 23 | 4.4 |
| 24 | 5.005 |
| 25 | 5.13 |
| 26 | 4.765 |
| 27 | 4.665 |
| 28 | 4.405 |
| 29 | 4.88 |
| 31 | 4.625 |
| 32 | 4.805 |
| 33 | 5.68 |
| 34 | 4.89 |
| 35 | 4.66 |
| 36 | 5.055 |
| 37 | 4.985 |
| 38 | 2.455 |
| 39 | 2.655 |
| 40 | 5.345 |
| 41 | 5.595 |
| 42 | 5.08 |
| 43 | 5.415 |
| 46 | 4.985 |
| 47 | 0.285 |
| 48 | 3.825 |
| 49 | 1.57 |
| 52 | 5.16 |
| 54 | 4.655 |
| 55 | 4.6 |
| 56 | 4.245 |
| 57 | 4.63 |
| 58 | 4.86 |
| 59 | 5.235 |
| 60 | 4.52 |
| 61 | 4.795 |
| 62 | 5.045 |
| 63 | 4.625 |
| 64 | 4.845 |
| 65 | 3.885 |
| 66 | 4.27 |
| 67 | 5.185 |
| 68 | 4.625 |
| 69 | 4.6 |
| Intermediate B | 0.002 |
| Intermediate C | 0.002 |

FaSSIF = Fasted-state simulated intestinal fluid.

### Example C: Dog PO Pharmacokinetic Studies (without P-Gp Inhibitor)

Doses are expressed as “mg-eq”, referring to the mass (in mg) of Intermediate B (for Compound 4) or Intermediate C (for Compound 3) equivalents (eq) as a fixed dose or relative to body weight (in kg).

### Dog PK Dosing

Male beagle dogs were fasted overnight. Food was returned approximately 4 hours postdose. Each animal received a single 6 μg/kg intramuscular injection of pentagastrin approximately 30 minutes prior to test article administration to stimulate gastric secretion. The intramuscular dose was administered in a thigh muscle using a needle and syringe.

Solution or suspension oral doses were administered at a dose of 4 mg-eq/kg via gavage and the dosing tube was flushed with ˜10 mL water prior to removal. Individual doses were calculated based on body weights recorded on the day of dose administration. Solid or capsule oral doses were administered at a dose of 40 mg-eq fixed, by hand by deep throat deposition. Following each dose, the animals were offered approximately 5 mL of water to assist in swallowing. This was done by depositing ˜1-3 mL at a time into the back of the throat and holding the mouth closed until swallowing was observed. This was repeated until all 5 mL of water has been given. Once swallowed, 25 mL water was administered by gavage for a total of 30 mL water administered with each dose. All animals were observed at dosing and each scheduled collection. For tablet formulations, the specified dose was administered orally followed by approximately 10 mL of water via syringe. This was followed by an additional 20 mL of water administered via oral gavage. The total flush volume was approximately 30 mL. Individual doses were administered on a fixed basis. In all cases, food was returned at approximately 4 hours after dosing. All animals were observed at dosing and each scheduled collection.

Serial blood samples (through 168 h) were collected into pre-chilled K₂EDTA with the appropriate volume of 40 mM dichlorvos added to result in a final dichlorvos concentration of 2 mM and stored on wet ice until processed. Whole blood was processed to plasma by centrifugation (3500 rpm for 10 minutes at 5° C.) within 30 minutes of collection. Plasma samples were transferred into Micronic 96 well tubes and stored at −70° C. as soon as possible and remained at −70° C. until shipped for bioanalysis.

### Bioanalysis of Plasma Samples

To a 20 μL aliquot of each plasma sample with exception of the matrix blanks, 120 μL of 100 ng/mL Carbamazepine and Chrysin in acetonitrile (ACN) was added. The matrix blank samples received 120 μL of acetonitrile only. The precipitated proteins were removed by centrifugation and 100 μL of supernatant was transferred into a clean 96-well plate. A 100 μL aliquot of water was added to each sample. An aliquot of 2-2.5 μL was injected into an Applied Biosystems API-6500 LC/MS/MS system, eluting with a gradient of water and acetonitrile (containing 0.1% formic acid).

AUC_{inf} was calculated as area under the plasma concentration vs. time curve from 0 h to infinity.

Bioavailability (% F) was calculated by comparing plasma concentration via PO dose (oral) vs. plasma concentration via IV dose (intravenous) using the following equation:

$$\% F = \frac{PO AUCinf}{IV AUCinf} \times \frac{IV Dose}{PO Dose} \times 100$$

### Example D: Dog IV Pharmacokinetic Studies

Three male beagle dogs were fasted overnight. Each animal was administered an intravenous dose of the test compound in the formulation indicated in Table 3 as an approximately 30-minute infusion via an indwelling catheter in a cephalic vein. After dose administration, but prior to removal from the animal, the dose apparatus (i.e., the indwelling catheter) was flushed with approximately 1 mL of saline. Individual doses were calculated based on body weights recorded on the day of dose administration. Food returned at approximately 4 hours after dosing.

Serial blood samples (through 168 h) were collected into pre-chilled K₂EDTA with the appropriate volume of 40 mM dichlorvos added to result in a final dichlorvos concentration of 2 mM and stored on wet ice until processed. Whole blood was processed to plasma by centrifugation (3500 rpm for 10 minutes at 5° C.) within 30 minutes of collection. Plasma samples were transferred into Micronic 96 well tubes and stored at −70° C. as soon as possible and remained at −70° C. until shipped for bioanalysis.

To a 20 μL aliquot of each plasma sample with exception of the matrix blanks, 120 μL of 100 ng/mL Carbamazepine and Chrysin in acetonitrile (ACN) was added. The matrix blank samples received 120 μL of acetonitrile only. The precipitated proteins were removed by centrifugation and 100 μL of supernatant was transferred into a clean 96-well plate. A 100 μL aliquot of water was added to each sample. An aliquot of 2-2.5 μL was injected into an Applied Biosystems API-6500 LC/MS/MS system, eluting with a gradient of water and acetonitrile (containing 0.1% formic acid). AUC_{inf} was calculated as area under the plasma concentration vs. time curve from 0 h to infinity.

### Example E: Dog PO Pharmacokinetic Studies (With P-gp Inhibitor)

Three male beagle dogs were fasted overnight. Each animal was administered a 5 mg-eq/kg oral dose of encequidar mesylate immediately followed by a 0.1 mL/kg intramuscular injection of pentagastrin (60 μg/mL, 6 μg/kg) approximately 30 minutes prior to oral administration of the test compound. Encequidar mesylate was administered as a solution formulation. For solution or suspension formulations, prior to withdrawing the gavage tube, the tube was flushed with approximately 10 mL of water. Individual doses were calculated based on body weights recorded on the day of dose administration. For tablet formulations, the dose was administered orally followed by approximately 10 mL of water via syringe. This was followed by an additional 20 mL of water administered via oral gavage. The total flush volume was approximately 30 mL. Individual doses were administered on a fixed basis. In all cases, food was returned at approximately 4 hours after dosing. All animals were observed at dosing and each scheduled collection. Bioanalysis was carried out according to Example C.

AUC_{inf} was calculated as area under the plasma concentration vs. time curve from 0 h to infinity. Bioavailability (% F) was calculated by comparing plasma concentration via PO dose (oral) vs. plasma concentration via IV dose (intravenous) using the following equation:

$$\% F = \frac{PO AUCinf}{IV AUCinf} \times \frac{IV Dose}{PO Dose} \times 100$$

Doses expressed as “mg-eq”, refer to the mass (in mg) of parent compound equivalents (eq) as a fixed dose or relative to body weight (in kg).

### PK Data:

TABLE 3
Dog IV PK Data of Example D

| | Dog | Compound | IV Dose | Compound | AUC_{INF} |
|---|---|---|---|---|---|
| Entry | Group # | Dosed | (mg/kg) | Measured | (μM · h) |
| 1 | Group 1 | Intermediate B | 1.0 | Intermediate B | 1400 ± 226 |
| 2 | Group 2 | Intermediate C | 1.0 | Intermediate C | 707 ± 98 |
| 3 | Group 3 | Intermediate C | 0.5 | Intermediate C | 250 ± 42 |
| 4 | Group 4 | Intermediate C | 1.0 | Intermediate C | 722 ± 98 |

TABLE 4
Dog PO PK Data of Example C (without P-gp inhibitor)

| | Dog | Compound | | | Compound | AUC_{INF} | Cmax | |
|---|---|---|---|---|---|---|---|---|
| Entry | Group # | Dosed | PO Dose | | Measured | (μM · h) | (μM) | % F |
| 1 | Group 1 | Intermediate B | 4 | mg/kg | Intermediate B | 1940 ± 435 | 39.6 ± 3.8 | 35.3 ± 10.0 |
| 2 | Group 2 | Intermediate C | 4 | mg/kg | Intermediate C | 1040 ± 177 | 28.5 ± 2.0 | 36.4 ± 1.6 |
| 3 | Group 3 | Compound 3 | 300 | mg-eq fixed | Intermediate C | 1860 ± 470 | 74.7 ± 21.4 | 12.9 ± 3.4 |

TABLE 5
Dog PO PK Data of Example E (with P-gp inhibitor)

| | Dog | Compound | | | Compound | AUC_{INF} | Cmax | |
|---|---|---|---|---|---|---|---|---|
| Entry | Group # | Dosed | PO Dose | | Measured | (μM · h) | (μM) | % F |
| 1 | Group 1 | Intermediate B | 4 | mg/kg | Intermediate B | 3980 ± 762 | 80.8 ± 4.9 | 70.3 ± 1.8 |
| 2 | Group 2 | Intermediate C | 4 | mg/kg | Intermediate C | 2300 ± 171 | 62.0 ± 3.5 | 82.3 ± 13.4 |
| 3 | Group 4 | Compound 3 | 300 | mg-eq fixed | Intermediate C | 9370 ± 72.2 | 261 ± 21.7 | 45.8 ± 11.3 |

TABLE 6
Oral Bioavailability of Intermediate B or Intermediate C
in Dog Following a 4 mg-eq/kg Solution Dose of
Test Compound According to Example C.

| | | | Dog % F of Intermediate |
|---|---|---|---|
| | Compound | Compound | B or Intermediate C |
| Entry | Dosed | Measured | (4 mg-eq/kg solution) |
| 1 | 3 | Intermediate C | 11 |
| 2 | 7 | Intermediate B | 22.2 |

TABLE 7
Oral Bioavailability of Intermediate B or Intermediate C
in Dog Following a 40 mg-eq Powder-in-Capsule Fixed
Dose of Test Compound According to Example C.

| | | | Dog % F of Intermediate |
|---|---|---|---|
| | Compound | Compound | B or Intermediate C |
| Entry | Dosed | Measured | (40 mg-cq PIC) |
| 1 | 3 | Intermediate C | 31 |
| 2 | 7 | Intermediate B | 23.7 |
| 3 | 10 | Intermediate B | 18.6 |
| 4 | 12 | Intermediate B | 19.1 |

TABLE 8
Oral Bioavailability of Intermediate B or Intermediate C
in Dog Following a 175 mg-eq Tablet Fixed
Dose of Test Compound According to Example C.

| | | | Dog % F of Intermediate |
|---|---|---|---|
| | Compound | Compound | B or Intermediate C |
| Entry | Dosed | Measured | (175 mg-eq Tablet) |
| 1 | 3 | Intermediate C | 20.7 |
| 2 | 12 | Intermediate B | 36.1 |
| 3 | 17 | Intermediate B | 29.3 |
| 4 | 18 | Intermediate B | 33.2 |
| 5 | 19 | Intermediate B | 28.5 |
| 6 | 22 | Intermediate B | 25.5 |
| 7 | 23 | Intermediate C | 37.3 |
| 8 | 28 | Intermediate B | 38.8 |
| 9 | 29 | Intermediate C | 30.5 |
| 10 | 32 | Intermediate C | 28.6 |
| 11 | 42 | Intermediate B | 9.1 |
| 12 | 50 | Intermediate B | 4.6 |

TABLE 9
Oral Bioavailability of Intermediate B or Intermediate C in Dog
Following a 300 mg-eq Powder-in-Capsule or Tablet Fixed
Dose of Test Compound According to Example C.

| | | | Dog % F of Intermediate |
|---|---|---|---|
| | Compound | Compound | B or Intermediate C |
| Entry | Dosed | Measured | (300 mg-eq PIC or Tablet) |
| 1 | 3 | Intermediate C | 12.9 |
| 2 | 4 | Intermediate B | 13.1 |
| 3 | 8 | Intermediate B | 8.3 |

TABLE 10
Oral Bioavailability of Intermediate B or Intermediate C
in Dog Following a 350 mg-eq Tablet Fixed Dose
of Test Compound According to Example C.

| | | | Dog % F of Intermediate |
|---|---|---|---|
| | Compound | Compound | B or Intermediate C |
| Entry | Dosed | Measured | (350 mg-eq Tablet) |
| 1 | 17 | Intermediate B | 31.7 |
| 2 | 23 | Intermediate C | 24.7 |
| 3 | 28 | Intermediate B | 35.7 |
| 4 | 29 | Intermediate C | 38 |

TABLE 11
Oral Bioavailability of Intermediate B or Intermediate C in Dog
Following a 175 mg Tablet Fixed Dose According to Example C.

| Entry | Compound Dosed | Dog % F (175 mg Tablet) |
|---|---|---|
| 1 | Intermediate B | 9.2 |
| 2 | Intermediate C | 2.9 |

The compounds disclosed herein generally show improved oral bioavailability of Intermediate B or Intermediate C in dogs relative to when Intermediate B or Intermediate C is dosed.

The PK results in Tables 4 and 5 demonstrate that administration of a PGP inhibitor (e.g. encequidar mesylate) in combination with a compound described herein (e.g. Compound 3, Intermediate B or Intermediate C) increases the exposure, maximum plasma concentration, and oral bioavailability of the compound or its metabolite. For example, AUC_{inf}, C_{MAX} and oral bioavailability of Intermediate C are increased when its prodrug Compound 3 is administered in combination with encequidar mesylate (comparing Table 5 Entry 3 with Table 4 Entry 3). AUC_{inf}, C_{MAX} and oral bioavailability of Intermediate C is increased when Intermediate C is administered with encequidar mesylate (comparing Table 5 Entry 2 with Table 4 Entry 2). AUC_{inf}, C_{MAX} and oral bioavailability of Intermediate B is increased when Intermediate B is administered with encequidar mesylate (comparing Table 5 Entry 1 with Table 4 Entry 1).

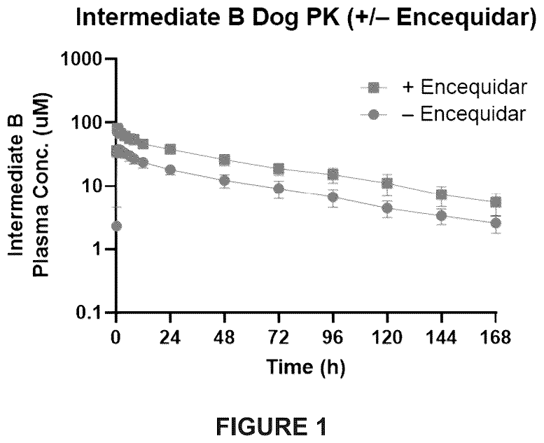

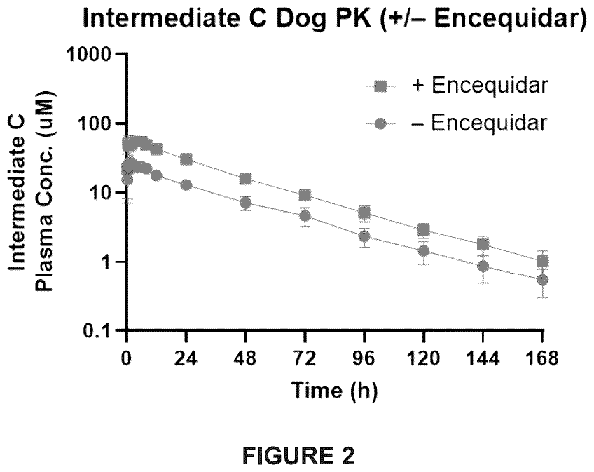

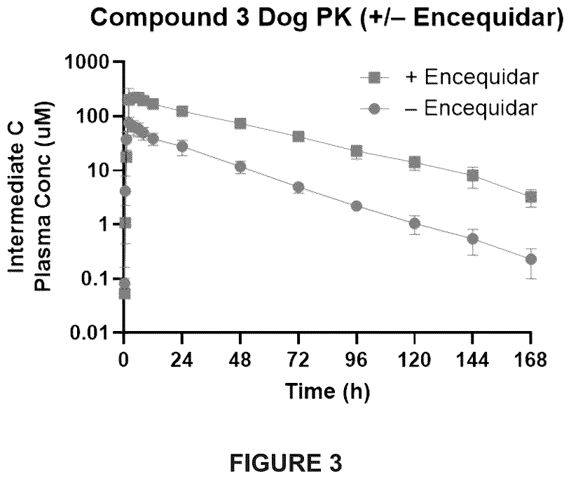

The increase in exposure and oral bioavailability of a compound described herein when coadministered with a PGP inhibitor are also illustrated in the Figures. In particular, FIG. 1 shows that the AUCinf, Cmax, and oral bioavailability of Intermediate B are increased when beagle dogs are pretreated with encequidar mesylate prior to oral administration of Intermediate B compared to when they are not pretreated with encequidar mesylate. FIG. 2 shows that the AUCinf, Cmax, and oral bioavailability of Intermediate C are increased when beagle dogs are pretreated with encequidar mesylate prior to oral administration of Intermediate C compared to when they are not pretreated with encequidar mesylate. Lastly, FIG. 3 shows that the AUCinf, Cmax, and oral bioavailability of Intermediate C are increased when beagle dogs are pretreated with encequidar mesylate prior to oral administration of Compound 3 compared to when they are not pretreated with encequidar mesylate.

All of the U.S. patents, U.S. patent application publications, U.S. patent applications, foreign patents, foreign patent applications and non-patent publications referred to in this specification are incorporated herein by reference, in their entirety to the extent non inconsistent with the present description.

From the forgoing it will be appreciated that, although specific embodiments have been described herein for purposes of illustration, various modifications may be made without deviating from the spirit and scope of the disclosure. Accordingly, the disclosure is not limited except as by the appended claims.

1. A compound of Formula I: 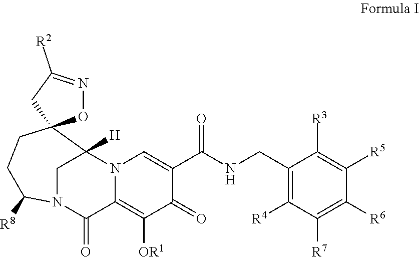
or a pharmaceutically acceptable salt thereof, wherein:
R¹ is —(CR^{1A}R^{1B}O)ₐ(Y)_{b}(CR^{1C}R^{1D})_{d}X;
wherein a is 0 or 1;
b is 0 or 1;
d is 0, 1, 2, 3, 4 or 5;
R^{1A} is H or C₁₋₃alkyl;
R^{1B} is H or C₁₋₃alkyl;
each R^{1C} is independently H or C₁₋₃alkyl;
each R^{1D} is independently H or C₁₋₃alkyl; or
optionally R^{1C} and R^{1D} on the same carbon atom are joined to form a spiro cyclopropyl group;
Y is —C(O)—, —C(O)O—, —C(O)NH— or —C(O)NR^{1H}—;
R^{1H} is C₁₋₄alkyl optionally substituted with one or two substituents independently selected from the group consisting of —COOH, —OH, —NH₂, —CONH₂, —P(O)(OH)₂, and —S(O)₂(OH);
X is selected from the group consisting of:
(a) —O—P(O)(OR^{1E})₂,
wherein each R^{1E} is independently H or phenyl;
(b) —N(RIF)₂,
wherein each R^{1F} is independently H, COO(CR^{1I}R^{1J})ₑOPO(OH)₂, or C₁₋₄alkyl; wherein the C₁₋₄ alkyl is optionally substituted with one or two substituents independently selected from the group consisting of —COOH, —S(O)₂(OH), —P(O)(OH)₂, —OH, —NH₂, and —CONH₂;
e is 1, 2, or 3;
each R^{1I} is independently H or C₁₋₃alkyl;
each R^{1J} is independently H or C₁₋₃alkyl; or
optionally R^{1I} and R^{1J} on the same carbon atom are joined to form a spiro cyclopropyl group; and
(c) —N⁺(R^{1G})₃Z⁻,
wherein each R^{1G} is independently H or C₁₋₄alkyl; wherein the C₁₋₄ alkyl is optionally substituted with one or two substituents independently selected from the group consisting of —COOH, —OH, —NH₂, and —CONH₂;
Z⁻ is a counterion;
R² is C₁₋₃ alkyl or C₁₋₃ alkoxy;
each R³, R⁴, R⁵, R⁶ and R⁷ is independently H or halo; and
R⁸ is H or C₁₋₃alkyl.

2. The compound of claim 1, or the pharmaceutically acceptable salt thereof, wherein Z— is selected from the group consisting of acetate, ascorbate, aspartate, besylate, benzoate, bromide, bicarbonate, carbonate, cinnamate, citrate, chloride, formate, fumarate, gluconate, glutamate, glycolate, lactate, malate, maleate, malonate, mandelate, mesylate, nicotinate, nitrate, oxalate, dihydrogen phosphate, hydrogen phosphate, phosphate, propionate, tosylate, pyroglutamate, salicylate, succinate, bisulfate, sulfate, tartrate, thiocyanate, triflate, and trifluoroacetate.

3. The compound of claim 1, or the pharmaceutically acceptable salt thereof, wherein X is —O—P(O)(OR^{1E})₂.

4.-5. (canceled)

6. The compound of claim 1, or the pharmaceutically acceptable salt thereof, wherein X is —N(R^{1F})₂.

7. The compound of claim 1, or the pharmaceutically acceptable salt thereof, wherein each R^{1F} is independently —COO(CR^{1I}R^{1J})ₑOPO(OH)₂ or C₁₋₄alkyl; wherein the C₁₋₄ alkyl is optionally substituted with one or two —COOH.

8.-12. (canceled)

13. The compound of claim 6, or the pharmaceutically acceptable salt thereof, wherein each R^{1F} is independently —COO(CH₂)₂OPO(OH)₂, —COOCH₂OPO(OH)₂, —CH₃, or —CH₂COOH.

14. (canceled)

15. The compound of claim 1, or the pharmaceutically acceptable salt thereof, wherein X is —N⁺(R^{1G})₃Z⁻.

16. The compound of claim 15, or the pharmaceutically acceptable salt thereof, wherein R^{1G} is —CH₃.

17.-20. (canceled)

21. The compound of claim 1, or the pharmaceutically acceptable salt thereof, wherein Y is —C(O)—, —C(O)O—, —C(O)NH— or —C(O)NCH₃—.

22.-25. (canceled)

26. The compound of claim 1, or the pharmaceutically acceptable salt thereof, wherein Y is —C(O)NR^{1H}—.

27. The compound of claim 26, or the pharmaceutically acceptable salt thereof, wherein R^{1H} is C₁₋₂alkyl optionally substituted with —COOH, —P(O)(OH)₂ or —S(O)₂(OH).

28.-31. (canceled)

32. The compound of claim 1, or the pharmaceutically acceptable salt thereof, wherein R⁴, R⁵ and R⁷ are each H.

33. The compound of claim 1, or the pharmaceutically acceptable salt thereof, wherein R³ and R⁶ are each independently a halo.

34. (canceled)

35. The compound of claim 1, or the pharmaceutically acceptable salt thereof, wherein R⁸ is C₁₋₃alkyl.

36. (canceled)

37. The compound of claim 1, or the pharmaceutically acceptable salt thereof, wherein R² is methyl or methoxy.

38.-39. (canceled)

40. The compound of claim 1, or the pharmaceutically acceptable salt thereof, wherein R¹ is selected from the group consisting of:

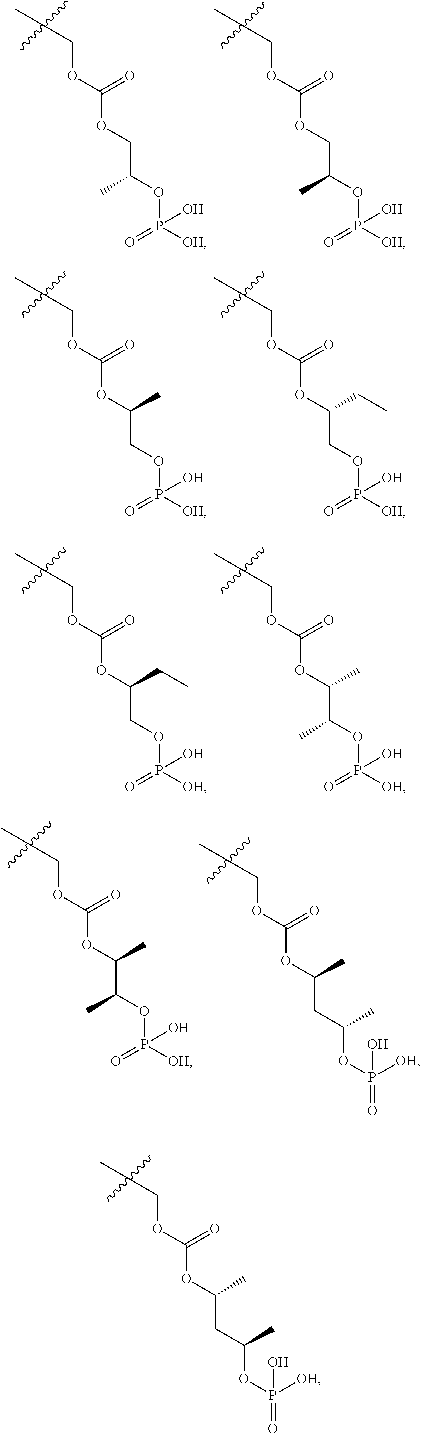

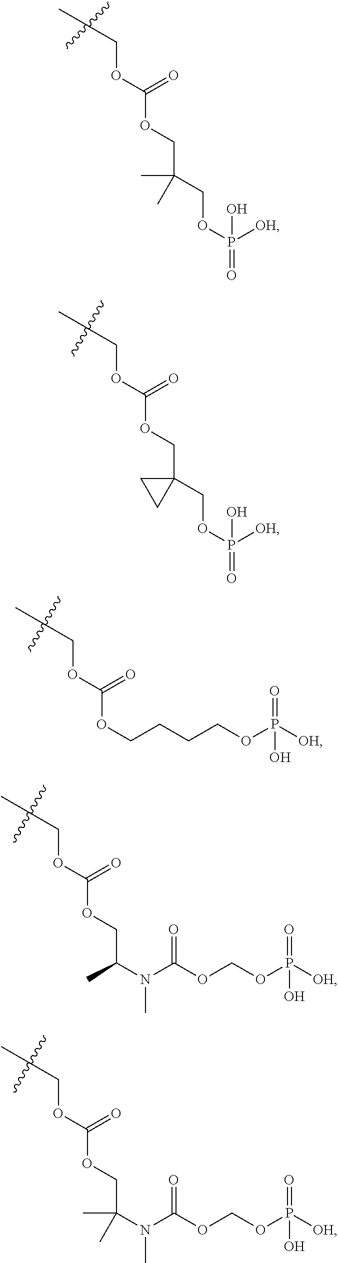 [SMILES: *COC(=O)OCC(C)(C)COP(=O)(O)O ; *COC(=O)OCC1(COP(=O)(O)O)CC1 ; *COC(=O)OCCCCOP(=O)(O)O ; *COC(=O)OC[C@H](C)N(C)C(=O)OCOP(=O)(O)O ; *COC(=O)OCC(C)(C)N(C)C(=O)OCOP(=O)(O)O]

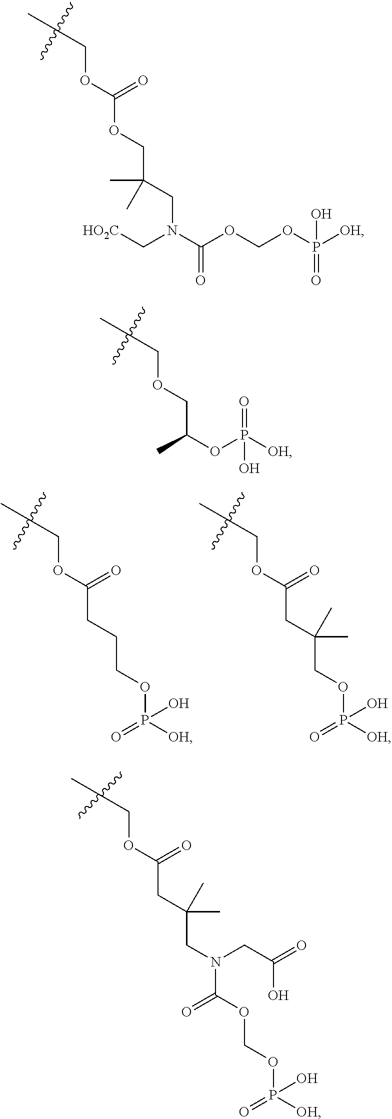 [SMILES: *COC(=O)OCC(C)(C)CN(CC(=O)O)C(=O)OCOP(=O)(O)O ; *COC[C@H](C)OP(=O)(O)O ; *COC(=O)CCCOP(=O)(O)O ; *COC(=O)CC(C)(C)COP(=O)(O)O ; *COC(=O)CC(C)(C)CN(CC(=O)O)C(=O)OCOP(=O)(O)O]

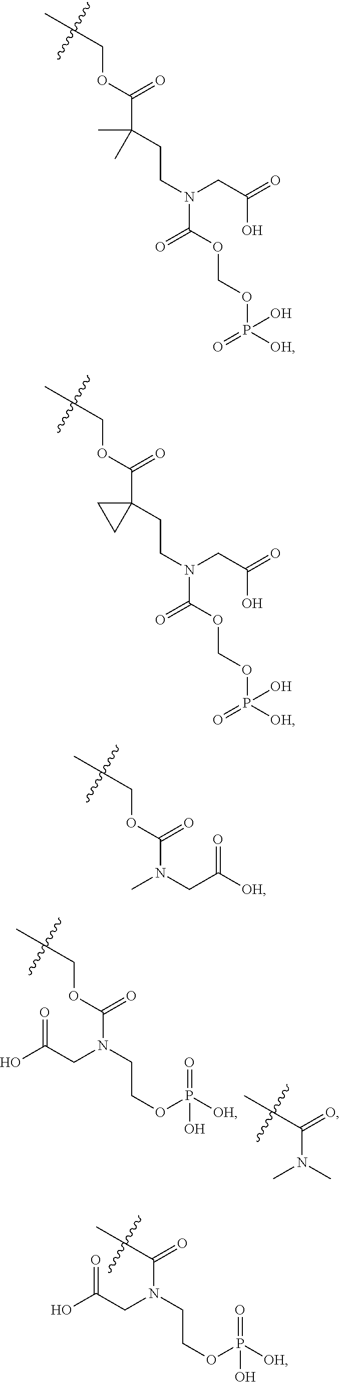

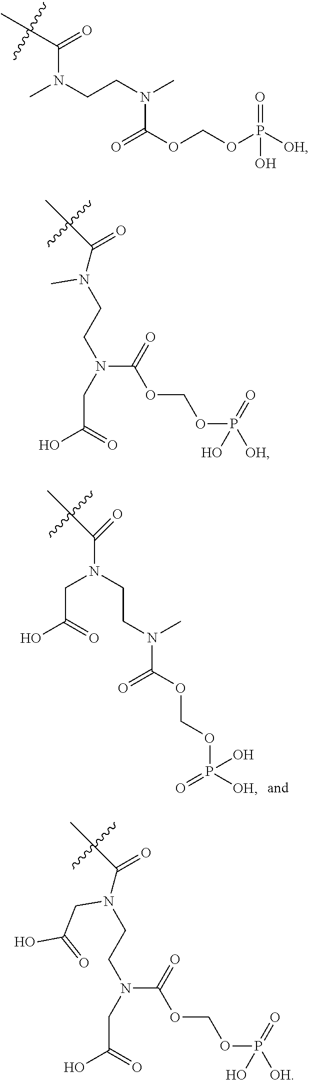 [SMILES: *C(=O)N(C)CCN(C)C(=O)OCOP(=O)(O)O ; *C(=O)N(C)CCN(CC(=O)O)C(=O)OCOP(=O)(O)O ; *C(=O)N(CCN(C)C(=O)OCOP(=O)(O)O)CC(=O)O ; *C(=O)N(CCN(CC(=O)O)C(=O)OCOP(=O)(O)O)CC(=O)O]

41. The compound of claim 1, or the pharmaceutically acceptable salt thereof, wherein R¹ is selected from the group consisting of:

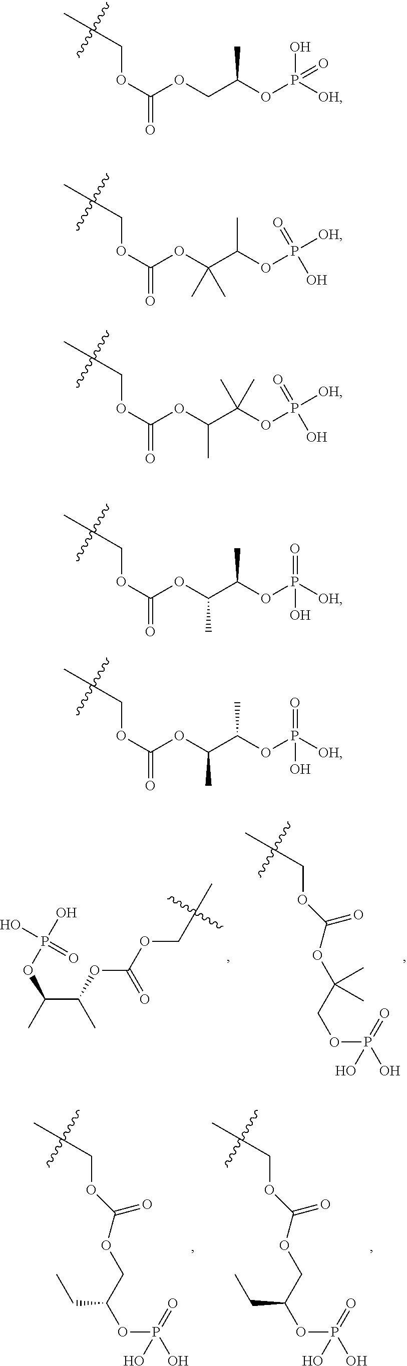

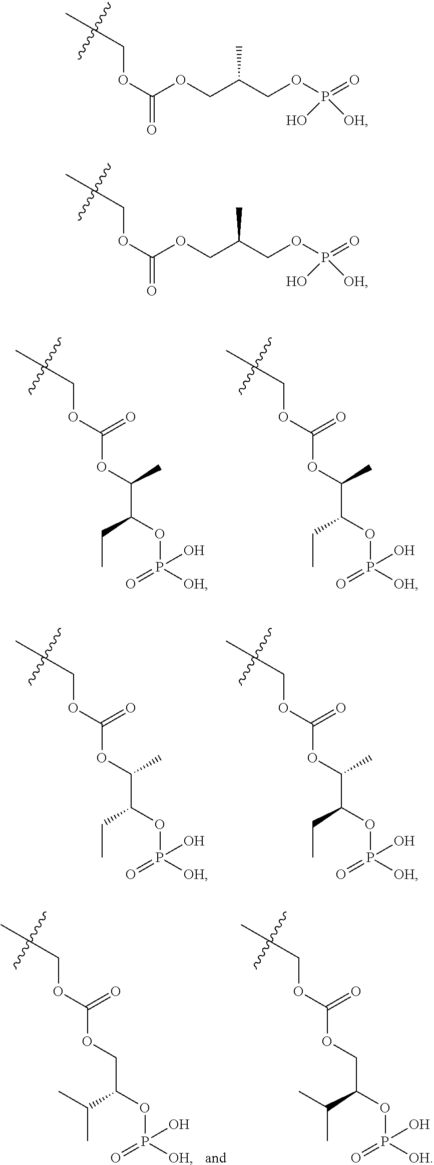

42. The compound of claim 1, or the pharmaceutically acceptable salt thereof, wherein R¹ is selected from the group consisting of:

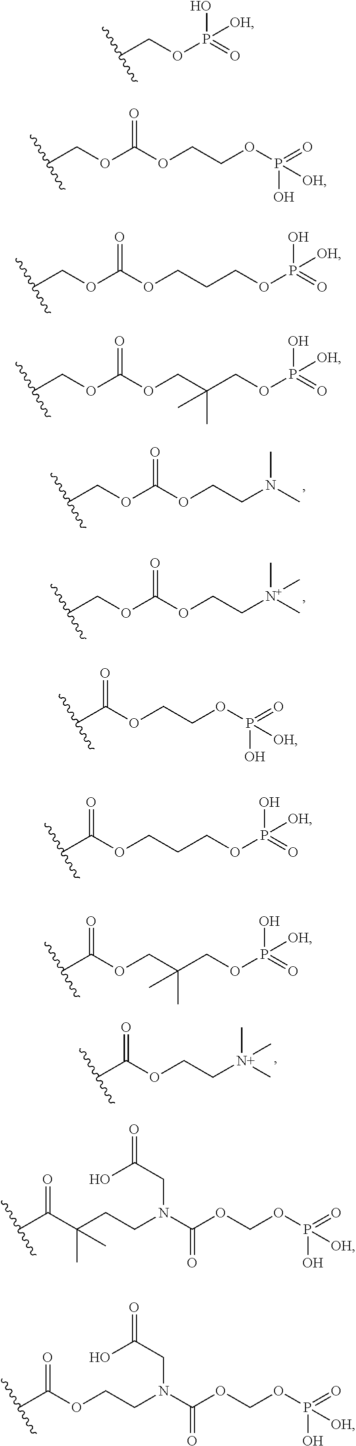

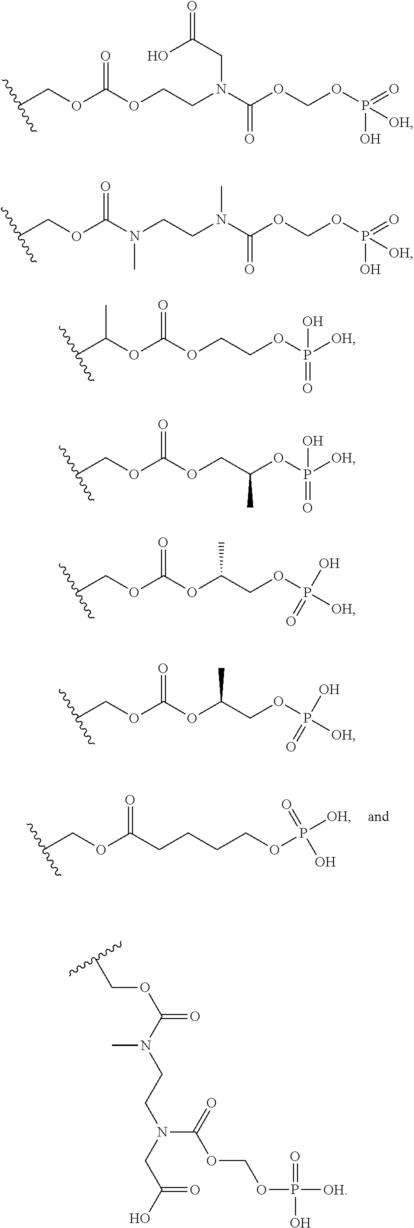

43.-45. (canceled)

46. A pharmaceutical composition comprising a therapeutically effective amount of a compound of claim 1, or pharmaceutically acceptable salt thereof, and a pharmaceutically acceptable excipient.

47.-56. (canceled)

57. A method of treating an HIV infection in a human having or at risk of having the infection, comprising administering to the human a therapeutically effective amount of a compound of claim 1, or a pharmaceutically acceptable salt thereof.

58.-176. (canceled)
